(19) United States
(12) Patent Application Publication
(10) Pub. No.: US 2024/0425497 A1
(43) Pub. Date: Dec. 26, 2024

(54) Cbl-b Inhibitors and Methods of Use Thereof

(71) Applicant: ARCUS BIOSCIENCES, INC., Hayward, CA (US)

(72) Inventors: Srikanth Kumar Gangam, Fremont, CA (US); Clayton Hardman, San Francisco, CA (US); Kenneth Victor Lawson, San Francisco, CA (US); Manmohan Reddy Leleti, Dublin, CA (US); Dongdong Liu, Fremont, CA (US); Artur Karenovich Mailyan, Livermore, CA (US); Masa Podunavac, Hayward, CA (US); Shiwei Qu, Alameda, CA (US); Xianglin Yin, Hayward, CA (US); Kai Yu, Hayward, CA (US); Tzu-Yu Yu, Hayward, CA (US)

(73) Assignee: ARCUS BIOSCIENCES, INC., Hayward, CA (US)

(21) Appl. No.: 18/654,773

(22) Filed: May 3, 2024

Related U.S. Application Data
(60) Provisional application No. 63/464,400, filed on May 5, 2023.
(60) Provisional application No. 63/623,147, filed on Jan. 19, 2024.

(51) Int. Cl. C07D 471/04; A61K 31/4545; A61K 31/519; A61K 31/5377; A61K 45/06; C07D 401/14; C07D 487/04

(52) U.S. Cl. CPC C07D 471/04; A61K 31/4545; A61K 31/519; A61K 31/5377; A61K 45/06; C07D 401/14; C07D 487/04

(57) ABSTRACT

Disclosed herein are compounds that are Cbl-b inhibitors having a structure according to Formula I, and compositions containing those compounds. Methods of preparing the compounds, and methods of using the compounds for the treatment of diseases, disorders, or conditions are also described.

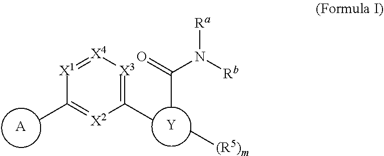

### CROSS-REFERENCE TO RELATED APPLICATIONS

This application claims the benefit of priority to U.S. Provisional Patent Application No. 63/464,400, filed on May 5, 2023, and U.S. Provisional Patent Application No. 63/623,147, filed on Jan. 19, 2024, the entire content of each of which is incorporated by reference herein.

### BACKGROUND

The following discussion is provided to aid the reader in understanding the disclosure and is not admitted to describe or constitute prior art thereto.

Crucial to the proper regulation of immune responses is a balance between immune activating and inhibitory signals. One cellular mechanism that regulates diverse aspects of the immune system is ubiquitination. Ubiquitination involves covalent conjugation of monoubiquitin or polyubiquitin chains onto amino acid residues of target proteins. Protein ubiquitination can alter the activity and/or stability of a molecule, and in some instances can also alter localization of the molecule into different cellular compartments. The ubiquitination process is catalyzed by sequential actions of ubiquitin-activating (E1), ubiquitin-conjugating (E2) and ubiquitin-ligating (E3) enzymes. The process of protein ubiquitination is counteracted by deubiquitinases (DUBs), a large family of proteases that cleaves ubiquitin chains. Mammalian cells express more than 600 E3 ligases and about 100 DUBs, which display substrate specificities and regulate specific cellular functions.

An increasing number of E3 ligases and DUBs have been identified as important regulators of immune responses. For example, small-molecule inhibitors that are antagonists of the IAP family of E3 ligases, including cIAP1, cIAP2, and X-linked IAP (XIAP), have been developed as small-molecule mimetics of the endogenous IAP inhibitor Smac. Small molecule inhibitors have also been developed against MDM2, an E3 ligase that promotes tumor growth and progression by mediating ubiquitin-dependent degradation of the tumor suppressor p53 and p53-independent functions. Casitas B-lineage lymphoma (Cbl) proteins, a family of E3 ubiquitin ligases, have been previously identified as potential targets; and so has VHL E3 complex, which mediates ubiquitin-dependent degradation of HIF1α and controls metabolic activities and effector function of T cells. Small molecule inhibitors for several DUBs have also been developed, and some of them have been shown to inhibit tumor growth in animal models.

While these potential targets provide exciting opportunities for the immunotherapy field, there remains a need in the art for effective inhibitors.

### SUMMARY

Casitas B-lineage lymphoma (Cbl) proteins are a family of E3 ubiquitin ligases. The mammalian Cbl family contains three homologs—c-Cbl, Cbl-b, and Cbl-3. Cbl-b and c-Cbl share some structural similarities but may have distinct physiological functions.

In one aspect, this disclosure is directed to a compound having a structure according to Formula I:

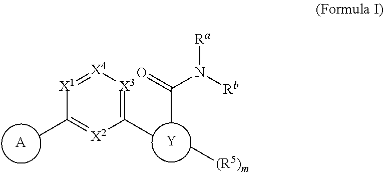

or a pharmaceutically acceptable salt thereof, wherein:
A has a formula selected from the group consisting of:

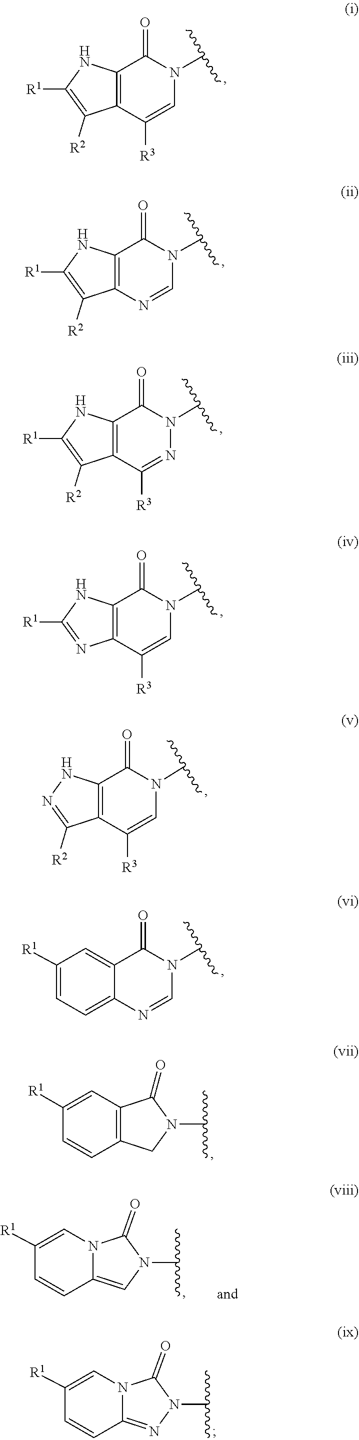

wherein:
R¹ is selected from the group consisting of —H, —C₁-C₆ haloalkyl, —C₁-C₆ hydroxyalkyl, —C(O)NH₂, —C(O)—(C₁-C₆-alkyl), -(Q¹)-NR^{1a}R^{1b}, -(Q¹)-(C₃-C₇ cycloalkyl), -(Q¹)-(5- to 6-membered heteroaryl), and -(Q¹)-(4- to 8-membered heterocycloalkyl); wherein said 5- to 6-membered heteroaryl has 1-3 ring heteroatoms independently selected from N, O, and S; said 4- to 8-membered heterocycloalkyl has 1-3 ring heteroatom or heteroatom groups independently selected from N, O, S, and S(O)₂; and said C₃-C₇ cycloalkyl, 4- to 8-membered heterocycloalkyl, and 5- to 6-membered heteroaryl are unsubstituted or substituted with 1-2 substituents independently selected from halo, —OH, —C₁-C₃ alkyl, and —C₁-C₃ alkoxy;
Q¹ is absent, unsubstituted —(C₁-C₃ alkylene)-, or —(C₁-C₃ alkylene)- substituted with 1-3 R^{q};
each R^{q} is independently halo, —OH, or —NH₂;
R^{1a} and R^{1b} are independently selected from the group consisting of H, —C₁-C₆ alkyl, —C₁-C₆ haloalkyl, phenyl, —(C₁-C₃ alkylene)-O—(C₁-C₃ alkyl), —C₃-C₆ cycloalkyl, —(C₁-C₃ alkylene)-(C₃-C₆ cycloalkyl), —S(O)₂(C₁-C₆ alkyl), 5- to 6-membered heteroaryl having 1-3 ring heteroatoms independently selected from N, O, and S, and 4- to 8-membered heterocycloalkyl having 1-3 ring heteroatom or heteroatom groups independently selected from N, O, S, and S(O)₂; wherein said phenyl, —(C₁-C₃ alkylene)-O—(C₁-C₃ alkyl), —C₃-C₆ cycloalkyl, —(C₁-C₃ alkylene)-(C₃-C₆ cycloalkyl), 5- to 6-membered heteroaryl, and 4- to 8-membered heterocycloalkyl are unsubstituted or substituted with 1-3 R^{1c};
each R^{1c}, when present, is independently halo, —OH, —C₁-C₃ alkyl, —C₁-C₃ hydroxyalkyl, or —C₁-C₃ haloalkyl;
R², when present, is —H, halo, —CN, —C₁-C₃ alkyl, —C₁-C₃ haloalkyl, —C₃-C₄ cycloalkyl, —S(O)₂(C₁-C₃ alkyl), —C(O)—NR^{2a}R^{2b}, or 5- to 6-membered heteroaryl having 1-3 ring heteroatoms independently selected from N, O, and S;
R^{2a} and R^{2b} are independently —H, or —C₁-C₃ alkyl;
R³, when present, is —H, —CN, halo, —C₁-C₆ alkyl, —C₁-C₆ haloalkyl, —C₁-C₆ hydroxyalkyl, —C₂-C₃ alkenyl, —C₃-C₄ cycloalkyl, —S(O)₂(C₁-C₆ alkyl), —C(O)OH, or 5- to 6-membered heteroaryl having 1 to 4 ring heteroatoms independently selected from N, O, and S; and said 5- to 6-membered heteroaryl is unsubstituted or substituted with 1-3 substituents independently selected from —C₁-C₃ alkyl;
X¹, X² and X³ are each independently N or CH;
X⁴ is N or CR⁴;
R⁴, when present, is —H, halo, —CN, —OH, —C₁-C₆ alkyl, —C₁-C₆ haloalkyl, —C₁-C₆ alkoxy, —NR^{4a}R^{4b}, or —C₃-C₈ cycloalkyl;
R^{4a} and R^{4b} are independently —H, —C₁-C₃ alkyl, or —(C₁-C₃ alkylene)-NR^{4c}R^{4d};
R^{4c} and R^{4d} are independently —H, or —C₁-C₃ alkyl;
Y is phenyl, or 5- to 6-membered heteroaryl having 1-3 ring heteroatoms independently selected from N, O, and S;
m is 0, 1, 2, or 3;
each R⁵ when present, is independently halo, —CN, —C₁-C₆ alkyl, —C₁-C₆ haloalkyl, or —C₁-C₆ alkoxy;
R^{a} and R^{b} are each independently H, —C₁-C₆ alkyl, —C₁-C₆ haloalkyl, phenyl, or —(C₁-C₃ alkylene)-O—(C₁-C₃ alkyl); or
R^{a} and R^{b} taken together with the N atom to which they are attached form a 4- to 8-membered heterocycloalkyl optionally having one additional ring heteroatom selected from N, O, and S; wherein said 4- to 8-membered heterocycloalkyl is unsubstituted or substituted with 1-3 substituents independently selected from the group consisting of halo, —CN, —C₁-C₆ alkyl, and —C₁-C₆ alkoxy.

In another aspect, this disclosure is directed to methods of inhibiting Cbl-b in a subject comprising administering to the subject an effective amount of a compound described herein.

In another aspect, this disclosure is directed to methods of increasing immune cell activity in a subject comprising administering to the subject an effective amount of a compound described herein.

In yet another aspect, this disclosure provides methods for treating a disease, disorder, or condition mediated at least in part by Cbl-b in a subject, comprising administering to the subject a therapeutically effective amount of a compound described herein. Diseases, disorders, and conditions mediated by Cbl-b include cancer and cancer-related disorders.

Certain aspects of the present disclosure further comprise the administration of one or more additional therapeutic agents as set forth herein below.

### DETAILED DESCRIPTION OF THE DISCLOSURE

Before the present disclosure is further described, it is to be understood that the disclosure is not limited to the particular embodiments set forth herein, and it is also to be understood that the terminology used herein is for the purpose of describing particular embodiments only, and is not intended to be limiting.

### Definitions

Unless otherwise defined, all terms of art, notations and other scientific terms or terminology used herein are intended to have the meanings commonly understood by those of skill in the art to which this disclosure pertains.

The term “about” as used herein has its original meaning of approximately and is to provide literal support for the exact number that it precedes, as well as a number that is near to or approximately the number that the term precedes. In general, the term “about” refers to the usual error range for the respective value readily known to the skilled person in this technical field. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The term “alkyl”, by itself or as part of another substituent, means, unless otherwise stated, a saturated monovalent hydrocarbon radical, having, in some embodiments, one to eight (e.g., C₁-C₈ alkyl), or one to six (e.g., C₁-C₆ alkyl), or one to three (e.g., C₁-C₃ alkyl) carbon atoms, respectively. The term “alkyl” encompasses straight and branched-chain hydrocarbon groups. Examples of alkyl groups include, but are not limited to, methyl (Me), ethyl (Et), n-propyl, isopropyl, n-butyl, t-butyl, isobutyl, sec-butyl, isopentyl, tert-pentyl, n-pentyl, isohexyl, n-hexyl, n-heptyl, 4-isopropylheptane, n-octyl, and the like. In some embodiments, the alkyl groups are C₁-C₄ alkyl groups (e.g., methyl, ethyl, isopropyl, or t-butyl). In some embodiments, the alkyl groups are C₁-C₃ alkyl groups (e.g., methyl, ethyl, n-propyl, or isopropyl).

The term “alkenyl”, as used herein, refers to a straight or branched monovalent hydrocarbon radical having, in some embodiments, two to eight carbon atoms (e.g., C₂-C₈ alkenyl), or two to six carbon atoms (e.g., C₂-C₆ alkenyl), or two to three carbon atoms (e.g., C₂-C₃ alkenyl), and having at least one carbon-carbon double bond. Examples of alkenyl groups include, but are not limited to, ethenyl, propenyl, isobutenyl, butadienyl and the like.

The term “alkylene” refers to a straight or branched, saturated, hydrocarbon radical having, in some embodiments, one to six (e.g., C₁-C₆ alkylene), one to four (e.g., C₁-C₄ alkylene), one to three (e.g., C₁-C₃ alkylene), or one to two (e.g., C₁-C₂ alkylene) carbon atoms, and linking at least two other groups, i.e., a divalent hydrocarbon radical. The two moieties linked to the alkylene can be attached to the same carbon atom (i.e., geminal), or different carbon atoms of the alkylene group. For instance, a straight chain alkylene can be the bivalent radical of —(CH₂)ₙ—, where n is 1, 2, 3, 4, 5 or 6 (i.e., a C₁-C₆ alkylene). Representative alkylene groups include, but are not limited to, methylene, ethylene, propylene, isopropylene, butylene, isobutylene, secbutylene, pentylene, hexylene and the like. In some embodiments, the alkylene groups are C₁-C₂ alkylene groups (e.g., methylene, or ethylene). In some embodiments, the alkylene groups are C₁-C₄ alkylene groups (e.g., methylene, ethylene, propylene, isopropylene, butylene, isobutylene, secbutylene, and the like).

As used herein, the term “alkoxy” refers to an alkyl group, as defined herein, that is attached to the remainder of the molecule via an oxygen atom (e.g., —O—C₁-C₁₂ alkyl, —O—C₁-C₈ alkyl, —O—C₁-C₆ alkyl, or —O—C₁-C₃ alkyl). Non-limiting examples of alkoxy groups include methoxy, ethoxy, n-propoxy, iso-propoxy, n-butoxy, sec-butoxy, n-pentoxy, n-hexoxy, and the like. In some embodiments, the alkoxy groups are C₁-C₃ alkoxy groups (e.g., methoxy, ethoxy, n-propoxy, or iso-propoxy).

The term “cycloalkyl” refers to a monocyclic, bicyclic or polycyclic hydrocarbon ring system having, in some embodiments, 3 to 14 carbon atoms (e.g., C₃-C₁₄ cycloalkyl), or 3 to 10 carbon atoms (e.g., C₃-C₁₀ cycloalkyl), or 3 to 8 carbon atoms (e.g., C₃-C₈ cycloalkyl), or 3 to 6 carbon atoms (e.g., C₃-C₆ cycloalkyl) or 3 to 4 carbon atoms (e.g., C₃-C₄ cycloalkyl). Cycloalkyl groups can be saturated or characterized by one or more points of unsaturation (i.e., carbon-carbon double and/or triple bonds), provided that the points of unsaturation do not result in an aromatic system. Examples of monocyclic cycloalkyl groups include, but are not limited to, cyclopropyl, cyclobutyl, cyclopentyl, cyclohexyl, cyclohexenyl, cycloheptyl, cycloheptenyl, cycloheptadienyl, cyclooctyl, cyclooctenyl, cyclooctadienyl and the like. The rings of bicyclic and polycyclic cycloalkyl groups can be fused, bridged, or spirocyclic. Non-limiting examples of bicyclic, spirocyclic and polycyclic cycloalkyl groups include bicyclo[2.2.1]heptane, bicyclo[2.2.2]octane, adamantyl, indanyl, spiro[5.5]undecane, spiro[2.2]pentane, spiro[2.2]pentadiene, spiro[2.3]hexane, spiro[2.5]octane, spiro[2.2]pentadiene, and the like. In some embodiments, the cycloalkyl groups of the present disclosure are monocyclic C₃-C₆ cycloalkyl moieties (e.g., cyclopropyl, cyclobutyl, cyclopentyl, or cyclohexyl). In some embodiments, the cycloalkyl groups of the present disclosure are monocyclic C₃-C₄ cycloalkyl moieties (e.g., cyclopropyl, or cyclobutyl).

The term “heterocycloalkyl” refers to a non-aromatic monocyclic, bicyclic or polycyclic cycloalkyl ring having, in some embodiments, 3 to 14 members (e.g., 3- to 14-membered heterocycle), or 3 to 10 members (e.g., 3- to 10-membered heterocycle), or 3 to 8 members (e.g., 3- to 8-membered heterocycle), or 3 to 6 members (e.g., 3- to 6-membered heterocycle), or 5 to 6 members (e.g., 5- to 6-membered heterocycle), and having from one to five, one to four, one to three, one to two or one heteroatom or heteroatom group(s) independently selected from nitrogen (N), oxygen (O), sulfur (S), sulfoxide (S(O)), and sulfone (S(O)₂). Heterocycloalkyl groups are saturated or characterized by one or more points of unsaturation (e.g., one or more carbon-carbon double bonds, carbon-carbon triple bonds, carbon-nitrogen double bonds, and/or nitrogen-nitrogen double bonds), provided that the points of unsaturation do not result in an aromatic system. The rings of bicyclic and polycyclic heterocycloalkyl groups can be fused, bridged, or spirocyclic. Non-limiting examples of heterocycloalkyl groups include aziridine, oxirane, thiirane, pyrrolidine, imidazolidine, pyrazolidine, dioxolane, phthalimide, piperidine, 1,4-dioxane, morpholine, thiomorpholine, thiomorpholine-S-oxide, thiomorpholine-S,S-oxide, piperazine, 3,4,5,6-tetrahydropyridazine, tetrahydropyran, pyran, decahydroisoquinoline, 3-pyrroline, thiopyran, tetrahydrofuran, tetrahydrothiophene, tetrahydro-1,1-dioxido-2H-thiopyran, quinuclidine, 1,4-oxazepane, 2-azabicyclo[4.1.0]heptane, 2-oxa-5-azabicyclo[2.2.1]heptane, 2-azabicyclo[2.2.1]heptane, 2,5-diazabicyclo[2.2.1]heptane, 6-oxa-3-azabicyclo[3.1.1]heptane, 3-oxa-6-azabicyclo[3.1.1]heptane, 2,5-diazabicyclo[2.2.1]heptane, 2-thia-6-azaspiro[3.3]heptane 2,2-dioxide, 2,6-diazaspiro[3.3]heptane, 2-azaspiro[3.3]heptane, 1-oxaspiro[3.3]heptane, 5-azaspiro[2.4]heptane, 6-azaspiro[3.4]octane, 6-azaspiro[2.5]octane, 4-oxa-7-azaspiro[2.5]octane, 3-oxa-8-azabicyclo[3.2.1]octane, and the like. A heterocycloalkyl group can be attached to the remainder of the molecule through a ring carbon atom, or a ring heteroatom, when chemically permissible. In some embodiments, the heterocycloalkyl groups of the present disclosure are monocyclic 4- to 8-membered heterocycloalkyl moieties having one or two heteroatom or heteroatom groups independently selected from N, O, S and S(O)₂ (e.g., azetidine, piperidine, piperazine, morpholine, pyrrolidine, imidazolidine, pyrazolidine, tetrahydrofuran, tetrahydropyran, 1,4-oxazepane, 6-oxa-3-azabicyclo[3.1.1]heptane, 3-oxa-6-azabicyclo[3.1.1]heptane, 2-thia-6-azaspiro[3.3]heptane 2,2-dioxide, and the like).

The term “aryl” refers to an aromatic ring system containing one ring, or two or three rings fused together, and having, in some embodiments, six to fourteen (i.e., C₆-C₁₄ aryl), or six to ten (i.e., C₆-C₁₀ aryl), or six (i.e., C₆ aryl) carbon atoms. Non-limiting examples of aryl groups include phenyl, naphthyl and anthracenyl. In some embodiments, aryl groups are phenyl.

The term “heteroaryl” refers to monocyclic or fused bicyclic aromatic groups (or rings) having, in some embodiments, from 5 to 14 (i.e., 5- to 14-membered heteroaryl), or from 5 to 10 (i.e., 5- to 10-membered heteroaryl), or from 5 to 6 (i.e., 5- to 6-membered heteroaryl) members (i.e., ring vertices), and containing from one to five, one to four, one to three, one to two or one heteroatom independently selected from nitrogen (N), oxygen (O), and sulfur (S). A heteroaryl group can be attached to the remainder of the molecule through a carbon atom or a heteroatom of the heteroaryl group, when chemically permissible. Non-limiting examples of heteroaryl groups include pyridyl, pyridazinyl, pyrazinyl, pyrimidinyl, triazinyl, purinyl, thienopyridinyl, thienopyrimidinyl, pyrazolopyrimidinyl, imidazopyridines, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like. In some embodiments, the heteroaryl groups of the present disclosure are monocyclic 5- to 6-membered heteroaryl moieties having 1-3 heteroatoms independently selected from N, O, and S (e.g., pyridinyl, pyrimidinyl, pyridazinyl, triazolyl, imidazolyl, pyrazolyl, oxazolyl, oxadiazolyl, or thiazolyl). In some embodiments, the heteroaryl groups of the present disclosure are monocyclic 5- to 6-membered heteroaryl moieties having 1-2 ring nitrogen atoms (e.g., pyridinyl, pyrimidinyl, pyridazinyl, imidazolyl, or pyrazolyl).

As used herein, a wavy line, “ [symbol] ”, that intersects a single, double or triple bond in any chemical structure depicted herein, represents that the point of attachment of the single, double, or triple bond to the remainder of the molecule is through either one of the atoms that make up the single, double or triple bond. Additionally, a bond extending from a substituent to the center of a ring (e.g., a phenyl ring) is meant to indicate attachment of that substituent to the ring at any of the available ring vertices, i.e., such that attachment of the substituent to the ring results in a chemically stable arrangement.

The term “halogen,” by itself or as part of another substituent, means, unless otherwise stated, a fluorine, chlorine, bromine, or iodine atom. Additionally, the term “haloalkyl” refers to an alkyl group as defined herein, that are substituted with one or more halogen(s) (e.g., 1-3 halogen(s)). For example, the term “C₁-C₄ haloalkyl” is meant to include trifluoromethyl, difluoromethyl, 2,2,2-trifluoroethyl, 4-chlorobutyl, 3-bromopropyl, and the like.

The term “hydroxyalkyl” refers to an alkyl group, as defined herein, that is substituted with one or more hydroxyl groups (e.g., 1-3 hydroxyl groups). Exemplary hydroxyalkyl groups include methanol, ethanol, 1,2-propanediol, 1,2-hexanediol, glycerol, and the like.

The compounds of the present disclosure can be present in their neutral form, or as a pharmaceutically acceptable salt, isomer, polymorph or solvate thereof, and may be present in a crystalline form, amorphous form or mixtures thereof.

As referred to herein, “pharmaceutically acceptable salt” is meant to include salts of the compounds according to this disclosure that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present disclosure may contain both basic and acidic functionalities that allow the compounds to be converted into either base or acid addition salts.

The neutral forms of the compounds may be regenerated by contacting the salt with a base or acid and isolating the parent compound.

This disclosure also contemplates isomers of the compounds described herein (e.g., stereoisomers, and atropisomers). For example, certain compounds of the present disclosure possess asymmetric carbon atoms (chiral centers); or hindered rotation about a single bond; the racemates, diastereomers, enantiomers, and atropisomers (e.g., Rₐ, Sₐ, P and M isomers) of which are all intended to be encompassed within the scope of the present disclosure. Stereoisomeric forms may be defined, in terms of absolute stereochemistry, as (R) or (S), and/or depicted uses dashes and/or wedges. When a stereochemical depiction (e.g., using dashes, [symbol] , and/or wedges, [symbol] ) is shown in a chemical structure, or a stereochemical assignment (e.g., using (R) and (S) notation) is made in a chemical name, it is meant to indicate that the depicted isomer is present and substantially free of one or more other isomer(s) (e.g., enantiomers and diastereomers, when present). “Substantially free of” other isomer(s) indicates at least an 70/30 ratio of the indicated isomer to the other isomer(s), more preferably 80/20, 90/10, or 95/5 or more. In some embodiments, the indicated isomer will be present in an amount of at least 99%. A chemical bond to an asymmetric carbon that is depicted as a solid line ( [symbol] ) or a wavy line ( [symbol] ) indicates that all possible stereoisomers (e.g., enantiomers, diastereomers, racemic mixtures, etc.) are included. In such instances, the compound may be present as a racemic mixture, scalemic mixture, or a mixture of diastereomers.

The compounds of the present disclosure may also contain unnatural proportions of atomic isotopes at one or more of the atoms that constitute such compounds. Unnatural proportions of an isotope may be defined as ranging from the amount found in nature to an amount consisting of 100% of the atom in question. For example, the compounds may incorporate radioactive isotopes, such as for example tritium (³H), iodine-125 (¹²⁵I) or carbon-14 (¹⁴C), or non-radioactive isotopes, such as deuterium (²H) or carbon-13 (¹³C). Such isotopic variations can provide additional utilities to those described elsewhere herein. For instance, isotopic variants of the compounds of the disclosure may find additional utility, including but not limited to, as diagnostic and/or imaging reagents, or as cytotoxic/radiotoxic therapeutic agents. Additionally, isotopic variants of the compounds of the disclosure can have altered pharmacokinetic and pharmacodynamic characteristics which can contribute to enhanced safety, tolerability or efficacy during treatment. In some embodiments, the compounds according to this disclosure are characterized by one or more deuterium atoms.

The terms “treat”, “treating”, treatment” and the like refer to a course of action that eliminates, reduces, suppresses, mitigates, ameliorates, or prevents the worsening of, either temporarily or permanently, a disease, disorder or condition to which the term applies, or at least one of the symptoms associated therewith. Treatment includes alleviation of symptoms, diminishment of extent of disease, inhibiting (e.g., arresting the development or further development of the disease, disorder or condition or clinical symptoms association therewith) an active disease, delaying or slowing of disease progression, improving the quality of life, and/or prolonging survival of a subject as compared to expected survival if not receiving treatment or as compared to a published standard of care therapy for a particular disease.

The term “in need of treatment” as used herein refers to a judgment made by a physician or similar professional that a subject requires or will benefit from treatment. This judgment is made based on a variety of factors that are in the realm of the physician's expertise, which may include a positive diagnosis of a disease, disorder or condition.

The terms “prevent”, “preventing”, “prevention”, “prophylaxis” and the like refer to a course of action initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state. Prevention also refers to a course of action initiated in a subject after the subject has been treated for a disease, disorder, condition or a symptom associated therewith in order to prevent relapse of that disease, disorder, condition or symptom.

The term “in need of prevention” as used herein refers to a judgment made by a physician or other caregiver that a subject requires or will benefit from preventative care. This judgment is made based on a variety of factors that are in the realm of a physician's or caregiver's expertise.

“Substantially pure” indicates that a component (e.g., a compound according to this disclosure) makes up greater than about 50% of the total content of the composition, and typically greater than about 60% of the total content. More typically, “substantially pure” refers to compositions in which at least 75%, at least 85%, at least 90% or more of the total composition is the component of interest. In some cases, the component of interest will make up greater than about 90%, or greater than about 95% of the total content of the composition.

Compounds that are selective may be particularly useful in the treatment of certain disorders or may offer a reduced likelihood of undesired side effects.

Compounds provided herein may have advantageous pharmacokinetic profiles including, for example, metabolic liabilities, permeability, bioavailability, low efflux, hepatocyte stability, clearance, inhibition against CYP, and/or inhibition against hERG.

### Compounds of the Disclosure

The present disclosure relates to compounds that inhibit the activity of Cbl-b.

In one aspect, this disclosure is directed to a compound having a structure according to Formula I:

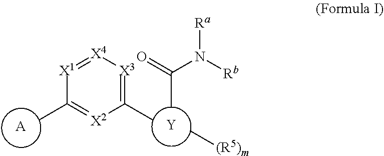

or a pharmaceutically acceptable salt thereof, wherein:
A has a formula selected from the group consisting of:

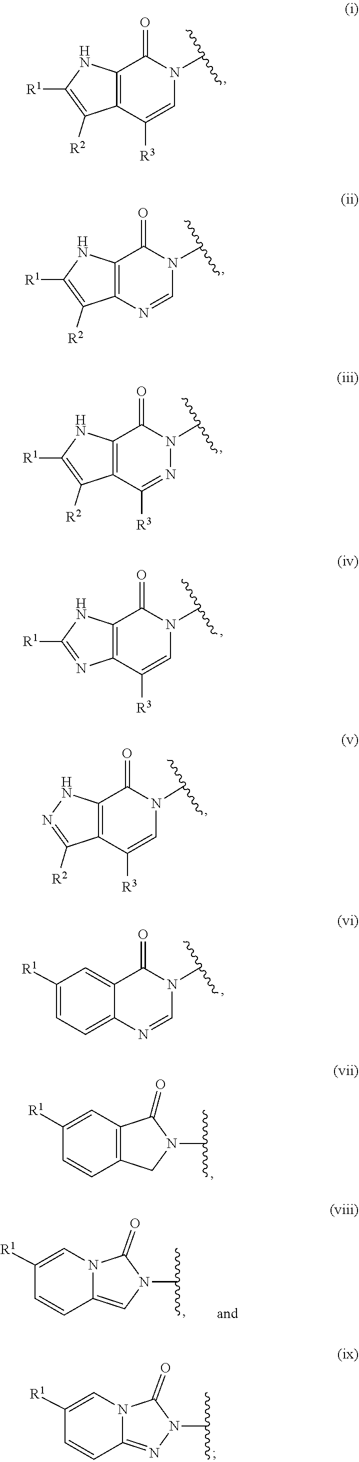

wherein:
R¹ is selected from the group consisting of —H, —C₁-C₆ haloalkyl, —C₁-C₆ hydroxyalkyl, —C(O)NH₂, —C(O)—(C₁-C₆-alkyl), -(Q¹)-NR^{1a}R^{1b}, -(Q)-(C₃-C₇ cycloalkyl), -(Q¹)-(5- to 6-membered heteroaryl), and -(Q¹)-(4- to 8-membered heterocycloalkyl); wherein said 5- to 6-membered heteroaryl has 1-3 ring heteroatoms independently selected from N, O, and S; said 4 to 8-membered heterocycloalkyl has 1-3 ring heteroatom or heteroatom groups independently selected from N, O, S, and S(O)₂; and said C₃-C₇ cycloalkyl, 4- to 8-membered heterocycloalkyl, and 5- to 6-membered heteroaryl are unsubstituted or substituted with 1-2 substituents independently selected from halo, —OH, —C₁-C₃ alkyl, and —C₁-C₃ alkoxy;
Q¹ is absent, unsubstituted —(C₁-C₃ alkylene)-, or —(C₁-C₃ alkylene)- substituted with 1-3 R^{q};
each R^{q} is independently halo, —OH, or —NH₂;
R^{1a} and R^{1b} are independently selected from the group consisting of H, —C₁-C₆ alkyl, —C₁-C₆ haloalkyl, phenyl, —(C₁-C₃ alkylene)-O—(C₁-C₃ alkyl), —C₃-C₆ cycloalkyl, —(C₁-C₃ alkylene)-(C₃-C₆ cycloalkyl), —S(O)₂(C₁-C₆ alkyl), 5- to 6-membered heteroaryl having 1-3 ring heteroatoms independently selected from N, O, and S, and 4- to 8-membered heterocycloalkyl having 1-3 ring heteroatom or heteroatom groups independently selected from N, O, S, and S(O)₂; wherein said phenyl, —(C₁-C₃ alkylene)-O—(C₁-C₃ alkyl), —C₃-C₆ cycloalkyl, —(C₁-C₃ alkylene)-(C₃-C₆ cycloalkyl), 5- to 6-membered heteroaryl, and 4- to 8-membered heterocycloalkyl are unsubstituted or substituted with 1-3 R^{1c};
each R^{1c}, when present, is independently halo, —OH, —C₁-C₃ alkyl, —C₁-C₃ hydroxyalkyl, or —C₁-C₃ haloalkyl;
R², when present, is —H, halo, —CN, —C₁-C₃ alkyl, —C₁-C₃ haloalkyl, —C₃-C₄ cycloalkyl, —S(O)₂(C₁-C₃ alkyl), —C(O)—NR^{2a}R^{2b}, or 5- to 6-membered heteroaryl having 1-3 ring heteroatoms independently selected from N, O, and S;
R^{2a} and R^{2b} are independently —H, or —C₁-C₃ alkyl;
R³, when present, is —H, —CN, halo, —C₁-C₆ alkyl, —C₁-C₆ haloalkyl, —C₁-C₆ hydroxyalkyl, —C₂-C₃ alkenyl, —C₃-C₄ cycloalkyl, —S(O)₂(C₁-C₆ alkyl), —C(O)OH, or 5- to 6-membered heteroaryl having 1 to 4 ring heteroatoms independently selected from N, O, and S; and said 5- to 6-membered heteroaryl is unsubstituted or substituted with 1-3 substituents independently selected from —C₁-C₃ alkyl;
X¹, X² and X³ are each independently N or CH;
X⁴ is N or CR⁴;
R⁴, when present, is —H, halo, —CN, —OH, —C₁-C₆ alkyl, —C₁-C₆ haloalkyl, —C₁-C₆ alkoxy, —NR^{4a}R^{4b}, or —C₃-C₈ cycloalkyl;
R^{4a} and R^{4b} are independently —H, —C₁-C₃ alkyl, or —(C₁-C₃ alkylene)-NR^{4a}R^{4b};
R^{4c} and R^{4d} are independently —H, or —C₁-C₃ alkyl;
Y is phenyl, or 5- to 6-membered heteroaryl having 1-3 ring heteroatoms independently selected from N, O, and S;
m is 0, 1, 2, or 3;
each R⁵ when present, is independently halo, —CN, —C₁-C₆ alkyl, —C₁-C₆ haloalkyl, or —C₁-C₆ alkoxy;
R^{a} and R^{b} are each independently H, —C₁-C₆ alkyl, —C₁-C₆ haloalkyl, phenyl, or —(C₁-C₃ alkylene)-O—(C₁-C₃ alkyl); or
R^{a} and R^{b} taken together with the N atom to which they are attached form a 4- to 8-membered heterocycloalkyl optionally having one additional ring heteroatom selected from N, O, and S; wherein said 4- to 8-membered heterocycloalkyl is unsubstituted or substituted with 1-3 substituents independently selected from the group consisting of halo, —CN, —C₁-C₆ alkyl, and —C₁-C₆ alkoxy.

In some embodiments, the compound has a structure according to Formula I:

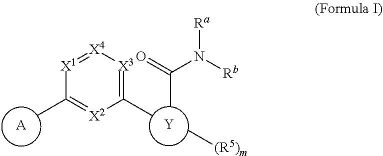

or a pharmaceutically acceptable salt thereof, wherein:
A has a formula selected from the group consisting of:

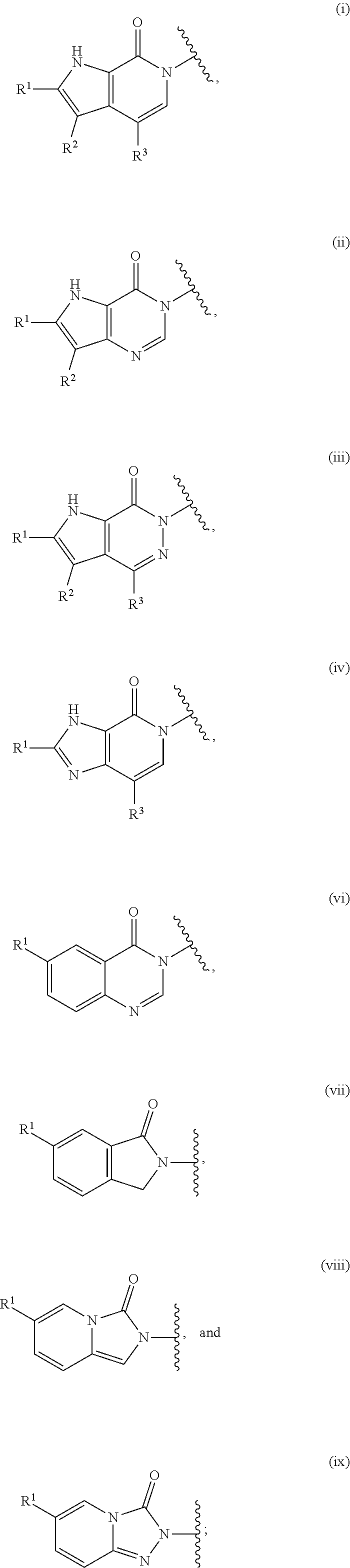

wherein:
R¹ is selected from the group consisting of —H, —C₁-C₆ haloalkyl, —C₁-C₆ hydroxyalkyl, —C(O)NH₂, —C(O)—(C₁-C₆-alkyl), -(Q¹)-NR^{1a}R^{1b}, -(Q)-(C₃-C₇ cycloalkyl), -(Q¹)-(5- to 6-membered heteroaryl), and -(Q¹)-(4- to 8-membered heterocycloalkyl); wherein said 5- to 6-membered heteroaryl has 1-3 ring heteroatoms independently selected from N, O, and S; said 4- to 8-membered heterocycloalkyl has 1-3 ring heteroatom or heteroatom groups independently selected from N, O, S, and S(O)₂; and said C₃-C₇ cycloalkyl, 4- to 8-membered heterocycloalkyl, and 5- to 6-membered heteroaryl are unsubstituted or substituted with 1-2 substituents independently selected from halo, —OH, —C₁-C₃ alkyl, and —C₁-C₃ alkoxy;
Q¹ is absent, unsubstituted —(C₁-C₃ alkylene)-, or —(C₁-C₃ alkylene)- substituted with 1-3 R^{q};
each R^{q} is independently halo, —OH, or —NH₂;
R^{1a} and R^{1b} are independently selected from the group consisting of H, —C₁-C₆ alkyl, —C₁-C₆ haloalkyl, phenyl, —(C₁-C₃ alkylene)-O—(C₁-C₃ alkyl), —C₃-C₆ cycloalkyl, —(C₁-C₃ alkylene)-(C₃-C₆ cycloalkyl), —S(O)₂(C₁-C₆ alkyl), 5- to 6-membered heteroaryl having 1-3 ring heteroatoms independently selected from N, O, and S, and 4- to 8-membered heterocycloalkyl having 1-3 ring heteroatom or heteroatom groups independently selected from N, O, S, and S(O)₂; wherein said phenyl, —(C₁-C₃ alkylene)-O—(C₁-C₃ alkyl), —C₃-C₆ cycloalkyl, —(C₁-C₃ alkylene)-(C₃-C₆ cycloalkyl), 5- to 6-membered heteroaryl, and 4- to 8-membered heterocycloalkyl are unsubstituted or substituted with 1-3 R^{1c};
each R^{1c}, when present, is independently halo, —OH, —C₁-C₃ alkyl, —C₁-C₃ hydroxyalkyl, or —C₁-C₃ haloalkyl;
R², when present, is —H, halo, —CN, —C₁-C₃ alkyl, —C₁-C₃ haloalkyl, —C₃-C₄ cycloalkyl, —S(O)₂(C₁-C₃ alkyl), —C(O)—NR^{2a}R^{2b}, or 5- to 6-membered heteroaryl having 1-3 ring heteroatoms independently selected from N, O, and S;
R^{2a} and R^{2b} are independently —H, or —C₁-C₃ alkyl;
R³, when present, is —H, —CN, halo, —C₁-C₆ alkyl, —C₁-C₆ haloalkyl, —C₁-C₆ hydroxyalkyl, —C₂-C₃ alkenyl, —C₃-C₄ cycloalkyl, —S(O)₂(C₁-C₆ alkyl), —C(O)OH, or 5- to 6-membered heteroaryl having 1 to 4 ring heteroatoms independently selected from N, O, and S; and the 5- to 6-membered heteroaryl is unsubstituted or substituted with 1-3 substituents independently selected from —C₁-C₃ alkyl;
X¹, X² and X³ are each independently N or CH;
X⁴ is N or CR⁴;
R⁴, when present, is —H, halo, —CN, —OH, —C₁-C₆ alkyl, —C₁-C₆ haloalkyl, —C₁-C₆ alkoxy, —NR^{4a}R⁴¹, or —C₃-C₈ cycloalkyl;
R^{4a} and R^{4b} are independently —H, —C₁-C₃ alkyl, or —(C₁-C₃ alkylene)-NR^{4a}R^{4b};
R^{4c} and R^{4d} are independently —H, or —C₁-C₃ alkyl;
Y is phenyl, or 5- to 6-membered heteroaryl having 1-3 ring heteroatoms independently selected from N, O, and S;
m is 0, 1, 2, or 3;
each R⁵ when present, is independently halo, —CN, —C₁-C₆ alkyl, —C₁-C₆ haloalkyl, or —C₁-C₆ alkoxy;
R^{a} and R^{b} are each independently H, —C₁-C₆ alkyl, —C₁-C₆ haloalkyl, phenyl, or —(C₁-C₃ alkylene)-O—(C₁-C₃ alkyl); or
R^{a} and R^{b} taken together with the N atom to which they are attached form a 4- to 8-membered heterocycloalkyl optionally having one additional ring heteroatom selected from N, O, and S; wherein said 4- to 8-membered heterocycloalkyl is unsubstituted or substituted with 1-3 substituents independently selected from the group consisting of halo, —CN, —C₁-C₆ alkyl, and —C₁-C₆ alkoxy.

In some embodiments, R¹ is —H, —C₁-C₆ hydroxyalkyl, -(Q¹)-NR^{1a}R^{1b}, -(Q)-(C₃-C₇ cycloalkyl), or -(Q¹)-(4- to 8-membered heterocycloalkyl) having 1-3 ring heteroatoms independently selected from N, O, and S; and said 4- to 8-membered heterocycloalkyl is unsubstituted or substituted with 1-2 substituents independently selected from —C₁-C₃ alkyl, and —C₁-C₃ alkoxy; Q¹ is absent or unsubstituted —(C₁-C₃ alkylene)-; R^{1a} and R^{1b} are independently —H, —C₁-C₆ alkyl, —C₁-C₆ haloalkyl, —(C₁-C₃ alkylene)-O—(C₁-C₃ alkyl), unsubstituted —C₃-C₆ cycloalkyl, or —C₃-C₆ cycloalkyl substituted with 1 R^{1c}; and R^{1c}, when present, is —OH or —C₁-C₃ alkyl.

In some embodiments, R¹ is —H, —C₁-C₆ hydroxyalkyl, -(Q¹)-NR^{1a}R^{1b}, or -(Q¹)-(4- to 8-membered heterocycloalkyl) having 1-3 ring heteroatoms independently selected from N, O, and S; and said 4- to 8-membered heterocycloalkyl is unsubstituted or substituted with 1-2 substituents independently selected from —C₁-C₃ alkyl, and —C₁-C₃ alkoxy.

In some embodiments, R¹ is —C₁-C₆ hydroxyalkyl, -(Q¹)-NR^{1a}R^{1b}, or -(Q¹)-(4- to 8-membered heterocycloalkyl) having 1-3 ring heteroatoms independently selected from N, O, and S; and said 4- to 8-membered heterocycloalkyl is unsubstituted or substituted with 1-2 substituents independently selected from —C₁-C₃ alkyl; Q¹ is unsubstituted —(C₁-C₃ alkylene)-; R^{1a} and R^{1b} are independently —H, unsubstituted —C₃-C₆ cycloalkyl, or —C₃-C₆ cycloalkyl substituted with 1 R^{1c}; and R^{1c}, when present, is —OH.

In some embodiments, R¹ is -(Q¹)-NR^{1a}R^{1b}, or -(Q¹)-(4- to 8-membered heterocycloalkyl) having 1-3 ring heteroatoms independently selected from N, and O; and said 4- to 8-membered heterocycloalkyl is substituted with 1-2 substituents independently selected from —C₁-C₃ alkyl.

In some embodiments, R¹ is —H. In some embodiments, R¹ is —C₁-C₆ haloalkyl. In some embodiments, R¹ is —C₁-C₆ hydroxyalkyl. In some embodiments, R¹ is —C(O)NH₂. In some embodiments, R¹ is —C(O)—(C₁-C₆-alkyl). In some embodiments, R¹ is -(Q¹)-NR^{1a}R^{1b}. In some embodiments, R¹ is -(Q¹)-(C₃-C₇ cycloalkyl), wherein said C₃-C₇ cycloalkyl is unsubstituted or substituted with 1-2 substituents independently selected from halo, —OH, —C₁-C₃ alkyl, and —C₁-C₃ alkoxy. In some embodiments, R¹ is -(Q¹)-(5- to 6-membered heteroaryl), wherein said 5- to 6-membered heteroaryl has 1-3 ring heteroatoms independently selected from N, O, and S; and said 5- to 6-membered heteroaryl is unsubstituted or substituted with 1-2 substituents independently selected from halo, —OH, —C₁-C₃ alkyl, and —C₁-C₃ alkoxy. In some embodiments, R¹ is -(Q¹)-(5- to 6-membered heteroaryl), wherein said 5- to 6-membered heteroaryl has 1-2 ring heteroatoms independently selected from N, and O; and said 5- to 6-membered heteroaryl is unsubstituted or substituted with 1-2 substituents independently selected from halo, —OH, —C₁-C₃ alkyl, and —C₁-C₃ alkoxy. In some embodiments, R¹ is -(Q¹)-(4- to 8-membered heterocycloalkyl); wherein said 4- to 8-membered heterocycloalkyl has 1-3 ring heteroatom or heteroatom groups independently selected from N, O, S, and S(O)₂; and said 4- to 8-membered heterocycloalkyl is unsubstituted or substituted with 1-2 substituents independently selected from halo, —OH, —C₁-C₃ alkyl, and —C₁-C₃ alkoxy. In some embodiments, R¹ is -(Q¹)-(4- to 8-membered heterocycloalkyl); wherein said 4- to 8-membered heterocycloalkyl has 1-2 ring heteroatoms independently selected from N, and O; and said 4- to 8-membered heterocycloalkyl is unsubstituted or substituted with 1-2 substituents independently selected from halo, —OH, —C₁-C₃ alkyl, and —C₁-C₃ alkoxy.

In some embodiments, R¹ is -(Q¹)-NR^{1a}R^{1b}, -(Q¹)-(C₃-C₇ cycloalkyl), or -(Q¹)-(4- to 8-membered heterocycloalkyl); and Q¹ is absent or unsubstituted —(C₁-C₃ alkylene)-. In some embodiments, R¹ is -(Q¹)-NR^{1a}R^{1b}, and Q¹ is unsubstituted —(C₁-C₃ alkylene)-. In some embodiments, R¹ is -(Q¹)-(C₃-C₇ cycloalkyl), and Q¹ is absent. In some embodiments, R¹ is -(Q¹)-(4- to 8-membered heterocycloalkyl), and Q¹ is absent or unsubstituted —(C₁-C₃ alkylene)-.

In some embodiments, Q¹ is absent. In some embodiments, Q¹ is unsubstituted —(C₁-C₃ alkylene)-. In some embodiments, Q¹ is —CH₂—. In some embodiments, Q¹ is —(C₁-C₃ alkylene)- substituted with 1-3 R^{q}.

In some embodiments, R^{1a} and R^{1b} are independently selected from the group consisting of H and —C₃-C₆ cycloalkyl, wherein said —C₃-C₆ cycloalkyl is unsubstituted or substituted with 1-3 R^{1c}.

In some embodiments, R^{1a} and R^{1b} are independently —H, —C₁-C₆ alkyl, —C₁-C₆ haloalkyl, —(C₁-C₃ alkylene)-O—(C₁-C₃ alkyl), unsubstituted —C₃-C₆ cycloalkyl, or —C₃-C₆ cycloalkyl substituted with 1 R^{1c}.

In some embodiments, R¹ is —H,

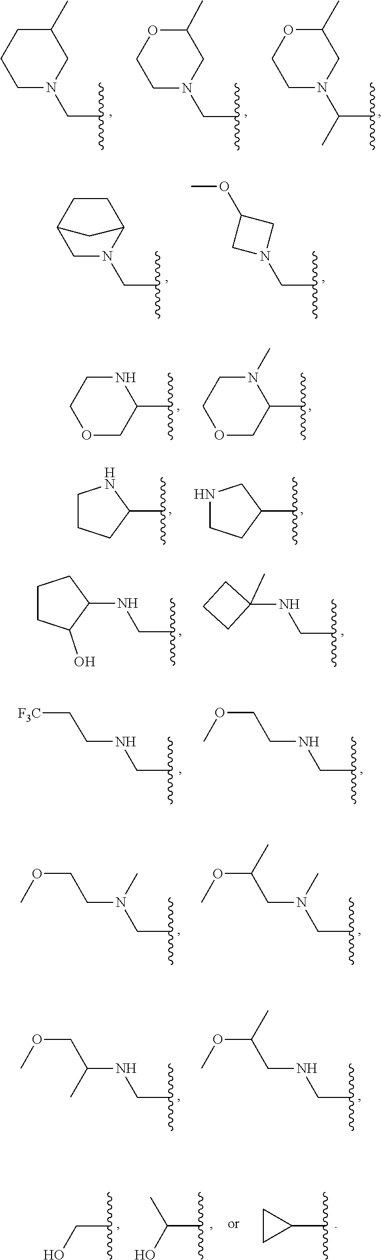

In some embodiments, R¹ is —H,

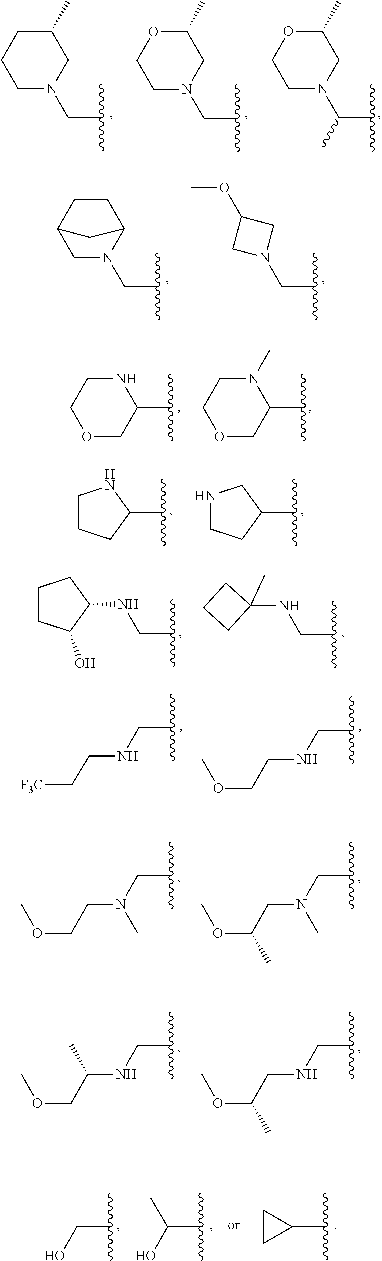

In some embodiments, R¹ is —H,

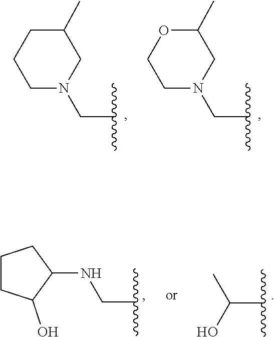

In some embodiments, R¹ is —H,

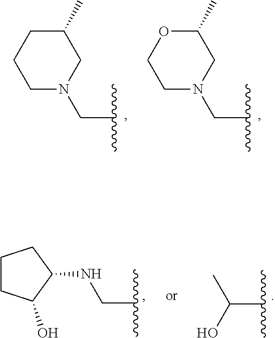

In some embodiments, R¹ is

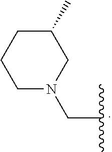 [SMILES: *CN1CCC[C@H](C)C1]

In some embodiments, R², when present, is —H. In some embodiments, R², when present, is halo. In some embodiments, R², when present, is —CN. In some embodiments, R², when present, is —H, halo, or —CN. In some embodiments, R², when present, is —H, —Cl, or —CN. In some embodiments, R², when present, is —C₁-C₃ alkyl. In some embodiments, R², when present, is —C₁-C₃ haloalkyl. In some embodiments, R², when present, is —C₃-C₄ cycloalkyl. In some embodiments, R², when present, is —S(O)₂(C₁-C₃ alkyl). In some embodiments, R², when present, is —C(O)—NR^{2a}R^{2b}, wherein R^{2a} and R^{2b} are independently —H or —C₁-C₃ alkyl. In some embodiments, R², when present, is 5- to 6-membered heteroaryl having 1-3 ring heteroatoms independently selected from N, O, and S.

In some embodiments, R³, when present, is —H. In some embodiments, R³, when present, is —CN. In some embodiments, R³, when present, is halo. In some embodiments, R³, when present, is —C₁-C₆ alkyl. In some embodiments, R³, when present, is —C₁-C₆ haloalkyl. In some embodiments, R³, when present, is —C₁-C₆ hydroxyalkyl. In some embodiments, R³, when present, is —C₂-C₃ alkenyl. In some embodiments, R³, when present, is —C₃-C₄ cycloalkyl. In some embodiments, R³, when present, is —S(O)₂(C₁-C₆ alkyl). In some embodiments, R³, when present, is —C(O)OH. In some embodiments, R³, when present, is a 5- to 6-membered heteroaryl having 1 to 4 ring heteroatoms independently selected from N, O, and S; and the 5- to 6-membered heteroaryl is unsubstituted or substituted with 1-3 substituents independently selected from —C₁-C₃ alkyl.

In some embodiments, R³, when present, is —CN, —C₁-C₆ alkyl, —C₁-C₆ haloalkyl, or —C₃-C₄ cycloalkyl. In some embodiments, R³, when present, is —CN, —CH₃, —CF₃, or cyclopropyl. In some embodiments, R³, when present, is —CN or —C₃-C₄ cycloalkyl. In some embodiments, R³, when present, is —CN or cyclopropyl. In some embodiments, R³, when present, is cyclopropyl.

In some embodiments, X¹ is N, X² is CH, and X³ is CH. In some embodiments, X¹, X², and X³ are CH.

In some embodiments, X¹ is N, X² is CH, X³ is CH, and X⁴ is CR⁴. In some embodiments, X¹, X², and X³ are CH, and X⁴ is N. In some embodiments, X¹, X², and X³ are CH, and X⁴ is CR⁴. In some embodiments, X¹, X², X³, and X⁴ are CH.

In some embodiments, the ring formed by X¹, X², X³, and X⁴ is selected from the group consisting of:

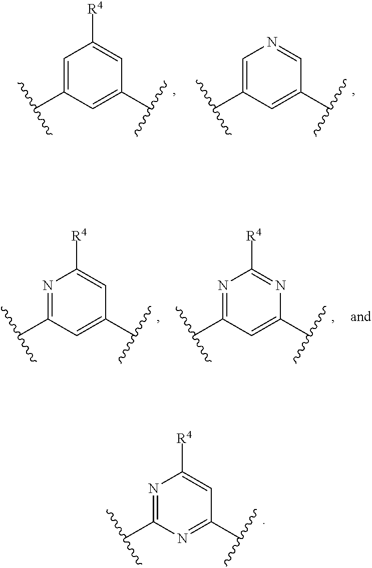

In some embodiments, the compound has a structure according to Formula Ia or Formula Ib:

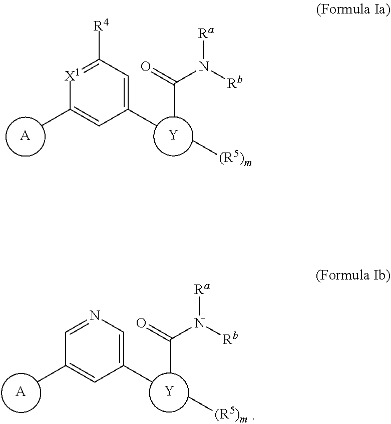

In some embodiments, the compound has a structure according to Formula Ia:

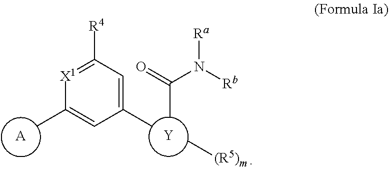

In some embodiments, the compound has a structure according to Formula Ib:

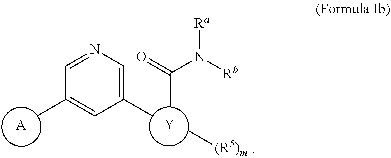

In some embodiments, the compound has a structure according to Formula Ic:

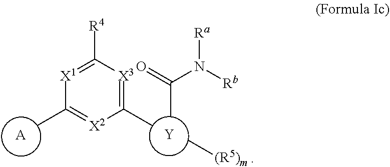

In some embodiments, R⁴, when present, is —CN, —C₁-C₆ haloalkyl, or —C₃-C₄ cycloalkyl. In some embodiments, R⁴, when present, is —CN, or —C₃-C₄ cycloalkyl.

In some embodiments, R⁴, when present, is —H. In some embodiments, R⁴, when present, is halo. In some embodiments, R⁴, when present, is —CN. In some embodiments, R⁴, when present, is —OH. In some embodiments, R⁴, when present, is —C₁-C₆ alkyl. In some embodiments, R⁴, when present, is —C₁-C₆ haloalkyl. In some embodiments, R⁴, when present, is —C₁-C₆ alkoxy. In some embodiments, R⁴, when present, is —NR^{4a}R^{4b}. In some embodiments, R⁴, when present, is —C₃-C₈ cycloalkyl.

In some embodiments, R⁴, when present, is —CF₃, —CN, or

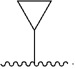 [SMILES: *C1CC1]

In some embodiments, R⁴, when present, is

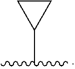 [SMILES: *C1CC1]

In some embodiments, Y is 5- to 6-membered heteroaryl having 1-3 ring heteroatoms independently selected from N, O, and S. In some embodiments, Y is phenyl, or a 6-membered heteroaryl having 1-3 ring nitrogen atoms. In some embodiments, Y is phenyl or pyridyl. In some embodiments, Y is phenyl. In some embodiments, Y is pyridyl. In some embodiments, Y is pyrazolyl. In some embodiments, Y is phenyl, pyrazolyl, or pyridyl.

In some embodiments, the compound has a structure according to Formula Id, Formula Ie, Formula If, or Formula Ig:

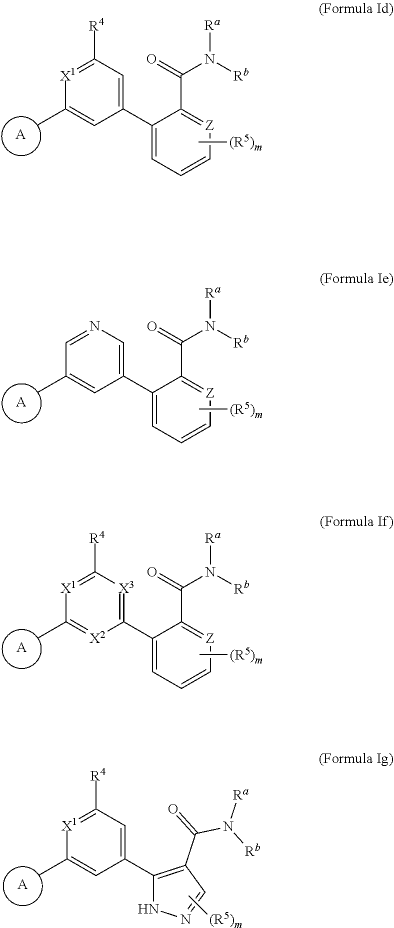

wherein Z, when present, is CH or N; and
m is 0, 1, or 2.

In some embodiments, the compound has a structure according to Formula Id or Formula Ie:

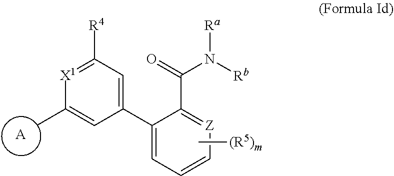

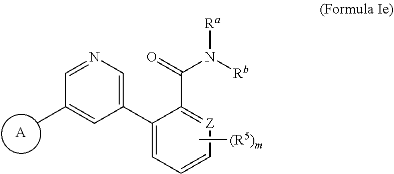

wherein Z is CH or N; and
m is 0, 1, or 2.

In some embodiments, the compound has a structure according to Formula Id:

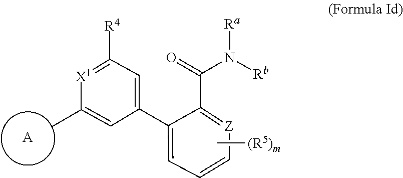

wherein Z is CH or N; and
m is 0, 1, or 2.

In some embodiments, the compound has a structure according to Formula Ie:

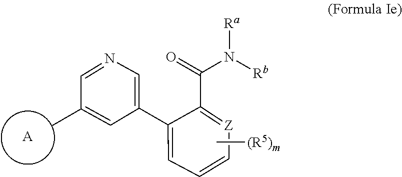

wherein Z is CH or N; and
m is 0, 1, or 2.

In some embodiments, the compound has a structure according to Formula If:

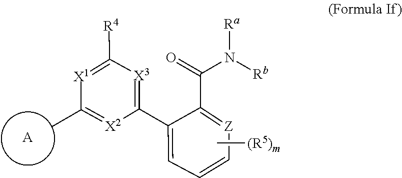

wherein Z, when present, is CH or N; and
m is 0, 1, or 2.

In some embodiments, the compound has a structure according to Formula Ig:

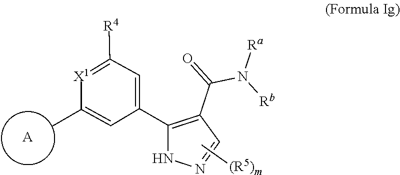

wherein Z, when present, is CH or N; and
m is 0, 1, or 2.

In some embodiments, the compound of Formula Ic has a structure according to Formula Id-1:

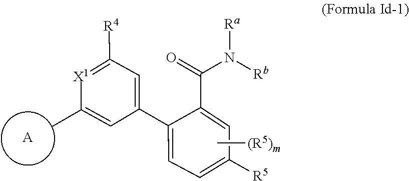

wherein m is 0 or 1.

In some embodiments, the compound of Formula Id has a structure according to Formula

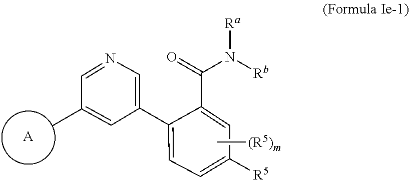

wherein m is 0 or 1.

In some embodiments, each R⁵ is independently halo. In some embodiments, each R⁵ is independently —F.

In some embodiments, each R⁵ is independently halo or —C₁-C₆ alkyl. In some embodiments, each R⁵ is independently F or CH₃.

In some embodiments, R^{a} and R^{b} are each independently H, —C₁-C₆ alkyl, —C₁-C₆ haloalkyl, phenyl, or —(C₁-C₃ alkylene)-O—(C₁-C₃ alkyl). In some embodiments, R^{a} and R^{b} are each independently —H, —CH₃, —CH₂CH₃, —CH₂CH₂—O—CH₂CH₃, —CH₂CF₃, or phenyl. In some embodiments, R^{a} and R^{b} are each independently —H, —CH₃, or —CH₂CH₃.

In some embodiments, R^{a} and R^{b} taken together with the N atom to which they are attached form a 4- to 8-membered heterocycloalkyl optionally having one additional ring heteroatom selected from N, O, and S; wherein said 4- to 8-membered heterocycloalkyl is unsubstituted or substituted with 1-3 substituents independently selected from the group consisting of halo, —CN, —C₁-C₆ alkyl, and —C₁-C₆ alkoxy. In some embodiments, R^{a} and R^{b} taken together with the N atom to which they are attached form a 4- to 6-membered heterocycloalkyl optionally having one additional ring heteroatom selected from N, and O; wherein said 4- to 8-membered heterocycloalkyl is unsubstituted or substituted with 1-3 substituents independently selected from the group consisting of halo, —CN, —C₁-C₆ alkyl, and —C₁-C₆ alkoxy.

In some embodiments, R^{a} and R^{b} taken together with the N atom to which they are attached form

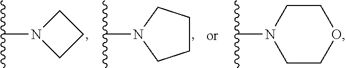

each of which is unsubstituted or substituted with 1-2 substituents independently selected from halo, —CN, —C₁-C₆ alkyl, and —C₁-C₆ alkoxy. In some embodiments, R^{a} and R^{b} taken together with the N atom to which they are attached form

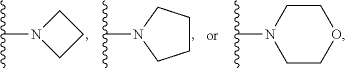

each of which is unsubstituted or substituted with 1-2 substituents independently selected from halo, —CN, —C₁-C₃ alkyl, and —C₁-C₃ alkoxy. In some embodiments, R^{a} and R^{b} taken together with the N atom to which they are attached form

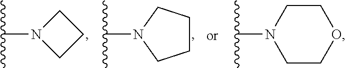

each of which is unsubstituted or substituted with 1-2 substituents independently selected from —F, —CN, —CH₃, and —OCH₃.

In some embodiments, R^{a} and R^{b} taken together with the N atom to which they are attached form

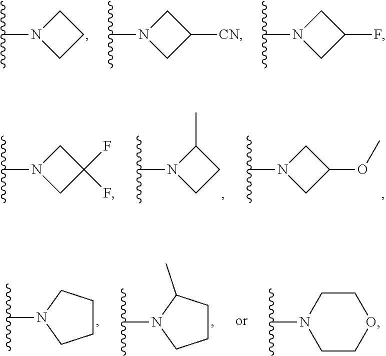

In some embodiments, A is

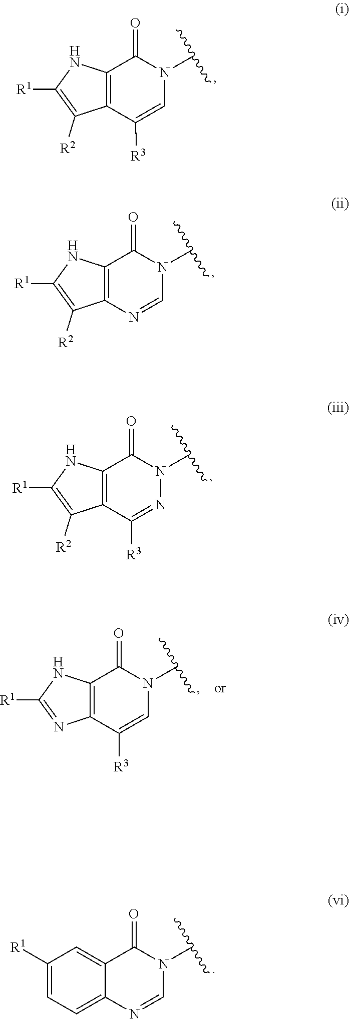

In some embodiments, A is

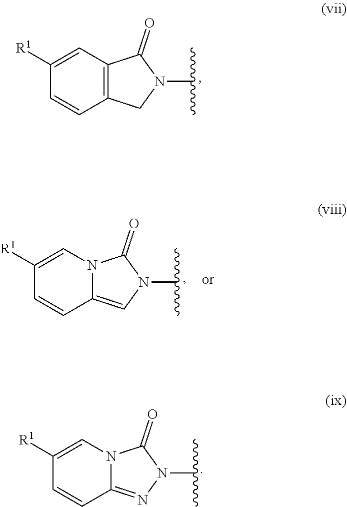

In some embodiments, A is

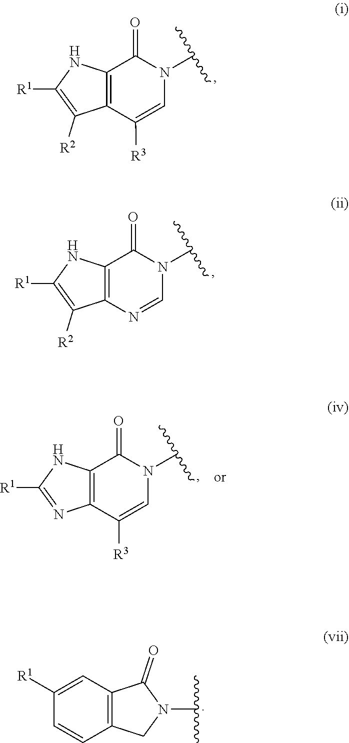

In some embodiments, A is:

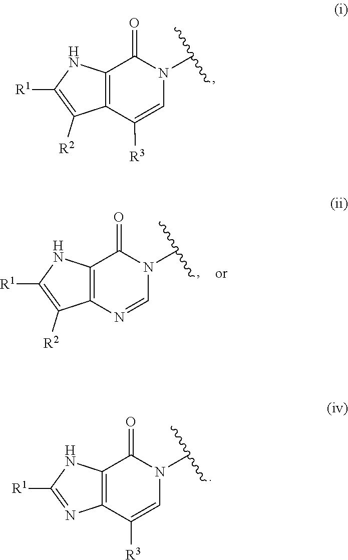

In some embodiments, A is

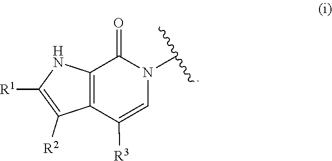

In some embodiments, A is

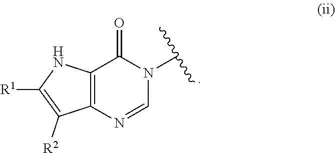

In some embodiments, A

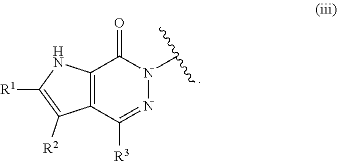

In some embodiments, A is

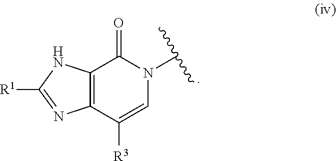

In some embodiments, A is

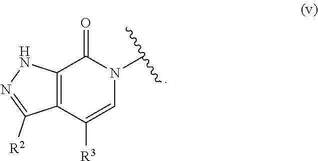

In some embodiments, A is

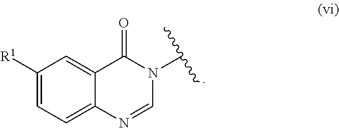

In some embodiments, A is

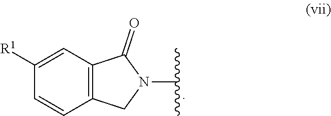

In some embodiments, A is

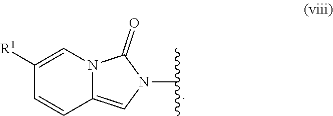

In some embodiments, A is

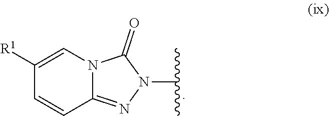

In some embodiments, this disclosure is directed to a compound of Formula I:

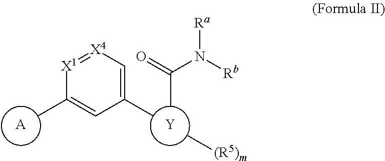

or a pharmaceutically acceptable salt thereof, wherein:
A is selected from the group consisting of:

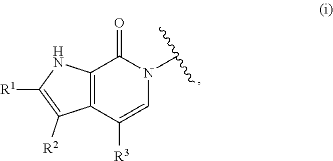

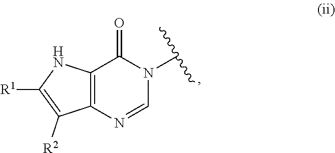

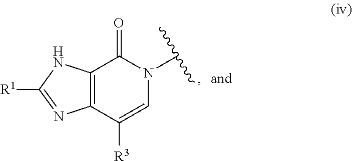

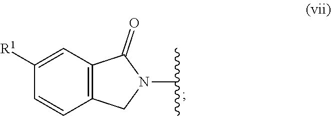

wherein:
R¹ is —C₁-C₆ hydroxyalkyl, -(Q¹)-NR^{1a}R^{1b}, or -(Q¹)-(4- to 8-membered heterocycloalkyl) having 1-3 ring heteroatoms independently selected from N, O, and S; and said 4- to 8-membered heterocycloalkyl is unsubstituted or substituted with 1-2 substituents independently selected from —C₁-C₃ alkyl;
Q¹ is unsubstituted —(C₁-C₃ alkylene)-;
R^{1a} and R^{1b} are independently —H, unsubstituted —C₃-C₆ cycloalkyl, or —C₃-C₆ cycloalkyl substituted with 1 R^{1c};
R^{1c}, when present, is —OH;
R², when present, is —H;
R³ is —CN or —C₃-C₄ cycloalkyl;
X¹ is CH or N;
X⁴ is CR⁴ or N;
R⁴, when present, is —CN, —C₁-C₆ haloalkyl, or —C₃-C₄ cycloalkyl;
Y is phenyl, or a 6-membered heteroaryl having 1-3 ring nitrogen atoms;
m is 0, 1, 2, or 3;
each R⁵ is halo; and
R^{a} and R^{b} are each independently —H, —C₁-C₆ alkyl, —C₁-C₆ haloalkyl, phenyl, or —(C₁-C₃ alkylene)-O—(C₁-C₃ alkyl); or
R^{a} and R^{b} taken together with the N atom to which they are attached form a 4- to 8-membered heterocycloalkyl optionally having one additional ring heteroatom selected from N, O, and S; and wherein said 4- to 8-membered heterocycloalkyl is unsubstituted or substituted with 1-3 substituents independently selected from the group consisting of halo, —CN, —C₁-C₆ alkyl, and —C₁-C₆ alkoxy.

In one or more embodiments, the compound, or pharmaceutically acceptable salt or solvate thereof, according to this disclosure is selected from the compounds provided in Table 1 or Table 2. In one or more embodiments, the compound according to this disclosure is selected from the compounds provided in Table 1 or Table 2.

TABLE 1

| Example | Structure |
|---|---|
| 1 | 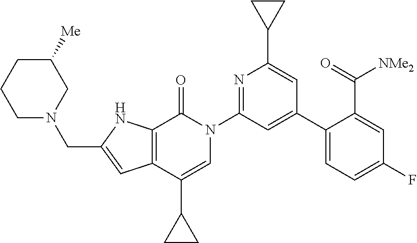 [SMILES: C[C@H]1CCCN(Cc2cc3c(C4CC4)cn(-c4cc(-c5ccc(F)cc5C(=O)N(C)C)cc(C5CC5)n4)c(=O)c3[nH]2)C1] |
| 2 | 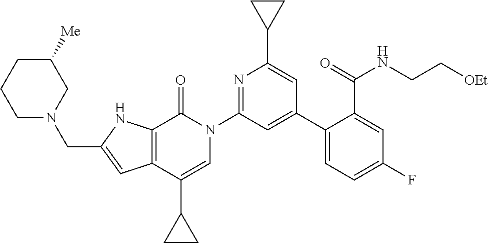 [SMILES: CCOCCNC(=O)c1cc(F)ccc1-c1cc(C2CC2)nc(-n2cc(C3CC3)c3cc(CN4CCC[C@H](C)C4)[nH]c3c2=O)c1] |
| 3 | 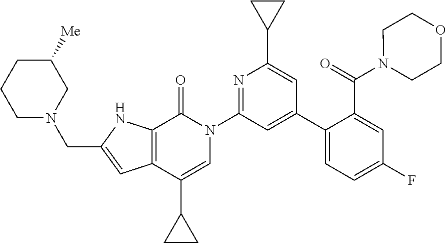 [SMILES: C[C@H]1CCCN(Cc2cc3c(C4CC4)cn(-c4cc(-c5ccc(F)cc5C(=O)N5CCOCC5)cc(C5CC5)n4)c(=O)c3[nH]2)C1] |
| 4 | 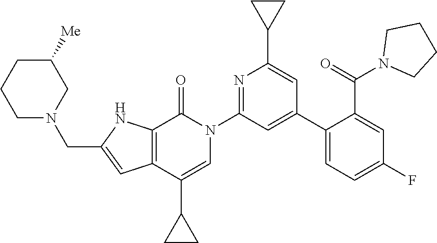 [SMILES: C[C@H]1CCCN(Cc2cc3c(C4CC4)cn(-c4cc(-c5ccc(F)cc5C(=O)N5CCCC5)cc(C5CC5)n4)c(=O)c3[nH]2)C1] |
| 5 | 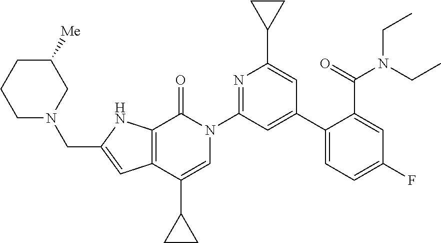 [SMILES: CCN(CC)C(=O)c1cc(F)ccc1-c1cc(C2CC2)nc(-n2cc(C3CC3)c3cc(CN4CCC[C@H](C)C4)[nH]c3c2=O)c1] |
| 6 | 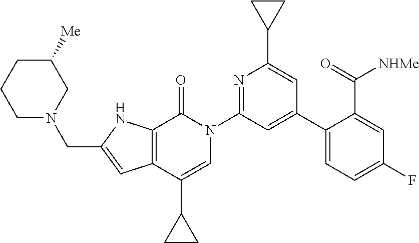 [SMILES: CNC(=O)c1cc(F)ccc1-c1cc(C2CC2)nc(-n2cc(C3CC3)c3cc(CN4CCC[C@H](C)C4)[nH]c3c2=O)c1] |
| 7 | 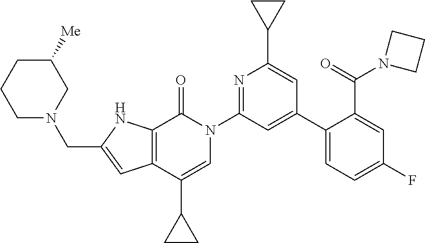 [SMILES: C[C@H]1CCCN(Cc2cc3c(C4CC4)cn(-c4cc(-c5ccc(F)cc5C(=O)N5CCC5)cc(C5CC5)n4)c(=O)c3[nH]2)C1] |
| 8 | 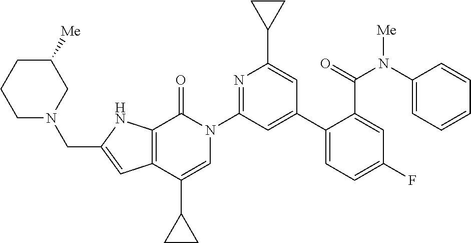 [SMILES: C[C@H]1CCCN(Cc2cc3c(C4CC4)cn(-c4cc(-c5ccc(F)cc5C(=O)N(C)c5ccccc5)cc(C5CC5)n4)c(=O)c3[nH]2)C1] |
| 9 | 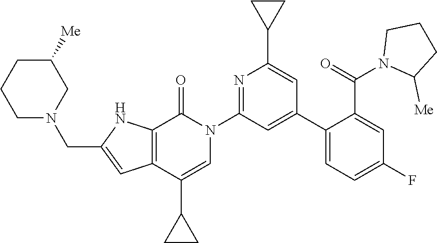 [SMILES: CC1CCCN1C(=O)c1cc(F)ccc1-c1cc(C2CC2)nc(-n2cc(C3CC3)c3cc(CN4CCC[C@H](C)C4)[nH]c3c2=O)c1] |
| 10 | 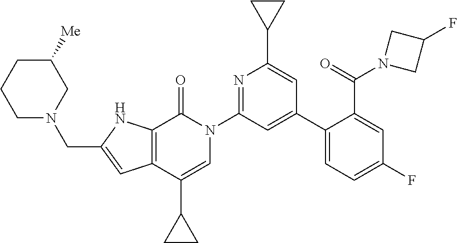 [SMILES: C[C@H]1CCCN(Cc2cc3c(C4CC4)cn(-c4cc(-c5ccc(F)cc5C(=O)N5CC(F)C5)cc(C5CC5)n4)c(=O)c3[nH]2)C1] |
| 11 | 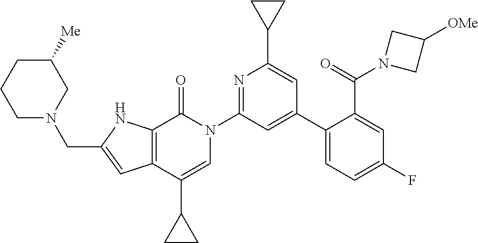 [SMILES: COC1CN(C(=O)c2cc(F)ccc2-c2cc(C3CC3)nc(-n3cc(C4CC4)c4cc(CN5CCC[C@H](C)C5)[nH]c4c3=O)c2)C1] |
| 12 | 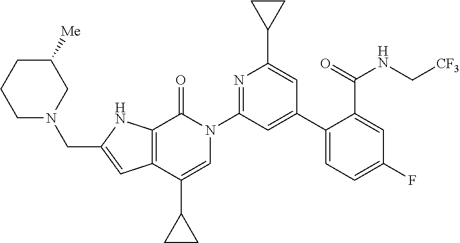 [SMILES: C[C@H]1CCCN(Cc2cc3c(C4CC4)cn(-c4cc(-c5ccc(F)cc5C(=O)NCC(F)(F)F)cc(C5CC5)n4)c(=O)c3[nH]2)C1] |
| 13 | 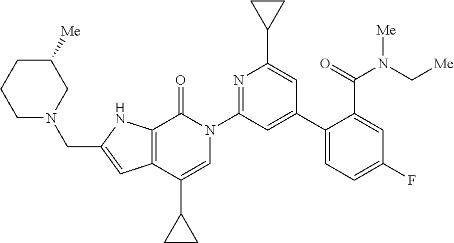 [SMILES: CCN(C)C(=O)c1cc(F)ccc1-c1cc(C2CC2)nc(-n2cc(C3CC3)c3cc(CN4CCC[C@H](C)C4)[nH]c3c2=O)c1] |
| 14 | 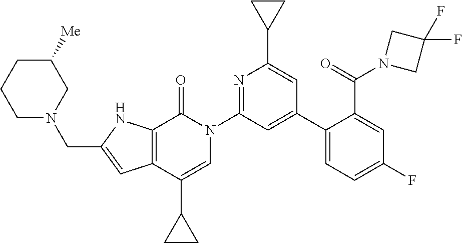 [SMILES: C[C@H]1CCCN(Cc2cc3c(C4CC4)cn(-c4cc(-c5ccc(F)cc5C(=O)N5CC(F)(F)C5)cc(C5CC5)n4)c(=O)c3[nH]2)C1] |
| 15 | 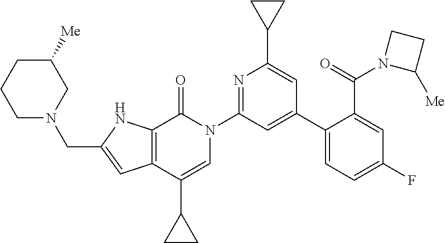 [SMILES: CC1CCN1C(=O)c1cc(F)ccc1-c1cc(C2CC2)nc(-n2cc(C3CC3)c3cc(CN4CCC[C@H](C)C4)[nH]c3c2=O)c1] |
| 16 | 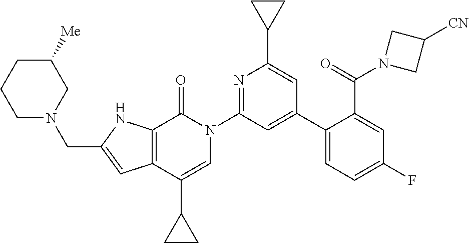 [SMILES: C[C@H]1CCCN(Cc2cc3c(C4CC4)cn(-c4cc(-c5ccc(F)cc5C(=O)N5CC(C#N)C5)cc(C5CC5)n4)c(=O)c3[nH]2)C1] |
| 17 | 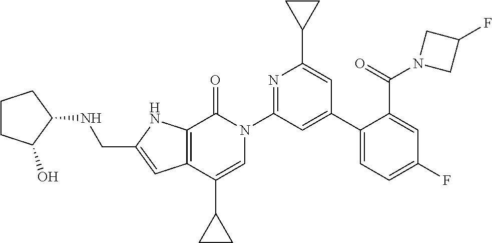 [SMILES: O=C(c1cc(F)ccc1-c1cc(C2CC2)nc(-n2cc(C3CC3)c3cc(CN[C@H]4CCC[C@H]4O)[nH]c3c2=O)c1)N1CC(F)C1] |
| 18 | 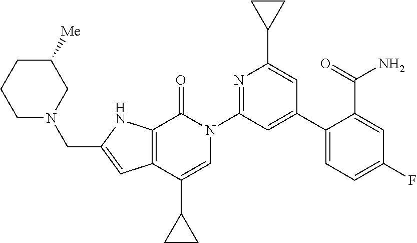 [SMILES: C[C@H]1CCCN(Cc2cc3c(C4CC4)cn(-c4cc(-c5ccc(F)cc5C(N)=O)cc(C5CC5)n4)c(=O)c3[nH]2)C1] |
| 19 | 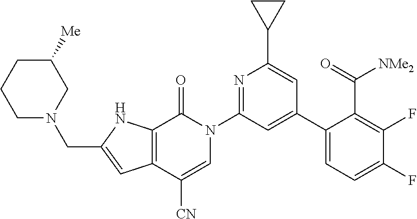 [SMILES: C[C@H]1CCCN(Cc2cc3c(C#N)cn(-c4cc(-c5ccc(F)c(F)c5C(=O)N(C)C)cc(C5CC5)n4)c(=O)c3[nH]2)C1] |
| 20 | 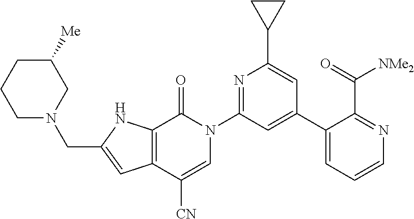 [SMILES: C[C@H]1CCCN(Cc2cc3c(C#N)cn(-c4cc(-c5cccnc5C(=O)N(C)C)cc(C5CC5)n4)c(=O)c3[nH]2)C1] |
| 21 | 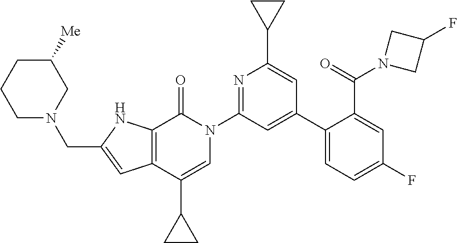 [SMILES: C[C@H]1CCCN(Cc2cc3c(C4CC4)cn(-c4cc(-c5ccc(F)cc5C(=O)N5CC(F)C5)cc(C5CC5)n4)c(=O)c3[nH]2)C1] |
| 22 | 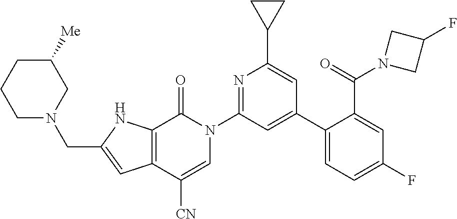 [SMILES: C[C@H]1CCCN(Cc2cc3c(C#N)cn(-c4cc(-c5ccc(F)cc5C(=O)N5CC(F)C5)cc(C5CC5)n4)c(=O)c3[nH]2)C1] |
| 23 | 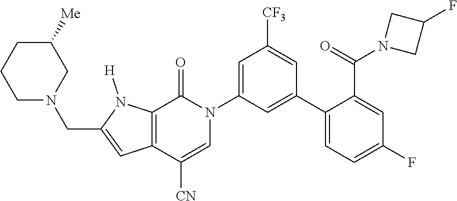 [SMILES: C[C@H]1CCCN(Cc2cc3c(C#N)cn(-c4cc(-c5ccc(F)cc5C(=O)N5CC(F)C5)cc(C(F)(F)F)c4)c(=O)c3[nH]2)C1] |
| 24 | 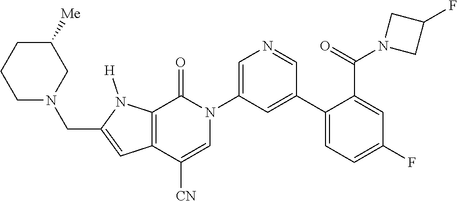 [SMILES: C[C@H]1CCCN(Cc2cc3c(C#N)cn(-c4cncc(-c5ccc(F)cc5C(=O)N5CC(F)C5)c4)c(=O)c3[nH]2)C1] |
| 25 | 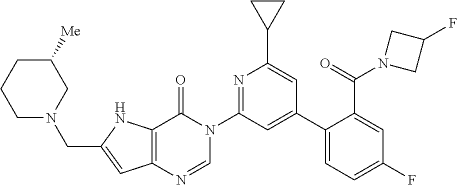 [SMILES: C[C@H]1CCCN(Cc2cc3ncn(-c4cc(-c5ccc(F)cc5C(=O)N5CC(F)C5)cc(C5CC5)n4)c(=O)c3[nH]2)C1] |
| 26 | 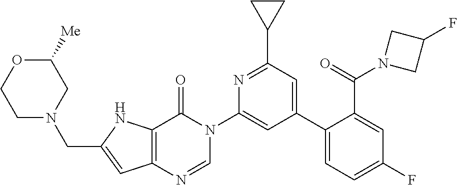 [SMILES: C[C@@H]1CN(Cc2cc3ncn(-c4cc(-c5ccc(F)cc5C(=O)N5CC(F)C5)cc(C5CC5)n4)c(=O)c3[nH]2)CCO1] |
| 27 | 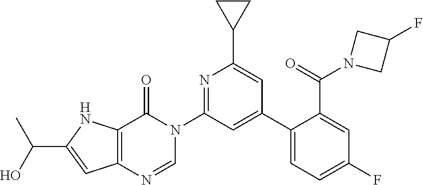 [SMILES: CC(O)c1cc2ncn(-c3cc(-c4ccc(F)cc4C(=O)N4CC(F)C4)cc(C4CC4)n3)c(=O)c2[nH]1] |
| 28 | 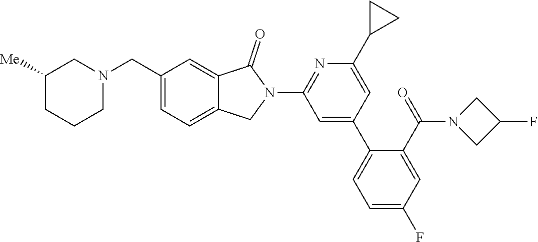 [SMILES: C[C@H]1CCCN(Cc2ccc3c(c2)C(=O)N(c2cc(-c4ccc(F)cc4C(=O)N4CC(F)C4)cc(C4CC4)n2)C3)C1] |
| 29 | 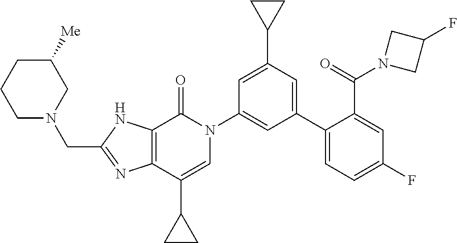 [SMILES: C[C@H]1CCCN(Cc2nc3c(C4CC4)cn(-c4cc(-c5ccc(F)cc5C(=O)N5CC(F)C5)cc(C5CC5)c4)c(=O)c3[nH]2)C1] |

TABLE 2

| Ex. | |
|---|---|
| No. | Structure |
| 30 | 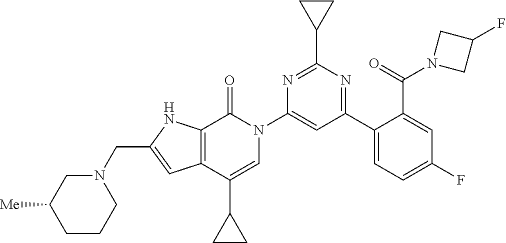 [SMILES: C[C@H]1CCCN(Cc2cc3c(C4CC4)cn(-c4cc(-c5ccc(F)cc5C(=O)N5CC(F)C5)nc(C5CC5)n4)c(=O)c3[nH]2)C1] |
| 31 | 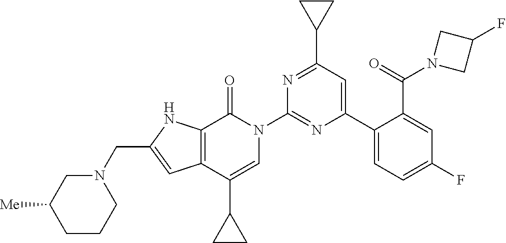 [SMILES: C[C@H]1CCCN(Cc2cc3c(C4CC4)cn(-c4nc(-c5ccc(F)cc5C(=O)N5CC(F)C5)cc(C5CC5)n4)c(=O)c3[nH]2)C1] |
| 32 | 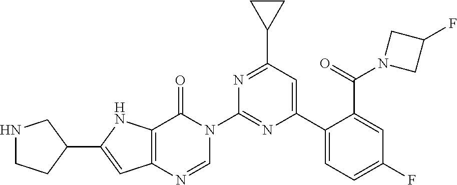 [SMILES: O=C(c1cc(F)ccc1-c1cc(C2CC2)nc(-n2cnc3cc(C4CCNC4)[nH]c3c2=O)n1)N1CC(F)C1] |
| 33 | 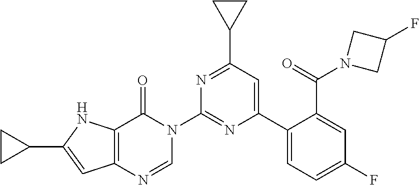 [SMILES: O=C(c1cc(F)ccc1-c1cc(C2CC2)nc(-n2cnc3cc(C4CC4)[nH]c3c2=O)n1)N1CC(F)C1] |
| 34 | 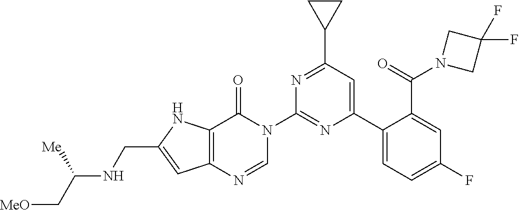 [SMILES: COC[C@H](C)NCc1cc2ncn(-c3nc(-c4ccc(F)cc4C(=O)N4CC(F)(F)C4)cc(C4CC4)n3)c(=O)c2[nH]1] |
| 35 | 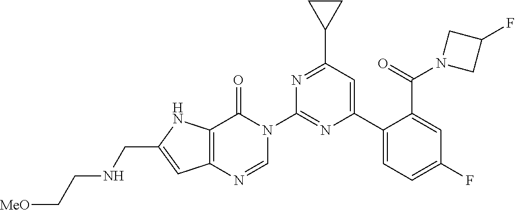 [SMILES: COCCNCc1cc2ncn(-c3nc(-c4ccc(F)cc4C(=O)N4CC(F)C4)cc(C4CC4)n3)c(=O)c2[nH]1] |
| 36 | 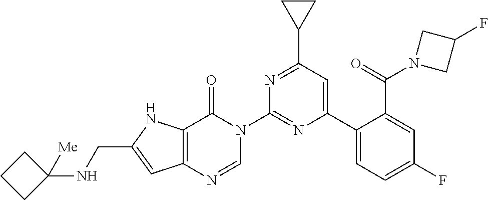 [SMILES: CC1(NCc2cc3ncn(-c4nc(-c5ccc(F)cc5C(=O)N5CC(F)C5)cc(C5CC5)n4)c(=O)c3[nH]2)CCC1] |
| 37 | 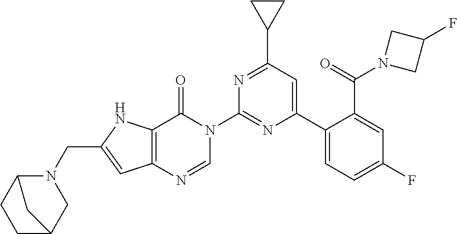 [SMILES: O=C(c1cc(F)ccc1-c1cc(C2CC2)nc(-n2cnc3cc(CN4CC5CCC4C5)[nH]c3c2=O)n1)N1CC(F)C1] |
| 38 | 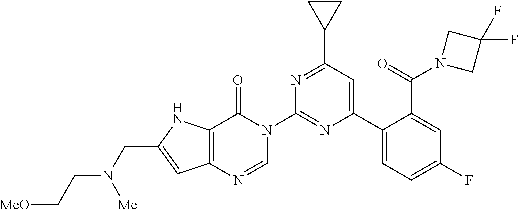 [SMILES: COCCN(C)Cc1cc2ncn(-c3nc(-c4ccc(F)cc4C(=O)N4CC(F)(F)C4)cc(C4CC4)n3)c(=O)c2[nH]1] |
| 39 | 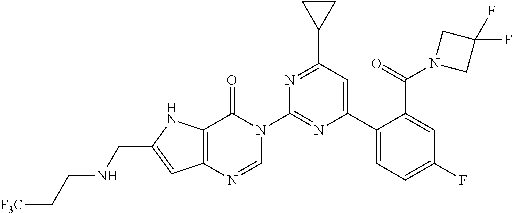 [SMILES: O=C(c1cc(F)ccc1-c1cc(C2CC2)nc(-n2cnc3cc(CNCCC(F)(F)F)[nH]c3c2=O)n1)N1CC(F)(F)C1] |
| 40 | 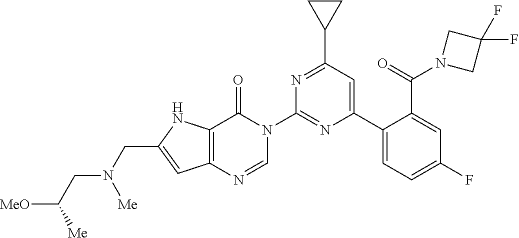 [SMILES: CO[C@@H](C)CN(C)Cc1cc2ncn(-c3nc(-c4ccc(F)cc4C(=O)N4CC(F)(F)C4)cc(C4CC4)n3)c(=O)c2[nH]1] |
| 41 | 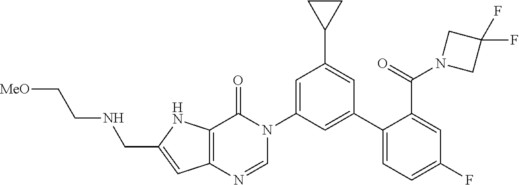 [SMILES: COCCNCc1cc2ncn(-c3cc(-c4ccc(F)cc4C(=O)N4CC(F)(F)C4)cc(C4CC4)c3)c(=O)c2[nH]1] |
| 42 | 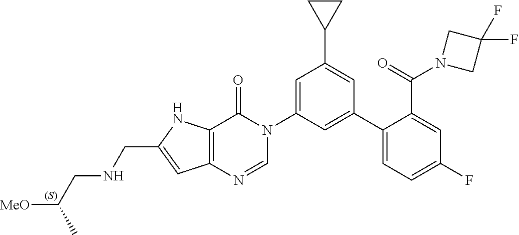 |
| 43 | 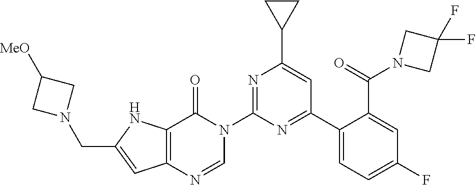 [SMILES: COC1CN(Cc2cc3ncn(-c4nc(-c5ccc(F)cc5C(=O)N5CC(F)(F)C5)cc(C5CC5)n4)c(=O)c3[nH]2)C1] |
| 44 | 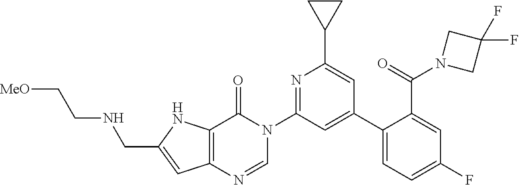 [SMILES: COCCNCc1cc2ncn(-c3cc(-c4ccc(F)cc4C(=O)N4CC(F)(F)C4)cc(C4CC4)n3)c(=O)c2[nH]1] |
| 45 | 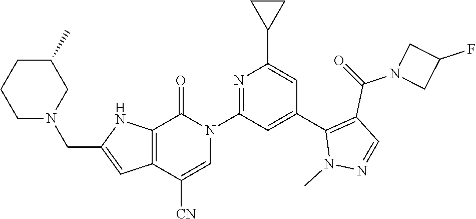 [SMILES: C[C@H]1CCCN(Cc2cc3c(C#N)cn(-c4cc(-c5c(C(=O)N6CC(F)C6)cnn5C)cc(C5CC5)n4)c(=O)c3[nH]2)C1] |
| 46 | 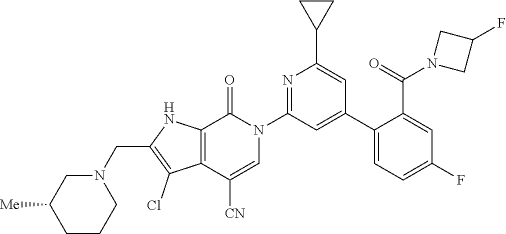 [SMILES: C[C@H]1CCCN(Cc2[nH]c3c(=O)n(-c4cc(-c5ccc(F)cc5C(=O)N5CC(F)C5)cc(C5CC5)n4)cc(C#N)c3c2Cl)C1] |
| 47 | 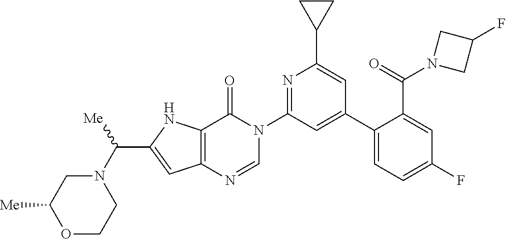 [SMILES: CC(c1cc2ncn(-c3cc(-c4ccc(F)cc4C(=O)N4CC(F)C4)cc(C4CC4)n3)c(=O)c2[nH]1)N1CCO[C@H](C)C1] |
| 48 | 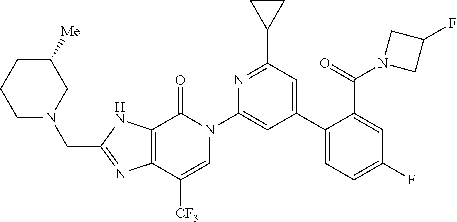 [SMILES: C[C@H]1CCCN(Cc2nc3c(C(F)(F)F)cn(-c4cc(-c5ccc(F)cc5C(=O)N5CC(F)C5)cc(C5CC5)n4)c(=O)c3[nH]2)C1] |
| 49 | 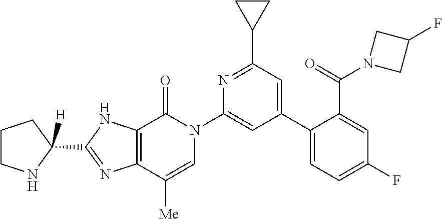 [SMILES: Cc1cn(-c2cc(-c3ccc(F)cc3C(=O)N3CC(F)C3)cc(C3CC3)n2)c(=O)c2[nH]c([C@H]3CCCN3)nc12] |
| 50 | 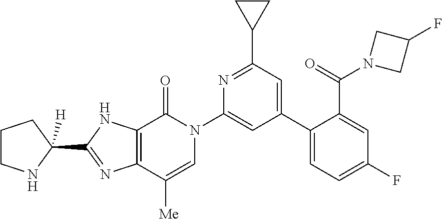 [SMILES: Cc1cn(-c2cc(-c3ccc(F)cc3C(=O)N3CC(F)C3)cc(C3CC3)n2)c(=O)c2[nH]c([C@@H]3CCCN3)nc12] |
| 51 | 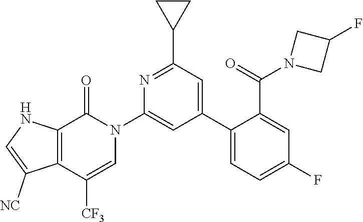 [SMILES: N#Cc1c[nH]c2c(=O)n(-c3cc(-c4ccc(F)cc4C(=O)N4CC(F)C4)cc(C4CC4)n3)cc(C(F)(F)F)c12] |
| 52 | 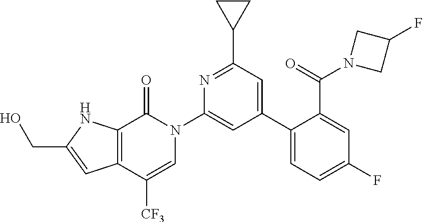 [SMILES: O=C(c1cc(F)ccc1-c1cc(C2CC2)nc(-n2cc(C(F)(F)F)c3cc(CO)[nH]c3c2=O)c1)N1CC(F)C1] |
| 53 | 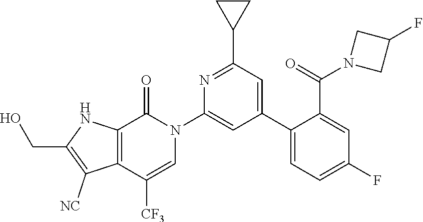 [SMILES: N#Cc1c(CO)[nH]c2c(=O)n(-c3cc(-c4ccc(F)cc4C(=O)N4CC(F)C4)cc(C4CC4)n3)cc(C(F)(F)F)c12] |
| 54 | 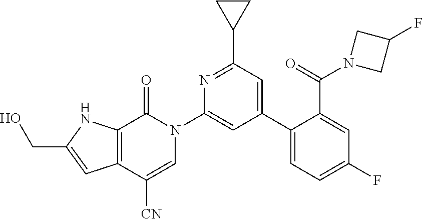 [SMILES: N#Cc1cn(-c2cc(-c3ccc(F)cc3C(=O)N3CC(F)C3)cc(C3CC3)n2)c(=O)c2[nH]c(CO)cc12] |
| 55 | 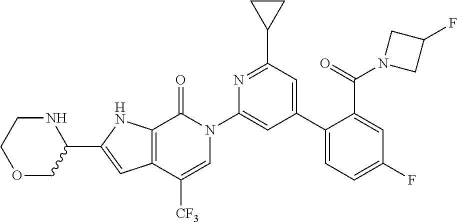 [SMILES: O=C(c1cc(F)ccc1-c1cc(C2CC2)nc(-n2cc(C(F)(F)F)c3cc(C4COCCN4)[nH]c3c2=O)c1)N1CC(F)C1] |
| 56 | 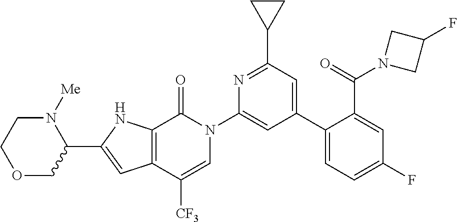 [SMILES: CN1CCOCC1c1cc2c(C(F)(F)F)cn(-c3cc(-c4ccc(F)cc4C(=O)N4CC(F)C4)cc(C4CC4)n3)c(=O)c2[nH]1] |

### Therapeutic and Prophylactic Uses

The present disclosure provides methods for using compounds described herein in the preparation of a medicament for inhibiting Cbl-b. As used herein, the terms “inhibit”, “inhibition” and the like refer to the ability of a compound to decrease the function or activity of a particular target, e.g., Cbl-b. The decrease is preferably at least 50% and may be, for example, at least about 55%, at least about 60%, at least about 65%, at least about 70%, at least about 75%, at least about 80%, at least about 85%, at least about 90%, or at least about 95%. The present disclosure also encompasses the use of the compounds described herein in the preparation of a medicament for the treatment or prevention of diseases, disorders, and/or conditions that would benefit from inhibition of Cbl-b. As one example, the present disclosure encompasses the use of the compounds described herein in the preparation of a medicament for the treatment of cancer. In another example, the present disclosure encompasses the use of the compounds described herein in the preparation of a medicament for the treatment of an infectious disease, optionally a viral infection. In some embodiments of the aforementioned methods, the compounds described herein are used in combination with at least one additional therapy, examples of which are set forth elsewhere herein.

Cbl-b is an E3 ubiquitin ligase that acts by ubiquitinating proteins leading to their degradation or altered subcellular localization. More specifically, Cbl-b acts by binding ubiquitin-conjugating enzyme (E2) loaded with ubiquitin and substrate to facilitate formation of an isopeptide bond between the C-terminal carboxyl of ubiquitin and the F-amino group of a substrate lysine side chain or free N-terminal amino group. Through this activity, Cbl-b functions, in one aspect, as a negative regulator of immune cell activation. For example, Cbl-b inhibits T cell activation through ubiquitination of intracellular signaling proteins, including but not limited to pTYR-containing proteins (e.g., ZAP-70, etc.), p85 regulatory subunit of phosphatidynlinositol 3 kinase (PI3K), PLCγ1, and PKCθ. Cbl-b is also believed to negatively regulate cytokine-induced or target-induced NK cell cytotoxicity and cytokine production. Cbl-b has also been implicated in immunosuppressive signaling pathways, such as PD-1, CTLA-4, CD155, and TGF-β.

As demonstrated herein, the use of compounds described herein potently inhibits Cbl-b activity, resulting in increased immune cell activity. Diseases, disorders, and/or conditions that would benefit from Cbl-b inhibition may include those where greater immune cell (e.g., T cell, NK cell, etc.) activation is desired and/or there is limited immune cell stimulation, for example, due to low antigen density, poor quality neoantigen, high PD-L1 expression, or combinations thereof.

Accordingly, in some embodiments, the compounds described herein are administered to a subject in need thereof in an amount effective to inhibit Cbl-b activity. In one example, a measure of Cbl-b inhibition may be decreased ubiquitination of intracellular signaling proteins targeted by Cbl-b. Non-limiting examples of intracellular signaling proteins targeted by Cbl-b include pTYR-containing proteins (e.g., ZAP-70, etc.), p85 regulatory subunit of phosphatidynlinositol 3 kinase (PI3K), PLCγ1, and PKCθ. Cbl-b activity may be assessed using primary immune cells (e.g., T cells, NK cells) obtained from a peripheral blood sample or a tissue sample (e.g., a tumor sample) that was obtained from the subject. Activity may be determined, for example, by comparison to a previous sample obtained from the subject (i.e., prior to administration of the compound) or by comparison to a reference value for a control group (e.g., standard of care, a placebo, etc.).

Alternatively or in addition, in some embodiments, the compounds described herein are administered to a subject in need thereof in an amount effective to increase immune cell expansion, proliferation, activation and/or activity, as compared to a suitable control (e.g., a subject receiving standard of care, a subject receiving no treatment or a placebo treatment, etc.). Immune cell expansion, proliferation, activation and activity may be assessed using cells obtained from a peripheral blood sample or a tissue sample (e.g., a tumor sample) that was obtained from the subject. Immune cell numbers in tissue or blood may be quantified (absolute numbers or relative numbers) by immunophenotyping, i.e., a process of using antibodies (or other antigen-specific reagent) to detect and quantify cell-associated antigens. Lymphoid cell markers may include but are not limited to CD3, CD4, CD8, CD16, CD25, CD39, CD45, CD56, CD103, CD127, and FOXP3. CD4 and CD8 can distinguish T cell with different effector functions (e.g., CD4+ T cells and CD8+ T cells). Co-expression of different cell markers can further distinguish sub-groups. For example, co-expression of CD39 and CD103 can differentiate tumor-specific T cells (CD8+CD39+CD103+ T cells) from bystander T cells in the tumor microenvironment (TME). For myeloid cells, suitable markers may include but are not limited to CD14, CD68, CD80, CD83, CD86, CD163, and CD206. Ki67 is a non-limiting example of a suitable marker of cell proliferation, such that an increase in Ki67 positive cells (e.g., CD8+ T cells, NK cells, etc.) as compared to a reference sample indicate cell proliferation. The term “activation” refers to the state of an immune cell that has been sufficiently primed to induce detectable effector functions (i.e., immune cell activity) upon stimulation. For example, T cells may be stimulated through the TCR/CD3 complex alone or with one or more secondary costimulatory signals. Non-limiting examples of measures of increased immune cell activity (i.e. effector function) may include increased expression, production and/or secretion of chemokines, pro-inflammatory cytokines and/or cytotoxic factors, increased cytotoxic activity, and increased gene expression and/or cell surface markers related to immune cell function and immune signaling. Examples of pro-inflammatory cytokines include, but are not limited to, IL-1a, IL-1b, IL-2, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of cytotoxic factors include, but are not limited to, granzyme A, granzyme B, soluble Fas ligand (sFasL), and perforin.

In some embodiments, the compounds described herein are administered to a subject in need thereof in an amount effective to increase T cell expansion, proliferation, activity, or any combination thereof. In certain embodiments, the T cells are CD8+ T cells, optionally tumor infiltrating CD8+ T cells and/or antigen experienced CD8+ T cells. In some embodiments, the T cells are CD8+CD39+CD103+ T cells. In embodiments directed to increased T cell activation and/or activity, measures of increased T cell activity may be increased T cell expression, production or secretion of chemokines, pro-inflammatory cytokines (e.g., IFNγ, TNF-α, IL-2, etc.) and/or cytotoxic factors (e.g. perforin, Granzyme B, etc.); increased pro-inflammatory cytokine levels in the tumor microenvironment; increased T cell receptor (TCR) signaling; increased glucose uptake; increased glycolysis; and increased killing of cancer cells. In some embodiments, the compounds described herein are administered to a subject in need thereof in an amount effective to increase activity, optionally wherein a measure of T cell activity is production and/or secretion of one or more pro-inflammatory cytokine, optionally wherein one or more pro-inflammatory cytokine is IFNγ, TNF-α, or IL-2.

In some embodiments, the compounds described herein are administered to a subject in need thereof in an amount effective to increase NK cell expansion, proliferation, activity, or any combination thereof. In embodiments directed to increased NK cell activity, measures of increased NK cell activity may be increased NK cell expression, production or secretion of chemokines, inflammatory cytokines (e.g., IFNγ, TNF-α, IL-2, etc.) and/or cytotoxic factors (e.g. perforin, Granzyme B, etc.); increased inflammatory cytokine levels in the tumor microenvironment; and increased killing of cancer cells.

Alternatively or in addition, in some embodiments, the compounds described herein are administered to a subject in need thereof to treat and/or prevent cancer or a cancer-related disease, disorder or condition. In some embodiments, the compounds described herein are administered to a subject in need thereof to treat cancer, optionally in combination with at least one additional therapy, examples of which are set forth elsewhere herein.

Alternatively or in addition, in some embodiments, the compounds described herein are administered to a subject in need thereof to treat and/or prevent an infection. In some embodiments, the compounds described herein are administered to a subject in need thereof to treat and/or prevent a viral infection. In some embodiments, the viral infection is a disease caused by hepatitis C virus (HCV), human papilloma virus (HPV), cytomegalovirus (CMV), herpes simplex virus (HSV), Epstein-Barr virus (EBV), varicella zoster virus, coxsackie virus, human immunodeficiency virus (HIV), or lymphocytic choriomeningitis virus (LCMV).

Alternatively or in addition, in some embodiments, the compounds described herein are brought into contact with an immune cell or a plurality of immune cells, in vitro or ex vivo, in an amount effective to increase proliferation, activation or activity of the immune cell(s). In some embodiments, the immune cell(s) may be allogenic immune cell(s) collected from one or more subjects. In some embodiments, the immune cell(s) may be autologous immune cell(s) collected from a subject in need of treatment. In certain embodiments, the cells may be “(re)programmed” allogenic immune cells produced from immune precursor cells (e.g., lymphoid progenitor cells, myeloid progenitor cells, common dendritic cell precursor cells, stem cells, induced pluripotent stem cells, etc.). In various embodiments, the immune cells may be genetically modified to target the cells to a specific antigen and/or enhance the cells' anti-tumor effects (e.g., engineered T cell receptor (TCR) cellular therapies, chimeric antigen receptor (CAR) cellular therapies, etc.). In some embodiments, the immune cell(s) are then administered to a subject in need thereof to treat and/or prevent cancer or a cancer-related disease, disorder or condition. In some embodiments, the immune cells are administered to a subject in need thereof to treat cancer, optionally in combination with at least one additional therapy, examples of which are set forth elsewhere herein.

In one or more embodiments, the compounds described herein are useful in the treatment and/or prophylaxis of cancer (e.g., carcinomas, sarcomas, leukemias, lymphomas, myelomas, etc.). In certain embodiments, the cancer may be locally advanced and/or unresectable, metastatic, or at risk of becoming metastatic. Alternatively, or in addition, the cancer may be recurrent or no longer responding to a treatment, such as a standard of care treatment known to one of skill in the art. In some embodiments, the cancer is resistant to treatment with immune checkpoint inhibitors (e.g., anti-PD-1 therapy), and/or chemotherapy (e.g., platinum-based chemotherapy). Exemplary types of cancer contemplated by this disclosure include cancer of the genitourinary tract (e.g., gynecologic, bladder, kidney, renal cell, penile, prostate, testicular, etc.), breast, gastrointestinal tract (e.g., esophagus, oropharynx, stomach, small or large intestines, colon, or rectum), bone, bone marrow, skin (e.g., melanoma), head and neck, liver, gall bladder, bile ducts, heart, lung, pancreas, salivary gland, adrenal gland, thyroid, brain (e.g., gliomas), ganglia, central nervous system (CNS), peripheral nervous system (PNS), the hematopoietic system (i.e., hematological malignancies), the immune system (e.g., spleen or thymus), and cancers associated with Von Hippel-Lindau disease (VHL).

In some embodiments, the compounds according to this disclosure are useful in the treatment and/or prophylaxis of hematological malignancies. Exemplary types of cancer affecting the hematopoietic system include leukemias, lymphomas and myelomas, including acute myeloid leukemia, adult T-cell leukemia, T-cell large granular lymphocyte leukemia, acute lymphoblastic leukemia, chronic lymphocytic leukemia, chronic myelogenous leukemia, acute monocytic leukemia, Hodgkin's and Non-Hodgkin's lymphoma, Diffuse large B Cell lymphoma, and multiple myeloma. In a specific embodiment, the compounds according to this disclosure are useful in the treatment of Diffuse large B Cell lymphoma, optionally Diffuse large B Cell lymphoma with Richter transformation.

In another embodiment, the compounds according to this disclosure are useful in the treatment and/or prophylaxis of solid tumors. The solid tumor may be, for example, ovarian cancer, endometrial cancer, breast cancer, lung cancer (small cell or non-small cell), colon cancer, prostate cancer, cervical cancer, biliary cancer, pancreatic cancer, gastric cancer, esophageal cancer, liver cancer (hepatocellular carcinoma), kidney cancer (renal cell carcinoma), head-and-neck tumors, mesothelioma, melanoma, sarcomas, central nervous system (CNS) hemangioblastomas, and brain tumors (e.g., gliomas, such as astrocytoma, oligodendroglioma and glioblastomas).

In another embodiment, the compounds according to this disclosure are useful in the treatment and/or prophylaxis of breast cancer, genitourinary cancer, gastrointestinal cancer, lung cancer, skin cancer, or a combination thereof.

In some embodiments, the compounds according to this disclosure are useful in the treatment of breast cancer. In further embodiments, the breast cancer is hormone receptor positive (e.g., Era-positive breast cancer, PR-positive breast cancer, Era-positive and PR-positive breast cancer), HER2 positive breast cancer, HER2 over-expressing breast cancer, or any combination thereof. In still further embodiments, the breast cancer is triple negative breast cancer (TNBC).

In some embodiments, the compounds according to this disclosure are useful in the treatment of genitourinary cancer. In further embodiments, the genitourinary cancer is gynecologic cancer. In still further embodiments, the gynecologic cancer is cervical cancer, ovarian cancer (e.g., epithelial ovarian cancer (EOC)), vaginal cancer, vulvar cancer, endometrial cancer, peritoneal cancer, or fallopian tube carcinoma. In still further embodiments, the genitourinary cancer is urothelial cancer. In still further embodiments, the genitourinary cancer is prostate cancer, optionally castration-resistant prostate cancer. In further embodiments, the genitourinary cancer is bladder cancer. In still further embodiments, the genitourinary cancer is peritoneal cancer, optionally primary peritoneal cancer.

In some embodiments, the compounds according to this disclosure are useful in the treatment of head and neck cancer. In further embodiments, the head and neck cancer is head and neck squamous cell carcinoma (HNSCC).

In some embodiments, the compounds according to this disclosure are useful in the treatment of skin cancer. In further embodiments, the skin cancer is melanoma.

In some embodiments, the compounds according to this disclosure are useful in the treatment of lung cancer. In further embodiments, the lung cancer is mesothelioma or non-small cell lung cancer (NSCLC). In still further embodiments, the NSCLC is lung squamous cell carcinoma or lung adenocarcinoma. In further embodiments, the mesothelioma is malignant pleural mesothelioma (MPM).

In some embodiments, the compounds according to this disclosure are useful in the treatment of gastrointestinal (GI) cancer. In some embodiments, the gastrointestinal cancer is upper GI cancer, such as esophageal or gastric cancer. In further embodiments, the upper GI cancer is an adenocarcinoma, a squamous cell carcinoma, or any combination thereof. In still further embodiments, the upper GI cancer is esophageal adenocarcinoma (EAC), esophageal squamous cell carcinoma (ESCC), gastroesophageal junction adenocarcinoma (GEJ), gastric adenocarcinoma (also referred to herein as “gastric cancer”) or any combination thereof. In some embodiments, the gastrointestinal cancer is lower GI cancer. In further embodiments, the lower GI cancer is colorectal cancer.

In some embodiments, the compounds according to this disclosure are useful in the treatment of a neuroendocrine tumor. In further embodiments, the neuroendocrine tumor is pancreatic neuroendocrine tumor, pheochromocytoma, paraganglioma, or a tumor of the adrenal gland.

In some embodiments, the compounds according to this disclosure are useful in the treatment of brain cancer. In further embodiments, the brain cancer is a glioma. In still further embodiments, the glioma is an astrocytoma, an oligodendroglioma, or a glioblastoma.

In some embodiments, the compounds according to this disclosure are useful in the treatment of kidney cancer. In further embodiments, the kidney cancer is renal cell carcinoma. In still further embodiments, the renal cell carcinoma is clear cell renal carcinoma.

In some embodiments, the compounds according to this disclosure are useful in the treatment of pancreatic cancer. In further embodiments, the pancreatic cancer is pancreatic neuroendocrine tumor or pancreatic adenocarcinoma.

In the aforementioned embodiments, the methods of the present disclosure may be practiced in an adjuvant setting or neoadjuvant setting, optionally in the treatment of locally advanced, unresectable, or metastatic cancer. Alternatively or in addition, the methods described herein may be indicated as a first line, second line, third line, or greater line of treatment, optionally in the treatment of locally advanced, unresectable, or metastatic cancer.

The present disclosure also provides methods of treating or preventing other cancer-related diseases, disorders or conditions. The use of the term(s) cancer-related diseases, disorders and conditions is meant to refer broadly to conditions that are associated, directly or indirectly, with cancer and non-cancerous proliferative disease, and includes, e.g., angiogenesis, precancerous conditions such as dysplasia, and non-cancerous proliferative diseases disorders or conditions, such as benign proliferative breast disease and papillomas. For clarity, the term(s) cancer-related disease, disorder and condition do not include cancer per se.

In general, the disclosed methods for treating or preventing cancer, or a cancer-related disease, disorder or condition, in a subject in need thereof comprise administering to the subject a compound disclosed herein, or a pharmaceutically acceptable salt thereof. In some embodiments, the present disclosure provides methods for treating or preventing cancer, or a cancer-related disease, disorder or condition with a compound disclosed herein, or a pharmaceutically acceptable salt thereof, and at least one additional therapy, examples of which are set forth elsewhere herein.

In particular embodiments of the present disclosure, the compounds are used to increase or enhance an immune response to an antigen by providing adjuvant activity. In a particular embodiment, at least one antigen or vaccine is administered to a subject in combination with at least one compound of the present disclosure to prolong an immune response to the antigen or vaccine. Therapeutic compositions are also provided which include at least one antigenic agent or vaccine component, including, but not limited to, viruses, bacteria, and fungi, or portions thereof, proteins, peptides, tumor-specific antigens, and nucleic acid vaccines, in combination with at least one compound of the present disclosure.

In some instances, the methods according to this disclosure may be provided in selected patients, for example subjects identified as having in a relevant tissue or sample, e.g., detectable PD-L1 expression, high microsatellite instability, high tumor mutational burden, or any combination thereof. In some instances, the subject is identified as having an oncogene driven cancer that has a mutation in at least one gene associated with the cancer.

In some embodiments, patients are selected by assessing the expression of relevant biomarkers, e.g., PD-L1 expression, microsatellite instability markers, etc., in a relevant sample, such as a peripheral blood sample or a tumor biopsy, using immunohistochemistry, immunophenotyping, PCR-based amplification, RNA sequencing, or other clinically validated assay. In one embodiment, the disclosure provides a method of treating cancer in a patient having (i) detectable PD-L1 expression, (ii) elevated PD-L1 expression, (iii) variability in the size of one, two, or more microsatellite repeats compared to normal cells, or (iv) any combination of (i) to (iii) by administering a compound as described herein. In another embodiment, the disclosure provides a method of treating cancer in a patient having (i) detectable PD-L1 expression, (ii) elevated PD-L1 expression, (iii) variability in the size of one, two, or more microsatellite repeats compared to normal cells, or (iv) any combination of (i) to (iii) by administering a therapeutically effective amount of a compound as described herein. In still another embodiment, the disclosure provides a method of administering a therapeutically effective amount of a compound as described herein to an individual for the treatment of cancer based on a determination of the relative amount of PD-L1 expression. In yet another embodiment, the disclosure provides a method of administering a therapeutically effective amount of a compound described herein to an individual for the treatment of cancer, the method comprising measuring PD-L1 expression and/or microsatellite instability in a sample obtained from an individual, for example by immunohistochemistry, immunophenotyping, PCR-based amplification, or other clinically validated test, and administering a therapeutically effective amount of the compound to the individual whose sample contained detectable PD-L1 expression.

### Routes of Administration

In some embodiments, pharmaceutical compositions containing a compound according to this disclosure may be in a form suitable for oral administration. Oral administration may involve swallowing the formulation thereby allowing the compound to be absorbed into the bloodstream in the gastrointestinal tract. Alternatively, oral administration may involve buccal, lingual or sublingual administration, thereby allowing the compound to be absorbed into the blood stream through oral mucosa.

In another embodiment, the pharmaceutical compositions containing a compound according to this disclosure may be in a form suitable for parenteral administration. Forms of parenteral administration include, but are not limited to, intravenous, intraarterial, intramuscular, intradermal, intraperitoneal, intrathecal, intracisternal, intracerebral, intracerebroventricular, intraventricular, and subcutaneous. Pharmaceutical compositions suitable for parenteral administration may be formulated using suitable aqueous or non-aqueous carriers. Depot injections, which are generally administered subcutaneously or intramuscularly, may also be utilized to release the compounds disclosed herein over a defined period of time.

Other routes of administration are also contemplated by this disclosure, including, but not limited to, nasal, vaginal, intraocular, rectal, topical (e.g., transdermal), and inhalation.

Particular embodiments of the present disclosure contemplate oral administration or parenteral administration.

### Pharmaceutical Compositions

The compounds of the present disclosure may be in the form of compositions suitable for administration to a subject. In general, such compositions are pharmaceutical compositions comprising a compound according to this disclosure or a pharmaceutically acceptable salt thereof and one or more pharmaceutically acceptable excipients. In some embodiments, the pharmaceutical composition comprises a compound according to this disclosure and one or more pharmaceutically acceptable excipients. In certain embodiments, the compound may be present in an effective amount. The pharmaceutical compositions may be used in the methods of the present disclosure; thus, for example, the pharmaceutical compositions comprising a compound according to this disclosure can be administered to a subject in order to practice the therapeutic and prophylactic methods and uses described herein.

The pharmaceutical compositions of the present disclosure can be formulated to be compatible with the intended method or route of administration. Routes of administration may include those known in the art. Exemplary routes of administration are oral and parenteral. Furthermore, the pharmaceutical compositions may be used in combination with one or more other therapies described herein in order to treat or prevent the diseases, disorders and conditions as contemplated by the present disclosure. In one embodiment, one or more other therapeutic agents contemplated by this disclosure are included in the same pharmaceutical composition that comprises the compound according to this disclosure. In another embodiment, the one or more other therapeutical agents are in a composition that is separate from the pharmaceutical composition comprising the compound according to this disclosure.

In one aspect, the compounds described herein may be administered orally. Oral administration may be via, for example, capsule or tablets. In making the pharmaceutical compositions that include the compounds of the present disclosure (e.g., a compound of Formula I), or a pharmaceutically acceptable salt thereof, the tablet or capsule includes at least one pharmaceutically acceptable excipient. Non-limiting examples of pharmaceutically acceptable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, sterile water, syrup, and methyl cellulose. Additional pharmaceutically acceptable excipients include lubricating agents such as talc, magnesium stearate, and mineral oil; wetting agents; emulsifying and suspending agents; preserving agents such as methyl and propylhydroxy-benzoates.

In another aspect, the compounds of the present disclosure, or a pharmaceutically acceptable salt thereof, may be administered parenterally, for example by intravenous injection. A pharmaceutical composition appropriate for parenteral administration may be formulated in solution for injection or may be reconstituted for injection in an appropriate system such as a physiological solution. Such solutions may include sterile water for injection, salts, buffers, and tonicity excipients in amounts appropriate to achieve isotonicity with the appropriate physiology.

The pharmaceutical compositions described herein may be stored in an appropriate sterile container or containers. In some embodiments, the container is designed to maintain stability for the pharmaceutical composition over a given period of time.

### Administering

In general, the disclosed methods comprise administering a compound described herein, or a composition thereof, in an effective amount to a subject in need thereof. An “effective amount” with reference to a Cbl-b inhibitor of the present disclosure means an amount of the compound that is sufficient to engage the target (e.g., by inhibiting the target) at a level that is indicative of the potency of the compound. For Cbl-b, target engagement can be determined by one or more biochemical or cellular assays resulting in an EC50, ED50, EC90, IC50, or similar value which can be used as one assessment of the potency of the compound. Assays for determining target engagement include, but are not limited to, those described in the Examples. The effective amount may be administered as a single quantity or as multiple, smaller quantities (e.g., as one tablet with “x” amount, as two tablets each with “x/2” amount, etc.).

In some embodiments, the disclosed methods comprise administering a therapeutically effective amount of a compound described herein to a subject in need thereof. As used herein, the phrase “therapeutically effective amount” with reference to compound disclosed herein means a dose regimen (i.e., amount and interval) of the compound that provides the specific pharmacological effect for which the compound is administered to a subject in need of such treatment. For prophylactic use, a therapeutically effective amount may be effective to eliminate or reduce the risk, lessen the severity, or delay the onset of the disease, including biochemical, histological and/or behavioral signs or symptoms of the disease. For treatment, a therapeutically effective amount may be effective to reduce, ameliorate, or eliminate one or more signs or symptoms associated with a disease, delay disease progression, prolong survival, decrease the dose of other medication(s) required to treat the disease, or a combination thereof. With respect to cancer specifically, a therapeutically effective amount may, for example, result in the killing of cancer cells, reduce cancer cell counts, reduce tumor burden, eliminate tumors or metastasis, or reduce metastatic spread. A therapeutically effective amount may vary based on, for example, one or more of the following: the age and weight of the subject, the subject's overall health, the stage of the subject's disease, the route of administration, and prior or concomitant treatments.

Administration may comprise one or more (e.g., one, two, or three or more) dosing cycles.

In certain embodiments, the compounds contemplated by the present disclosure may be administered (e.g., orally, parenterally, etc.) at about 0.01 mg/kg to about 50 mg/kg, or about 1 mg/kg to about 25 mg/kg, of subject's body weight per day, one or more times a day, a week, or a month, to obtain the desired effect. In some embodiments, once daily or twice daily administration is contemplated. In some embodiments, a suitable weight-based dose of a compound contemplated by the present disclosure is used to determine a dose that is administered independent of a subject's body weight. In certain embodiments, the compounds of the present disclosure are administered (e.g., orally, parenterally, etc.) at fixed dosage levels of about 1 mg to about 1000 mg, particularly 1, 3, 5, 10, 15, 20, 25, 50, 75, 100, 150, 200, 250, 300, 400, 500, 600, 750, 800, 900, or 1000 mg, one or more times a day, a week, or a month, to obtain the desired effect.

In certain embodiments, the compound is contained in a “unit dosage form”. The phrase “unit dosage form” refers to physically discrete units, each unit containing a predetermined amount of the compound, either alone or in combination with one or more additional agents, sufficient to produce the desired effect. It will be appreciated that the parameters of a unit dosage form will depend on the particular agent and the effect to be achieved.

### Combination Therapy

The present disclosure contemplates the use of compounds disclosed herein alone or in combination with one or more additional therapy. Each additional therapy can be a therapeutic agent or another treatment modality. In embodiments comprising one or more additional therapeutic agents, each agent may target a different, but complementary, mechanism of action. The additional therapeutic agents can be small chemical molecules; macromolecules such as proteins, antibodies, peptibodies, peptides, DNA, RNA or fragments of such macromolecules; or cellular or gene therapies. Non-limiting examples of additional treatment modalities include surgical resection of a tumor, bone marrow transplant, radiation therapy, and photodynamic therapy. The use of a compound disclosed herein in combination with one or more additional therapies may have a synergistic therapeutic or prophylactic effect on the underlying disease, disorder, or condition. In addition to or alternatively, the combination therapy may allow for a dose reduction of one or more of the therapies, thereby ameliorating, reducing or eliminating adverse effects associated with one or more of the agents.

In embodiments comprising one or more additional treatment modality, the compound can be administered before, after or during treatment with the additional treatment modality. In embodiments comprising one or more additional therapeutic agents, the therapeutic agents used in such combination therapy can be formulated as a single composition or as separate compositions. If administered separately, each therapeutic agent in the combination can be given at or around the same time, or at different times. Furthermore, the therapeutic agents are administered “in combination” even if they have different forms of administration (e.g., oral capsule and intravenous), they are given at different dosing intervals, one therapeutic agent is given at a constant dosing regimen while another is titrated up, titrated down or discontinued, or each therapeutic agent in the combination is independently titrated up, titrated down, increased or decreased in dosage, or discontinued and/or resumed during a subject's course of therapy. If the combination is formulated as separate compositions, in some embodiments, the separate compositions are provided together in a kit.

### Cancer Therapies

The present disclosure contemplates the use of the compounds described herein in combination with one or more additional therapies useful in the treatment of cancer.

In some embodiments, one or more of the additional therapies is an additional treatment modality. Exemplary treatment modalities include but are not limited to surgical resection of a tumor, bone marrow transplant, radiation therapy, and photodynamic therapy.

In some embodiments, one or more of the additional therapies is a therapeutic agent. Exemplary therapeutic agents include chemotherapeutic agents, radiopharmaceuticals, hormone therapies, epigenetic modulators, ATP-adenosine axis-targeting agents, targeted therapies, signal transduction inhibitors, RAS signaling inhibitors, PI3K inhibitors, arginase inhibitors, HIF inhibitors, AXL inhibitors, PAK4 inhibitors, immunotherapeutic agents, cellular therapies, gene therapies, immune checkpoint inhibitors, and agonists of stimulatory or co-stimulatory immune checkpoints.

In some embodiments, one or more of the additional therapeutic agents is a chemotherapeutic agent. Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, pomalidomide, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pemetrexed, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel, nab paclitaxel, and docetaxel; chlorambucil; gemcitabine; 6-thioguanine; mercaptopurine; methotrexate; platinum and platinum coordination complexes such as cisplatin, carboplatin and oxaliplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; proteasome inhibitors such as bortezomib, carfilzomib and ixazomib; topoisomerase inhibitors such as irinotecan, topotecan, etoposide, mitoxantrone, teniposide; difluoromethylornithine (DMFO); retinoic acid; esperamicins; capecitabine; anthracyclines and pharmaceutically acceptable salts, acids or derivatives of any of the above. In certain embodiments, combination therapy comprises a chemotherapy regimen that includes one or more chemotherapeutic agents. In one embodiment, combination therapy comprises a chemotherapeutic regimen comprising one or more of FOLFOX (folinic acid, fluorouracil, and oxaliplatin), FOLFIRI (e.g., folinic acid, fluorouracil, and irinotecan), FOLFIRINOX (folinic acid, fluorouracil, irinotecan, and oxaliplatin), CAPOX (capecitabine and oxaliplatin), a taxoid (e.g., docetaxel, paclitaxel, nab-paclitaxel, etc.), a fluoropyrimidine-containing chemotherapeutic agent (e.g., fluorouracil, capecitabine, floxuridine), a platinum-containing chemotherapeutic agent, and/or gemcitabine.

In some embodiments, one or more of the additional therapeutic agents is a radiopharmaceutical. A radiopharmaceutical is a form of internal radiation therapy in which a source of radiation (i.e., one or more radionuclide) is put inside a subject's body. The radiation source can be in solid or liquid form. Non-limiting examples of radiopharmaceuticals include sodium iodide I-131, radium-223 dichloride, lobenguane iodine-131, radioiodinated vesicles (e.g., saposin C-dioleoylphosphatidylserine (SapC-DOPS) nanovesicles), various forms of brachytherapy, and various forms of targeted radionuclides. Targeted radionuclides comprise a radionuclide associated (e.g., by covalent or ionic interactions) with a molecule (“a targeting agent”) that specifically binds to a target on a cell, typically a cancer cell or an immune cell. The targeting agent may be a small molecule, a saccharide (inclusive of oligosaccharides and polysaccharides), an antibody, a lipid, a protein, a peptide, a non-natural polymer, or an aptamer. In some embodiments, the targeting agent is a saccharide (inclusive of oligosaccharides and polysaccharides), a lipid, a protein, or a peptide and the target is a tumor-associated antigen (enriched but not specific to a cancer cell), a tumor-specific antigen (minimal to no expression in normal tissue), or a neo-antigen (an antigen specific to the genome of a cancer cell generated by non-synonymous mutations in the tumor cell genome). In some embodiments, the targeting agent is an antibody and the target is a tumor-associated antigen (i.e., an antigen enriched but not specific to a cancer cell), a tumor-specific antigen (i.e., an antigen with minimal to no expression in normal tissue), or a neo-antigen (i.e., an antigen specific to the genome of a cancer cell generated by non-synonymous mutations in the tumor cell genome). Non-limiting examples of targeted radionuclides include radionuclides attached to: somatostatin or peptide analogs thereof (e.g., 177Lu-Dotatate, etc.); prostate specific membrane antigen or peptide analogs thereof (e.g., 177Lu-PSMA-617, 225Ac-PSMA-617, 177Lu-PSMA-I&T, 177Lu-MIP-1095, etc.); a receptor's cognate ligand, peptide derived from the ligand, or variants thereof (e.g., 188Re-labeled VEGF₁₂₅₋₁₃₆ or variants thereof with higher affinity to VEGF receptor, etc.); antibodies targeting tumor antigens (e.g., 131I-tositumomab, 90Y-ibritumomab tiuxetan, CAM-H2-I131 (Precirix NV), I131-omburtamab, etc.).

In some embodiments, one or more of the additional therapeutic agents is a hormone therapy. Hormone therapies act to regulate or inhibit hormonal action on tumors. Examples of hormone therapies include, but are not limited to: selective estrogen receptor degraders such as fulvestrant, giredestrant, SAR439859, RG6171, AZD9833, rintodestrant, ZN-c5, LSZ102, D-0502, LY3484356, SHR9549; selective estrogen receptor modulators such as tamoxifen, raloxifene, 4-hydroxytamoxifen, trioxifene, keoxifene, toremifene; aromatase inhibitors such as anastrozole, exemestane, letrozole and other aromatase inhibiting 4(5)-imidazoles; gonadotropin-releasing hormone agonists such as nafarelin, triptoreiin, goserein; gonadotropin-releasing hormone antagonists such as degarelix; antiandrogens such as abiraterone, enzalutamide, apalutamide, darolutamide, flutamide, nilutamide, bicalutamide, leuprolide; 5α-reductase inhibitors such as finasteride, dutasteride; and the like. In certain embodiments, combination therapy comprises administration of a hormone or related hormonal agent. In one embodiment, combination therapy comprises administration of enzalutamide.

In some embodiments, one or more of the additional therapeutic agents is an epigenetic modulator. An epigenetic modulator alters an epigenetic mechanism controlling gene expression, and may be, for example, an inhibitor or activator of an epigenetic enzyme. Non-limiting examples of epigenetic modulators include DNA methyltransferase (DNMT) inhibitors, hypomethylating agents, and histone deacetylase (HDAC) inhibitors. In one or more embodiments, the compounds according to this disclosure are combined with DNA methyltransferase (DNMT) inhibitors or hypomethylating agents. Exemplary DNMT inhibitors include decitabine, zebularine and azacitadine. In one or more embodiments, combinations of the compounds according to this disclosure with a histone deacetylase (HDAC) inhibitor is also contemplated. Exemplary HDAC inhibitors include vorinostat, givinostat, abexinostat, panobinostat, belinostat and trichostatin A.

In some embodiments, one or more of the additional therapeutic agents is an ATP-adenosine axis-targeting agent. ATP-adenosine axis-targeting agents alter signaling mediated by adenine nucleosides and nucleotides (e.g., adenosine, AMP, ADP, ATP), for example by modulating the level of adenosine or targeting adenosine receptors. Adenosine and ATP, acting at different classes of receptors, often have opposite effects on inflammation, cell proliferation and cell death. For instance, ATP and other adenine nucleotides have antitumor effects via activation of the PS2Y1 receptor subtype, while accumulation of adenosine in the tumor microenvironment has been shown to inhibit the antitumor function of various immune cells and to augment the immunosuppressive activity of myeloid and regulatory T cells by binding to cell surface adenosine receptors. In certain embodiments, an ATP-adenosine axis-targeting agent is an inhibitor of an ectonucleotidase involved in the conversion of ATP to adenosine or an antagonist of adenosine receptor. Ectonucleotidases involved in the conversion of ATP to adenosine include the ectonucleoside triphosphate diphosphohydrolase 1 (ENTPD1, also known as CD39 or Cluster of Differentiation 39) and the ecto-5′-nucleotidase (NT5E or 5NT, also known as CD73 or Cluster of Differentiation 73). Exemplary small molecule CD73 inhibitors include CB-708, ORIC-533, LY3475070 and quemliclustat. Exemplary anti-CD39 and anti-CD73 antibodies include ES002023, TTX-030, IPH-5201, SRF-617, CPI-006, oleclumab (MEDI9447), NZV930, IPH5301, GS-1423, uliledlimab (TJD5, TJ004309), AB598, and BMS-986179. In one embodiment, the present disclosure contemplates combination of the compounds described herein with a CD73 inhibitor such as those described in WO 2017/120508, WO 2018/067424, WO 2018/094148, and WO 2020/046813. In further embodiments, the CD73 inhibitor is quemliclustat (AB680). Adenosine can bind to and activate four different G-protein coupled receptors: A₁R, A_{2A}R, A_{2B}R, and A₃R. A₂R antagonists include etrumadenant, inupadenant, taminadenant, caffeine citrate, NUV-1182, TT-702, DZD-2269, INCB-106385, EVOEXS-21546, AZD-4635, imaradenant, RVU-330, ciforadenant, PBF-509, PBF-999, PBF-1129, and CS-3005. In some embodiments, the present disclosure contemplates the combination of the compounds described herein with an A2_{A}R antagonist, an A2_{B}R antagonist, or an antagonist of A2_{A}R and A2_{B}R. In some embodiments, the present disclosure contemplates the combination of the compounds described herein with the adenosine receptor antagonists described in WO 2018/136700, WO 2018/204661, WO 2018/213377, or WO 2020/023846. In one embodiment, the adenosine receptor antagonist is etrumadenant.

In some embodiments, one or more of the additional therapeutic agents is a targeted therapy. In one aspect, a targeted therapy may comprise a targeting agent and a drug. The drug may be a chemotherapeutic agent, a radionuclide, a hormone therapy, or another small molecule drug attached to a targeting agent. The targeting agent may be a small molecule, a saccharide (inclusive of oligosaccharides and polysaccharides), an antibody, a lipid, a protein, a peptide, a non-natural polymer, or an aptamer. In some embodiments, the targeting agent is a saccharide (inclusive of oligosaccharides and polysaccharides), a lipid, a protein, or a peptide and the target is a tumor-associated antigen (enriched but not specific to a cancer cell), a tumor-specific antigen (minimal to no expression in normal tissue), or a neo-antigen (an antigen specific to the genome of a cancer cell generated by non-synonymous mutations in the tumor cell genome). In some embodiments, the targeting agent is an antibody and the target is a tumor-associated antigen, a tumor-specific antigen, or a neo-antigen. In some embodiments, the targeted therapy is an antibody-drug conjugate comprising an antibody and a drug, wherein the antibody specifically binds to HER2, HER3, nectin-4, or Trop-2. Specific examples of a targeted therapy comprising an antibody and a drug include but are not limited to patritumab deruxtecan, sacituzumab govitecan-hziy, telisotuzumab vedotin, and trastuzumab deruxtecan. Specific examples include but are not limited to patritumab deruxtecan and telisotuzumab vedotin. In other aspects, a targeted therapy may inhibit or interfere with a specific protein that helps a tumor grow and/or spread. Non-limiting examples of such targeted therapies include signal transduction inhibitors, RAS signaling inhibitors, inhibitors of oncogenic transcription factors, activators of oncogenic transcription factor repressors, angiogenesis inhibitors, immunotherapeutic agents, ATP-adenosine axis-targeting agents, AXL inhibitors, PARP inhibitors, PAK4 inhibitors, PI3K inhibitors, HIF-2a inhibitors, CD39 inhibitors, CD73 inhibitors, A2R antagonists, TIGIT antagonists, and PD-1 antagonists. ATP-adenosine axis-targeting agents are described above, while other agents are described in further detail below.

In some embodiments, one or more of the additional therapeutic agents is a signal transduction inhibitor. Signal transduction inhibitors are agents that selectively inhibit one or more steps in a signaling pathway. Signal transduction inhibitors (STIs) contemplated by the present disclosure include but are not limited to: (i) BCR-ABL kinase inhibitors (e.g., imatinib); (ii) epidermal growth factor receptor tyrosine kinase inhibitors (EGFR TKIs), including small molecule inhibitors (e.g., CLN-081, gefitinib, erlotinib, afatinib, icotinib, and osimertinib), and anti-EGFR antibodies; (iii) inhibitors of the human epidermal growth factor (HER) family of transmembrane tyrosine kinases, e.g., HER-2/neu receptor inhibitors (e.g., trastuzumab) and HER-3 receptor inhibitors; (iv) vascular endothelial growth factor receptor (VEGFR) inhibitors including small molecule inhibitors (e.g., axitinib, sunitinib and sorafenib), VEGF kinase inhibitors (e.g., lenvatinib, cabozantinib, pazopanib, tivozanib, XL092, etc.) and anti-VEGF antibodies (e.g., bevacizumab), and anti-VEGFR antibodies (e.g., ramucirumab); (v) inhibitors of AKT family kinases or the AKT pathway (e.g., rapamycin); (vi) inhibitors of mTOR, such as, for example, everolimus, sirolimus, temsirolimus; (vii) inhibitors of serine/threonine-protein kinase B-Raf (BRAF), such as, for example, vemurafenib, dabrafenib and encorafenib; (viii) inhibitors of rearranged during transfection (RET), including, for example, selpercatinib and pralsetinib; (ix) tyrosine-protein kinase Met (MET) inhibitors (e.g., tepotinib, tivantinib, cabozantinib and crizotinib); (x) anaplastic lymphoma kinase (ALK) inhibitors (e.g., ensartinib, ceritinib, lorlatinib, crizotinib, and brigatinib); (xi) inhibitors of the RAS signaling pathway (e.g., inhibitors of KRAS, HRAS, RAF, MEK, ERK) as described elsewhere herein; (xii) FLT-3 inhibitors (e.g., gilteritinib)(xiii) inhibitors of Trop-2; (xiv) inhibitors of the JAK/STAT pathway, e.g., JAK inhibitors including tofacitinib and ruxolitinib, or STAT inhibitors such as napabucasin; (xv) inhibitors of NF-κB; (xvi) cell cycle kinase inhibitors (e.g., flavopiridol); (xvii) phosphatidyl inositol kinase (PI3K) inhibitors; (xiii) protein kinase B (AKT) inhibitors (e.g., capivasertib, miransertib); (xviii) platelet-derived growth factor receptor (PDGFR) inhibitors (e.g., imatinib, sunitinib, regorafenib, avapritinib, lenvatinib, nintedanib, famitinib, ponatinib, axitinib, repretinib, etc.); (xix) insulin-like growth factor receptor (IGFR) inhibitors (e.g., erlotinib, afatinib, gefitinib, osimertinib, dacomitinib); (xx) fibroblast growth factor receptor (FGFR) inhibitors (e.g., futibatinib, erdafitinib, pemigatinib); and (xxi) receptor tyrosine kinase KIT inhibitors (e.g., imatinib, sorafenib, sunitinib, masitinib, repretinib, avapritinib). In one or more embodiments, the additional therapeutic agent comprises an inhibitor of EGFR, VEGFR, HER-2, HER-3, BRAF, RET, MET, ALK, RAS (e.g., KRAS, MEK, ERK), FLT-3, JAK, STAT, NF-κB, PI3K, AKT, FGFR, KIT, or any combinations thereof.

In some embodiments, one or more of the additional therapeutic agents is a RAS signaling inhibitor. Oncogenic mutations in the RAS family of genes, e.g., HRAS, KRAS, and NRAS, are associated with a variety of cancers. For example, mutations of G12C, G12D, G12V, G12A, G13D, Q61H, G13C and G12S, among others, in the KRAS family of genes have been observed in multiple tumor types. Direct and indirect inhibition strategies have been investigated for the inhibition of mutant RAS signaling. Indirect inhibitors target effectors other than RAS in the RAS signaling pathway, and include, but are not limited to, inhibitors of RAF, MEK, ERK, PI3K, PTEN, SOS (e.g., SOS1), mTORC1, SHP2 (PTPN11), and AKT. Non-limiting examples of indirect inhibitors under development include RMC-4630, RMC-5845, RMC-6291, RMC-6236, JAB-3068, JAB-3312, TNO155, RLY-1971, BI1701963. Direct inhibitors of RAS mutants have also been explored, and generally target the KRAS-GTP complex or the KRAS-GDP complex. Exemplary direct RAS inhibitors under development include, but are not limited to, sotorasib (AMG510), adagrasib (MRTX849), mRNA-5671 and ARS1620. In some embodiments, the one or more RAS signaling inhibitors are selected from the group consisting of RAF inhibitors, MEK inhibitors, ERK inhibitors, PI3K inhibitors, PTEN inhibitors, SOS1 inhibitors, mTORC1 inhibitors, SHP2 inhibitors, and AKT inhibitors. In other embodiments the one or more RAS signaling inhibitors directly inhibit RAS mutants.

In some embodiments one or more of the additional therapeutic agents is an inhibitor of a phosphatidylinositol 3-kinase (PI3K), particularly an inhibitor of the PI3Kγ isoform. PI3Kγ inhibitors can stimulate an anti-cancer immune response through the modulation of myeloid cells, such as by inhibiting suppressive myeloid cells, dampening immune-suppressive tumor-infiltrating macrophages or by stimulating macrophages and dendritic cells to make cytokines that contribute to effective T cell responses thereby decreasing cancer development and spread. Exemplary PI3Kγ inhibitors include copanlisib, duvelisib, AT-104, ZX-101, tenalisib, eganelisib, SF-1126, AZD3458, and pictilisib. In some embodiments, the compounds according to this disclosure are combined with one or more PI3Kγ inhibitors described in WO 2020/0247496A1.

In some embodiments, one or more of the additional therapeutic agents is an inhibitor of arginase. Arginase has been shown to be either responsible for or participate in inflammation-triggered immune dysfunction, tumor immune escape, immunosuppression and immunopathology of infectious disease. Exemplary arginase compounds include CB-1158 and OAT-1746. In some embodiments, the compounds according to this disclosure are combined with one or more arginase inhibitors described in WO/2019/173188 and WO 2020/102646.

In some embodiments, one or more of the additional therapeutic agents is an inhibitor of an oncogenic transcription factor or an activator of an oncogenic transcription factor repressor. Suitable agents may act at the expression level (e.g., RNAi, siRNA, etc.), through physical degradation, at the protein/protein level, at the protein/DNA level, or by binding in an activation/inhibition pocket. Non-limiting examples include inhibitors of one or more subunit of the MLL complex (e.g., HDAC, DOT1L, BRD4, Menin, LEDGF, WDR5, KDM4C (JMJD2C) and PRMT1), inhibitors of hypoxia-inducible factor (HIF) transcription factor, and the like.

In some embodiments, one or more of the additional therapeutic agents is an inhibitor of a hypoxia-inducible factor (HIF) transcription factor, particularly HIF-2a. Exemplary HIF-2a inhibitors include belzutifan, ARO-HIF2, PT-2385, AB521, NKT-2152, DFF332, and those described in WO 2021113436, WO 2021188769, and WO 2023077046. In some embodiments, the HIF-2a inhibitor is AB521.

In some embodiments, one or more of the additional therapeutic agents is an inhibitor of anexelekto (AXL). The AXL signaling pathway is associated with tumor growth and metastasis, and is believed to mediate resistance to a variety of cancer therapies. There are a variety of AXL inhibitors under development that also inhibit other kinases in the TAM family (i.e., TYRO3, MERTK), as well as other receptor tyrosine kinases including MET, FLT3, RON and AURORA, among others. Exemplary multikinase inhibitors include sitravatinib, rebastinib, glesatinib, gilteritinib, merestinib, cabozantinib, foretinib, BMS777607, LY2801653, S49076, and RXDX-106. AXL specific inhibitors have also been developed, e.g., small molecule inhibitors including DS-1205, SGI-7079, SLC-391, dubermatinib, bemcentinib, DP3975, and AB801; anti-AXL antibodies such as ADCT-601; and antibody drug conjugates (ADCs) such as BA3011. Another strategy to inhibit AXL signaling involves targeting AXL's ligand, GAS6. For example, batiraxcept is under development as is a Fc fusion protein that binds the GAS6 ligand thereby inhibiting AXL signaling. In some embodiments, the compounds according to this disclosure are combined with one or more AXL inhibitors described in WO2022246177, WO2022246179, or WO2024006726. In some embodiments, the AXL inhibitor is AB801.

In some embodiments, one or more of the additional therapeutic agents is an inhibitor of p21-activated kinase 4 (PAK4). PAK4 overexpression has been shown across a variety of cancer types, notably including those resistant to PD-1 therapies.

In some embodiments, one or more of the additional therapeutic agents is (i) an agent that inhibits the enzyme poly (ADP-ribose) polymerase (e.g., olaparib, niraparib and rucaparib, etc.); (ii) an inhibitor of the Bcl-2 family of proteins (e.g., venetoclax, navitoclax, etc.); (iii) an inhibitor of MCL-1; (iv) an inhibitor of the CD47-SIRPα pathway (e.g., an anti-CD47 antibody); (v) an isocitrate dehydrogenase (IDH) inhibitor, e.g., IDH-1 or IDH-2 inhibitor (e.g., ivosidenib, enasidenib, etc.).

In some embodiments, one or more of the additional therapeutic agents is an immunotherapeutic agent. Immunotherapeutic agents treat a disease by stimulating or suppressing the immune system. Immunotherapeutic agents useful in the treatment of cancers typically elicit or amplify an immune response to cancer cells. Non-limiting examples of suitable immunotherapeutic agents include: immunomodulators; cellular immunotherapies; vaccines; gene therapies; ATP-adenosine axis-targeting agents; immune checkpoint modulators; and certain signal transduction inhibitors. ATP-adenosine axis-targeting agents and signal transduction inhibitors are described above. Immunomodulators, cellular immunotherapies, vaccines, gene therapies, and immune checkpoint modulators are described further below.

In some embodiments, one or more of the additional therapeutic agents is an immunotherapeutic agent, more specifically a cytokine or chemokine, such as, IL-1, IL-2, IL-12, IL-18, ELC/CCL19, SLC/CCL21, MCP-1, IL-4, TNF, IL-15, MDC, IFNα, IFNβ, IFNγ, M-CSF, IL-3, GM-CSF, IL-13, and anti-IL-10; bacterial lipopolysaccharides (LPS); an organic or inorganic adjuvant that activates antigen-presenting cells and promote the presentation of antigen epitopes on major histocompatibility complex molecules agonists including, but not limited to Toll-like receptor (TLR) agonists, antagonists of the mevalonate pathway, agonists of STING; indoleamine 2,3-dioxygenase 1 (IDO1) inhibitors and immune-stimulatory oligonucleotides, as well as other T cell adjuvants.

In some embodiments, one or more of the additional therapeutic agents is an immunotherapeutic agent, more specifically a cellular therapy. Cellular therapies are a form of treatment in which viable cells are administered to a subject. In certain embodiments, one or more of the additional therapeutic agents is a cellular immunotherapy that activates or suppresses the immune system. Cellular immunotherapies useful in the treatment of cancers typically elicit or amplify an immune response. The cells can be autologous or allogenic immune cells (e.g., monocytes, macrophages, dendritic cells, NK cells, T cells, etc.) collected from one or more subject. Alternatively, the cells can be “(re)programmed” allogenic immune cells produced from immune precursor cells (e.g., lymphoid progenitor cells, myeloid progenitor cells, common dendritic cell precursor cells, stem cells, induced pluripotent stem cells, etc.). In some embodiments, such cells may be an expanded subset of cells with distinct effector functions and/or maturation markers (e.g., adaptive memory NK cells, tumor infiltrating lymphocytes, immature dendritic cells, monocyte-derived dendritic cells, plasmacytoid dendritic cells, conventional dendritic cells (sometimes referred to as classical dendritic cells), M1 macrophages, M2 macrophages, etc.), may be genetically modified to target the cells to a specific antigen and/or enhance the cells' anti-tumor effects (e.g., engineered T cell receptor (TCR) cellular therapies, chimeric antigen receptor (CAR) cellular therapies, lymph node homing of antigen-loaded dendritic cells, etc.), may be engineered to express of have increased expression of a tumor-associated antigen, or may be any combination thereof. Non-limiting types of cellular therapies include CAR-T cell therapy, CAR-NK cell therapy, TCR therapy, and dendritic cell vaccines. Exemplary cellular immunotherapies include sipuleucel-T, tisagenlecleucel, lisocabtagene maraleucel, idecabtagene vicleucel, brexucabtagene autoleucel, and axicabtagene ciloleucel, as well as CTX110, JCAR015, JCAR017, MB-CART19.1, MB-CART20.1, MB-CART2019.1, UniCAR02-T-CD123, BMCA-CAR-T, JNJ-68284528, BNT211, and NK-92/5.28.z.

In some embodiments, one or more of the additional therapeutic agents is an immunotherapeutic agent, more specifically a gene therapy. Gene therapies comprise recombinant nucleic acids administered to a subject or to a subject's cells ex vivo in order to modify the expression of an endogenous gene or to result in heterologous expression of a protein (e.g., small interfering RNA (siRNA) agents, double-stranded RNA (dsRNA) agents, micro RNA (miRNA) agents, viral or bacterial gene delivery, etc.), as well as gene editing therapies that may or may not comprise a nucleic acid component (e.g., meganucleases, zinc finger nucleases, TAL nucleases, CRISPR/Cas nucleases, etc.), oncolytic viruses, and the like. Non-limiting examples of gene therapies that may be useful in cancer treatment include Gendicine® (rAd-p53), Oncorine® (rAD5-H101), talimogene laherparepvec, Mx-dnGl, ARO-HIF2 (Arrowhead), quaratusugene ozeplasmid (Immunogene), CTX110 (CRISPR Therapeutics), CTX120 (CRISPR Therapeutics), and CTX130 (CRISPR Therapeutics).

In some embodiments, one or more of the additional therapeutic agents is an immunotherapeutic agent, more specifically an agent that modulates an immune checkpoint. Immune checkpoints are a set of inhibitory and stimulatory pathways that directly affect the function of immune cells (e.g., B cells, T cells, NK cells, etc.). Immune checkpoints engage when proteins on the surface of immune cells recognize and bind to their cognate ligands. The present invention contemplates the use of compounds described herein in combination with agonists of stimulatory or co-stimulatory pathways and/or antagonists of inhibitory pathways. Agonists of stimulatory or co-stimulatory pathways and antagonists of inhibitory pathways may have utility as agents to overcome distinct immune suppressive pathways within the tumor microenvironment, inhibit T regulatory cells, reverse/prevent T cell anergy or exhaustion, trigger innate immune activation and/or inflammation at tumor sites, or combinations thereof.

In some embodiments, one or more of the additional therapeutic agents is an immune checkpoint inhibitor. As used herein, the term “immune checkpoint inhibitor” refers to an antagonist of an inhibitory or co-inhibitory immune checkpoint. The terms “immune checkpoint inhibitor”, “checkpoint inhibitor” and “CP” may be used herein interchangeably. Immune checkpoint inhibitors may antagonize an inhibitory or co-inhibitory immune checkpoint by interfering with receptor-ligand binding and/or altering receptor signaling. Examples of immune checkpoints (ligands and receptors), some of which are selectively upregulated in various types of cancer cells, that can be antagonized include PD-1 (programmed cell death protein 1); PD-L1 (PD1 ligand); BTLA (B and T lymphocyte attenuator); CTLA-4 (cytotoxic T-lymphocyte associated antigen 4); TIM-3 (T cell immunoglobulin and mucin domain containing protein 3); LAG-3 (lymphocyte activation gene 3); TIGIT (T cell immunoreceptor with Ig and ITIM domains); CD276 (B7-H3), PD-L2, Galectin 9, CEACAM-1, CD69, Galectin-1, CD113, GPR56, VISTA, 2B4, CD48, GARP, PD1H, LAIR1, TIM-1, and TIM-4, and Killer Inhibitory Receptors, which can be divided into two classes based on their structural features: i) killer cell immunoglobulin-like receptors (KIRs), and ii) C-type lectin receptors (members of the type II transmembrane receptor family). Also contemplated are other less well-defined immune checkpoints that have been described in the literature, including both receptors (e.g., the 2B4 (also known as CD244) receptor) and ligands (e.g., certain B7 family inhibitory ligands such B7-H3 (also known as CD276) and B7-H4 (also known as B7-S1, B7x and VCTN1)). [See Pardoll, (April 2012) Nature Rev. Cancer 12:252-64].

In some embodiments, an immune checkpoint inhibitor is a CTLA-4 antagonist. In further embodiments, the CTLA-4 antagonist can be an antagonistic CTLA-4 antibody. Suitable antagonistic CTLA-4 antibodies include, for example, monospecific antibodies such as ipilimumab or tremelimumab, as well as bispecific antibodies such as MEDI5752 and KN046.

In some embodiments, an immune checkpoint inhibitor is a PD-1 antagonist. In further embodiments, the PD-1 antagonist can be an antagonistic PD-1 antibody, small molecule or peptide. Suitable antagonistic PD-1 antibodies include, for example, monospecific antibodies such as balstilimab, budigalimab, camrelizumab, cosibelimab, dostarlimab, cemiplimab, ezabenlimab, MEDI-0680 (AMP-514; WO2012/145493), nivolumab, pembrolizumab, pidilizumab (CT-011), pimivalimab, retifanlimab, sasanlimab, spartalizumab, sintilimab, tislelizumab, toripalimab, and zimberelimab; as well as bi-specific antibodies such as LY3434172, IBI321, ivonescimab, rilvegostomig, tebotelimab, and tobemstomig. In still further embodiments, the PD-1 antagonist can be a recombinant protein composed of the extracellular domain of PD-L2 (B7-DC) fused to the Fc portion of IgGl (AMP-224). In certain embodiments, an immune checkpoint inhibitor is zimberelimab.

In some embodiments, an immune checkpoint inhibitor is a PD-L1 antagonist. In further embodiments, the PD-L1 antagonist can be an antagonistic PD-L1 antibody. Suitable antagonistic PD-L1 antibodies include, for example, monospecific antibodies such as avelumab, atezolizumab, durvalumab, BMS-936559, and envafolimab as well as bi-specific antibodies such as LY3434172 and KN046.

In some embodiments, an immune checkpoint inhibitor is a TIGIT antagonist. In further embodiments, the TIGIT antagonist can be an antagonistic TIGIT antibody. Suitable antagonistic anti-TIGIT antibodies include monospecific antibodies such as AGEN1327, AB308 (WO2021247591), BMS 986207, COM902, domvanalimab, belrestotug, etigilimab, IBI-929, JS006, dargistotug, ociperlimab, SEA-TGT, tiragolumab, vibostolimab; as well as bi-specific antibodies such as AGEN1777 and rilvegostomig. In certain embodiments, an immune checkpoint inhibitor is an antagonistic anti-TIGIT antibody disclosed in WO2017152088 or WO2021247591. In certain embodiments, an immune checkpoint inhibitor is domvanalimab or AB308.

In some embodiments, an immune checkpoint inhibitor is a LAG-3 antagonist. In further embodiments, the LAG-3 antagonist can be an antagonistic LAG-3 antibody. Suitable antagonistic LAG-3 antibodies include, for example, BMS-986016 (WO10/19570, WO14/08218), or IMP-731 or IMP-321 (WO08/132601, WO09/44273).

In certain embodiments, an immune checkpoint inhibitor is a B7-H3 antagonist. In further embodiments, the B7-H3 antagonist is an antagonistic B7-H3 antibody. Suitable antagonist B7-H3 antibodies include, for example, enoblituzumab (WO11/109400), omburtumab, DS-7300a, ABBV-155, and SHR-A1811.

In some embodiments, an immune checkpoint inhibitor is a TIM-3 antagonist. In further embodiments, the TIM-3 antagonist can be an antagonistic TIM-3 antibody. Suitable antagonistic TIM-3 antibodies include, for example, sabatolimab, BMS-986258, and RG7769/RO7121661.

In some embodiments, one or more of the additional therapeutic agents activates a stimulatory or co-stimulatory immune checkpoint. Examples of stimulatory or co-stimulatory immune checkpoints (ligands andreceptors) include B7-1, B7-2, CD28, 4-1BB (CD137), 4-1BBL, ICOS, ICOS-L, OX40, OX40L, GITR, GITRL, CD70, CD27, CD40, DR3 and CD2.

In some embodiments, an agent that activates a stimulatory or co-stimulatory immune checkpoint is a CD137 (4-1BB) agonist. In further embodiments, the CD137 agonist can be an agonistic CD137 antibody. Suitable CD137 antibodies include, for example, urelumab and utomilumab (WO12/32433). In some embodiments, an agent that activates a stimulatory or co-stimulatory immune checkpoint is a GITR agonist. In further embodiments, the GITR agonist can be an agonistic GITR antibody. Suitable GITR antibodies include, for example, BMS-986153, BMS-986156, TRX-518 (WO06/105021, WO09/009116) and MK-4166 (WO11/028683). In some embodiments, an agent that activates a stimulatory or co-stimulatory immune checkpoint is an OX40 agonist. In further embodiments, the OX40 agonist can be an agonistic OX40 antibody. Suitable OX40 antibodies include, for example, MEDI-6383, MEDI-6469, MEDI-0562, PF-04518600, GSK3174998, BMS-986178, and MOXR0916. In some embodiments, an agent that activates a stimulatory or co-stimulatory immune checkpoint is a CD40 agonist. In further embodiments, the CD40 agonist can be an agonistic CD40 antibody. In some embodiments, an agent that activates a stimulatory or co-stimulatory immune checkpoint is a CD27 agonist. In further embodiments, the CD27 agonist can be an agonistic CD27 antibody. Suitable CD27 antibodies include, for example, varlilumab.

In some embodiments, one or more of the additional therapies is an immunotherapeutic agent, more specifically an intracellular signaling molecule that influences immune cell function. For example, one or more of the additional therapies may be an inhibitor of hematopoietic progenitor kinase 1 (HPK1). HPK1 is serine/threonine kinase that functions as a negative regulator of activation signals generated by the T cell antigen receptor. As another example, one or more of the additional therapies may be an inhibitor of diacylglycerol kinase (DGK). In some embodiments, the inhibitor is a small molecule. Non-limiting examples of small molecule HPK1 inhibitors in clinical development include NDI-101150, PRJ1-3024, PF-07265028, GRC 54276, CFI-402411 and BGB-15025. Non-limiting examples of small molecule DGK inhibitors include ASP1570, BAY2965501.

In some embodiments, one or more of the additional therapeutic agents is an agent that inhibits or depletes immune-suppressive immune cells. For example, to inhibit or deplete immunosuppressive macrophages or monocytes the agent may be CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264), or CSF-1R antagonists disclosed in WO14/036357. In another example, to inhibit or deplete regulatory T cells (Tregs), the agent may be an anti-CD25 antibody or immunotoxin targeting CD25.

In some embodiments, each additional therapeutic agent can independently be a chemotherapeutic agent, a radiopharmaceutical, a hormone therapy, an epigenetic modulator, a targeted agent, an immunotherapeutic agent, a cellular therapy, or a gene therapy. For example, in one embodiment, the present disclosure contemplates the use of the compounds described herein in combination with one or more chemotherapeutic agent and optionally one or more additional therapeutic agents, wherein each additional therapeutic agent is independently a radiopharmaceutical, a hormone therapy, a targeted agent, an immunotherapeutic agent, a cellular therapy, or a gene therapy. In another embodiment, the present disclosure contemplates the use of the compounds described herein in combination with one or more chemotherapeutic agent and optionally one or more additional therapeutic agents, wherein each additional therapeutic agent is independently a targeted agent, an immunotherapeutic agent, or a cellular therapy. In another embodiment, the present disclosure contemplates the use of the compounds described herein in combination with one or more immunotherapeutic agents and optionally one or more additional therapeutic agent, wherein each additional therapeutic agent is independently a radiopharmaceutical, a hormone therapy, a targeted agent, a chemotherapeutic agent, a cellular therapy, or a gene therapy. In another embodiment, the present disclosure contemplates the use of the compounds described herein in combination with one or more immunotherapeutic agents and optionally one or more additional therapeutic agents, wherein each additional therapeutic agent is independently a chemotherapeutic agent, a targeted agent, or a cellular therapy. In another embodiment, the present disclosure contemplates the use of the compounds described herein in combination with one or more immune checkpoint inhibitors and/or one or more ATP-adenosine axis-targeting agents, and optionally one or more additional therapeutic agents, wherein each additional therapeutic agent is independently a chemotherapeutic agent, a targeted agent, an immunotherapeutic agent, or a cellular therapy. In further embodiments of the above (a) the targeted agent can be a PI3K inhibitor, an arginase inhibitor, a HIF2α inhibitor, an AXL inhibitor, or a PAK4 inhibitor; (b) the immunotherapeutic agent is an ATP-adenosine axis-targeting agent or an immune checkpoint inhibitor; (c) the ATP-adenosine axis-targeting agent is an A2_{A}R and/or A2_{B}R antagonist, a CD73 inhibitor, or a CD39 inhibitor; (d) the ATP-adenosine axis-targeting agent is etrumadenant, quemliclustat, or AB598; (e) the immunotherapeutic agent is an anti-PD-1 antagonist antibody or an anti-TIGIT antagonist antibody; (f) the immunotherapeutic agent is zimberelimab, domvanalimab, or AB308; or (g) any combination thereof. In still further embodiments of the above, the present disclosure contemplates the use of the compounds described herein in combination with domvanalimab, etrumadenant, quemliclustat, zimberelimab, AB308, AB521, AB598, AB610, AB801 or any combination thereof.

Selection of the additional therapeutic agent(s) may be informed by current standard of care for a particular cancer and/or mutational status of a subject's cancer and/or stage of disease. Detailed standard of care guidelines are published, for example, by National Comprehensive Cancer Network (NCCN). See, for instance, NCCN Colon Cancer v1.2022, NCCN Hepatobiliary Cancer v.12022, NCCN Kidney Cancer, v3.2022, NCCN NSCLC v3.2022, NCCN Pancreatic Adenocarcinoma v.12022, NCCN Esophageal and Esophagogastric Junction Cancers v2.2022, NCCN Gastric Cancer v2.2022, Cervical Cancer v1.2022, Ovarian Cancer/Fallopian Tube Cancer/Primary Peritoneal Cancer v1.2022.

### EXPERIMENTAL

The following examples are put forth so as to provide those of ordinary skill in the art with a complete disclosure and description of how to make and use the present disclosure, and are not intended to limit the scope of what the inventors regard as their invention. Efforts have been made to ensure accuracy with respect to numbers used (e.g., amounts, temperature, etc.), but some experimental errors and deviations should be accounted for.

All reactions were performed using a Teflon-coated magnetic stir bar at the indicated temperature and were conducted under an inert atmosphere when stated. Purchased starting materials and reagents were generally used as received. Reactions were monitored by TLC (silica gel 60 with fluorescence F254, visualized with a short wave/long wave UV lamp) and/or LCMS (AGILENT® 1100 or 1200 series LCMS with UV detection at 254 or 280 nm using a binary solvent system [0.1% formic acid in MeCN/0.1% formic acid in H₂O] using one of the following columns: AGILENT® Eclipse Plus C18 [3.5 μm, 4.6 mm i.d.×100 mm], WATERS™ XSelect HSS C18 [3.5 μm, 2.1 mm i.d.×75 mm]). Flash chromatography was conducted on silica gel using an automated system (COMBIFLASH® RF+ manufactured by Teledyne ISCO), with detection wavelengths of 254 and 280 nm, and optionally equipped with an evaporative light scattering detector. Reverse phase preparative HPLC was conducted on an AGILENT® 1260 or 1290 Infinity series HPLC. Samples were eluted using a binary solvent system (MeCN/H₂O with an acid modifier as needed—for example 0.1% TFA or 0.1% formic acid) with gradient elution on a Gemini C18 110 Å column (21.2 mm i.d.×250 mm) with variable wavelength detection. Final compounds obtained through preparative HPLC were concentrated through lyophilization. All assayed compounds were purified to >95% purity as determined by ¹H NMR or LCMS (AGILENT® 1100 or 1200 series LCMS with UV detection at 254 or 280 nm using a binary solvent system [0.1% formic acid in MeCN/0.1% formic acid in H₂O] using one of the following columns: AGILENT® Eclipse Plus C18 [3.5 μm, 4.6 mm i.d.×100 mm], WATERS™ XSelect HSS C18 [3.5 μm, 2.1 mm i.d.×75 mm]). ¹H NMR spectra were recorded on a Varian 400 MHz NMR spectrometer equipped with an Oxford AS400 magnet or a BRUKER® AVANCE NEO 400 MHz NMR. Chemical shifts (δ) are reported as parts per million (ppm) relative to residual undeuterated solvent as an internal reference. The abbreviations s, br s, d, t, q, dd, dt, ddd, and m stand for singlet, broad singlet, doublet, triplet, quartet, doublet of doublets, doublet of triplets, doublet of doublet of doublets, and multiplet, respectively.

Unless indicated otherwise, temperature is in degrees Celsius (° C.), and pressure is at or near atmospheric. Standard abbreviations are used, including the following: rt=room temperature; min(s)=minute(s); h=hour(s); mg=milligram; g=gram; kg=kilogram; L=microliter; ml or mL=milliliter; M=molar; mol=mole; mmol=millimole; N=normality; sat.=saturated; aq.=aqueous; psi=pounds per square inch; calcd=calculated; equiv.=equivalents; DCM=dichloromethane; THF=tetrahydrofuran; EtOAc=ethyl acetate; TFA=trifluoroacetic acid; MeCN=acetonitrile; DMF=N,N-dimethylformamide; DMSO=dimethyl sulfoxide; DMA=dimethylacetamide; NMP=N-methyl-2-pyrrolidone; CHCl₃=chloroform; CDCl₃=deuterated chloroform; iPrOH=isopropyl alcohol; EtOH=ethanol; MeOH=methanol; HFIP=hexafluoroisopropanol; N₂=nitrogen gas; H₂O₂=hydrogen peroxide; DIPEA and i-Pr₂NEt=N,N-diisopropylethylamine; DMEDA=N,N-dimethylethane-1,2-diamine; SEMCl=2-(trimethylsilyl)ethoxymethyl chloride; TES-Cl=chlorotriethylsilane; Et₃N=triethylamine; NaBH₃CN=sodium cyanoborohydride; NaBH₄=sodium borohydride; NaBH(OAc)₃=sodium triacetoxyborohydride; NH₄Cl=ammonium chloride; NH₃=ammonia; Na₂SO₄=sodium sulfate; NaHCO₃=sodium bicarbonate; NaH=sodium hydride; NaOH=sodium hydroxide; Na₂S₂O₃=sodium thiosulfate; KI=potassium iodide; K₂CO₃=potassium carbonate; K₃PO₄=potassium phosphate; nBuLi=n-butyllithium; LiTMP=lithium tetramethylpiperidide; ClCO₂Et and ClCOOEt=ethyl chloroformate; LDA=lithium diisopropylamide; B₂pin₂=bis(pinacolato)diboron; NCS=N-chlorosuccinimide; NIS=N-iodosuccinimide; CuCN=copper(I) cyanide; Cu(OTf)₂=copper(II) trifluoromethanesulfonate; CuI=copper iodide; CuBr₂=copper(II) bromide; MeMgBr-methylmagnesium bromide; HCl=hydrochloric acid; TMSCl=trimethylsilyl chloride; MsCl=methanesulfonyl chloride; HATU=1-[bis(dimethylamino)methylene]-1H-1,2,3-triazolo[4,5-b]pyridinium 3-oxide hexafluorophosphate; Pd/C=palladium on carbon; Pd(dppf)Cl₂=[1,1′-bis(diphenylphosphino)ferrocene]palladium(II) dichloride; t-BuXPhosPd G3=[(2-di-tert-butylphosphino-2′,4′,6′-triisopropyl-1,1′-biphenyl)-2-(2′-amino-1,1′-biphenyl)]palladium(II) methanesulfonate; Xphos Pd G3=(2-dicyclohexylphosphino-2′,4′,6′-triisopropyl-1,1′-biphenyl)[2-(2′-amino-1,1′-biphenyl)]palladium(II) methanesulfonate; Xphos=dicyclohexyl[2′,4′,6′-tris(propan-2-yl)[1,1′-biphenyl]-2-yl]phosphane; Pd(PPh₃)₄=tetrakis(triphenylphosphine)palladium(0); MnO₂=manganese dioxide; SnCl₂=tin(II) chloride; Zn(CN)₂=zinc cyanide; Boc₂O=di-tert-butyl dicarbonate; MS 4A=molecular sieves, 4A; MHz=megahertz; Hz=hertz; ppm=parts per million; ESI MS=electrospray ionization mass spectrometry; LCMS=liquid chromatography-mass spectrometry; NMR=nuclear magnetic resonance; HPLC=high pressure liquid chromatography.

### Example 1: 2-[2-cyclopropyl-6-[4-cyclopropyl-2-[[(3S)-3-methylpiperidin-1-yl]methyl]-7-oxo-1H-pyrrolo[2,3-c]pyridin-6-yl]pyridin-4-yl]-5-fluoro-N,N-dimethylbenzamide

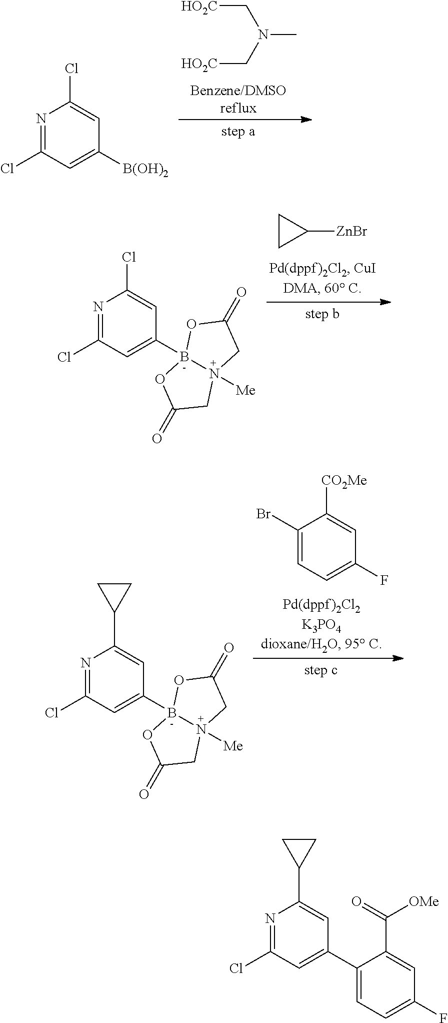

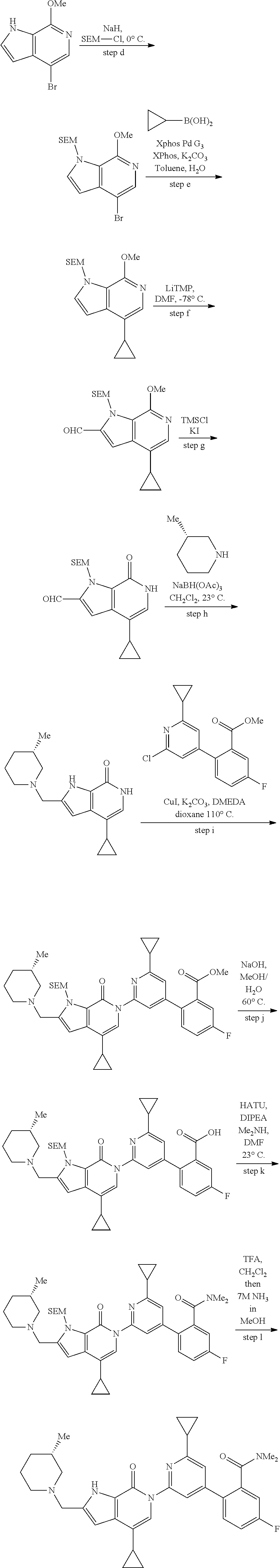

Step a: To a 2 L round bottom flask was added (2,6-dichloropyridin-4-yl)boronic acid (13.8 g, 72.2 mmol, 1.0 equiv.) and 2-[carboxymethyl(methyl)amino]acetic acid (10.6 g, 72.2 mmol, 1.0 equiv.). The reagents were dissolved in a 9:1 mixture of benzene/DMSO (720 mL), a Dean-Stark apparatus was attached to the round bottom flask, and the reaction mixture was heated to a vigorous reflux for 16 hours. The reaction mixture was cooled to room temperature and benzene was evaporated under reduced pressure. The remaining DMSO solution was poured into 600 mL of water. The resulting solid was collected by filtration, washed with water (2×300 mL), and dried on the filter until constant weight was obtained to afford the corresponding MIDA-boronate. ¹H NMR (400 MHz, DMSO-d₆) δ 7.54 (s, 2H), 4.39 (d, J=17.2 Hz, 2H), 4.18 (d, J=17.1 Hz, 2H), 2.66 (s, 3H).

Step b: A solution of the product from step a (21.6 g, 71.3 mmol, 1.0 equiv.) in DMA (50 mL) was sparged with N₂ for 15 minutes at which point CuI (552 mg, 2.9 mmol, 0.04 equiv.) and Pd(dppf)₂Cl₂ (1.54 g, 2.1 mmol, 0.03 equiv.) were added. Bromo(cyclopropyl)zinc (0.5 M in THF, 107 mmol, 214 mL, 1.5 equiv.) was added via cannula in a continuous stream, and the reaction mixture was heated to 60° C. and stirred for 2 hours under N₂. Once complete consumption of the starting material was observed via ¹H NMR monitoring the reaction mixture was cooled to room temperature and poured into saturated aqueous NH₄Cl (1 L) and extracted with EtOAc (2×350 mL). The combined organics were dried over Na₂SO₄, filtered, and concentrated under vacuum. The crude residue was triturated with dichloromethane to induce the precipitation of the desired product that was subsequently collected by filtration. ¹H NMR (400 MHz, DMSO-d₆) δ 7.32 (d, J=0.9 Hz, 1H), 7.19 (d, J=0.8 Hz, 1H), 4.37 (d, J=17.2 Hz, 2H), 4.18 (d, J=17.2 Hz, 2H), 2.61 (s, 3H), 2.11 (tt, J=8.1, 4.8 Hz, 1H), 1.02-0.95 (m, 2H), 0.93-0.86 (m, 2H). ESI MS [M+H]⁺ for C₁₃H₁₄BClN₂O₄, calcd 309.1, found 309.0.

Step c: A 40 mL vial was charged with 1-(2-chloro-6-cyclopropylpyridin-4-yl)-5-methyl-2,8-dioxa-5-azonia-1-boranuidabicyclo[3.3.0]octane-3,7-dione (300 mg, 1.0 mmol, 1 equiv.), methyl 2-bromo-5-fluorobenzoate (233 mg, 1 mmol. 1 equiv.) and K₃PO₄ (636 mg, 3.0 mmol, 3 equiv.). The reagents were suspended in the 4:1 mixture of dioxane/water (10 mL) and the resulting solution was sparged with N₂ for 10 minutes. Then, Pd(dppf)Cl₂ (73 mg, 0.1 mmol, 10%) was added, and the mixture was heated to 95° C. for 6 hours. The reaction mixture was partitioned between EtOAc and water. The organic phase was separated, and the aqueous phase was additionally extracted three times with EtOAc. The combined organics were dried over Na₂SO₄ and concentrated to dryness under reduced pressure. The crude residue was purified via silica gel flash column chromatography (0 to 50% EtOAc/hexane) to afford methyl 2-(2-chloro-6-cyclopropylpyridin-4-yl)-5-fluorobenzoate.

Step d: To a solution of 4-bromo-7-methoxy-1H-pyrrolo[2,3-c]pyridine (8.0, 35.2 mmol, 1.0 equiv.) in THF (100 ml, 0.3 M) was add NaH (2.54 g, 105.7 mmol, 3.0 equiv.) and SEMCl (6.5 g, 38.8 mmol, 1.1 equiv.) at 0° C. The resulting mixture was stirred at 23° C. for 2 h. The reaction mixture was quenched with H₂O, the organic phase was separated, and the aqueous layer was extracted with EtOAc. The combined organic phase was dried over Na₂SO₄, concentrated and the crude residue was purified by column chromatography (SiO₂, EtOAc in hexanes, 20 to 80%) to produce 2-[(4-bromo-7-methoxypyrrolo[2,3-c]pyridin-1-yl)methoxy]ethyl-trimethylsilane.

Step e: The product of step d (6.60 g, 18.4 mmol, 1.0 equiv.), cyclopropylboronic acid (2.0 g, 23.0 mmol, 1.25 equiv.) and K₂CO₃ (7.60 g, 55.3 mmol, 3.0 equiv.) were dissolved in toluene/H₂O (60 mL/12 ml, 0.25 M). The mixture was purged for 2 mins under N₂. Then, Xphos Pd G3 (780 mg, 0.9 mmol, 0.05 equiv.) and Xphos (703 mg, 1.5 mmol, 0.08 equiv.) were added into the solution. The mixture was stirred at 90° C. for 12 h. The reaction was cooled to room temperature and quenched with H₂O. The organic phase was separated, and the aqueous layer was extracted with EtOAc. The combined organic phase was dried over Na₂SO₄ and concentrated. The crude residue was purified by column chromatography (SiO₂, EtOAc in hexanes, 20 to 80%) to afford 2-[(4-cyclopropyl-7-methoxypyrrolo[2,3-c]pyridin-1-yl)methoxy]ethyl-trimethylsilane.

Step f: To a solution of 2,2,6,6-tetramethylpiperidine (1.53 ml, 9.0 mmol, 1.6 equiv.) in THF (50 mL, 0.18 M) was added dropwise n-butyllithium solution (3.6 ml, 9.0 mmol, 1.6 equiv., 2.5 M) at −78° C. The resulting mixture was stirred at −78° C. for 5 min. To the formed LiTMP solution was added the product of step b (1.7881 g, 5.6141 mmol, 1.0 equiv.) at −78° C. in THF. The reaction was stirred at −78° C. for 1 h. Then DMF (0.8 ml, 10.1 mmol, 1.8 equiv.) was added, and the mixture was stirred for 30 min at −78° C. The reaction was quenched with sat. aq. NH₄Cl solution. The organic phase was separated, and the aqueous layer was extracted with EtOAc. The combined organic phase was dried over Na₂SO₄ and concentrated to afford 2-[(4-cyclopropyl-7-methoxypyrrolo[2,3-c]pyridin-1-yl)methoxy]ethyl-trimethylsilane that was used for the next step without purification.

Step g: To a solution of the product from step f (1.95 g, 5.6 mmol, 1.0 equiv.) and KI (1.50 g, 9.0 mmol, 1.6 equiv.) in MeCN (50 mL, 0.1 M) was added TMSCl (1.0 g, 8.96 mmol, 1.6 equiv.) followed by water (0.1 ml). The resulting mixture was stirred at 23° C. for 12 h. The mixture was quenched with H₂O and diluted with EtOAc. The organic phase was separated, and the aqueous phase was extracted with EtOAc, the combined organic extract was washed with brine, dried over Na₂SO₄ and concentrated under reduced pressure. The crude residue was purified by column chromatography (SiO₂, MeOH in DCM, 0 to 10%) to give 4-cyclopropyl-7-oxo-1-(2-trimethylsilylethoxymethyl)-6H-pyrrolo[2,3-c]pyridine-2-carbaldehyde.

Step h: To the aldehyde of step g (1.6 g, 5 mmol, 1.0 equiv.) in DCM (50 mL, 0.1 M) was added (S)-3-methylpiperidine hydrochloride (0.67 g, 5 mmol, 1.0 equiv.) and i-Pr₂NEt (1.74 mL, 10 mmol, 2.0 equiv.). The mixture was stirred at 23° C. for 10 mins, then NaBH(OAc)₃ (1.59 g, 7.5 mmol, 1.5 equiv.) was added, and the mixture was stirred at 23° C. for additional 12 h. The reaction was quenched with aq. sat. NaHCO₃, the organic phase was separated, and the aqueous layer was extracted with dichloromethane. The combined organic phase was dried over Na₂SO₄ and concentrated. The crude residue was purified by column chromatography (SiO₂, MeOH in DCM, 0 to 10%) to give 4-cyclopropyl-2-[[(3S)-3-methylpiperidin-1-yl]methyl]-1,6-dihydropyrrolo[2,3-c]pyridin-7-one.

Step i: To a solution of methyl 2-(2-chloro-6-cyclopropylpyridin-4-yl)-5-fluorobenzoate (210 mg, 0.68 mmol, 1.0 equiv.) and 4-cyclopropyl-2-[[(3S)-3-methylpiperidin-1-yl]methyl]-1,6-dihydropyrrolo[2,3-c]pyridin-7-one (286 mg, 0.68 mmol, 1.0 equiv.) in dioxane (13.6 mL, 0.05 M) was added CuI (130 mg, 0.68 mmol, 1 equiv.), 1,2-dimethylethylenediamine (120 mg, 1.36 mmol, 2.0 equiv.) and K₂CO₃ (281 mg, 2.04 mmol, 3.0 equiv.). The resulting solution was stirred at 110° C. for 18 h, the reaction was quenched with aq. sat. NH₄Cl and diluted with water and EtOAc. The organic phase was separated, the aqueous layer was additionally extracted with EtOAc. The combined organic phase was dried over Na₂SO₄, concentrated and the crude residue was purified by column chromatography (EtOAc in hexane, 0 to 100%) to afford the methyl 2-[2-cyclopropyl-6-[4-cyclopropyl-2-[[(3S)-3-methylpiperidin-1-yl]methyl]-7-oxo-1-(2-trimethyl-silylethoxymethyl)pyrrolo[2,3-c]pyridin-6-yl]pyridin-4-yl]-5-fluorobenzoate.

Step j: To a solution of the product from step i (180 mg, 0.26 mmol, 1 equiv.) in MeOH/H₂O (1:1, 1.5 mL), was added NaOH (52.63 mg, 1.3 mmol, 5 equiv.). The resulting mixture was stirred at 60° C. for 4 h. Then the reaction was allowed to cool to ambient temperature and acidified with 1M HCl to pH-2-3. The product was extracted with EtOAc three times. The combined organics were dried over Na₂SO₄ and concentrated under reduced pressure to produce 2-[2-cyclopropyl-6-[4-cyclopropyl-2-[[(3S)-3-methylpiperidin-1-yl]methyl]-7-oxo-1-(2-trimethylsilylethoxymethyl)pyrrolo[2,3-c]pyridin-6-yl]pyridin-4-yl]-5-fluorobenzoic acid that was used in the subsequent step without further purification.

Step k: To a solution of a product from step j (90 mg, 0.13 mmol, 1 equiv.) in THF (1.2 mL, 0.1 M), DIPEA (45 mL, 0.26 mmol, 2 equiv.) HATU (72 mg, 0.19 mmol, 1.5 equiv.) and dimethylamine (2M in THF, 0.13 mL, 2 equiv.) were added. The reaction mixture was stirred overnight, then quenched with water and diluted with EtOAc. The organic phase was separated and washed with brine, dried over Na₂SO₄ and concentrated under vacuum. The crude residue was purified by silica gel flash column chromatography (0 to 20% MeOH/DCM) to afford 2-[2-cyclopropyl-6-[4-cyclopropyl-2-[[(3S)-3-methylpiperidin-1-yl]methyl]-7-oxo-1-(2-trimethyl-silylethoxymethyl)pyrrolo[2,3-c]pyridin-6-yl]pyridin-4-yl]-5-fluoro-N,N-dimethylbenzamide.

Step l: The amide product from step k (69 mg, 0.1 mmol, 1 equiv) was dissolved in dichloromethane (0.7 mL) and TFA (0.7 mL). The mixture was stirred for 3 h and concentrated to dryness under vacuum. The residual TFA was removed by co-evaporation with dichloromethane. Then the residue was dissolved in methanolic solution of ammonia (3 mL, 7 N), and the mixture was stirred for 3 h at room temperature. The reaction mixture was concentrated, and the crude product was purified by preparative HPLC (20% to 90% MeCN/water, 0.1% TFA) to afford the title compound. ¹H NMR (400 MHz, CDCl₃) δ 9.63 (s, 1H), 7.71 (d, J=1.4 Hz, 1H), 7.49-7.42 (m, 1H), 7.30 (d, J=1.2 Hz, 1H), 7.19-7.12 (m, 3H), 6.36 (d, J=1.8 Hz, 1H), 3.59 (d, J=3.4 Hz, 2H), 2.94 (s, 3H), 2.83-2.71 (m, 2H), 2.61 (s, 3H), 2.14-2.06 (m, 1H), 1.99-1.85 (m, 3H), 1.72-1.52 (m, 5H), 1.08-0.99 (m, 4H), 0.91-0.85 (m, 2H), 0.82 (d, J=5.5 Hz, 3H), 0.72-0.66 (m, 2H). ESI MS [M+H]+ for C₃₄H₃₈FN₅O₂, calcd 568.3, found 568.3.

### Example 2: 2-[2-cyclopropyl-6-[4-cyclopropyl-2-[[(3S)-3-methylpiperidin-1-yl]methyl]-7-oxo-1H-pyrrolo[2,3-c]pyridin-6-yl]pyridin-4-yl]-N-(2-ethoxyethyl)-5-fluorobenzamide

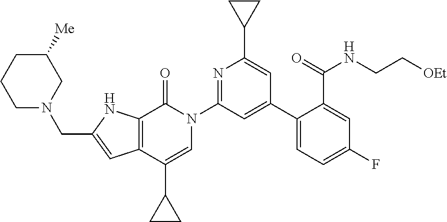 [SMILES: CCOCCNC(=O)c1cc(F)ccc1-c1cc(C2CC2)nc(-n2cc(C3CC3)c3cc(CN4CCC[C@H](C)C4)[nH]c3c2=O)c1]

The title compound was prepared in a similar fashion to that described for Example 1 from 2-[2-cyclopropyl-6-[4-cyclopropyl-2-[[(3S)-3-methylpiperidin-1-yl]methyl]-7-oxo-1-(2-trimethylsilylethoxymethyl)pyrrolo[2,3-c]pyridin-6-yl]pyridin-4-yl]-5-fluorobenzoic acid and 2-ethoxyethylamine. ¹H NMR (400 MHz, CDCl₃) δ 10.23 (s, 1H), 7.76 (d, J=1.3 Hz, 1H), 7.42 (dd, J=8.5, 5.3 Hz, 1H), 7.35 (dd, J=8.7, 2.7 Hz, 1H), 7.30 (d, J=1.1 Hz, 1H), 7.22-7.14 (m, 2H), 6.63 (s, 1H), 6.33 (s, 1H), 3.60 (d, J=2.3 Hz, 2H), 3.50-3.42 (m, 2H), 3.36-3.25 (m, 4H), 2.81-2.67 (m, 2H), 2.14-2.03 (m, 1H), 1.97-1.79 (m, 4H), 1.67-1.41 (m, 4H), 1.10-1.05 (m, 2H), 1.05-1.01 (m, 4H), 0.91-0.85 (m, 3H), 0.79 (d, J=6.6 Hz, 3H), 0.70-0.64 (m, 2H). ESI MS [M+H]⁺ for C₃₆H₄₂FN₅O₃, calcd 612.2, found 612.2.

### Example 3: 2-{[(S)-3-methyl-1-piperidyl]methyl}-4-cyclopropyl-6-[6-cyclopropyl-4-(4-fluoro-2-morpholinocarbonylphenyl)-2-pyridyl]-1,6-dihydro-1,6-diaza-7-indenone

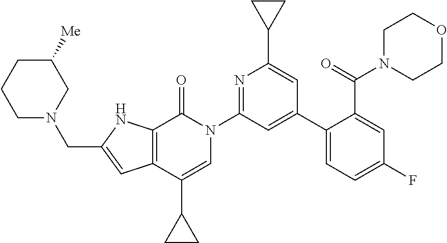 [SMILES: C[C@H]1CCCN(Cc2cc3c(C4CC4)cn(-c4cc(-c5ccc(F)cc5C(=O)N5CCOCC5)cc(C5CC5)n4)c(=O)c3[nH]2)C1]

The title compound was prepared in a similar fashion to that described for Example 1 from 2-[2-cyclopropyl-6-[4-cyclopropyl-2-[[(3S)-3-methylpiperidin-1-yl]methyl]-7-oxo-1-(2-trimethylsilylethoxymethyl)pyrrolo[2,3-c]pyridin-6-yl]pyridin-4-yl]-5-fluorobenzoic acid and morpholine. ¹H NMR (400 MHz, CDCl₃) δ 9.61 (s, 1H), 7.77 (d, J=1.4 Hz, 1H), 7.48 (dd, J=8.5, 5.2 Hz, 1H), 7.34 (d, J=1.2 Hz, 1H), 7.23-7.13 (m, 3H), 6.36 (d, J=1.7 Hz, 1H), 3.76-3.53 (m, 6H), 3.46-3.36 (m, 2H), 3.12-2.88 (m, 2H), 2.87-2.66 (m, 3H), 2.10 (td, J=7.8, 3.9 Hz, 1H), 1.97-1.84 (m, 2H), 1.73-1.51 (m, 5H), 1.12-0.99 (m, 4H), 0.91-0.87 (m, 2H), 0.82 (d, J=5.5 Hz, 3H), 0.70 (dt, J=5.6, 3.0 Hz, 2H). ESI MS [M+H]⁺ for C₃₆H₄₀FN₅O₃, calcd 610.3, found 610.3.

### Example 4: 2-{[(S)-3-methyl-1-piperidyl]methyl}-4-cyclopropyl-6-(6-cyclopropyl-4-{4-fluoro-2-[(1-pyrrolidinyl)carbonyl]phenyl}-2-pyridyl)-1,6-dihydro-1,6-diaza-7-indenone

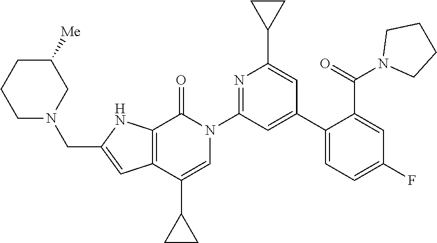 [SMILES: C[C@H]1CCCN(Cc2cc3c(C4CC4)cn(-c4cc(-c5ccc(F)cc5C(=O)N5CCCC5)cc(C5CC5)n4)c(=O)c3[nH]2)C1]

The title compound was prepared in a similar fashion to that described for Example 1 from 2-[2-cyclopropyl-6-[4-cyclopropyl-2-[[(3S)-3-methylpiperidin-1-yl]methyl]-7-oxo-1-(2-trimethylsilylethoxymethyl)pyrrolo[2,3-c]pyridin-6-yl]pyridin-4-yl]-5-fluorobenzoic acid and pyrrolidine. ¹H NMR (400 MHz, CDCl₃) δ 9.52 (s, 1H), 7.74 (d, J=1.4 Hz, 1H), 7.51-7.45 (m, 1H), 7.29 (d, J=1.2 Hz, 1H), 7.21 (d, J=1.4 Hz, 1H), 7.19-7.13 (m, 2H), 6.35 (s, 1H), 3.59 (d, J=3.2 Hz, 2H), 3.48 (t, J=7.0 Hz, 2H), 2.94 (t, J=6.7 Hz, 2H), 2.84-2.72 (m, 2H), 2.11 (tt, J=7.9, 5.0 Hz, 1H), 1.98-1.85 (m, 2H), 1.81-1.69 (m, 3H), 1.69-1.56 (m, 3H), 1.09-0.98 (m, 4H), 0.93-0.85 (m, 3H), 0.83 (d, J=5.6 Hz, 4H), 0.72-0.64 (m, 2H). ESI MS [M+H]⁺ for C₃₆H₄₀FN₅O₂ calcd 594.3, found 594.3.

### Example 5: N,N-diethyl2-[2-(2-{[(S)-3-methyl-1-piperidyl]methyl}-4-cyclopropyl-7-oxo-1,6-dihydro-1,6-diaza-6-indenyl)-6-cyclopropyl-4-pyridyl]-5-fluorobenzamide

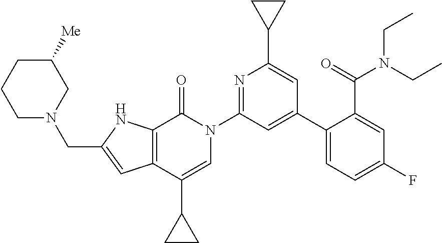 [SMILES: CCN(CC)C(=O)c1cc(F)ccc1-c1cc(C2CC2)nc(-n2cc(C3CC3)c3cc(CN4CCC[C@H](C)C4)[nH]c3c2=O)c1]

The title compound was prepared in a similar fashion to that described for Example 1 from 2-[2-cyclopropyl-6-[4-cyclopropyl-2-[[(3S)-3-methylpiperidin-1-yl]methyl]-7-oxo-1-(2-trimethylsilylethoxymethyl)pyrrolo[2,3-c]pyridin-6-yl]pyridin-4-yl]-5-fluorobenzoic acid and diethylamine. ¹H NMR (400 MHz, CDCl₃) δ 9.64 (s, 1H), 7.71 (s, 1H), 7.46 (dd, J=8.6, 5.3 Hz, 1H), 7.29 (s, 1H), 7.26 (s, 1H), 7.19-7.08 (m, 2H), 6.35 (s, 1H), 3.80 (s, 1H), 3.64-3.52 (m, 2H), 3.16-2.83 (m, 3H), 2.81-2.69 (m, 2H), 2.11-2.03 (m, 1H), 1.96-1.85 (m, 2H), 1.73-1.49 (m, 6H), 1.05-0.92 (m, 6H), 0.91-0.85 (m, 6H), 0.82 (d, J=5.7 Hz, 3H), 0.71-0.62 (m, 2H). ESI MS [M+H]⁺ for C₃₆H₄₂FN₅O₂ calcd 596.3, found 596.4.

### Example 6: N-methyl2-[2-(2-{[(S)-3-methyl-1-piperidyl]methyl}-4-cyclopropyl-7-oxo-1,6-dihydro-1,6-diaza-6-indenyl)-6-cyclopropyl-4-pyridyl]-5-fluorobenzamide

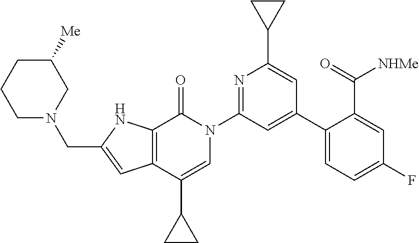 [SMILES: CNC(=O)c1cc(F)ccc1-c1cc(C2CC2)nc(-n2cc(C3CC3)c3cc(CN4CCC[C@H](C)C4)[nH]c3c2=O)c1]

The title compound was prepared in a similar fashion to that described for Example 1 from 2-[2-cyclopropyl-6-[4-cyclopropyl-2-[[(3S)-3-methylpiperidin-1-yl]methyl]-7-oxo-1-(2-trimethylsilylethoxymethyl)pyrrolo[2,3-c]pyridin-6-yl]pyridin-4-yl]-5-fluorobenzoic acid and methylamine. ¹H NMR (400 MHz, CDCl₃) δ 11.45 (s, 1H), 7.86-7.81 (m, 1H), 7.54 (s, 1H), 7.39 (dd, J=8.6, 5.2 Hz, 1H), 7.35-7.30 (m, 2H), 7.23 (s, 1H), 7.16 (td, J=8.3, 2.7 Hz, 1H), 6.28 (s, 1H), 3.67-3.51 (m, 2H), 2.81 (d, J=4.7 Hz, 3H), 2.75-2.61 (m, 2H), 2.16-2.07 (m, 1H), 1.97-1.89 (m, 2H), 1.70-1.41 (m, 3H), 1.16-0.97 (m, 6H), 0.92-0.86 (m, 2H), 0.74-0.59 (m, 6H). ESI MS [M+H]⁺ for C₃₃H₃₆FN₅O₂ calcd 554.2, found 554.3.

### Example 7: 2-{[(S)-3-methyl-1-piperidyl]methyl}-6-(4-{2-[(1-azetidinyl)carbonyl]-4-fluorophenyl}-6-cyclopropyl-2-pyridyl)-4-cyclopropyl-1,6-dihydro-1,6-diaza-7-indenone

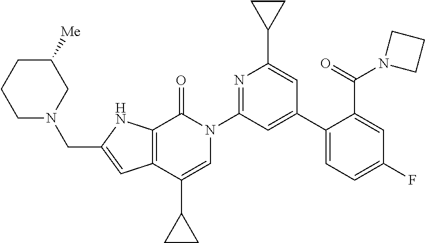 [SMILES: C[C@H]1CCCN(Cc2cc3c(C4CC4)cn(-c4cc(-c5ccc(F)cc5C(=O)N5CCC5)cc(C5CC5)n4)c(=O)c3[nH]2)C1]

The title compound was prepared in a similar fashion to that described for Example 1 from 2-[2-cyclopropyl-6-[4-cyclopropyl-2-[[(3S)-3-methylpiperidin-1-yl]methyl]-7-oxo-1-(2-trimethylsilylethoxymethyl)pyrrolo[2,3-c]pyridin-6-yl]pyridin-4-yl]-5-fluorobenzoic acid and azetidine. ¹H NMR (400 MHz, CDCl₃) δ 9.56 (s, 1H), 7.77 (d, J=1.4 Hz, 1H), 7.44 (dd, J=8.6, 5.3 Hz, 1H), 7.34 (d, J=1.2 Hz, 1H), 7.25-7.11 (m, 3H), 6.36 (d, J=1.9 Hz, 1H), 4.03 (t, J=7.8 Hz, 2H), 3.68 (t, J=7.8 Hz, 2H), 3.66-3.53 (m, 2H), 2.84-2.71 (m, 2H), 2.20-2.04 (m, 3H), 1.98-1.87 (m, 2H), 1.74-1.49 (m, 5H), 1.12-1.00 (m, 4H), 0.92-0.84 (m, 3H), 0.83 (d, J=5.1 Hz, 3H), 0.72-0.67 (m, 2H). ESI MS [M+H]⁺ for C₃₅H₃₈FN₅O₂ calcd 580.3, found 580.3.

### Example 8: N-methyl-N-phenyl-2-[2-(2-{[(S)-3-methyl-1-piperidyl]methyl}-4-cyclopropyl-7-oxo-1,6-dihydro-1,6-diaza-6-indenyl)-6-cyclopropyl-4-pyridyl]-5-fluorobenzamide

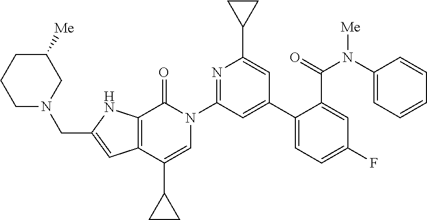 [SMILES: C[C@H]1CCCN(Cc2cc3c(C4CC4)cn(-c4cc(-c5ccc(F)cc5C(=O)N(C)c5ccccc5)cc(C5CC5)n4)c(=O)c3[nH]2)C1]

The title compound was prepared in a similar fashion to that described for Example 1 from 2-[2-cyclopropyl-6-[4-cyclopropyl-2-[[(3S)-3-methylpiperidin-1-yl]methyl]-7-oxo-1-(2-trimethylsilylethoxymethyl)pyrrolo[2,3-c]pyridin-6-yl]pyridin-4-yl]-5-fluorobenzoic acid and N-methylaniline. ¹H NMR (400 MHz, CDCl₃) δ 9.66 (s, 1H), 7.33 (dd, J=8.7, 2.8 Hz, 1H), 7.22 (s, 1H), 7.17-7.12 (m, 2H), 7.03 (td, J=8.3, 2.7 Hz, 1H), 6.98-6.93 (m, 2H), 6.71 (s, 1H), 6.51-6.31 (m, 3H), 3.64-3.58 (m, 2H), 3.39 (s, 3H), 2.85-2.72 (m, 2H), 2.11-2.03 (m, 1H), 1.96-1.87 (m, 2H), 1.74-1.53 (m, 5H), 1.07-1.01 (m, 4H), 0.91-0.80 (m, 7H), 0.72-0.66 (m, 2H). ESI MS [M+H]⁺ for C₃₉H₄₀FN₅O₂ calcd 630.3, found 630.3.

### Example 9: 2-{[(S)-3-methyl-1-piperidyl]methyl}-4-cyclopropyl-6-(6-cyclopropyl-4-{4-fluoro-2-[(2-methyl-1-pyrrolidinyl)carbonyl]phenyl}-2-pyridyl)-1,6-dihydro-1,6-diaza-7-indenone

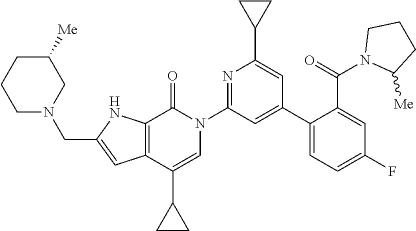 [SMILES: CC1CCCN1C(=O)c1cc(F)ccc1-c1cc(C2CC2)nc(-n2cc(C3CC3)c3cc(CN4CCC[C@H](C)C4)[nH]c3c2=O)c1]

The title compound was prepared as an inseparable diastereomeric mixture in 3:1 ratio in a similar fashion to that described for Example 1 from 2-[2-cyclopropyl-6-[4-cyclopropyl-2-[[(3S)-3-methylpiperidin-1-yl]methyl]-7-oxo-1-(2-trimethylsilylethoxymethyl)pyrrolo[2,3-c]pyridin-6-yl]pyridin-4-yl]-5-fluorobenzoic acid and 2-methylpyrrolidine. ¹H NMR (400 MHz, CDCl₃) δ 9.56 (s, 1.4H), 7.75 (d, J=1.4 Hz, 1H), 7.73 (s, 0.3H), 7.50-7.43 (m, 1.3H), 7.31 (d, J=1.3 Hz, 1H), 7.28-7.26 (m, 1H), 7.23 (d, J=1.4 Hz, 0.3H), 7.22-7.18 (m, 0.6H), 7.17-7.13 (m, 2H), 7.13-7.11 (m, 0.3H), 6.35 (s, 1.3H), 4.28-4.16 (m, 1H), 3.61-3.37 (m, 4H), 3.00-2.87 (m, 2H), 2.83-2.69 (m, 3H), 2.19-2.01 (m, 2H), 1.99-1.85 (m, 4H), 1.81-1.51 (m, 12H), 1.46-1.34 (m, 2H), 1.15-1.05 (m, 4H), 1.05-0.96 (m, 5H), 0.92-0.86 (m, 4H), 0.84-0.78 (m, 6H), 0.72-0.63 (m, 2.8H). ESI MS [M+H]⁺ for C₃₇H₄₃FN₅O₂ calcd 608.3, found 608.3.

### Example 10: 2-{[(S)-3-methyl-1-piperidyl]methyl}-4-cyclopropyl-6-(6-cyclopropyl-4-{4-fluoro-2-[(3-fluoro-1-azetidinyl)carbonyl]phenyl}-2-pyridyl)-1,6-dihydro-1,6-diaza-7-indenone

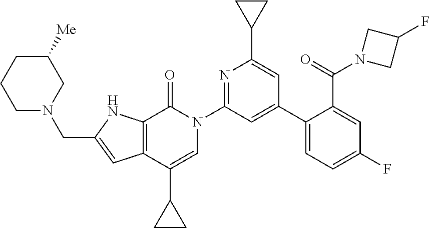 [SMILES: C[C@H]1CCCN(Cc2cc3c(C4CC4)cn(-c4cc(-c5ccc(F)cc5C(=O)N5CC(F)C5)cc(C5CC5)n4)c(=O)c3[nH]2)C1]

The title compound was prepared in a similar fashion to that described for Example 1 from 2-[2-cyclopropyl-6-[4-cyclopropyl-2-[[(3S)-3-methylpiperidin-1-yl]methyl]-7-oxo-1-(2-trimethylsilylethoxymethyl)pyrrolo[2,3-c]pyridin-6-yl]pyridin-4-yl]-5-fluorobenzoic acid and 3-fluoroazetidine. ¹H NMR (400 MHz, CDCl₃) δ 9.63 (s, 1H), 7.76 (d, J=1.4 Hz, 1H), 7.47 (dd, J=8.5, 5.2 Hz, 1H), 7.32 (d, J=1.2 Hz, 1H), 7.25-7.19 (m, 2H), 7.17 (d, J=1.5 Hz, 1H), 6.36 (s, 1H), 5.28-5.02 (m, 1H), 4.41-4.27 (m, 1H), 4.19-4.06 (m, 1H), 4.01-3.87 (m, 1H), 3.85-3.71 (m, 1H), 3.67-3.53 (m, 2H), 2.88-2.68 (m, 2H), 2.19-2.06 (m, 1H), 1.98-1.85 (m, 2H), 1.77-1.54 (m, 5H), 1.13-1.02 (m, 4H), 0.91-0.86 (m, 3H), 0.83 (d, J=5.0 Hz, 3H), 0.71-0.66 (m, 2H). ESI MS [M+H]⁺ for C₃₅H₃₇F₂N₅O₂ calcd 598.2 found 598.2.

### Example 11: 2-{[(S)-3-methyl-1-piperidyl]methyl}-4-cyclopropyl-6-(6-cyclopropyl-4-{4-fluoro-2-[(3-methoxy-1-azetidinyl)carbonyl]phenyl}-2-pyridyl)-1,6-dihydro-1,6-diaza-7-indenone

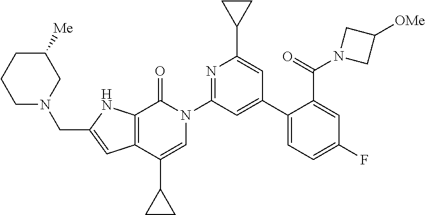 [SMILES: COC1CN(C(=O)c2cc(F)ccc2-c2cc(C3CC3)nc(-n3cc(C4CC4)c4cc(CN5CCC[C@H](C)C5)[nH]c4c3=O)c2)C1]

The title compound was prepared in a similar fashion to that described for Example 1 from 2-[2-cyclopropyl-6-[4-cyclopropyl-2-[[(3S)-3-methylpiperidin-1-yl]methyl]-7-oxo-1-(2-trimethylsilylethoxymethyl)pyrrolo[2,3-c]pyridin-6-yl]pyridin-4-yl]-5-fluorobenzoic acid and 3-meothoxyazetidine. ¹H NMR (400 MHz, CDCl₃) δ 9.58 (s, 1H), 7.76 (d, J=1.4 Hz, 1H), 7.47 (dd, J=8.5, 5.2 Hz, 1H), 7.32 (d, J=1.2 Hz, 1H), 7.24-7.14 (m, 3H), 6.36 (s, 1H), 4.23-4.14 (m, 1H), 4.05-3.98 (m, 1H), 3.92-3.85 (m, 1H), 3.83-3.74 (m, 1H), 3.67-3.56 (m, 2H), 3.56-3.52 (m, 1H), 3.15 (s, 3H), 2.85-2.71 (m, 2H), 2.16-2.08 (m, 1H), 2.00-1.87 (m, 2H), 1.75-1.52 (m, 5H), 1.13-0.99 (m, 4H), 0.91-0.82 (m, 6H), 0.72-0.65 (m, 2H). ESI MS [M+H]⁺ for C₃₆H₄₀FN₅O₃ calcd 610.3 found 610.2.

### Example 12: N-2,2,2-trifluoroethyl2-[2-(2-{[(S)-3-methyl-1-piperidyl]methyl}-4-cyclopropyl-7-oxo-1,6-dihydro-1,6-diaza-6-indenyl)-6-cyclopropyl-4-pyridyl]-5-fluorobenzamide

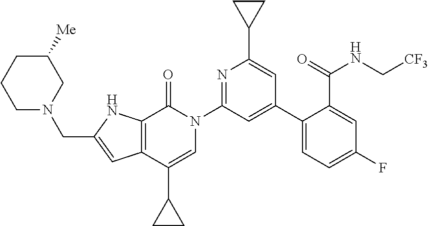 [SMILES: C[C@H]1CCCN(Cc2cc3c(C4CC4)cn(-c4cc(-c5ccc(F)cc5C(=O)NCC(F)(F)F)cc(C5CC5)n4)c(=O)c3[nH]2)C1]

The title compound was prepared in a similar fashion to that described for Example 1 from 2-[2-cyclopropyl-6-[4-cyclopropyl-2-[[(3S)-3-methylpiperidin-1-yl]methyl]-7-oxo-1-(2-trimethylsilylethoxymethyl)pyrrolo[2,3-c]pyridin-6-yl]pyridin-4-yl]-5-fluorobenzoic acid and 2,2,2-trifluoroethylamine. ¹H NMR (400 MHz, CDCl₃) δ 11.01 (s, 1H), 7.82 (d, J=1.3 Hz, 1H), 7.43-7.36 (m, 2H), 7.34 (s, 1H), 7.24-7.18 (m, 1H), 7.16 (s, 1H), 6.33 (s, 1H), 4.02-3.82 (m, 2H), 3.71-3.54 (m, 2H), 2.90-2.63 (m, 2H), 2.12-1.84 (m, 4H), 1.67-1.47 (m, 3H), 1.41-1.19 (m, 3H), 1.11-0.98 (m, 4H), 0.91-0.84 (m, 2H), 0.76 (d, J=6.6 Hz, 3H), 0.71-0.62 (m, 2H). ESI MS [M+H]⁺ for C₃₄H₃₅F₄N₅O₂ calcd 622.2 found 622.2.

### Example 13: N-ethyl-N-methyl2-[2-(2-{[(S)-3-methyl-1-piperidyl]methyl}-4-cyclopropyl-7-oxo-1,6-dihydro-1,6-diaza-6-indenyl)-6-cyclopropyl-4-pyridyl]-5-fluorobenzamide

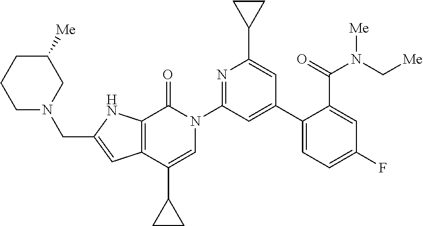 [SMILES: CCN(C)C(=O)c1cc(F)ccc1-c1cc(C2CC2)nc(-n2cc(C3CC3)c3cc(CN4CCC[C@H](C)C4)[nH]c3c2=O)c1]

The title compound was prepared in a similar fashion to that described for Example 1 from 2-[2-cyclopropyl-6-[4-cyclopropyl-2-[[(3S)-3-methylpiperidin-1-yl]methyl]-7-oxo-1-(2-trimethylsilylethoxymethyl)pyrrolo[2,3-c]pyridin-6-yl]pyridin-4-yl]-5-fluorobenzoic acid and ethylmethylamine. ¹H NMR (400 MHz, CDCl₃) δ 9.49 (s, 1H), 7.73-7.69 (m, 1H), 7.50-7.40 (m, 1H), 7.31-7.28 (m, 1H), 7.21 (dd, J=4.9, 1.5 Hz, 1H), 7.18-7.09 (m, 2H), 6.35 (s, 1H), 3.59 (d, J=3.4 Hz, 2H), 2.93 (s, 1H), 2.84-2.71 (m, 2H), 2.59 (s, 2H), 2.14-2.05 (m, 1H), 1.97-1.85 (m, 2H), 1.75-1.49 (m, 6H), 1.31 (m, 1H), 1.12-0.97 (m, 4H), 0.93 (t, J=7.2 Hz, 2H), 0.90-0.85 (m, 4H), 0.85-0.81 (m, 3H), 0.72-0.65 (m, 2H). ESI MS [M+H]⁺ for C₃₅H₄₀FN₅O₂ calcd 582.3 found 582.3.

### Example 14: 2-{[(S)-3-methyl-1-piperidyl]methyl}-4-cyclopropyl-6-(6-cyclopropyl-4-{2-[(3,3-difluoro-1-azetidinyl)carbonyl]-4-fluorophenyl}-2-pyridyl)-1,6-dihydro-1,6-diaza-7-indenone

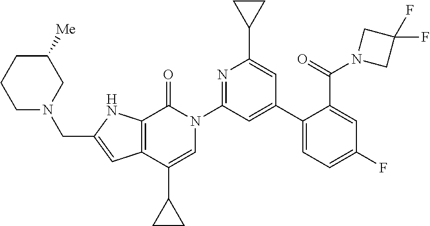 [SMILES: C[C@H]1CCCN(Cc2cc3c(C4CC4)cn(-c4cc(-c5ccc(F)cc5C(=O)N5CC(F)(F)C5)cc(C5CC5)n4)c(=O)c3[nH]2)C1]

The title compound was prepared in a similar fashion to that described for Example 1 from 2-[2-cyclopropyl-6-[4-cyclopropyl-2-[[(3S)-3-methylpiperidin-1-yl]methyl]-7-oxo-1-(2-trimethylsilylethoxymethyl)pyrrolo[2,3-c]pyridin-6-yl]pyridin-4-yl]-5-fluorobenzoic acid and 3,3-dilfluoroazetidine. ¹H NMR (400 MHz, CDCl₃) δ 9.50 (s, 1H), 7.78 (d, J=1.4 Hz, 1H), 7.50 (dd, J=8.5, 5.2 Hz, 1H), 7.33 (d, J=1.2 Hz, 1H), 7.26-7.19 (m, 2H), 7.14 (d, J=1.4 Hz, 1H), 6.35 (s, 1H), 4.39 (t, J=11.6 Hz, 2H), 3.96 (t, J=11.6 Hz, 2H), 3.65-3.53 (m, 2H), 2.86-2.69 (m, 1H), 2.15-2.06 (m, 1H), 1.98-1.84 (m, 2H), 1.75-1.52 (m, 5H), 1.14-1.01 (m, 4H), 0.93-0.86 (m, 4H), 0.83 (d, J=5.6 Hz, 3H), 0.73-0.65 (m, 2H). ESI MS [M+H]⁺ for C₃₅H₃₆F₃N₅O₂ calcd 616.2 found 616.2.

### Example 15: 2-{[(S)-3-methyl-1-piperidyl]methyl}-4-cyclopropyl-6-(6-cyclopropyl-4-{4-fluoro-2-[(2-methyl-1-azetidinyl)carbonyl]phenyl}-2-pyridyl)-1,6-dihydro-1,6-diaza-7-indenone

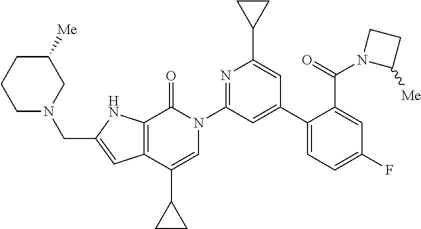 [SMILES: CC1CCN1C(=O)c1cc(F)ccc1-c1cc(C2CC2)nc(-n2cc(C3CC3)c3cc(CN4CCC[C@H](C)C4)[nH]c3c2=O)c1]

The title compound was prepared as an inseparable mixture of diastereomers in 2:1 ratio in a similar fashion to that described for Example 1 from 2-[2-cyclopropyl-6-[4-cyclopropyl-2-[[(3S)-3-methylpiperidin-1-yl]methyl]-7-oxo-1-(2-trimethylsilylethoxymethyl)pyrrolo[2,3-c]pyridin-6-yl]pyridin-4-yl]-5-fluorobenzoic acid and 2-methylazetidine. ¹H NMR (400 MHz, CDCl₃) δ 9.50 (s, 1.4H), 7.79 (d, J=1.4 Hz, 0.5H), 7.78 (d, J=1.5 Hz, 1H), 7.53-7.46 (m, 0.5H), 7.43 (dd, J=8.5, 5.3 Hz, 1H), 7.37-7.35 (m, 1H), 7.34-7.32 (m, 0.5H), 7.24 (d, J=1.4 Hz, 1H), 7.24-7.22 (m, 0.5H), 7.21-7.19 (m, 1H), 7.19-7.12 (m, 2H), 6.36 (s, 0.5H), 6.35 (s, 1H), 4.53-4.43 (m, 1H), 4.08-3.82 (m, 2H), 3.65-3.61 (m, 0.7H), 3.60-3.57 (m, 3H), 3.57-3.49 (m, 1.4H), 2.86-2.70 (m, 4H), 2.34-2.20 (m, 1.5H), 2.17-2.06 (m, 2H), 2.00-1.83 (m, 3.4H), 1.76-1.65 (m, 3H), 1.64-1.51 (m, 6H), 1.31 (d, J=6.3 Hz, 3H), 1.29-1.27 (m, 1.4H), 1.14-1.08 (m, 2H), 1.07-0.98 (m, 4H), 0.93-0.88 (m, 4H), 0.88-0.86 (m, 2H), 0.85-0.83 (m, OH), 0.82 (d, J=4.7 Hz, 3H), 0.73-0.63 (m, 3H). ESI MS [M+H]⁺ for C₃₆H₄₁FN₅O₂ calcd 594.3 found 594.3.

### Example 16: 1-{2-[2-(2-{[(S)-3-methyl-1-piperidyl]methyl}-4-cyclopropyl-7-oxo-1,6-dihydro-1,6-diaza-6-indenyl)-6-cyclopropyl-4-pyridyl]-5-fluorobenzoyl}-3-azetidinecarbonitrile

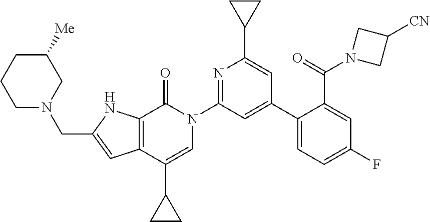 [SMILES: C[C@H]1CCCN(Cc2cc3c(C4CC4)cn(-c4cc(-c5ccc(F)cc5C(=O)N5CC(C#N)C5)cc(C5CC5)n4)c(=O)c3[nH]2)C1]

The title compound was prepared in a similar fashion to that described for Example 1 from 2-[2-cyclopropyl-6-[4-cyclopropyl-2-[[(3S)-3-methylpiperidin-1-yl]methyl]-7-oxo-1-(2-trimethylsilylethoxymethyl)pyrrolo[2,3-c]pyridin-6-yl]pyridin-4-yl]-5-fluorobenzoic acid and azetidine-3-carbonitrile. ¹H NMR (400 MHz, CDCl₃) δ 9.58 (s, 1H), 7.74 (s, 1H), 7.48-7.41 (m, 1H), 7.37 (s, 1H), 7.23-7.17 (m, 2H), 7.16-7.14 (m, 1H), 6.34 (s, 1H), 4.42-4.29 (m, 2H), 4.03 (d, J=7.7 Hz, 2H), 3.63-3.43 (m, 3H), 2.83-2.69 (m, 2H), 2.14-2.05 (m, 1H), 1.96-1.84 (m, 2H), 1.79-1.46 (m, 6H), 1.13-1.00 (m, 4H), 0.89-0.80 (m, 7H), 0.69-0.64 (m, 2H). ESI MS [M+H]⁺ for C₃₆H₃₇FN₆O₂ calcd 605.3 found 605.3.

### Example 17: 2-{[(1S,2R)-2-hydroxycyclopentylamino]methyl}-4-cyclopropyl-6-(6-cyclopropyl-4-{4-fluoro-2-[(3-fluoro-1-azetidinyl)carbonyl]phenyl}-2-pyridyl)-1,6-dihydro-1,6-diaza-7-indenone

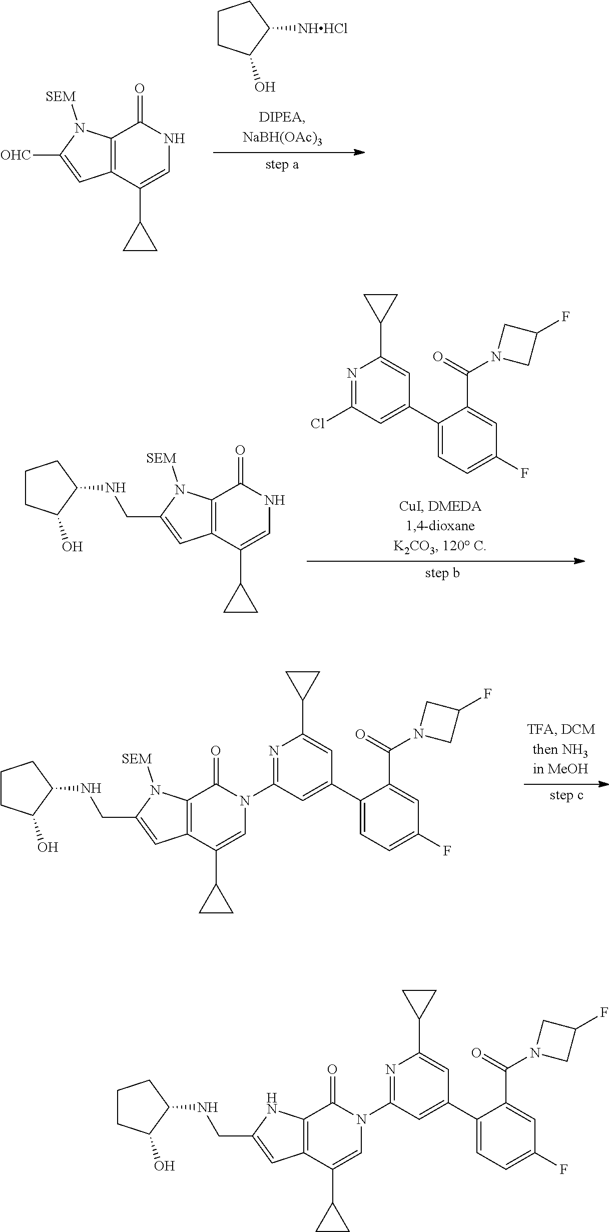

Step a: To a solution of the 4-cyclopropyl-7-oxo-1-(2-trimethylsilylethoxymethyl)-6H-pyrrolo[2,3-c]pyridine-2-carbaldehyde (498 mg, 1.5 mmol, 1.0 equiv., obtained according to example 1, step f) in dichloromethane (5 mL) was added (1S,2R)-2-aminocyclopentanol hydrochloride (203 mg, 1.5 mmol, 1.5 equiv.) and i-pr₂NEt (0.52 mL, 3.0 mmol, 2.0 equiv.). The resulting mixture was stirred at room temperature for 30 min before adding NaBH(OAc)₃ (633 mg, 3.0 mmol, 2.0 equiv.). The reaction mixture was then stirred at room temperature for 12 h before being quenched with H₂O. The organic phase was separated, and the aqueous layer was extracted with dichloromethane twice. The combined organic phase was dried over Na₂SO₄, concentrated, and the crude residue was purified by column chromatography (SiO₂, MeOH in dichloromethane, 0 to 10%) to afford the amine product.

Step b: To a solution of the product from step a (54.2 mg, 0.13 mmol, 1.0 equiv.) and [2-(2-chloro-6-cyclopropylpyridin-4-yl)-5-fluorophenyl]-(3-fluoroazetidin-1-yl)methanone (45.9 mg, 0.13 mmol, 1.2 equiv., obtained similar to example 21, step b) in dioxane (2.6 mL, 0.05M) was added CuI (25 mg, 0.13 mmol, 1.0 equiv.), N,N′-dimethylethylenediamine (23.0 mg, 0.26 mmol, 2.0 equiv.), and K₂CO₃ (54 mg, 0.34 mmol, 3.0 equiv.). The resulting mixture was heated at 110° C. for 12 h. After cooling to room temperature, the reaction mixture was diluted with EtOAc and washed sequentially with Aq. sat. NH₄Cl, water and brine. The organic extract was dried over Na₂SO₄ and concentrated to dryness under vacuum. The residue was then treated with TFA/DCM (v/v 1:1, 3 mL) at room temperature for 4 h and then concentrated.

Step c: The product from step b was dissolved in TFA/DCM mixture (v/v 1:1, 3 mL) and stirred at room temperature for 4 h. The resulting solution was concentrated to dryness, the residual TFA was removed by co-evaporation with additional DCM. Then the crude material was dissolved in 7M NH₃ in methanol (4 mL) for 1 h. Upon concentration to dryness the crude residue was directly purified by reverse phase HPLC to afford the title compound. ¹H NMR (400 MHz, CDCl₃) δ 11.32 (s, 1H), 7.66 (s, 1H), 7.53-7.45 (m, 1H), 7.25-7.13 (m, 4H), 6.38 (s, 1H), 5.27-5.03 (m, 1H), 4.39-4.25 (m, 1H), 4.17-4.04 (m, 1H), 4.01-3.64 (m, 5H), 2.87-2.76 (m, 1H), 2.17-2.07 (m, 1H), 1.93-1.86 (m, 1H), 1.79-1.64 (m, 5H), 1.53-1.40 (m, 1H), 1.15-1.01 (m, 4H), 0.95-0.80 (m, 4H), 0.72-0.63 (m, 2H). ESI MS [M+H]⁺ for C₃₄H₃₅F₂N₅O₃ calcd 600.2 found 600.2.

### Example 18: 2-[2-(2-{[(S)-3-methyl-1-piperidyl]methyl}-4-cyclopropyl-7-oxo-1,6-dihydro-1,6-diaza-6-indenyl)-6-cyclopropyl-4-pyridyl]-5-fluorobenzamide

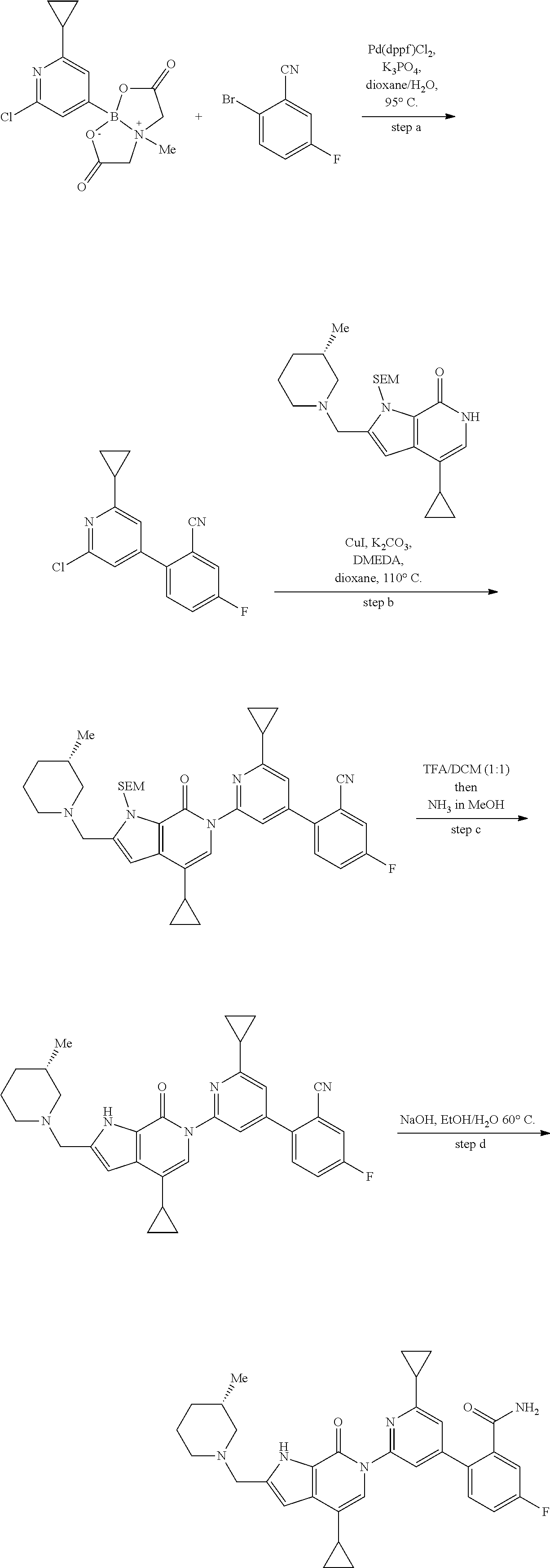

Steps a-c were performed in a similar fashion to that described in Example 1.

Step d: To a solution of 2-[2-cyclopropyl-6-[4-cyclopropyl-2-[[(3S)-3-methylpiperidin-1-yl]methyl]-7-oxo-1H-pyrrolo[2,3-c]pyridin-6-yl]pyridin-4-yl]-5-fluorobenzonitrile (52 mg, 0.1 mmol) in EtOH/H₂O (1:1, 1 mL) was added NaOH (20 mg, 0.5 mmol, 5.0 equiv.), and the resulting solution was stirred for 4 h at 60° C. Upon complete consumption of the starting material the reaction mixture was quenched with 1 M HCl and concentrated to remove MeOH under vacuum. The product was extracted with EtOAc (3×20 mL). The combined organic phase was washed with brine, dried over Na₂SO₄, and concentrated under reduced pressure. The crude residue was purified by prep-HPLC. ¹H NMR (400 MHz, CDCl₃) δ 11.30 (s, 1H), 7.70 (d, J=1.4 Hz, 1H), 7.39 (dd, J=8.8, 2.7 Hz, 1H), 7.32 (dd, J=8.5, 5.3 Hz, 1H), 7.27 (d, J=1.2 Hz, 1H), 7.19 (d, J=1.4 Hz, 1H), 7.15 (td, J=8.3, 2.7 Hz, 1H), 6.76 (s, 1H), 6.38 (s, 1H), 6.30 (s, 1H), 3.69 (s, 2H), 2.84 (dd, J=24.5, 11.1 Hz, 3H), 2.12-1.95 (m, 2H), 1.94-1.85 (m, 1H), 1.76-1.40 (m, 5H), 1.11-0.98 (m, 4H), 0.91-0.84 (m, 2H), 0.79 (d, J=6.4 Hz, 3H), 0.71-0.64 (m, 2H). ESI MS [M+H]⁺ for C₃₂H₃₄FN₅O₂ calcd 540.2 found 540.3.

### Example 19: N,N-dimethyl6-[2-(2-{[(S)-3-methyl-1-piperidyl]methyl}-4-cyano-7-oxo-1,6-dihydro-1,6-diaza-6-indenyl)-6-cyclopropyl-4-pyridyl]-2,3-difluorobenzamide

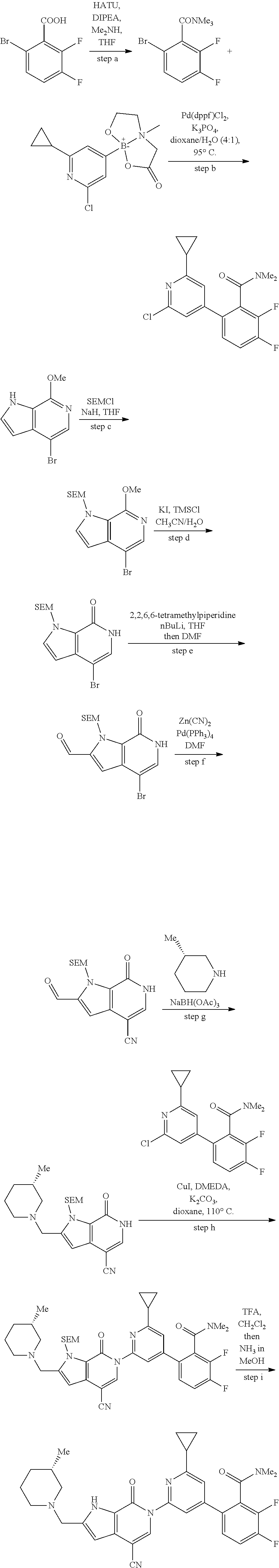

Step a: To a solution of a 6-bromo-2,3-difluorobenzoic acid (711 mg, 3.0 mmol, 1 equiv.) in DMF (15 mL, 0.2 M) were added i-pr₂NEt (1.06 mL, 6.0 mmol, 2 equiv.), HATU (1.7 g, 4.5 mmol, 1.5 equiv.) and dimethylamine (2M in THF, 3 mL, 2 equiv.). The reaction mixture was stirred overnight at room temperature and diluted with EtOAc and water. The organic phase was separated, washed with brine, dried over Na₂SO₄ and concentrated to dryness under reduced pressure. The crude residue was purified by silica gel flash column chromatography (0 to 20% MeOH/DCM) to afford as a 6-bromo-2,3-difluoro-N,N-dimethylbenzamide.

Step b: The reaction was performed in a similar fashion to Example 1, step c.

Step c: To a 1-L round bottom flask was loaded 4-bromo-7-methoxy-1H-pyrrolo[2,3-c]pyridine (10.0 g, 44.0 mmol) and THF (220 mL). The resulting mixture was cooled to 0° C. NaH (60% in mineral oil, 1.936 g, 48.4 mmol) was then added. The mixture was stirred at 0° C. for 30 min, followed by the addition of SEMCl (8.1 g, 48.4 mmol). The cooling bath was removed, and the reaction was stirred at room temperature for 3.5 hours. The reaction mixture was then poured into Aq. sat. NH₄Cl, and the product was extracted with EtOAc. The organic extract was dried over Na₂SO₄, concentrated under reduced pressure and purified by flash chromatography (SiO₂, 0-15% EtOAc/hexanes) to yield 2-[(4-bromo-7-methoxypyrrolo[2,3-c]pyridin-1-yl)methoxy]ethyl-trimethylsilane.

Step d: To a solution of 2-[(4-bromo-7-methoxypyrrolo[2,3-c]pyridin-1-yl)methoxy]ethyl-trimethylsilane (14.6 g, 41.0 mmol) in acetonitrile (205 mL) was added KI (7.5 g, 45.1 mmol), TMSCl (4.9 g, 45.1 mmol) and water (0.05 mL). The cloudy mixture was stirred at room temperature for 4.5 h. Once complete consumption of the starting material was observed by TLC analysis the mixture was quenched by addition of water and Aq. sat Na₂S₂O₃ (1:1, v/v). The product was extracted with EtOAc, combined organic extract was washed with brine, dried over Na₂SO₄ and concentrated to dryness under reduced pressure. The dry residue was purified by flash chromatography (SiO₂, 0-90% EtOAc/hexanes) to furnish the desired product.

Step e: To a solution of 2,2,6,6-tetramethylpiperidine (1.2 mL, 7.0 mmol, 2.4 equiv.) in dry THF (10 mL) nBuLi (2.7 mL, 6.9 mmol, 2.35 equiv, 2.5 M solution in hexanes) was added dropwise at −78 C under an atmosphere of nitrogen. After stirring for 5 min the resulting mixture was transferred to an ice bath and stirred for additional 15 min. The resulting solution of LiTMP was added dropwise to a solution of pyrrolopyridone substrate from step d (1.0 g, 2.9 mmol, 1 equiv.) in THF (20 mL) at −78 C. The reaction mixture was stirred at −78 C for 1 h followed by the addition of DMF (0.7 mL, 8.7 mmol, 3 equiv.). Then acetone/dry ice bath was replaced with an ice bath, and the reaction mixture was stirred at 0° C. for 1 h. The resulting mixture was quenched by addition of Aq. sat. NH₄Cl (30 mL), and the product was extracted with EtOAc (3×30 mL). Combined organic extract was washed with brine, dried over Na₂SO₄ and concentrated to dryness. ¹H NMR analysis indicated clean reaction resulting in 50% conversion to the desired aldehyde. The dry residue was fractionated by column chromatography (SiO₂, 0→10% EtOAc in dichloromethane, 10 min, then 10% EtOAc in DCM till complete elution of the product, then 10→80% EtOAc in DCM for the elution of recovered starting material) to produce the formylated product (0.46 g, 1.25 mmol, 43% yield) and recovered starting material (0.45 g, 1.3 mmol).

Step f: A mixture of bromide from step e (2.5 g 6.7 mmol, 1 equiv.), Zn(CN)₂ (0.8 g, 6.7 mmol, 1 equiv.), Pd(PPh₃)₄ (0.4 g, 0.34 mmol, 0.05 quiv.) and DMF (13.4 mL, 0.5M) was loaded in 40 mL vial equipped with a stirring bar. The reaction mixture was degassed by applying vacuum and backfilling with dry nitrogen 3 times followed by heating at 100° C. for 1.5 h. Once TLC analysis indicated complete consumption of starting material the reaction was cooled to room temperature and partitioned between water (70 mL) and EtOAc (70 mL). The resulting white precipitate was removed by filtration through Celite® pad, and the organic layer was separated. The aqueous phase was additionally extracted with EtOAc (2×30 mL). The combined organic extract was washed with water and brine, dried over Na₂SO₄ and concentrated to dryness. The crude material was purified (SiO₂, 0-90% EtOAc/hexanes) to produce the desired product (1.7 g, 5.3 mmol, 79% yield) as a yellowish solid.

Step g: To a solution of 2-formyl-7-oxo-1-(2-trimethylsilylethoxymethyl)-6H-pyrrolo[2,3-c]pyridine-4-carbonitrile (1.0 g, 3.15 mmol) in dichloromethane (15 mL), was added (S)-3-methylpiperidine hydrochloride (638 mg, 4.73 mmol, 1.5 equiv.) and i-pr₂NEt (1.1 mL, 6.30 mmol, 2.0 equiv.) and the mixture was stirred at 23° C. for 10 mins. NaBH(OAc)₃ (1.3 g, 6.30 mmol, 2.0 equiv.) was added and the mixture was stirred at 23° C. for 12 h. The reaction was diluted with Aq. sat. NaHCO₃ and EtOAc. The organic phase was separated, and the aqueous layer was additionally extracted with EtOAc. The combined organic phase was dried over Na₂SO₄, concentrated under vacuum to dryness, and the crude residue was purified by column chromatography (SiO₂, MeOH in DCM, 0 to 15%) to afford 2-[[(3S)-3-methylpiperidin-1-yl]methyl]-7-oxo-1-(2-trimethylsilylethoxymethyl)-6H-pyrrolo[2,3-c]pyridine-4-carbonitrile.

Steps h-i were performed in a similar fashion to Example 1, step i and l, respectively. ¹H NMR (400 MHz, CDCl₃) δ 9.65 (s, 1H), 8.16 (d, J=1.1 Hz, 1H), 7.69 (t, J=1.4 Hz, 1H), 7.33-7.27 (m, 1H), 7.26-7.21 (m, 1H), 6.39 (s, 1H), 3.67-3.54 (m, 2H), 3.03 (s, 3H), 2.83-2.65 (m, 5H), 2.16-2.06 (m, 1H), 2.03-1.92 (m, 1H), 1.79-1.50 (m, 5H), 1.11-1.04 (m, 4H), 0.85 (d, J=5.6 Hz, 5H). ESI MS [M+H]⁺ for C₃₂H₃₃F₂N₆O₂ calcd 571.2 found 571.3.

### Example 20: N,N-dimethyl-2′-(2-{[(S)-3-methyl-1-piperidyl]methyl}-4-cyano-7-oxo-1,6-dihydro-1,6-diaza-6-indenyl)-6′-cyclopropyl-[3,4′-bipyridyl]-2-carboxamide

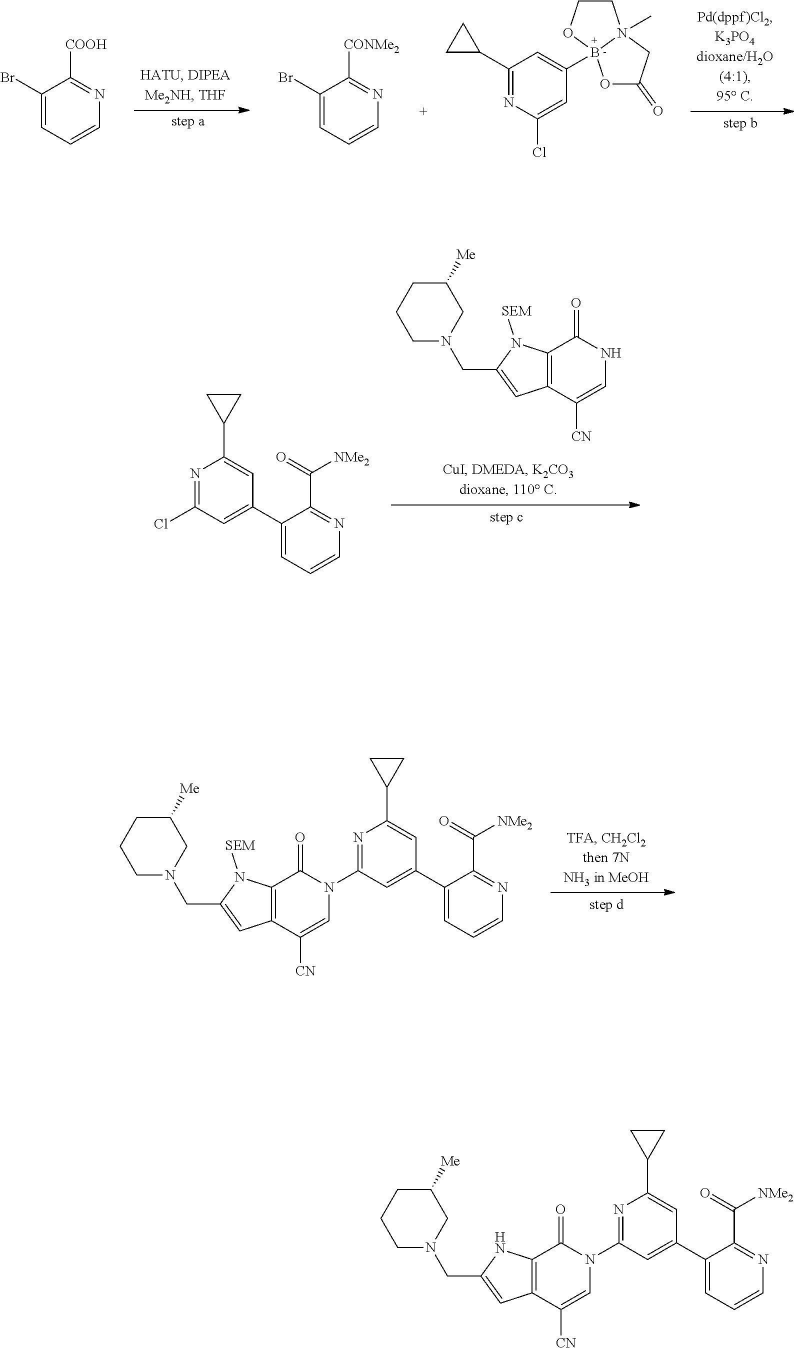

The title compound was prepared in a similar fashion to that described for Example 19. ¹H NMR (400 MHz, CDCl₃) δ 9.75 (s, 1H), 8.70 (dd, J=4.8, 1.5 Hz, 1H), 8.18 (d, J=1.2 Hz, 1H), 7.85 (dd, J=7.9, 1.6 Hz, 1H), 7.75 (d, J=1.3 Hz, 1H), 7.47-7.41 (m, 1H), 7.32 (d, J=1.4 Hz, 1H), 6.39 (s, 1H), 3.67-3.54 (m, 2H), 3.04 (s, 3H), 2.82-2.67 (m, 5H), 2.16-2.07 (m, 1H), 2.02-1.93 (m, 1H), 1.77-1.50 (m, 5H), 1.08 (d, J=5.9 Hz, 4H), 0.93-0.77 (m, 5H). ESI MS [M+H]⁺ for C₃₁H₃₃N₇O₂ calcd 536.2 found 536.2.

### Example 21: 2-{[(S)-3-methyl-1-piperidyl]methyl}-6-(6-cyclopropyl-4-{4-fluoro-2-[(3-fluoro-1-azetidinyl)carbonyl]phenyl}-2-pyridyl)-7-oxo-1,6-dihydro-1,6-diaza-4-indenecarbonitrile

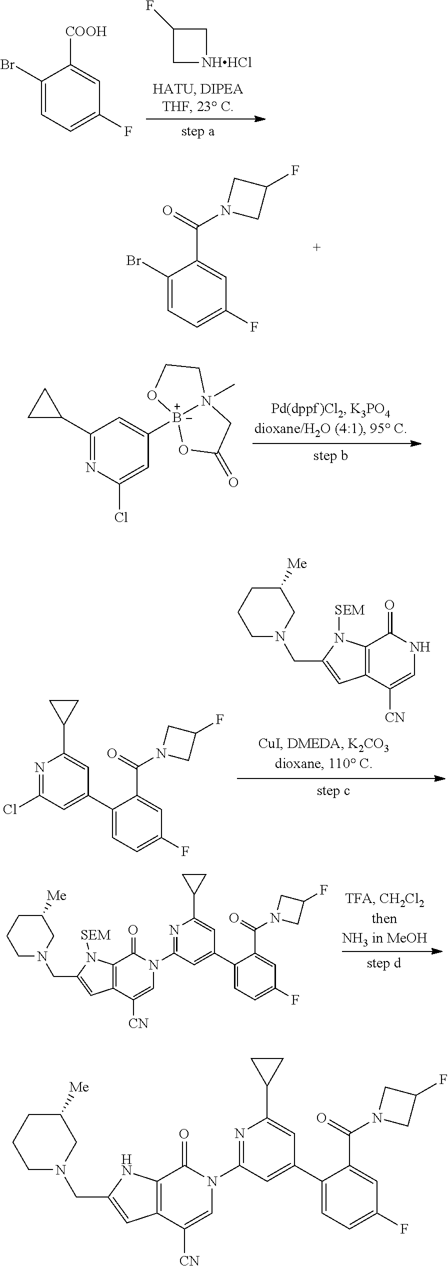

The title compound was prepared in a similar fashion to that described for Example 19. ¹H NMR (400 MHz, CDCl₃) δ 9.83 (s, 1H), 8.16 (s, 1H), 7.72 (s, 1H), 7.51-7.43 (m, 1H), 7.27 (d, J=1.4 Hz, 1H), 7.26-7.20 (m, 2H), 6.39 (s, 1H), 5.27-5.07 (m, 1H), 4.47-4.32 (m, 1H), 4.22-4.09 (m, 1H), 4.00-3.86 (m, 1H), 3.86-3.72 (m, 1H), 3.67-3.55 (m, 2H), 2.85-2.69 (m, 2H), 2.16-2.08 (m, 1H), 2.03-1.93 (m, 1H), 1.76-1.54 (m, 5H), 1.15-1.04 (m, 4H), 0.95-0.78 (m, 4H). ESI MS [M+H]⁺ for C₃₃H₃₂F₂N₆O₂ calcd 583.2 found 583.2.

### Example 22: 2-{[(S)-3-methyl-1-piperidyl]methyl}-6-{5-cyano-4′-fluoro-2′-[(3-fluoro-1-azetidinyl)carbonyl]-3-biphenylyl}-7-oxo-1,6-dihydro-1,6-diaza-4-indenecarbonitrile

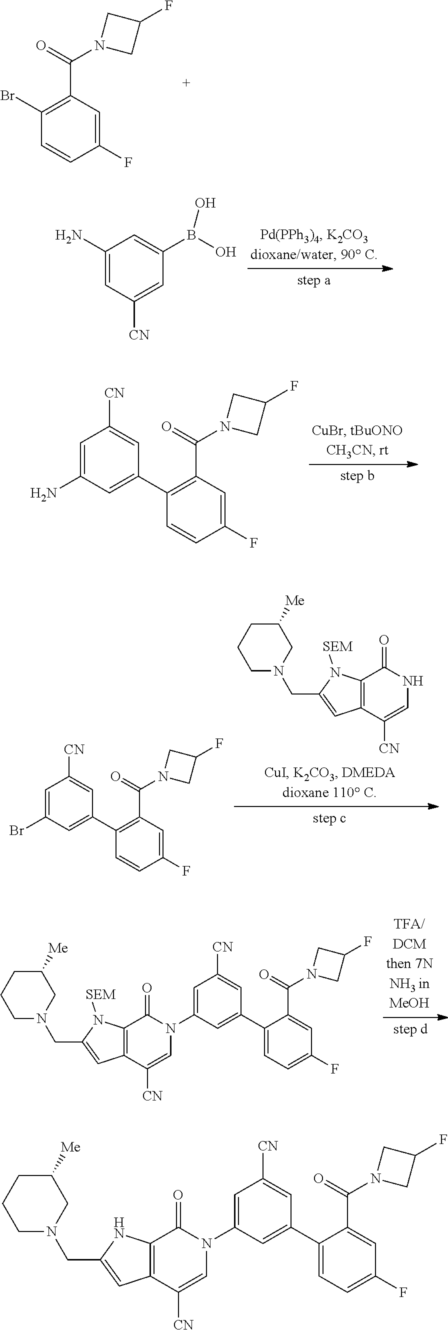

Step a: A 40 mL vial was charged with (2-bromo-5-fluorophenyl)-(3-fluoroazetidin-1-yl)methanone (500 mg, 1.82 mmol, 1 equiv.), (3-amino-5-cyanophenyl)boronic acid (295.5 mg, 1.82 mmol. 1.0 equiv.) and K₂CO₃ (753.4 mg, 5.4 mmol, 3 equiv.). The reagents were suspended in the 3:1 mixture of dioxane/water (10 mL), and the resulting solution was sparged with N₂ for 10 minutes. Then, Pd(PPh₃)₄ (210 mg, 0.182 mmol, 0.1 equiv.) was added, and the mixture was heated to 95° C. for 4 hours. The reaction mixture was partitioned between EtOAc and water, the organic phase was separated, and the aqueous layer was additionally extracted with EtOAc. The combined organic phase was dried over Na₂SO₄ and concentrated to dryness under vacuum. The crude residue was purified via silica gel flash column chromatography (0 to 100% EtOAc/hexane) to afford the desired coupling product.

Step b: The solution of amide from step a (200 mg, 0.63 mmol, 1.0 equiv.) and tert-butyl nitrite (144 mg, 1.26 mmol, 2.0 equiv.) in MeCN (6.3 mL, 0.1 M) was added CuBr (108 mg, 0.75 mmol, 1.2 equiv.). The reaction mixture was stirred overnight at 60° C. It was cooled to room temperature and quenched with water. The product was extracted with dichloromethane. The combined organic extract was washed with brine, dried over Na₂SO₄, and concentrated under reduced pressure. The crude residue was purified by column chromatography (EtOAc in hexanes, 0 to 50%) to yield 3-bromo-5-[4-fluoro-2-(3-fluoroazetidine-1-carbonyl)phenyl]benzonitrile as a desired product.

Step c: To a solution of the 2-[[(3S)-3-methylpiperidin-1-yl]methyl]-7-oxo-1-(2-trimethylsilylethoxymethyl)-6H-pyrrolo[2,3-c]pyridine-4-carbonitrile (75 mg, 0.187 mmol, 1.0 equiv.) and 3-bromo-5-[4-fluoro-2-(3-fluoroazetidine-1-carbonyl)phenyl]benzonitrile (84.6 mg, 0.22 mmol, 1.2 equiv., obtained according to example 19, step g) in dioxane (3.2 mL, 0.05M) was added CuI (35 mg, 0.187 mmol, 1.0 equiv.), N,N′-dimethylethylenediamine (33.0 mg, 0.37 mmol, 2.0 equiv.), and K₂CO₃ (77 mg, 0.56 mmol, 3.0 equiv.). The resulting mixture was heated at 110° C. for 6 h. After cooling to room temperature, the reaction mixture was diluted with EtOAc and sequentially washed with aq. NH₄Cl, water and brine. The organic extract was dried over Na₂SO₄ and concentrated to dryness under vacuum to afford the coupling product that was used for the next step without purification.

Step d: The product from step b was dissolved in a mixture of TFA/dichloromethane (3 mL, 1:1 v/v) and stirred at room temperature for 4 h. The resulting mixture was concentrated to dryness under reduced pressure. The residual TFA was removed by co-evaporation with dichloromethane. The dry residue was dissolved in 7N NH₃ in MeOH (4 mL) and stirred at room temperature for 1 h. The solvent was removed, and the crude residue was purified by reversed phase HPLC to afford the title compound. ¹H NMR (400 MHz, CDCl₃) δ 7.82 (t, J=1.5 Hz, 1H), 7.76 (t, J=1.9 Hz, 1H), 7.75-7.72 (m, 1H), 7.55 (s, 1H), 7.47-7.42 (m, 1H), 7.30-7.27 (m, 1H), 7.25-7.22 (m, 1H), 6.44 (s, 1H), 5.28-5.07 (m, 1H), 4.46-4.30 (m, 1H), 4.22-4.08 (m, 1H), 3.95-3.67 (m, 2H), 3.65 (d, J=3.6 Hz, 2H), 2.88-2.71 (m, 2H), 2.07-1.97 (m, 1H), 1.78-1.58 (m, 5H), 0.93-0.81 (m, 5H). ESI MS [M+H]⁺ for C₃₂H₂₈F₂N₆O₂ calcd 567.2 found 567.2.

### Example 23: 2-{[(S)-3-methyl-1-piperidyl]methyl}-6-{4′-fluoro-2′-[(3-fluoro-1-azetidinyl)carbonyl]-5-(trifluoromethyl)-3-biphenylyl}-7-oxo-1,6-dihydro-1,6-diaza-4-indenecarbonitrile

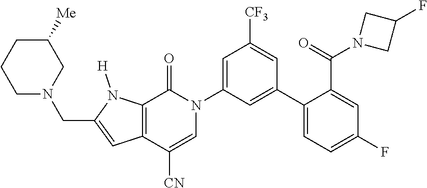 [SMILES: C[C@H]1CCCN(Cc2cc3c(C#N)cn(-c4cc(-c5ccc(F)cc5C(=O)N5CC(F)C5)cc(C(F)(F)F)c4)c(=O)c3[nH]2)C1]

The title compound was prepared in a similar fashion to that described for Example 22 from 2-[[(3S)-3-methylpiperidin-1-yl]methyl]-7-oxo-1-(2-trimethylsilylethoxymethyl)-6H-pyrrolo[2,3-c]pyridine-4-carbonitrile and ethylmethylamine and [2-[3-bromo-5-(trifluoromethyl)phenyl]-5-fluorophenyl]-(3-fluoroazetidin-1-yl)methanone. ¹H NMR (400 MHz, CDCl₃) δ 7.83-7.79 (m, 1H), 7.74 (t, J=1.8 Hz, 1H), 7.69-7.67 (m, 1H), 7.58 (s, 1H), 7.50-7.46 (m, 1H), 7.30-7.27 (m, 1H), 7.26-7.23 (m, 1H), 6.43 (s, 1H), 5.26-5.04 (m, 1H), 4.43-4.25 (m, 1H), 4.20-4.04 (m, 1H), 3.91-3.58 (m, 4H), 2.86-2.67 (m, 2H), 2.05-1.94 (m, 1H), 1.79-1.54 (m, 5H), 0.94-0.78 (m, 5H). ESI MS [M+H]⁺ for C₃₂H₂₈F₅N₅O₂ calcd 610.2 found 610.2.

### Example 24: 2-{[(S)-3-methyl-1-piperidyl]methyl}-6-(5-{4-fluoro-2-[(3-fluoro-1-azetidinyl)carbonyl]phenyl}-3-pyridyl)-7-oxo-1,6-dihydro-1,6-diaza-4-indenecarbonitrile

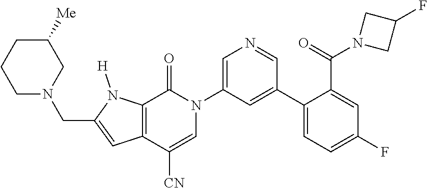 [SMILES: C[C@H]1CCCN(Cc2cc3c(C#N)cn(-c4cncc(-c5ccc(F)cc5C(=O)N5CC(F)C5)c4)c(=O)c3[nH]2)C1]

The title compound was prepared in a similar fashion to that described for Example 22 from 2-[[(3S)-3-methylpiperidin-1-yl]methyl]-7-oxo-1-(2-trimethylsilylethoxymethyl)-6H-pyrrolo[2,3-c]pyridine-4-carbonitrile and ethylmethylamine and [2-(5-bromopyridin-3-yl)-5-fluorophenyl]-(3-fluoroazetidin-1-yl)methanone. ¹H NMR (400 MHz, CDCl₃) δ 9.77 (s, 1H), 8.79 (d, J=2.1 Hz, 1H), 8.68 (d, J=2.4 Hz, 1H), 7.93 (t, J=2.2 Hz, 1H), 7.57 (s, 1H), 7.53-7.46 (m, 1H), 7.31-7.26 (m, 2H), 6.44 (s, 1H), 5.26-5.04 (m, 1H), 4.46-4.09 (m, 1H), 3.90-3.53 (m, 4H), 2.85-2.69 (m, 2H), 2.05-1.94 (m, 1H), 1.77-1.56 (m, 5H), 0.92-0.82 (m, 5H). ESI MS [M+H]⁺ for C₃₂H₂₈F₂N₆O₂ calcd 543.2 found 543.2.

### Example 25: 2-{[(S)-3-methyl-1-piperidyl]methyl}-6-(6-cyclopropyl-4-{4-fluoro-2-[(3-fluoro-1-azetidinyl)carbonyl]phenyl}-2-pyridyl)-1,6-dihydro-1,4,6-triaza-7-indenone

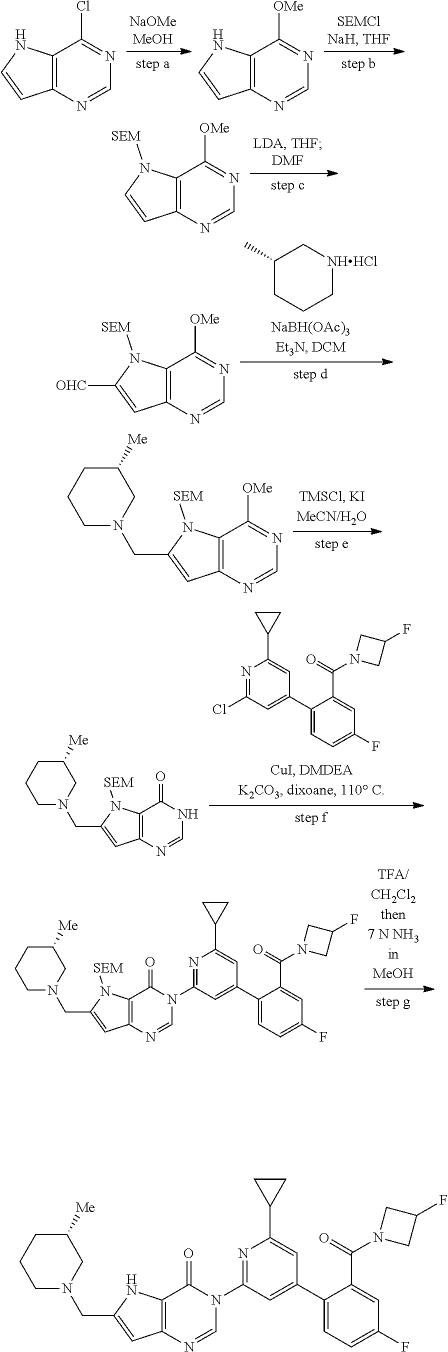

Step a: To a solution of 4-chloro-5H-pyrrolo[3,2-d]pyrimidine (1.00 g, 6.5 mmol, 1.0 equiv.) in methanol (20 mL) was added sodium methoxide (540 mg, 10 mmol, 1.5 equiv.). The resulting mixture was heated at 90° C. overnight. The reaction was cooled to rt and concentrated to dryness. The residue was directly purified by column chromatography (SiO₂, MeOH in DCM, 0 to 10%) to furnish the product.

Step b: To a solution of the product from step a (500 mg, 3.4 mmol, 1.0 equiv.) in THF (7 mL) was added NaH (60 wt % in mineral oil, 148 mg, 3.7 mmol, 1.1 equiv.) at 0° C. The resulting mixture was stirred at 0° C. for 10 min before the addition of 2-(trimethylsilyl)ethoxymethyl chloride (737 mg, 0.78 mL, 4.4 mmol, 1.3 equiv.). The reaction mixture was then warmed to room temperature and left to stir overnight. It was then quenched with water and diluted with EtOAc. The organic phase was separated, washed with brine, dried over Na₂SO₄, and concentrated. The crude residue was purified by column chromatography (SiO₂, EtOAc in hexanes, 0 to 40%) to afford the desired product.

Step c: To a solution of the product from step b (279 mg, 1.0 mmol, 1.0 equiv.) in THF (4 mL) was added lithium diisopropylamide (2M in THF, 0.55 mL, 1.1 mmol, 1.1 equiv.) at −78° C. The resulting solution was stirred at this temperature for another 30 min, then DMF (0.54 mL, 512 mg, 7.0 mmol, 5.0 equiv.) was added. After another 30 min at −78° C., the reaction mixture was quenched with saturated NH₄Cl aqueous solution and warmed to room temperature. The organic phase was separated, and the aqueous phase was extracted with EtOAc twice. The combined organic solution was washed with brine, dried over Na₂SO₄, and concentrated. The crude product was directly used for the next step.

Step d: To a solution of the crude product from step c (˜1.0 mmol, 1.0 equiv.) in dichloromethane (5 mL) was added (S)-3-methylpiperidine hydrochloride (203 mg, 1.5 mmol, 1.5 equiv.) and Et₃N (0.28 mL, 202 mg, 2.0 mmol, 2.0 equiv.). The resulting mixture was stirred at room temperature for 30 min before adding NaBH(OAc)₃ (424 mg, 2.0 mmol, 2.0 equiv.). The reaction mixture was then stirred at room temperature for another 45 min before being quenched with H₂O. The organic phase was separated, and the aqueous layer was extracted with dichloromethane twice. The combined organic phase was dried over Na₂SO₄, concentrated, and the crude residue was purified by column chromatography (SiO₂, MeOH in DCM, 0 to 10%) to give 2-[[4-methoxy-6-[[(3S)-3-methylpiperidin-1-yl]methyl]pyrrolo[3,2-d]pyrimidin-5-yl]methoxy]ethyl-trimethylsilane.

Step e: To a mixture of the product from step d (358 mg, 0.92 mmol, 1.0 equiv.) in MeCN/H₂O (4:1 v/v, 4 mL) was added TMSCl (0.19 mL, 160 mg, 1.5 mmol, 1.6 equiv.) and KI (244 mg, 1.5 mmol, 1.6 equiv.). The resulting mixture was stirred at room temperature overnight. LCMS analysis showed full conversion of the starting material. The reaction mixture was concentrated to dryness, and the crude product was directly purified by column chromatography (SiO₂, MeOH in DCM, 0 to 10%) to give 6-[[(3S)-3-methylpiperidin-1-yl]methyl]-5-(2-trimethylsilylethoxymethyl)-3H-pyrrolo[3,2-d]pyrimidin-4-one.

Step f: To a solution of the product from step e (50 mg, 0.13 mmol, 1.0 equiv.) and [2-(2-chloro-6-cyclopropylpyridin-4-yl)-5-fluorophenyl]-(3-fluoroazetidin-1-yl)methanone (45.9 mg, 0.13 mmol, 1.2 equiv.) in dioxane (2.6 mL, 0.05M) was added CuI (25 mg, 0.13 mmol, 1.0 equiv.), N,N′-dimethylethylenediamine (23.0 mg, 0.26 mmol, 2.0 equiv.), and K₂CO₃ (54 mg, 0.34 mmol, 3.0 equiv.). The resulting mixture was heated at 110° C. for 12 h. Once cooled to room temperature the reaction mixture was diluted with EtOAc and washed sequentially with aq. NH₄Cl, water and brine. The organic extract was dried over Na₂SO₄ and concentrated under reduced pressure to yield the corresponding coupling product that was used for the next step without purification.

Step g: The product from step h was dissolved in TFA/DCM mixture (v/v 1:1, 3 mL) and stirred at room temperature for 4 h. The resulting solution was concentrated to dryness, the residual TFA was removed by co-evaporation with additional DCM. Then the crude material was dissolved in 7M NH₃ in methanol (4 mL) for 1 h. Upon concentration to dryness the crude residue was directly purified by reverse phase HPLC to afford the title compound. ¹H NMR (400 MHz, CDCl₃) δ 9.40 (s, 1H), 8.38 (s, 1H), 7.63 (d, J=1.3 Hz, 1H), 7.54-7.41 (m, 1H), 7.29 (d, J=1.4 Hz, 1H), 7.26-7.17 (m, 2H), 6.36 (s, 1H), 5.27-5.04 (m, 1H), 4.47-4.31 (m, 1H), 4.24-4.10 (m, 1H), 3.98-3.73 (m, 2H), 3.67-3.51 (m, 2H), 2.85-2.67 (m, 2H), 2.17-2.06 (m, 1H), 2.02-1.92 (m, 1H), 1.78-1.50 (m, 5H), 1.14-1.02 (m, 4H), 0.89-0.78 (m, 4H). ESI MS [M+H]⁺ for C₃₁H₃₂F₂N₆O₂ calcd 559.2 found 559.2.

### Example 26: 3-[6-cyclopropyl-4-[4-fluoro-2-(3-fluoroazetidine-1-carbonyl)phenyl]pyridin-2-yl]-6-[[(2R)-2-methylmorpholin-4-yl]methyl]-5H-pyrrolo[3,2-d]pyrimidin-4-one

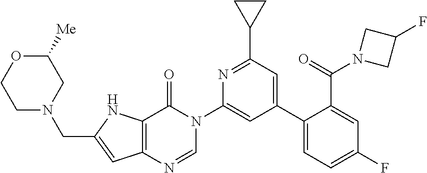 [SMILES: C[C@@H]1CN(Cc2cc3ncn(-c4cc(-c5ccc(F)cc5C(=O)N5CC(F)C5)cc(C5CC5)n4)c(=O)c3[nH]2)CCO1]

The title compound was prepared according to the protocol described for the example 25 using (2R)-2-methylmorpholine hydrochloride for reductive amination on step d. ¹H NMR (400 MHz, CDCl₃) δ 8.42 (s, 1H), 7.63 (s, 1H), 7.46 (dd, J=8.4, 5.3 Hz, 1H), 7.29 (d, J=1.4 Hz, 1H), 7.26-7.20 (m, 2H), 6.50 (s, 1H), 5.32-5.11 (m, 1H), 4.42 (ddd, J=19.4, 11.9, 6.0 Hz, 1H), 4.15 (dd, J=24.3, 12.0 Hz, 1H), 4.09-3.73 (m, 7H), 3.08-2.91 (m, 2H), 2.60-2.49 (m, 1H), 2.21 (t, J=11.0 Hz, 1H), 2.11 (ddd, J=13.0, 8.0, 4.9 Hz, 1H), 1.17 (dd, J=6.4, 2.5 Hz, 3H), 1.12-1.02 (m, 4H). ESI MS [M+H]⁺ for C₃₀H₃₀F₂N₆O₃, calcd 561.2, found 561.2.

### Example 27: 6-(6-cyclopropyl-4-{4-fluoro-2-[(3-fluoro-1-azetidinyl)carbonyl]phenyl}-2-pyridyl)-2-(1-hydroxyethyl)-1,6-dihydro-1,4,6-triaza-7-indenone

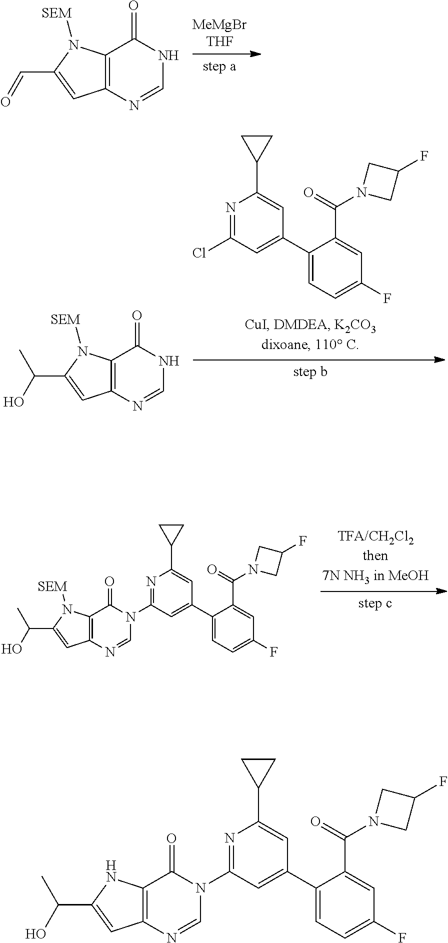 [SMILES: C[Si](C)(C)CCOCn1c(C=O)cc2nc[nH]c(=O)c21 ; O=C(c1cc(F)ccc1-c1cc(Cl)nc(C2CC2)c1)N1CC(F)C1 ; CC(O)c1cc2nc[nH]c(=O)c2n1COCC[Si](C)(C)C ; CC(O)c1cc2ncn(-c3cc(-c4ccc(F)cc4C(=O)N4CC(F)C4)cc(C4CC4)n3)c(=O)c2n1COCC[Si](C)(C)C ; CC(O)c1cc2ncn(-c3cc(-c4ccc(F)cc4C(=O)N4CC(F)C4)cc(C4CC4)n3)c(=O)c2[nH]1]

Step a: To a solution of 4-oxo-5-(2-trimethylsilylethoxymethyl)-3H-pyrrolo[3,2-d]pyrimidine-6-carbaldehyde (1.0 g, 3.4 mmol, 1 equiv., obtained according to example 25, step c) in THF (17 mL) MeMgBr (1.7 mL, 1.5 equiv., 3 M solution in Et₂O) was added dropwise at 0° C. The resulting cloudy mixture was stirred at 0° C. for 20 min. TLC analysis indicated complete consumption of the starting material. The mixture was quenched by careful addition of aq. sat. NH₄Cl, diluted with EtOAc. The organic extract was separated, and the aqueous layer was additionally extracted with EtOAc. Combined EtOAc solution was washed with brine, dried over Na₂SO₄ and concentrated to dryness. The dry residue was fractionated by column chromatography (SiO₂, EtOAc in dichloromethane, 0 to 100%) to afford the desired product.

Step b: To a solution of the 6-(1-hydroxyethyl)-5-(2-trimethylsilylethoxymethyl)-3H-pyrrolo[3,2-d]pyrimidin-4-one (40 mg, 0.13 mmol, 1.0 equiv.) and [2-(2-chloro-6-cyclopropylpyridin-4-yl)-5-fluorophenyl]-(3-fluoroazetidin-1-yl)methanone (45.9 mg, 0.13 mmol, 1.2 equiv., obtained according to example 21, step b) in dioxane (2.6 mL, 0.05M) was added CuI (25 mg, 0.13 mmol, 1.0 equiv.), N,N′-dimethylethylenediamine (23.0 mg, 0.26 mmol, 2.0 equiv.), and K₂CO₃ (54 mg, 0.34 mmol, 3.0 equiv.). The resulting mixture was heated at 110° C. for 12 h. After cooling to room temperature, the reaction mixture was diluted with EtOAc and washed with water twice. The organic phase was then washed with brine, dried over Na₂SO₄ and concentrated to yield the coupling product that was used for the next step without purification.

Step c: The product from step b was dissolved in a mixture of TFA/dichloromethane (3 mL, 1:1 v/v) and stirred at room temperature for 4 h. The resulting mixture was concentrated to dryness under reduced pressure. The residual TFA was removed by co-evaporation with dichloromethane. The dry residue was dissolved in 7N NH₃ in MeOH (4 mL) and stirred at room temperature for 1 h. The solvent was removed, and the crude residue was purified by reversed phase HPLC to afford the title compound. ¹H NMR (400 MHz, CDCl₃) δ 10.86 (s, 1H), 8.31 (s, 1H), 7.58 (d, J=1.4 Hz, 1H), 7.54-7.47 (m, 1H), 7.29 (d, J=1.3 Hz, 1H), 7.24-7.20 (m, 2H), 6.32 (d, J=2.1 Hz, 1H), 5.23-4.97 (m, 2H), 4.40-4.27 (m, 1H), 4.19-4.06 (m, 2H), 3.98-3.87 (m, 1H), 3.83-3.72 (m, 1H), 2.15-2.08 (m, 1H), 1.49 (d, J=6.5 Hz, 3H), 1.12-1.01 (m, 4H). ESI MS [M+H]⁺ for C₂₆H₂₃F₂N₅O₃ calcd 492.1 found 492.1.

### Example 28: 6-{[(S)-3-methyl-1-piperidyl]methyl}-2-(6-cyclopropyl-4-{4-fluoro-2-[(3-fluoro-1-azetidinyl)carbonyl]phenyl}-2-pyridyl)-1-isoindolinone

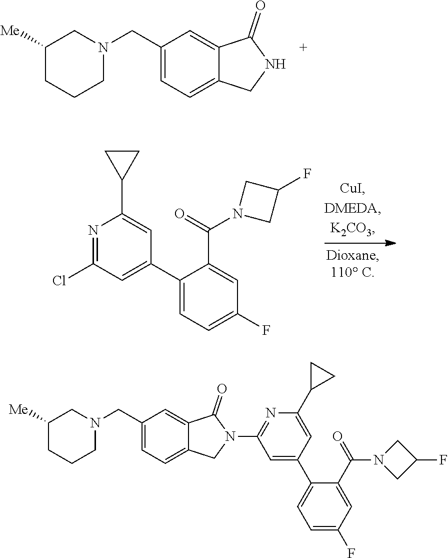 [SMILES: C[C@H]1CCCN(Cc2ccc3c(c2)C(=O)NC3)C1 ; O=C(c1cc(F)ccc1-c1cc(Cl)nc(C2CC2)c1)N1CC(F)C1 ; C[C@H]1CCCN(Cc2ccc3c(c2)C(=O)N(c2cc(-c4ccc(F)cc4C(=O)N4CC(F)C4)cc(C4CC4)n2)C3)C1]

To a solution of the a 6-[[(3S)-3-methylpiperidin-1-yl]methyl]-2,3-dihydroisoindol-1-one (31.7 mg, 0.13 mmol, 1.0 equiv.) and [2-(2-chloro-6-cyclopropylpyridin-4-yl)-5-fluorophenyl]-(3-fluoroazetidin-1-yl)methanone (45.9 mg, 0.13 mmol, 1.2 equiv., obtained similar to example 21, step b) in dioxane (2.6 mL, 0.05M) was added CuI (25 mg, 0.13 mmol, 1.0 equiv.), N,N′-dimethylethylenediamine (23.0 mg, 0.26 mmol, 2.0 equiv.), and K₂CO₃ (54 mg, 0.34 mmol, 3.0 equiv.). The resulting mixture was heated at 110° C. for 12 h. After cooling to room temperature, the reaction mixture was diluted with EtOAc and washed sequentially with aq. sat. NH₄Cl, water and brine. The organic extract was dried over Na₂SO₄ and concentrated to dryness under reduced pressure. The crude residue was then purified by reverse phase HPLC to yield the title compound. ¹H NMR (400 MHz, CDCl₃) δ 8.43 (d, J=1.4 Hz, 1H), 7.80 (s, 1H), 7.62 (d, J=7.8 Hz, 1H), 7.56-7.46 (m, 2H), 7.25-7.15 (m, 2H), 7.06 (d, J=1.5 Hz, 1H), 5.58-5.38 (m, 1H), 5.13-4.96 (m, 2H), 4.49-4.21 (m, 2H), 4.20-3.98 (m, 2H), 3.57 (s, 2H), 2.85-2.71 (m, 2H), 2.09-2.01 (m, 1H), 1.95-1.86 (m, 1H), 1.74-1.53 (m, 5H), 1.14-1.05 (m, 2H), 1.03-0.97 (m, 2H), 0.92-0.76 (m, 5H). ESI MS [M+H]⁺ for C₃₃H₃₄F₂N₄O₂ calcd 557.2 found 557.3.

### Example 29: 7-cyclopropyl-5-[3-cyclopropyl-5-[4-fluoro-2-(3-fluoroazetidine-1-carbonyl)phenyl]phenyl]-2-[[(3S)-3-methylpiperidin-1-yl]methyl]-3H-imidazo[4,5-c]pyridin-4-one

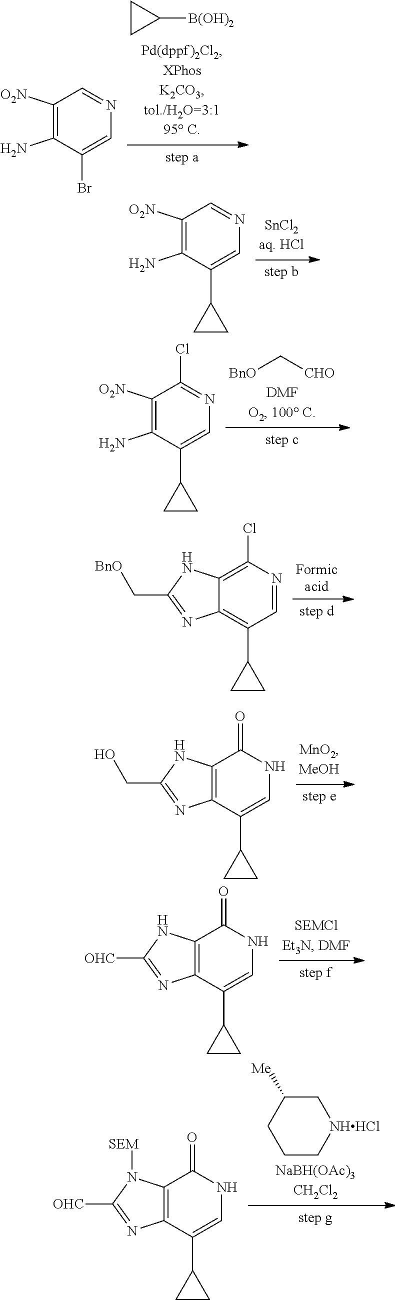

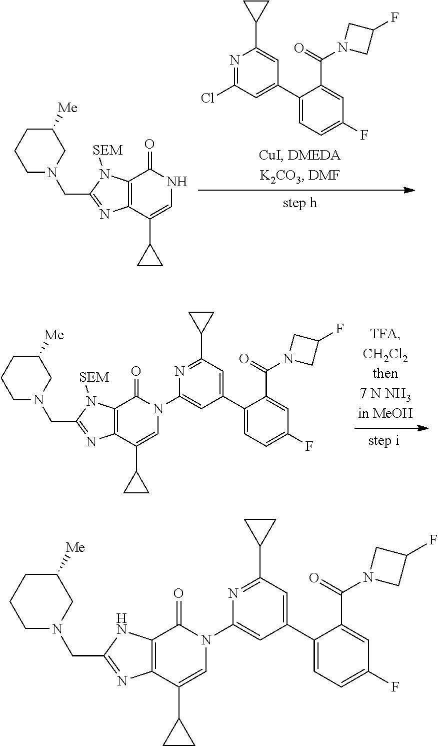 [SMILES: C[C@H]1CCCN(Cc2nc3c(C4CC4)c[nH]c(=O)c3n2COCC[Si](C)(C)C)C1 ; O=C(c1cc(F)ccc1-c1cc(Cl)nc(C2CC2)c1)N1CC(F)C1 ; C[C@H]1CCCN(Cc2nc3c(C4CC4)cn(-c4cc(-c5ccc(F)cc5C(=O)N5CC(F)C5)cc(C5CC5)n4)c(=O)c3n2COCC[Si](C)(C)C)C1 ; C[C@H]1CCCN(Cc2nc3c(C4CC4)cn(-c4cc(-c5ccc(F)cc5C(=O)N5CC(F)C5)cc(C5CC5)n4)c(=O)c3[nH]2)C1]

Step a: 3-Bromo-5-nitro-4-pyridinamine (218 mg, 1.0 mmol, 1 equiv.), cyclopropylboronic acid (129 mg, 1.5 mmol, 1.5 equiv.), Xphos (38.0 mg, 0.08 mmol. 0.08 equiv.) and K₂CO₃ (414 mg, 3.0 mmol, 3 equiv.) were dissolved in a mixture of 8 mL toluene and 2 mL water. The reaction was sparged with nitrogen for 10 mins before adding Pd(dppf)Cl₂ (36.6 mg, 0.05 mmol). Then the mixture was heated at 95° C. overnight under stirring. After cooling to room temperature, the reaction was concentrated to dryness under reduced pressure. The obtained residue was fractionated by column chromatography (SiO₂, 0-80% EtOAc/hexanes) to afford the desired product.

Step b: To the solution of the product from step a (156 mg, 0.87 mmol, 1 equiv) in concentrated HCl (3 mL) preheated to 90° C. SnCl₂ (658 mg, 3.5 mmol, 4 equiv.) in 1.5 mL concentrated HCl was added dropwise. Once the addition was complete, the reaction vial was sealed, and the mixture was heated to 130° C. for 3 hours. After cooling to room temperature, the reaction was neutralized to pH˜7 by adding 2M NaOH. The product was extracted with CHCl₃:iPrOH=3:1 (3×20 mL), and the combined organic phase was dried over Na₂SO₄ and concentrated to dryness under vacuum. The crude residue was purified by column chromatography (SiO₂, 40-100% EtOAc/hexanes) to afford the desired product.

Step c: To a solution of the product from step b (920 mg, 5.0 mmol, 1 equiv.) in DMF (10 mL, 0.5 M) was added 2-phenylmethoxy-acetaldehyde (750 mg, 5.0 mmol, 1 equiv.). The mixture was sparged with oxygen for 10 mins and heated with oxygen balloon in a sealed vial at 100° C. overnight. After cooling to room temperature, the reaction mixture was concentrated to dryness, and the residue was used for the next step without purification.

Step d: The product from step c was dissolved in 5 mL formic acid and the reaction was stirred at 90° C. overnight. After cooling to room temperature, the reaction was concentrated to dryness. Purification by column chromatography (SiO₂, 0-10% MeOH/DCM) afforded the desired pyridone product.

Step e: To the solution of the product from step d (505 mg, 2.5 mmol, 1 equiv.) in MeOH (12.5 mL, 0.2 M) MnO₂ (652 mg, 7.5 mmol, 3 equiv.) was added. The reaction mixture was stirred at 50° C. overnight. The crude mixture was cooled to room temperature and passed through a pad of Celite® to remove solids. The filtrate was concentrated to dryness under reduced pressure to afford the crude aldehyde product that was used for the next step without purification.

Step f: To the solution of the crude product from step e in DMF (12.5 mL, 0.2 M), Et₃N (0.76 g, 7.5 mmol, 3 equiv.) and SEMCl (622 mg, 3.75 mmol, 1.5 equiv.) were sequentially added. The resulting solution was stirred at room temperature overnight. The reaction mixture was concentrated to dryness under reduced pressure, and the crude residue was purified by column chromatography (SiO₂, 40-100% EtOAc/hexanes) to afford the desired SEM-protected product.

Step g: The product from step f (542 mg, 1.6 mmol, 1 equiv.) was dissolved in dichloromethane (15 mL). Then (3S)—3-methyl-piperidine hydrochloride (310 mg, 2.3 mmol, 1.4 eq.) and Et₃N (317 mg, 3.1 mmol, 1.9 equiv.) were added. The reaction mixture was stirred at room temperature for 30 mins before NaBH(OAc)₃ (488 mg, 2.3 mmol, 1.4 equiv.) was added. After stirring for an additional 1 hour the reaction was quenched with water, and the product was extracted with dichloromethane (3×30 mL). The combined organic extract was dried over Na₂SO₄ and concentrated to dryness under reduced pressure. The crude product was purified by column chromatography (SiO₂, 0-10% MeOH in dichloromethane) to give the desired product.

Step h: To a solution of the product from step g (41.6 mg, 0.1 mmol), [2-(2-chloro-6-cyclopropylpyridin-4-yl)-5-fluorophenyl]-(3-fluoroazetidin-1-yl)methanone (35.0 mg, 0.1 mmol, 1 equiv., obtained similar to example 21, step b) and DMEDA (21 μL, 0.2 mmol, 2 equiv.) in DMF (2.0 mL, 0.05 M), K₂CO₃ (41.4 mg, 0.3 mmol, 3 equiv.) was added. The mixture was sparged with nitrogen for 10 mins before the addition of CuI (19.0 mg, 0.1 mmol, 1 equiv.). The resulting mixture was stirred in a sealed vial at 100° C. overnight. After cooling to room temperature, the mixture was quenched with sat. aq. NH₄Cl and extracted with EtOAc (3×15 mL). The combined organic phase was washed with water twice, dried over Na₂SO₄ and concentrated to dryness under vacuum. The residue was purified by column chromatography (SiO₂, 0-5% MeOH/DCM) to afford the desired product.

Step i: A solution of the product of step h (20.0 mg, 0.03 mmol) in dichloromethane (1.0 mL) and TFA (1.0 mL) was stirred for 1 hour at room temperature. Then it was concentrated to dryness under vacuum, and the residue was dissolved in 7N NH₃ in MeOH (1 mL). After stirring at room temperature for 1 h the reaction was concentrated to dryness. The crude product was purified by reversed phase HPLC. ¹H NMR (400 MHz, CDCl₃) δ 8.16-8.05 (m, 1H), 7.67 (s, 1H), 7.57-7.34 (m, 3H), 7.21 (ddd, J=10.8, 5.3, 3.1 Hz, 3H), 5.19 (d, J=56.7 Hz, 1H), 4.37 (ddd, J=19.3, 11.9, 6.0 Hz, 1H), 4.13 (dd, J=23.9, 12.0 Hz, 1H), 3.97 (dt, J=18.8, 8.2 Hz, 1H), 3.81 (dd, J=23.5, 10.9 Hz, 1H), 2.23-2.05 (m, 1H), 1.98 (d, J=28.1 Hz, 3H), 1.04-1.59 (m, 5H), 1.14-1.02 (m, 5H), 0.98 (d, J=7.9 Hz, 3H), 0.93 (d, J=6.0 Hz, 3H). ESI MS [M+H]⁺ for C₃₂H₃₄F₂N₅O₂, calcd 598.3, found 598.2.

### Example 30: 4-cyclopropyl-6-[2-cyclopropyl-6-[4-fluoro-2-(3-fluoroazetidine-1-carbonyl)phenyl]pyrimidin-4-yl]-2-[[(3S)-3-methylpiperidin-1-yl]methyl]—1H-pyrrolo[2,3-c]pyridin-7-one

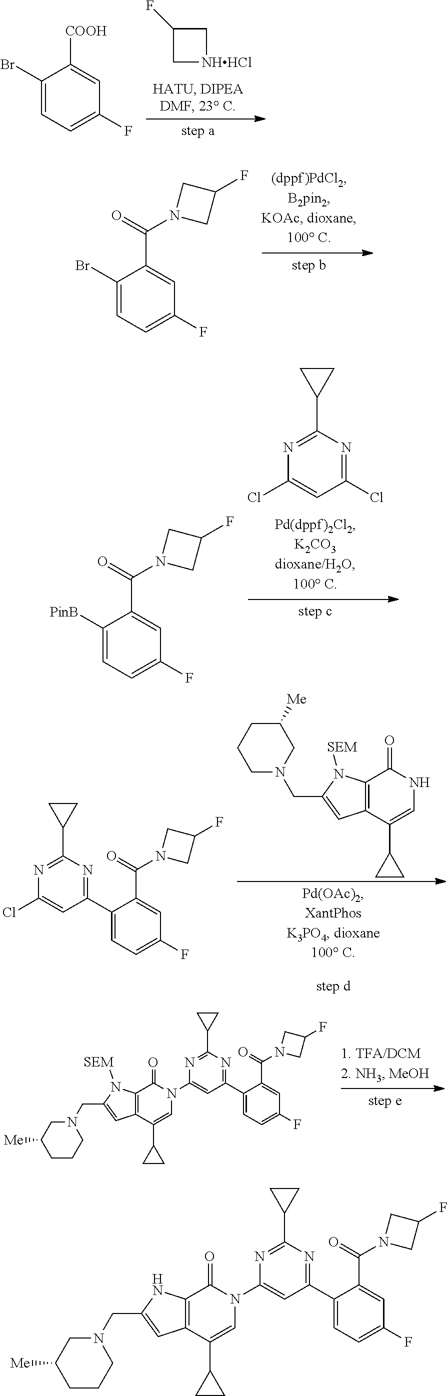

Step a: To a solution of a 2-bromo-5-fluorobenzoic acid (2.1 g, 9.6 mmol, 1.0 equiv.) in DMF (20 mL, 0.5 M) were added i-Pr₂NEt (5.02 mL, 28.8 mmol, 3.0 equiv.), HATU (5.48 g, 14.4 mmol, 1.5 equiv.) and 3-fluoroazetidine hydrochloride (1.29 g, 11.5 mmol, 1.2 equiv.). The reaction mixture was stirred overnight at room temperature, then diluted with EtOAc (50 mL) and water (50 mL). The organic phase was separated, sequentially washed with water (50 mL) and brine (50 mL), dried over Na₂SO₄ and concentrated to dryness under reduced pressure. The crude residue was purified by column chromatography (SiO₂, 0-100% EtOAc gradient in hexanes) to afford (2-bromo-5-fluorophenyl)-(3-fluoroazetidin-1-yl)methanone.

Step b: A mixture of the product from step a (500 mg, 1.81 mmol, 1.0 equiv.), bis(pinacolato)diboron (552 mg, 2.17 mmol, 1.2 equiv.), [1,1′-bis(diphenylphosphino)ferrocene]dichloropalladium(II) (133 mg, 0.181 mmol, 0.1 equiv.) and KOAc (356 mg, 3.62 mmol, 2.0 equiv.) was placed under nitrogen. Degassed dioxane (9 mL, 0.2 M) was added, and the reaction mixture was stirred at 100° C. for 2 hours. The obtained solution was cooled to 23° C. and diluted with EtOAc (50 mL) and water (30 mL). The cloudy biphasic solution was filtered through a pad of Celite® to remove solids. The organic phase was separated, dried over Na₂SO₄ and concentrated to dryness under reduced pressure. The crude residue was purified by column chromatography (SiO₂, 0-100% EtOAc gradient in hexanes) to afford (3-fluoroazetidin-1-yl)-[5-fluoro-2-(4,4,5,5-tetramethyl-1,3,2-dioxaborolan-2-yl)phenyl]methanone.

Step c: To a solution of product from step b (254.7 mg, 0.788 mmol, 1.0 equiv.), 4,6-dichloro-2-cyclopropylpyrimidine (299 mg, 1.58 mmol, 2.0 equiv.) and K₂CO₃ (218 mg, 1.58 mmol, 2.0 equiv.) in dioxane/water mixture (5 mL, 4:1 v/v) was added Pd(dppf)Cl₂ (58 mg, 0.0788 mmol, 0.1 equiv.). The mixture was degassed under vacuum and backfilled with nitrogen (repeated 2 times) and stirred at 100° C. for 16 h. The resulting mixture was cooled to room temperature, diluted with EtOAc (30 mL) and brine (10 ml). The organic phase was separated, dried over Na₂SO₄, and the solvent was removed under reduced pressure. The material was purified by column chromatography (SiO₂, 0-100% EtOAc gradient in hexane) to afford [2-(6-chloro-2-cyclopropylpyrimidin-4-yl)-5-fluorophenyl]-(3-fluoroazetidin-1-yl)methanone.

Step d: To a mixture of product from step c (81 mg, 0.232 mmol, 1.0 equiv.), 4-cyclopropyl-2-[[(3S)-3-methylpiperidin-1-yl]methyl]-1-(2-trimethylsilylethoxymethyl)-6H-pyrrolo[2,3-c]pyridin-7-one (97 mg, 0.232 mmol, 1.0 equiv., obtained according to example 1) in dioxane (1.2 mL) was added Pd(OAc)₂ (11 mg, 0.0464 mmol, 20 mol %), XantPhos (54 mg, 0.0928 mmol, 40 mol %) and K₃PO₄ (148 mg, 0.696 mmol, 3.0 equiv.). The resulting mixture was degassed under vacuum and backfilled with nitrogen 2 rimes, then heated at 100° C. for 2.5 h under nitrogen atmosphere. Once LCMS showed a complete conversion of coupling partners, the reaction was cooled to room temperature and diluted with EtOAc (10 mL). The organic extract was sequentially washed with water (10 mL) and brine (10 mL), dried over Na₂SO₄ and concentrated under vacuum. The crude residue was purified by column chromatography (SiO₂, 0 to 20% MeOH gradient in dichlorormethane) to afford 4-cyclopropyl-6-[2-cyclopropyl-6-[4-fluoro-2-(3-fluoroazetidine-1-carbonyl)phenyl]pyrimidin-4-yl]-2-[[(3S)-3-methylpiperidin-1-yl]methyl]-1-(2-trimethylsilylethoxymethyl)pyrrolo[2,3-c]pyridin-7-one.

Step e: To a solution of the product from step d in dichloromethane (2 mL) was added trifluoroacetic acid (1 mL). The resulting solution was stirred at 23° C. for 1 h, then the solvent was evaporated, and the crude residue was dissolved in 7M NH₃ in MeOH (3 mL). After stirring for 30 min the mixture was concentrated to dryness, and the crude product was purified by prep-HPLC (SiO₂ C18, 10 to 80% CH3CN in water with 0.1% trifluoroacetic acid) to furnish the title compound. ¹H NMR (400 MHz, DMSO-d₆) δ 12.12 (s, 1H), 8.13 (s, 1H), 7.84 (dd, J=8.6, 5.3 Hz, 1H), 7.48-7.36 (m, 3H), 6.33 (d, J=1.9 Hz, 1H), 5.66-5.28 (m, 1H), 4.45-4.17 (m, 2H), 4.17-3.95 (m, 2H), 3.55 (s, 2H), 2.75 (t, J=9.0 Hz, 2H), 2.26 (dt, J=12.6, 6.3 Hz, 1H), 1.98-1.79 (m, 2H), 1.65-1.52 (m, 4H), 1.50-1.35 (m, 1H), 1.09 (d, J=8.1 Hz, 4H), 0.89-0.70 (m, 6H), 0.62 (td, J=5.7, 3.9 Hz, 2H). ESI MS [M+H]⁺ for C₃₄H₃₇F₂N₆O₂, calcd 599.3, found 599.3.

### Example 31: 4-cyclopropyl-6-[4-cyclopropyl-6-[4-fluoro-2-(3-fluoroazetidine-1-carbonyl)phenyl]pyrimidin-2-yl]-2-[[(3S)-3-methylpiperidin-1-yl]methyl]—1H-pyrrolo[2,3-c]pyridin-7-one

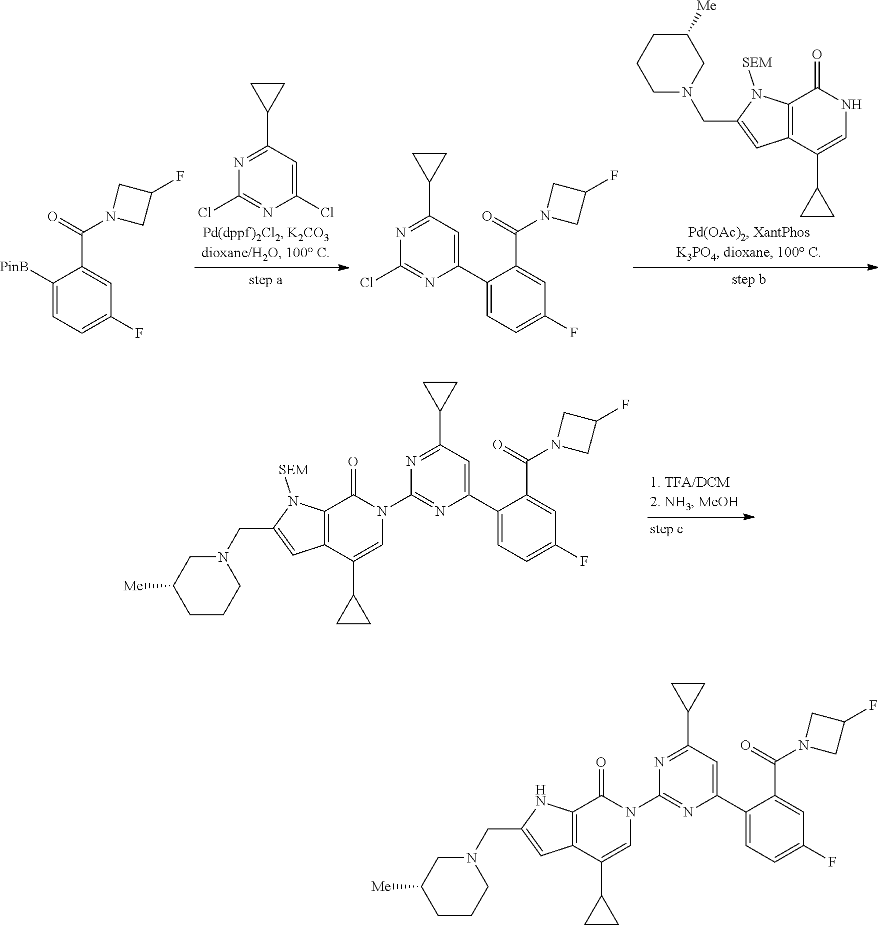

Step a: To a solution of (3-fluoroazetidin-1-yl)-[5-fluoro-2-(4,4,5,5-tetramethyl-1,3,2-dioxaborolan-2-yl)phenyl]methanone (162 mg, 0.501 mmol, 1.0 equiv., obtained according to Example 30 steps a-b), 2,4-dichloro-6-cyclopropylpyrimidine (189 mg, 1.00 mmol, 2.0 equiv.) and K₂CO₃ (138 mg, 1.00 mmol, 2.0 equiv.) in dioxane/water mixture (3 mL, 4:1 v/v) was added Pd(dppf)Cl₂ (37 mg, 0.05 mmol, 0.1 equiv.). The mixture was degassed under vacuum and backfilled with nitrogen (repeated 2 times) and stirred at 100° C. for 6 h. The resulting mixture was cooled to room temperature, diluted with EtOAc (30 mL) and brine (10 ml). The organic phase was separated, dried over Na₂SO₄, and the solvent was removed under reduced pressure. The material was purified by column chromatography (SiO₂, 0-100% EtOAc gradient in hexane) to afford [2-(2-chloro-6-cyclopropylpyrimidin-4-yl)-5-fluorophenyl]-(3-fluoroazetidin-1-yl)methanone.

Steps b,c: These steps were performed in a similar fashion to steps b,c of the example 30 to afford the title compound. ¹H NMR (400 MHz, DMSO-d₆) δ 12.09 (s, 1H), 8.07 (dd, J=8.7, 5.3 Hz, 1H), 7.89 (s, 1H), 7.50 (td, J=8.6, 2.7 Hz, 1H), 7.35 (dd, J=8.7, 2.7 Hz, 1H), 6.91 (s, 1H), 6.39 (s, 1H), 5.21-4.69 (m, 1H), 4.28-4.13 (m, 1H), 4.05-3.77 (m, 3H), 3.77-3.45 (m, 2H), 2.81 (s, 2H), 2.36-2.20 (m, 1H), 1.92 (tt, J=7.5, 4.2 Hz, 2H), 1.71-1.39 (m, 5H), 1.25-1.07 (m, 4H), 0.88-0.78 (m, 6H), 0.64 (t, J=2.6 Hz, 2H). ESI MS [M+H]⁺ for C₃₄H₃₇F₂N₆O₂, calcd 599.3, found 599.3.

### Example 32: 3-[6-cyclopropyl-4-[4-fluoro-2-(3-fluoroazetidine-1-carbonyl)phenyl]pyridin-2-yl]-6-pyrrolidin-3-yl-5H-pyrrolo[3,2-d]pyrimidin-4-one

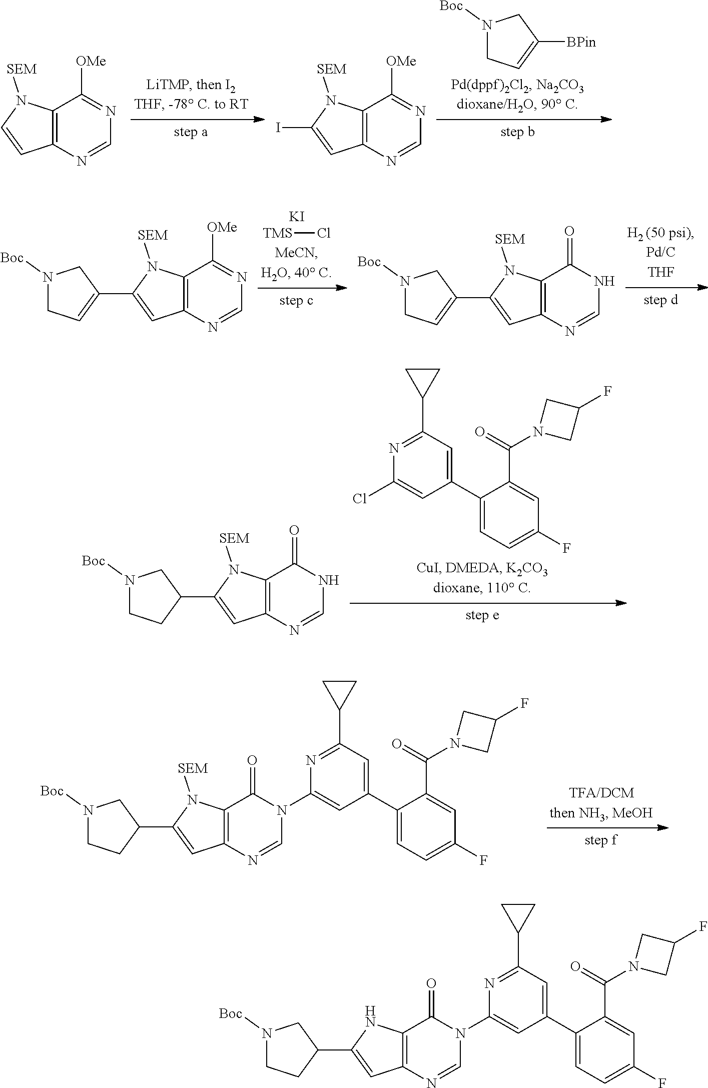

Step a: 2,2,6,6-Tetramethylpiperidine (2.8 mL, 16.4 mmol, 1.55 equiv.) was added to THF (100 mL) and the resulting solution was cooled to −78° C. n-BuLi (1.6 M in hexanes, 9.9 mL, 15.9 mmol, 1.50 equiv.) was added dropwise over 10 min, and the reaction mixture was stirred for 30 minutes. 2-[(4-Methoxypyrrolo[3,2-d]pyrimidin-5-yl)methoxy]ethyl-trimethylsilane (prepared according to Example 25, 2.96 g, 10.6 mmol, 1.0 equiv.) was then added dropwise over 10 min as a solution in THF (10 mL), and the reaction was stirred for additional 2 h at −78° C. Elemental iodine (5.4 g, 21.2 mmol, 2.0 equiv.) was added dropwise over 2 min as a solution in THF (20 mL), and the reaction mixture was stirred for 30 minutes at which point the dry ice bath was removed. The reaction mixture was allowed to warm to room temperature and quenched with saturated aqueous NH₄Cl (40 mL) after 5 min. The resulting biphasic solution was partitioned between EtOAc (200 mL) and H₂O (250 mL). The aqueous phase was extracted with EtOAc (100 mL), and the combined organics were dried over Na₂SO₄, filtered, and concentrated under vacuum. The crude residue was purified by column chromatography (SiO₂, 0 to 100% EtOAc gradient in hexanes) to afford the desired iodination product.

Step b: To a solution of the product from step a (250 mg, 0.62 mmol, 1.0 equiv.) in dioxane (6 mL) were added tert-butyl 3-(4,4,5,5-tetramethyl-1,3,2-dioxaborolan-2-yl)-2,5-dihydropyrrole-1-carboxylate (273 mg, 0.93 mmol, 1.5 equiv.) and 1.0 M aqueous Na₂CO₃ (1.85 mL). The reaction mixture was sparged with N₂ for 10 minutes. Pd(dppf)₂Cl₂ (44 mg, 0.06 mmol, 0.1 equiv.) was added, and the reaction mixture was heated to 90° C. and stirred for 90 minutes. At that point it was cooled to room temperature and quenched with a 1:1 mixture of water/brine (100 mL). The product was extracted with EtOAc (2×50 mL). The combined organics were dried over Na₂SO₄, filtered, and concentrated under vacuum. The crude residue was purified by column chromatography (SiO₂, 0 to 100% EtOAc gradient in hexanes) to afford the desired product.

Step c: To a solution of the product from step b (263 mg, 0.59 mmol, 1.0 equiv.) in acetonitrile (6 mL) was added KI (156 mg, 0.94 mmol, 1.6 equiv.), TMSCl (120 μL, 0.94 mmol, 1.6 equiv.) and water (32 μL, 1.77 mmol, 3.0 equiv.). The reaction mixture was stirred at 40° C. for 16 hours at which point it was quenched with a 1:1 mixture of water and saturated aqueous Na₂S₂O₃ (100 mL). The product was extracted with EtOAc (2×50 mL). The combined organics were dried over Na₂SO₄, filtered, and concentrated under vacuum. The crude residue was purified by column chromatography (SiO₂, 0 to 25% MeOH gradient in dichloromethane) to afford the desired product.

Step d: A Parr shaker was charged with the product from step c (120 mg, 0.28 mmol, 1.0 equiv.) in THF (10 mL). Pd/C (200 mg, 10 wt % Pd) was added and the Parr shaker was evacuated/backfilled with 50 psi of H₂ three times. The reaction mixture was agitated under 50 psi of hydrogen overnight. Hydrogen pressure was released, the reaction mixture was sparged with nitrogen for 10 min, then filtered through a Celite® pad to remove solids. The filtrate was concentrated to dryness under vacuum, and the crude residue was used without any further purification for the next step.

Step e: To a suspension of the product from step d (46 mg, 0.11 mmol, 1.0 equiv.), [2-(2-chloro-6-cyclopropylpyridin-4-yl)-5-fluorophenyl]-(3-fluoroazetidin-1-yl)methanone (prepared according to Example 21, 37 mg, 0.11 mmol, 1.0 equiv.), and K₂CO₃ (44 mg, 0.33 mmol, 3.0 equiv.) in dioxane (2 mL) was added CuI (21 mg, 0.11 mmol, 1.0 equiv.) and DMEDA (23 uL, 0.22 mmol, 2.0 equiv.). The reaction mixture was sparged with nitrogen for 10 min, the vial was sealed and heated at 110° C. for 16 hours. The resulting mixture was cooled to room temperature and quenched with aq. sat. NH₄Cl (3 mL) and diluted with EtOAc (10 mL). The organic phase was separated, and the aqueous phase was extracted with EtOAc (2×5 mL). The combined organics were dried over Na₂SO₄, filtered, and concentrated under vacuum. The crude residue was purified by column chromatography (SiO₂, 0 to 20% MeOH gradient in dichloromethane) to afford the desired product.

Step f: To a solution of the product from step e (52 mg, 0.07 mmol, 1.0 equiv.) in dichloromethane (1 mL) was added TFA (1 mL). The reaction mixture was heated to 30° C. and stirred for 1 hour at which point it was cooled to 23° C., diluted with toluene (5 mL) and concentrated to dryness under reduced pressure. The crude residue was dissolved in 7M NH₃ in MeOH (2 mL), and the resulting solution was stirred at 30° C. for 1 hour. The reaction was directly concentrated and purified by reverse phase prep-HPLC (SiO₂ C18 column, 5 to 50% CH₃CN gradient in water with 0.1% formic acid) to afford the desired product. ¹H NMR (400 MHz, CDCl₃) δ 8.36 (s, 1H), 7.61 (d, J=1.3 Hz, 1H), 7.47 (dd, J=8.5, 5.2 Hz, 1H), 7.30-7.18 (m, 3H), 6.29 (s, 1H), 5.26-5.01 (m, 1H), 4.47-4.32 (m, 1H), 4.23-4.04 (m, 1H), 3.99-3.84 (m, 1H), 3.84-3.67 (m, 1H), 3.48-3.38 (m, 1H), 3.26 (dd, J=10.4, 7.1 Hz, 1H), 3.22-3.12 (m, 1H), 3.10-2.94 (m, 2H), 2.24 (dtd, J=13.0, 8.4, 4.7 Hz, 1H), 2.10 (tt, J=7.9, 4.9 Hz, 1H), 1.98-1.81 (m, 1H), 1.13-1.00 (m, 4H). ESI MS [M+H]⁺ for C₂₈H₂₆F₂N₆O₂ calcd 517.2 found 517.2.

### Example 33: 6-cyclopropyl-3-[6-cyclopropyl-4-[4-fluoro-2-(3-fluoroazetidine-1-carbonyl)phenyl]pyridin-2-yl]-5H-pyrrolo[3,2-d]pyrimidin-4-one

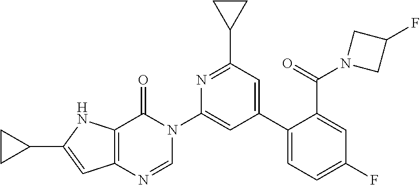 [SMILES: O=C(c1cc(F)ccc1-c1cc(C2CC2)nc(-n2cnc3cc(C4CC4)[nH]c3c2=O)c1)N1CC(F)C1]

The title compound was prepared in a similar fashion to that described for example 32 using cyclopropylboronic acid for step b. ¹H NMR (400 MHz, CDCl₃) δ 9.66 (s, 1H), 8.36 (s, 1H), 7.60 (d, J=1.5 Hz, 1H), 7.50-7.43 (m, 1H), 7.28 (d, J=1.4 Hz, 1H), 7.25-7.20 (m, 2H), 6.20 (dd, J=2.4, 0.5 Hz, 1H), 5.27-4.99 (m, 1H), 4.45-4.28 (m, 1H), 4.23-4.08 (m, 1H), 4.00-3.86 (m, 1H), 3.85-3.73 (m, 1H), 2.19-2.04 (m, 1H), 2.01-1.90 (m, 1H), 1.15-0.97 (m, 6H), 0.87-0.79 (m, 2H). ESI MS [M+H]⁺ for C₂₇H₂₃F₂N₅O₂ calcd 488.2 found 488.1.

### Example 34: 3-[6-cyclopropyl-4-[2-(3,3-difluoroazetidine-1-carbonyl)-4-fluorophenyl]pyridin-2-yl]-6-[[[(2S)-1-methoxypropan-2-yl]amino]methyl]-5H-pyrrolo[3,2-d]pyrimidin-4-one

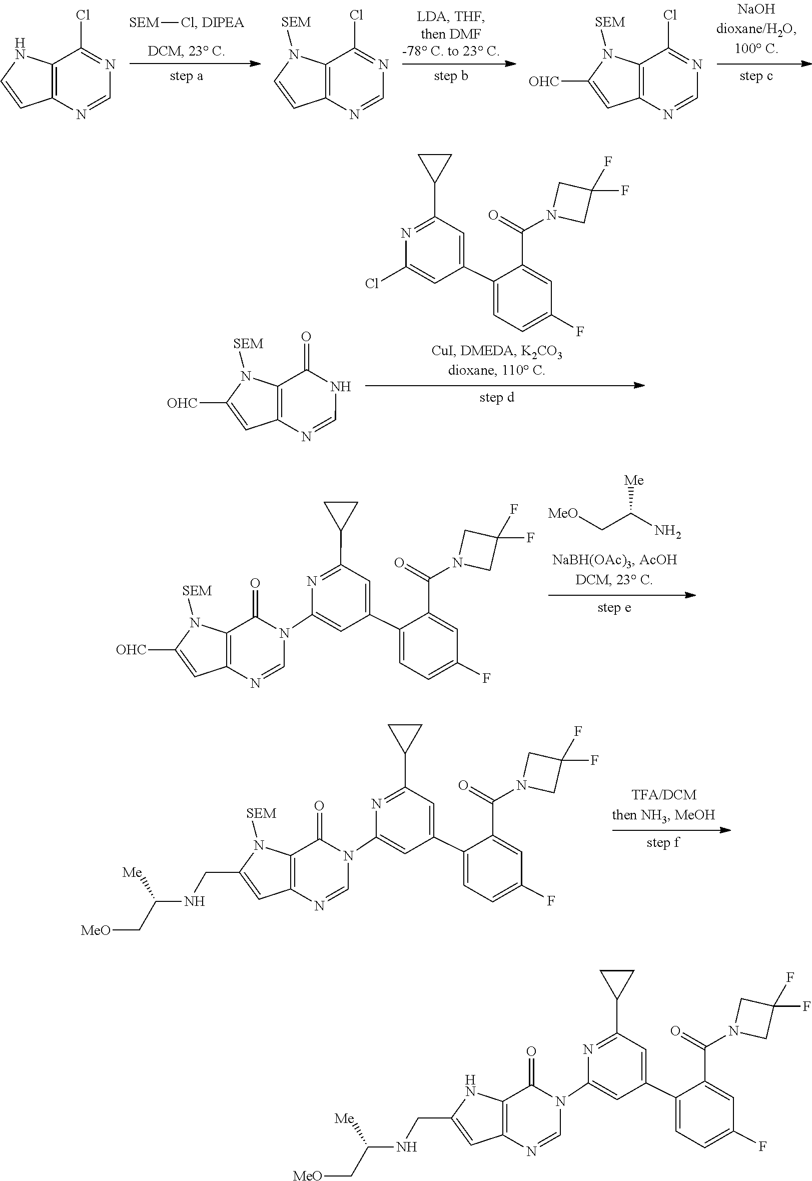

Step a: To a solution of 4-chloro-5H-pyrrolo[3,2-d]pyrimidine (8.5 g, 55.3 mmol, 1.0 equiv.) in dichloromethane (220 mL) was added i-Pr₂NEt (14.5 mL, 83.0 mmol, 1.5 equiv.) followed by SEMCl (10.8 mL, 60.8 mmol, 1.1 equiv.). The reaction mixture was stirred at room temperature for 1 hour at which point it was quenched with a 1:1 mixture of water/brine (500 mL) and extracted with dichloromethane (2×100 mL). The combined organics were dried over Na₂SO₄, filtered, and concentrated to dryness under reduced pressure. The crude residue was purified by column chromatography (SiO₂, 0 to 60% EtOAc gradient in hexanes) to afford the desired product.

Step b: LDA (2.0 M in THF, 3.3 mL, 6.0 mmol, 1.25 equiv.) was diluted with THF (27 mL) under nitrogen atmosphere and the resulting solution was cooled to −78° C. The product of step a (1.5 g, 5.3 mmol, 1.0 equiv.) was added dropwise to the reaction mixture as a solution in THF (5 mL) over 5 min. The reaction mixture was stirred at −78° C. for 1.5 hours, at which point DMF (0.65 mL, 7.9 mmol, 1.5 equiv.) was added dropwise over 1 min to the reaction mixture. The reaction was stirred for an additional 30 minutes at −78° C., the dry ice bath was removed, and the reaction was allowed to warm to room temperature and stir for 30 minutes. The resulting solution was quenched with saturated aqueous NH₄Cl (20 mL) and partitioned between EtOAc (100 mL) and H₂O (100 mL). The organic phase was separated, and the aqueous phase was extracted with EtOAc (100 mL). The combined organic extract was dried over Na₂SO₄, filtered, and concentrated under reduced pressure. The crude residue was purified by column chromatography (SiO₂, 0 to 100% EtOAc gradient in hexanes) to afford the desired product.

Step c: To a solution of the product from step b (1.03 g, 3.3 mmol, 1.0 equiv.) in dioxane (15 mL was added 1.0 M aqueous NaOH (15 mL). The reaction mixture was heated at 100° C. for 45 min, then cooled to room temperature, quenched with sat. aq. NH₄Cl (30 mL), diluted with water (50 mL) and extracted with EtOAc (2×100 mL). The combined organics were dried over Na₂SO₄, filtered, and the solvent was evaporated under reduced pressure. The crude product was purified by column chromatography (SiO₂, 0-100% EtOAc gradient in dichloromethane) to afford the product.

Step d: To a suspension of the product from step c (200 mg, 0.64 mmol, 1.0 equiv.), [2-(2-chloro-6-cyclopropylpyridin-4-yl)-5-fluorophenyl]-(3,3-difluoroazetidin-1-yl)methanone (235 mg, 0.64 mmol, 1.0 equiv., prepared according to Example 21 using 3,3-difluoroazetidine hydrochloride on step a), and K₂CO₃ (267 mg, 1.93 mmol, 3.0 equiv.) in dioxane (13 mL) was added CuI (122 mg, 0.64 mmol, 1.0 equiv.) and DMEDA (140 μL, 1.28 mmol, 2.0 equiv.). The reaction mixture was sparged with nitrogen for 10 min and heated at 110° C. under vigorous stirring for 16 hours. The resulting suspension was cooled to 23° C., quenched with aq. sat. NH₄Cl (10 mL) and extracted with EtOAc (2×15 mL). The combined organics were dried over Na₂SO₄, filtered, and concentrated to dryness under vacuum. The crude residue was purified by column chromatography (SiO₂, 0 to 20% MeOH gradient in dichloromethane) to afford the desired product.

Step e: To a solution of the product of step d (30 mg, 0.05 mmol, 1.0 equiv.) in dichloromethane (1 mL) was added (2S)-1-methoxypropan-2-amine (9 mg, 0.10 mmol, 2.0 equiv.), NaBH(OAc)₃ (30 mg, 0.13 mmol, 2.5 equiv.), and AcOH (6 μL, 0.10 mmol, 2.0 equiv.). The reaction was stirred for 16 hours at room temperature, diluted with saturated aqueous NaHCO₃ (20 mL), and the product was extracted with dichloromethane (2×10 mL). The combined organic extract was dried over Na₂SO₄, filtered, and concentrated to dryness under vacuum. The crude material was used directly in the next step without further purification.

Step f: To a solution of the product from step e (assume 0.05 mmol, 1.0 equiv.) in dichloromethane (1 mL) was added TFA (1 mL). The reaction mixture was stirred at 30° C. for 1 hour. The resulting mixture was cooled to 23° C., diluted with toluene (5 mL) and the solvent was removed under vacuum. The crude residue was dissolved in 7M NH₃ in MeOH (2 mL) and the resulting solution was stirred at 30° C. for 1 hour. Upon solvent evaporation the crude product was purified by reverse phase prep-HPLC (SiO₂ C18, 5 to 50% CH₃CN gradient in water with 0.1% formic acid) to afford the desired product. ¹H NMR (400 MHz, CDCl₃) δ 8.36 (s, 1H), 7.63 (d, J=1.4 Hz, 1H), 7.50 (ddd, J=7.9, 5.2, 1.1 Hz, 1H), 7.29-7.21 (m, 3H), 6.34 (s, 1H), 4.43 (t, J=12.0 Hz, 2H), 4.02 (d, J=4.2 Hz, 2H), 3.97 (t, J=11.5 Hz, 2H), 3.40-3.35 (m, 1H), 3.37 (s, 4H), 3.27 (dd, J=9.4, 7.6 Hz, 1H), 3.02-2.91 (m, 1H), 2.14-2.06 (m, 1H), 1.14-1.08 (m, 2H), 1.05 (d, J=6.4 Hz, 3H), 1.08-1.02 (m, 2H). ESI MS [M+H]⁺ for C₂₉H₂₉F₃N₆O₃ calcd 567.2 found 567.3.

### Example 35: 3-[6-cyclopropyl-4-[4-fluoro-2-(3-fluoroazetidine-1-carbonyl)phenyl]pyridin-2-yl]-6-[(2-methoxyethylamino)methyl]-5H-pyrrolo[3,2-d]pyrimidin-4-one

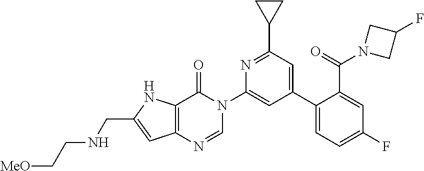 [SMILES: COCCNCc1cc2ncn(-c3cc(-c4ccc(F)cc4C(=O)N4CC(F)C4)cc(C4CC4)n3)c(=O)c2[nH]1]

The title compound was prepared in a similar fashion to that described for example 34 using [2-(2-chloro-6-cyclopropylpyridin-4-yl)-5-fluorophenyl]-(3-fluoroazetidin-1-yl)methanone (prepared according to Example 21) for step b, and 2-methoxyethanamine for step e. ¹H NMR (400 MHz, CDCl₃) δ 8.26 (s, 1H), 7.73 (d, J=1.4 Hz, 1H), 7.59-7.49 (m, 1H), 7.32-7.23 (m, 3H), 6.41 (s, 1H), 5.22-4.96 (m, 1H), 4.60-4.36 (m, 3H), 4.19 (dd, J=24.1, 12.0 Hz, 1H), 3.97-3.62 (m, 4H), 3.43 (s, 2H), 3.39 (s, 3H), 2.15-2.05 (m, 1H), 1.15-0.95 (m, 5H). ESI MS [M+H]⁺ for C₂₈H₂₈F₂N₆O₃ calcd 535.2 found 535.2.

### Example 36: 3-[6-cyclopropyl-4-[4-fluoro-2-(3-fluoroazetidine-1-carbonyl)phenyl]pyridin-2-yl]-6-[[(1-methylcyclobutyl)amino]methyl]-5H-pyrrolo[3,2-d]pyrimidin-4-one

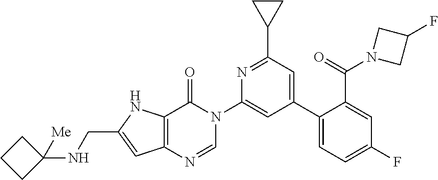 [SMILES: CC1(NCc2cc3ncn(-c4cc(-c5ccc(F)cc5C(=O)N5CC(F)C5)cc(C5CC5)n4)c(=O)c3[nH]2)CCC1]

The title compound was prepared in a similar fashion to that described for example 34 using [2-(2-chloro-6-cyclopropylpyridin-4-yl)-5-fluorophenyl]-(3-fluoroazetidin-1-yl)methanone (prepared according to Example 21) for step b, and 1-methylcyclobutan-1-amine for step e. ¹H NMR (400 MHz, CDCl₃) δ 8.35 (s, 1H), 7.66 (d, J=1.4 Hz, 1H), 7.50 (dd, J=9.3, 5.2 Hz, 1H), 7.27 (d, J=1.4 Hz, 1H), 7.27-7.20 (m, 2H), 6.43 (s, 1H), 5.27-4.98 (m, 1H), 4.41 (ddd, J=19.3, 12.0, 6.1 Hz, 1H), 4.15 (dd, J=23.9, 12.0 Hz, 1H), 4.04 (s, 2H), 3.96-3.68 (m, 2H), 2.24 (q, J=9.9, 9.3 Hz, 2H), 2.14-2.05 (m, 1H), 1.94-1.76 (m, 4H), 1.42 (s, 3H), 1.12-1.02 (m, 4H). ESI MS [M+H]⁺ for C₃₀H₃₀F₂N₆O₂ calcd 545.2 found 545.4.

### Example 37: 6-(2-azabicyclo[2.2.1]heptan-2-ylmethyl)-3-[6-cyclopropyl-4-[4-fluoro-2-(3-fluoroazetidine-1-carbonyl)phenyl]pyridin-2-yl]-5H-pyrrolo[3,2-d]pyrimidin-4-one

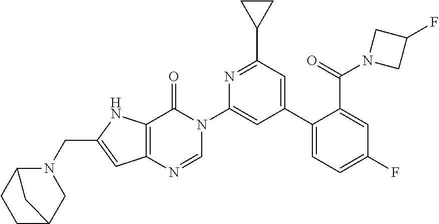 [SMILES: O=C(c1cc(F)ccc1-c1cc(C2CC2)nc(-n2cnc3cc(CN4CC5CCC4C5)[nH]c3c2=O)c1)N1CC(F)C1]

The title compound was prepared in a similar fashion to that described for example 34 using [2-(2-chloro-6-cyclopropylpyridin-4-yl)-5-fluorophenyl]-(3-fluoroazetidin-1-yl)methanone (prepared according to Example 21) for step b, and 2-azabicyclo[2.2.1]heptane for step e. ¹H NMR (400 MHz, CDCl₃) δ 8.45 (s, 1H), 7.68 (d, J=1.4 Hz, 1H), 7.49-7.44 (m, 1H), 7.28 (d, J=1.4 Hz, 1H), 7.25-7.19 (m, 2H), 6.40 (s, 1H), 5.36-5.09 (m, 1H), 4.40 (ddd, J=19.5, 12.1, 6.2 Hz, 1H), 4.14 (dd, J=24.0, 11.9 Hz, 1H), 4.06-3.77 (m, 4H), 3.47 (s, 1H), 2.88 (s, 1H), 2.58 (d, J=31.5 Hz, 1H), 2.21-2.03 (m, 1H), 1.85 (d, J=11.0 Hz, 2H), 1.47 (d, J=10.2 Hz, 2H), 1.16-0.93 (m, 4H). ESI MS [M+H]⁺ for C₃₁H₃₀F₂N₆O₂ calcd 557.2 found 557.3.

### Example 38: 3-[6-cyclopropyl-4-[2-(3,3-difluoroazetidine-1-carbonyl)-4-fluorophenyl]pyridin-2-yl]-6-[[2-methoxyethyl(methyl)amino]methyl]-5H-pyrrolo[3,2-d]pyrimidin-4-one

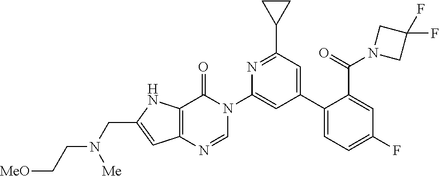 [SMILES: COCCN(C)Cc1cc2ncn(-c3cc(-c4ccc(F)cc4C(=O)N4CC(F)(F)C4)cc(C4CC4)n3)c(=O)c2[nH]1]

The title compound was prepared in a similar fashion to that described for example 34 using 2-methoxy-N-methylethanamine for step e. ¹H NMR (400 MHz, CDCl₃) δ 8.37 (s, 1H), 7.64 (d, J=1.4 Hz, 1H), 7.55-7.46 (m, 1H), 7.30-7.22 (m, 3H), 6.34 (s, 1H), 4.44 (t, J=12.0 Hz, 2H), 3.98 (t, J=11.6 Hz, 2H), 3.81 (s, 2H), 3.55 (t, J=5.1 Hz, 2H), 3.42 (s, 3H), 2.67 (t, J=5.1 Hz, 2H), 2.37 (s, 3H), 2.17-2.06 (m, 1H), 1.17-1.01 (m, 4H). ESI MS [M+H]⁺ for C₂₉H₂₉F₃N₆O₃ calcd 567.2 found 567.2.

### Example 39: 3-[6-cyclopropyl-4-[2-(3,3-difluoroazetidine-1-carbonyl)-4-fluorophenyl]pyridin-2-yl]-6-[(3,3,3-trifluoropropylamino)methyl]-5H-pyrrolo[3,2-d]pyrimidin-4-one

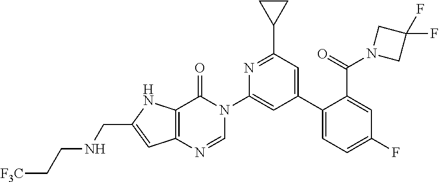 [SMILES: O=C(c1cc(F)ccc1-c1cc(C2CC2)nc(-n2cnc3cc(CNCCC(F)(F)F)[nH]c3c2=O)c1)N1CC(F)(F)C1]

The title compound was prepared in a similar fashion to that described for example 34 using 3,3,3-trifluoropropan-1-amine for step e. ¹H NMR (400 MHz, CDCl₃) δ 8.37 (s, 1H), 7.62 (d, J=1.4 Hz, 1H), 7.54-7.46 (m, 1H), 7.29-7.19 (m, 3H), 6.39 (s, 1H), 4.43 (t, J=11.9 Hz, 2H), 4.08-3.88 (m, 4H), 2.90 (t, J=7.0 Hz, 2H), 2.39-2.24 (m, 2H), 2.16-2.07 (m, 1H), 1.18-1.01 (m, 4H). ESI MS [M+H]⁺ for C₂₈H₂₄F₆N₆O₂ calcd 591.2 found 591.3.

### Example 40: 3-[6-cyclopropyl-4-[2-(3,3-difluoroazetidine-1-carbonyl)-4-fluorophenyl]pyridin-2-yl]-6-[[[(2S)-2-methoxypropyl]-methylamino]methyl]-5H-pyrrolo[3,2-d]pyrimidin-4-one

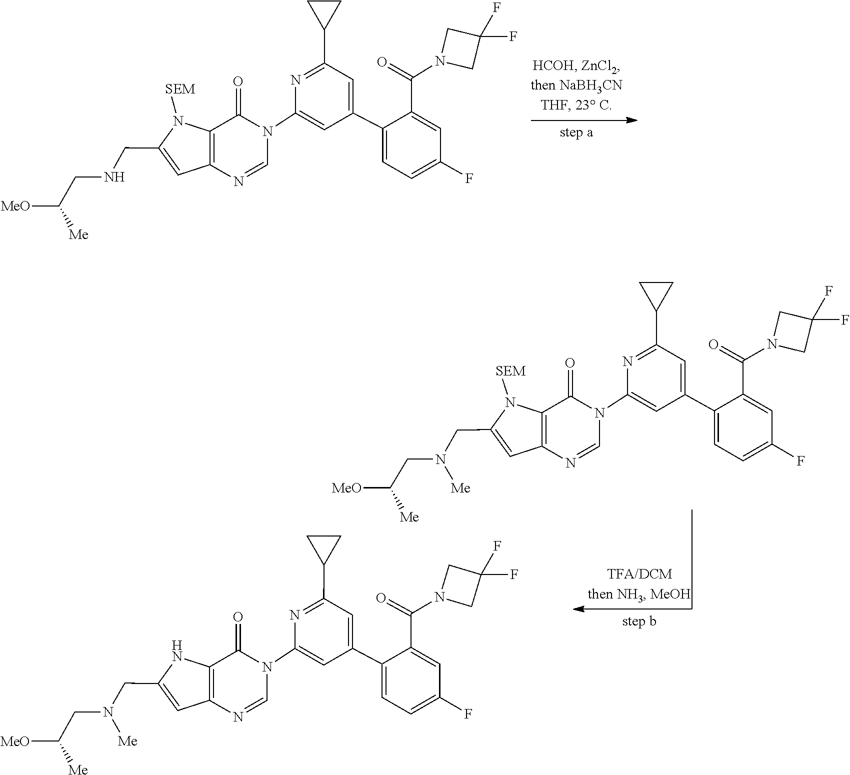 [SMILES: CO[C@@H](C)CNCc1cc2ncn(-c3cc(-c4ccc(F)cc4C(=O)N4CC(F)(F)C4)cc(C4CC4)n3)c(=O)c2n1COCC[Si](C)(C)C ; CO[C@@H](C)CN(C)Cc1cc2ncn(-c3cc(-c4ccc(F)cc4C(=O)N4CC(F)(F)C4)cc(C4CC4)n3)c(=O)c2n1COCC[Si](C)(C)C ; CO[C@@H](C)CN(C)Cc1cc2ncn(-c3cc(-c4ccc(F)cc4C(=O)N4CC(F)(F)C4)cc(C4CC4)n3)c(=O)c2[nH]1]

Step a: To a solution of 3-[6-cyclopropyl-4-[2-(3,3-difluoroazetidine-1-carbonyl)-4-fluorophenyl]pyridin-2-yl]-6-[[[(2S)-2-methoxypropyl]amino]methyl]-5-(2-trimethylsilylethoxymethyl)pyrrolo[3,2-d]pyrimidin-4-one (57 mg, 0.08 mmol, 1.0 equiv., prepared according to Example 21 using 3,3-difluoroazetidine hydrochloride on step a) was added paraformaldehyde (5 mg, 0.16 mmol, 2.0 equiv.) and ZnCl₂ (56 mg, 0.41 mmol, 5.0 equiv.). The reaction mixture was stirred at room temperature for 1 h before NaBH₃CN (16 mg, 0.25 mmol, 3.0 equiv.) was added. After an additional 1 h of stirring the reaction mixture was diluted with 1:1 mixture of water and brine (20 mL) and extracted with EtOAc (2×10 mL). The combined organic extract was dried over Na₂SO₄, filtered, and concentrated under vacuum. The crude product was used for the next step without further purification.

Step b: To a solution of the product from step a (assume 0.08 mmol, 1.0 equiv.) in dichloromethane (1 mL) was added trifluoroacetic acid (1 mL). The reaction mixture was stirred at 30° C. for 1 h. The mixture was cooled to 23° C., diluted with toluene (5 mL) and concentrated to dryness under vacuum. The crude residue was dissolved in 7M NH₃ in MeOH (2 mL) and the resulting solution was maintained at 30° C. for 1 h. Upon solvent evaporation under vacuum the crude product was directly purified by reverse phase prep-HPLC (SiO₂ C18 column, 5 to 50% CH₃CN gradient in water with 0.1% formic acid) to afford the desired product. ¹H NMR (400 MHz, CDCl₃) δ 8.37 (s, 1H), 7.64 (d, J=1.4 Hz, 1H), 7.50 (dd, J=9.3, 5.2 Hz, 1H), 7.28-7.22 (m, 3H), 6.35 (s, 1H), 4.43 (t, J=11.9 Hz, 2H), 3.98 (t, J=11.6 Hz, 2H), 3.91-3.78 (m, 2H), 3.65-3.54 (m, 1H), 3.42 (s, 3H), 2.61 (dd, J=13.4, 8.2 Hz, 1H), 2.44 (dd, J=14.3, 4.1 Hz, 1H), 2.40 (s, 3H), 2.16-2.05 (m, 1H), 1.14 (d, J=6.2 Hz, 3H), 1.12-1.08 (m, 2H), 1.05 (dt, J=8.2, 2.9 Hz, 2H). ESI MS [M+H]⁺ for C₃₀H₃₁F₃N₆O₃ calcd 581.3 found 581.3.

### Example 41: 6-{5-cyclopropyl-2′-[(3,3-difluoro-1-azetidinyl)carbonyl]-4′-fluoro-3-biphenylyl}-2-[(2-methoxyethylamino)methyl]-1,6-dihydro-1,4,6-triaza-7-indenone

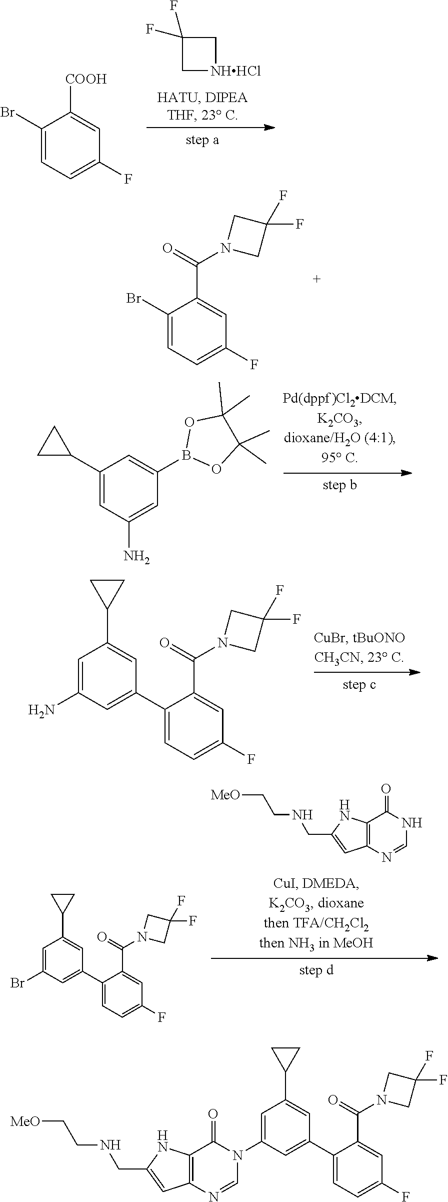

Step a: To a solution of a 2-bromo-5-fluorobenzoic acid (654 mg, 3.0 mmol, 1 equiv.) in THF (15 mL, 0.2 M) were added i-Pr₂NEt (1.06 mL, 6.0 mmol, 2 equiv.), HATU (1.7 g, 4.5 mmol, 1.5 equiv.) and 3,3-difluoroazetidine hydrochloride (0.78 g, 6 mmol, 2 equiv.). The reaction mixture was stirred overnight at room temperature, then diluted with EtOAc (40 mL) and water (40 mL). The organic phase was separated, sequentially washed with water (30 mL) and brine (30 mL), dried over Na₂SO₄ and concentrated to dryness under reduced pressure. The crude residue was purified by column chromatography (SiO₂, 0 to 100% EtOAc gradient in hexane) to afford the desired product.

Step b: The product from step a (533 mg, 1.82 mmol, 1 equiv.), 3-cyclopropyl-5-(4,4,5,5-tetramethyl-1,3,2-dioxaborolan-2-yl)aniline (471.6 mg, 1.82 mmol. 1.0 equiv.) and K₂CO₃ (753.4 mg, 5.4 mmol, 3 equiv.) were suspended in the mixture of dioxane/water (10 mL, 3:1 v/v). The resulting mixture was degassed by sparging with N₂ for 10 minutes. Pd(dppf)Cl₂—CH₂Cl₂ (148.5 mg, 0.182 mmol, 0.1 equiv.) was added, and the mixture was heated to 95° C. for 8 hours. The resulting solution was cooled to 23° C. and partitioned between EtOAc (30 mL) and water (30 mL). The organic phase was separated, and the aqueous layer was additionally extracted with EtOAc (2×15 mL). The combined organic phase was dried over Na₂SO₄ and concentrated to dryness under vacuum. The crude residue was purified via silica gel column chromatography (0 to 100% EtOAc/hexane) to afford the desired coupling product.

Step c: The reaction was performed in a similar fashion to Example 22, step b to afford the [2-(3-bromo-5-cyclopropylphenyl)-5-fluorophenyl]-(3,3-difluoroazetidin-1-yl)methanone as the desired product.

Step d: To a solution of the product from step c (53 mg, 0.13 mmol, 1.0 equiv.), and 6-[(2-methoxyethylamino)methyl]-5-(2-trimethylsilylethoxymethyl)-3H-pyrrolo[3,2-d]pyrimidin-4-one (45.7 mg, 0.13 mmol, 1.0 equiv., prepared as described in Example 25, using methoxyethanamine in step d) in dioxane (2.8 mL) were added CuI (24.8 mg, 0.13 mmol, 1.0 equiv.), N,N′-dimethylethylenediamine (22.9 mg, 0.26 mmol, 2.0 equiv.) and K₂CO₃ (52.5 mg, 0.38 mmol, 3.0 equiv.). The reaction mixture was degassed by purging N₂ for 5 minutes and stirred at 110° C. for 12 hours under vigorous stirring in a sealed vial. Upon cooling to room temperature, the mixture was diluted with EtOAc (10 mL), washed with aq. sat. NH₄Cl (10 mL), then brine (10 mL), filtered through Na₂SO₄, and concentrated. The residual material was then treated with trifluoroacetic acid/dichloromethane mixture (4 mL, 1:3 v/v) at room temperature for 3 h. The solvent was removed under vacuum followed by the addition of 7M NH₃ in methanol (3 mL) and stirring for 1 h at 23° C. Upon solvent evaporation the crude product was purified by prep-HPLC (SiO₂ C18, 10 to 90% CH₃CN in water with 0.1% formic acid) to afford the title compound. ¹H NMR (400 MHz, CDCl₃) δ 7.89 (s, 1H), 7.45 (dd, J=8.5, 5.2 Hz, 1H), 7.29-7.27 (m, 1H), 7.26-7.16 (m, 3H), 7.11 (t, J=1.8 Hz, 1H), 6.36 (s, 1H), 4.34 (t, J=11.9 Hz, 2H), 4.05-3.98 (m, 2H), 3.83 (t, J=11.6 Hz, 2H), 3.55-3.47 (m, 2H), 3.38 (s, 3H), 2.88-2.79 (m, 2H), 2.04-1.96 (m, 1H), 1.11-1.02 (m, 2H), 0.81-0.73 (m, 2H). C₂₉H₂₈F₃N₅O₃, calcd 551.2, found 551.2.

### Example 42: 6-{5-cyclopropyl-2′-[(3,3-difluoro-1-azetidinyl)carbonyl]-4′-fluoro-3-biphenylyl}-2-[(2-methoxyethylamino)methyl]-1,6-dihydro-1,4,6-triaza-7-indenone

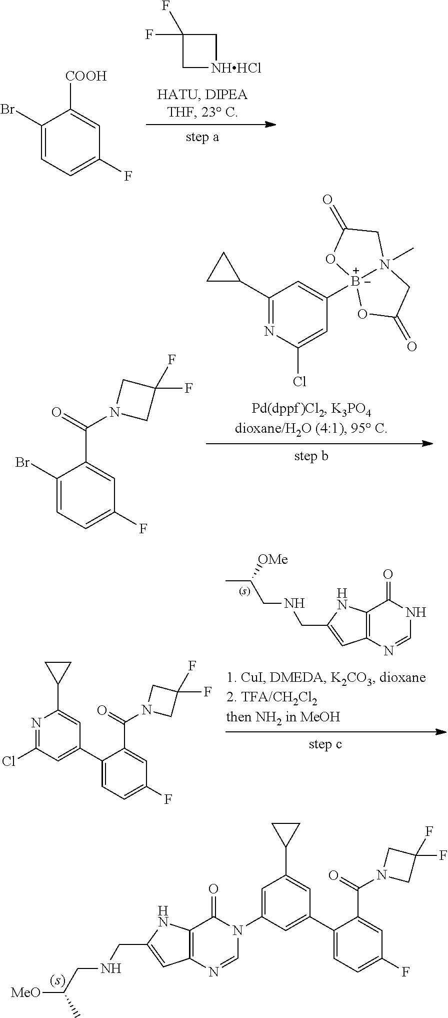

Steps a, b: These steps were performed in a similar fashion to example 21 steps a, b.

Step c: The reaction was performed in a similar fashion example 41, step d, using 6-[[[(2S)-2-methoxypropyl]amino]methyl]-3,5-dihydropyrrolo[3,2-d]pyrimidin-4-one (prepared in an analogous fashion to that described in example 25, using (2S)-2-methoxypropan-1-amine in step d) and [2-(2-chloro-6-cyclopropylpyridin-4-yl)-5-fluorophenyl]-(3,3-difluoroazetidin-1-yl)methanone to afford the title product. ¹H NMR (400 MHz, CDCl₃) δ 8.38 (s, 1H), 7.65 (d, J=1.4 Hz, 1H), 7.53-7.48 (m, 1H), 7.28 (d, J=2.2 Hz, 1H), 7.26 (s, 2H), 6.37 (s, 1H), 4.43 (t, J=11.9 Hz, 2H), 4.10-3.92 (m, 4H), 3.57-3.48 (m, 1H), 3.36 (s, 3H), 2.73-2.61 (m, 2H), 2.15-2.06 (m, 1H), 1.14 (d, J=6.2 Hz, 3H), 1.12-1.03 (m, 4H). C₂₉H₂₉F₃N₆O₃, calcd 567.2, found 567.2.

### Example 43: 6-(6-cyclopropyl-4-{2-[(3,3-difluoro-1-azetidinyl)carbonyl]-4-fluorophenyl}-2-pyridyl)-2-[(3-methoxy-1-azetidinyl)methyl]-1,6-dihydro-1,4,6-triaza-7-indenone

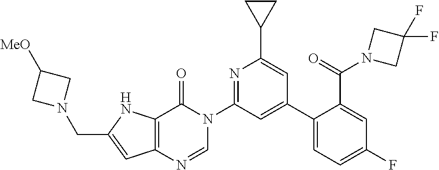 [SMILES: COC1CN(Cc2cc3ncn(-c4cc(-c5ccc(F)cc5C(=O)N5CC(F)(F)C5)cc(C5CC5)n4)c(=O)c3[nH]2)C1]

The titled compound was prepared in a similar fashion example 42 starting from 6-[(3-methoxyazetidin-1-yl)methyl]-3,5-dihydropyrrolo[3,2-d]pyrimidin-4-one (prepared according to example 25 using 3-methoxyazetidine in step d) and [2-(2-chloro-6-cyclopropylpyridin-4-yl)-5-fluorophenyl]-(3,3-difluoroazetidin-1-yl)methanone. ¹H NMR (400 MHz, CDCl₃) δ 9.55 (s, 1H), 8.38 (s, 1H), 7.63 (d, J=1.4 Hz, 1H), 7.53-7.46 (m, 1H), 7.30-7.26 (m, 2H), 7.26-7.23 (m, 1H), 6.37 (s, 1H), 4.44 (t, J=12.0 Hz, 2H), 4.09-4.02 (m, 1H), 4.02-3.92 (m, 2H), 3.78 (s, 2H), 3.70-3.61 (m, 2H), 3.27 (s, 3H), 3.10-3.01 (m, 2H), 2.15-2.06 (m, 1H), 1.12-1.02 (m, 4H). C₂₉H₂₇F₃N₆O₃, calcd 565.2, found 565.2.

### Example 44: 6-(6-cyclopropyl-4-{2-[(3,3-difluoro-1-azetidinyl)carbonyl]-4-fluorophenyl}-2-pyridyl)-2-[(2-methoxyethylamino)methyl]-1,6-dihydro-1,4,6-triaza-7-indenone

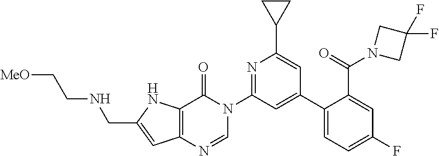 [SMILES: COCCNCc1cc2ncn(-c3cc(-c4ccc(F)cc4C(=O)N4CC(F)(F)C4)cc(C4CC4)n3)c(=O)c2[nH]1]

The title compound was prepared in a similar fashion example 42 starting from 6-[(2-methoxyethylamino)methyl]-5-(2-trimethylsilylethoxymethyl)-3H-pyrrolo[3,2-d]pyrimidin-4-one (prepared as described in Example 25, using methoxyethanamine in step d) and [2-(2-chloro-6-cyclopropylpyridin-4-yl)-5-fluorophenyl]-(3,3-difluoroazetidin-1-yl)methanone. ¹H NMR (400 MHz, CDCl₃) δ 8.38 (s, 1H), 7.64 (d, J=1.4 Hz, 1H), 7.53-7.46 (m, 1H), 7.28 (s, 1H), 7.25 (d, J=7.9 Hz, 2H), 6.36 (s, 1H), 4.43 (t, J=11.9 Hz, 2H), 4.05-3.89 (m, 4H), 3.51 (t, J=4.9 Hz, 2H), 3.38 (s, 3H), 2.83 (t, J=5.0 Hz, 2H), 2.14-2.07 (m, 1H), 1.13-1.02 (m, 4H). C₂₈H₂₇F₃N₆O₃, calcd 553.2, found 553.2.

### Example 45: 2-{[(S)-3-methyl-1-piperidyl]methyl}-6-(6-cyclopropyl-4-{4-[(3-fluoro-1-azetidinyl)carbonyl]-1-methyl-5-pyrazolyl}-2-pyridyl)-7-oxo-1,6-dihydro-1,6-diaza-4-indenecarbonitrile

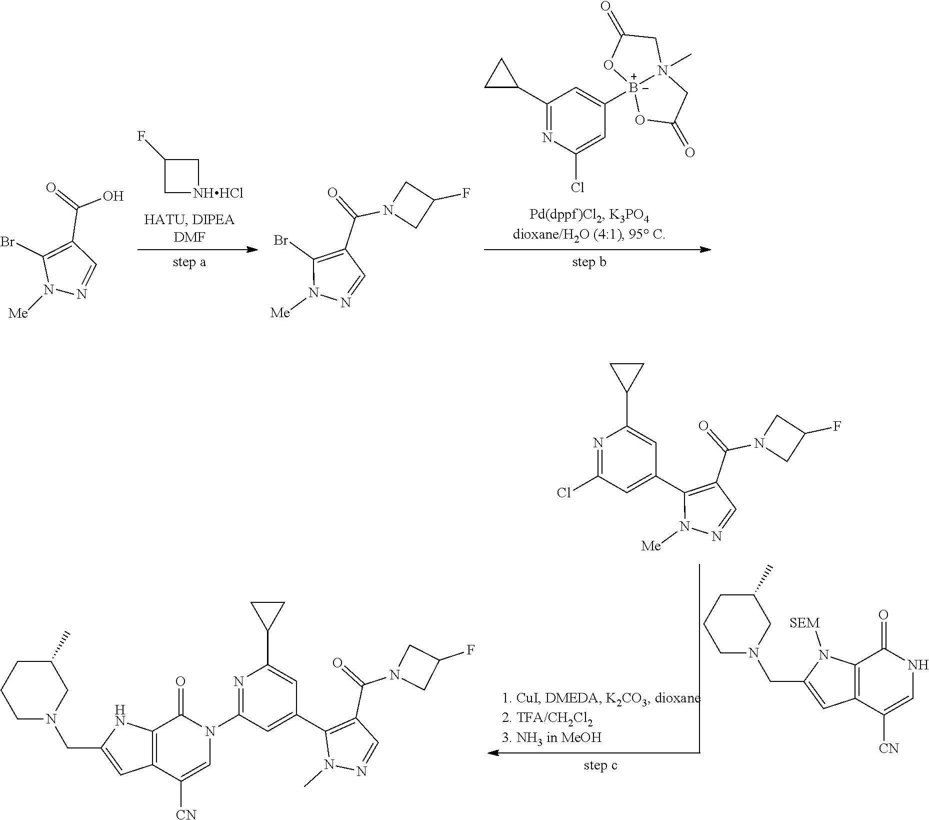

Step a: The reaction was performed in a similar fashion to example 21, step a to afford (5-bromo-1-methylpyrazol-4-yl)-(3-fluoroazetidin-1-yl)methanone as the product.

Step b: The reaction was performed in a similar fashion to example 21, step b to afford [5-(2-chloro-6-cyclopropylpyridin-4-yl)-1-methylpyrazol-4-yl]-(3-fluoroazetidin-1-yl)methanone as the product.

Step c: The reaction was performed in a similar fashion example 21, step c, using 2-[[(3S)-3-methylpiperidin-1-yl]methyl]-7-oxo-1-(2-trimethylsilylethoxymethyl)-6H-pyrrolo[2,3-c]pyridine-4-carbonitrile (prepared according to example 19) and [5-(2-chloro-6-cyclopropylpyridin-4-yl)-1-methylpyrazol-4-yl]-(3-fluoroazetidin-1-yl)methanone to afford the title product. ¹H NMR (400 MHz, CDCl₃) δ 9.68 (s, 1H), 8.16 (s, 1H), 7.75-7.67 (m, 2H), 7.34 (d, J=1.2 Hz, 1H), 6.39 (s, 1H), 5.41-5.20 (m, 1H), 4.50-4.16 (m, 4H), 3.91 (s, 3H), 3.66-3.55 (m, 2H), 2.82-2.68 (m, 2H), 2.18-2.09 (m, 1H), 2.02-1.93 (m, 1H), 1.78-1.44 (m, 6H), 1.14-1.08 (m, 4H), 0.84 (d, J=6.0 Hz, 3H). C₃₁H₃₃FN₈O₂, calcd 569.2, found 569.2.

### Example 46: 2-{[(S)-3-methyl-1-piperidyl]methyl}-3-chloro-6-(6-cyclopropyl-4-{4-fluoro-2-[(3-fluoro-1-azetidinyl)carbonyl]phenyl}-2-pyridyl)-7-oxo-1,6-dihydro-1,6-diaza-4-indenecarbonitrile

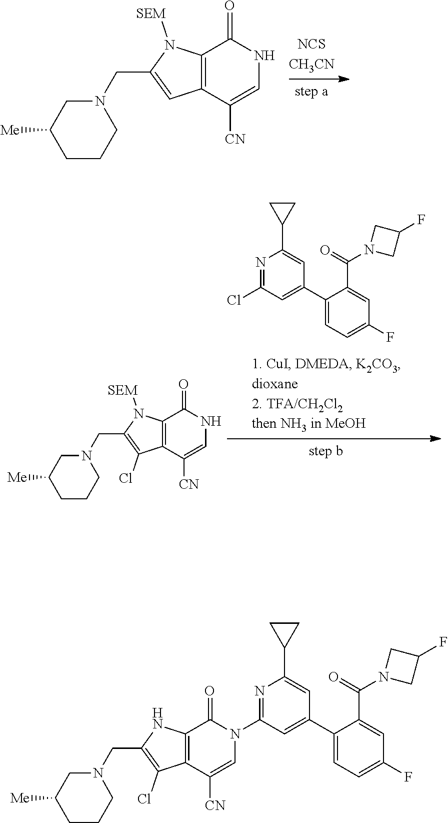 [SMILES: C[C@H]1CCCN(Cc2cc3c(C#N)c[nH]c(=O)c3n2COCC[Si](C)(C)C)C1 ; O=C(c1cc(F)ccc1-c1cc(Cl)nc(C2CC2)c1)N1CC(F)C1 ; C[C@H]1CCCN(Cc2c(Cl)c3c(C#N)c[nH]c(=O)c3n2COCC[Si](C)(C)C)C1 ; C[C@H]1CCCN(Cc2[nH]c3c(=O)n(-c4cc(-c5ccc(F)cc5C(=O)N5CC(F)C5)cc(C5CC5)n4)cc(C#N)c3c2Cl)C1]

Step a: To a solution of a 2-[[(3S)-3-methylpiperidin-1-yl]methyl]-7-oxo-1-(2-trimethylsilylethoxymethyl)-6H-pyrrolo[2,3-c]pyridine-4-carbonitrile (0.1 g, 0.37 mmol, 1.0 equiv., prepared according to example 19) in CH₃CN (4 mL) was added N-chlorosuccinimide (0.054 g, 0.40 mmol, 1.1 equiv.). The resulting mixture was stirred for 12 h at 23° C. After completion, the solution was concentrated to dryness under reduced pressure and diluted with EtOAc (50 mL) and water (50 mL). The organic phase was separated, and the aqueous layer was extracted with EtOAc (30 mL). The combined organics were dried over Na₂SO₄ and concentrated to dryness under reduced pressure. The crude residue was purified by column chromatography (SiO₂, 0-20% EtOAc gradient in hexanes) to afford the desired product.

Step b: The reaction was performed in a similar fashion example 41, step d, using [2-(2-chloro-6-cyclopropylpyridin-4-yl)-5-fluorophenyl]-(3-fluoroazetidin-1-yl)methanone (prepared according to example 21). ¹H NMR (400 MHz, CDCl₃) δ 8.24 (s, 1H), 7.73 (s, 1H), 7.50-7.44 (m, 1H), 7.28 (d, J=1.4 Hz, 1H), 7.23 (t, J=8.1 Hz, 2H), 5.33-5.11 (m, 1H), 4.48-4.31 (m, 1H), 4.26-4.10 (m, 1H), 4.01-3.89 (m, 1H), 3.88-3.71 (m, 3H), 2.97-2.76 (m, 2H), 2.23-2.07 (m, 2H), 2.04-1.56 (m, 6H), 1.09 (d, J=5.7 Hz, 3H), 0.90-0.86 (m, 4H). C₃₃H₃₁ClF₂N₆O₂, calcd 617.2, found 617.2.

### Example 47: 2-{1-[(R)-2-methyl-4-morpholinyl]ethyl}-6-(6-cyclopropyl-4-{4-fluoro-2-[(3-fluoro-1-azetidinyl)carbonyl]phenyl}-2-pyridyl)-1,6-dihydro-1,4,6-triaza-7-indenone

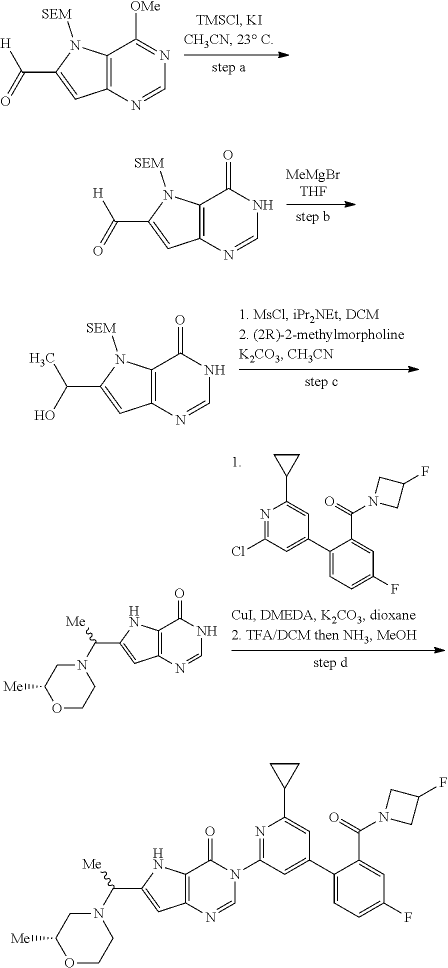

Step a: The reaction was performed in a similar fashion to that described for example 25, step e, using 4-methoxy-5-(2-trimethylsilylethoxymethyl)pyrrolo[3,2-d]pyrimidine-6-carbaldehyde.

Step b: To a solution of the product from step a (0.87 g, 3.0 mmol, 1.0 equiv.) in THF (15 ml, 0.2 M) was added MeMgBr (3 M in THF 1.5 mL, 4.5 mmol, 1.5 equiv) at 0° C. The resulting mixture was stirred at room temperature for 8 h, quenched with aq. sat. NH₄Cl (20 mL) and diluted with EtOAc (100 mL) and water (30 mL). The organic phase was separated, and the aqueous phase was additionally extracted with EtOAc (2×40 mL). The combined organic extract was washed with brine (20 mL), dried over Na₂SO₄ and concentrated to dryness under reduced pressure. The crude product was purified by column chromatography (SiO₂, 0-50% EtOAc gradient in hexanes) to afford the desired product.

Step c: To a solution of the product from step b (500 mg, 1.6 mmol, 1.0 mmol) and iPr₂NEt (0.42 mL, 2.4 mmol, 1.5 equiv) in dichloromethane (4 mL) was added MsCl (0.12 mL, 1.6 mmol, 1.0 equiv.) at 0° C. The resulting mixture was stirred at room temperature for 12 h. Once LCMS analysis indicated complete reaction the solvent was removed under reduced pressure, and the crude residue was dissolved in acetonitrile (4 mL). Potassium carbonate (0.44 g, 3.2 mmol, 2.0 eq.) and (2R)-2-methylmorpholine (0.48 mg, 4.8 mmol, 4.0 equiv.) were added, and the resulting mixture was stirred at 80° C. overnight. The mixture was cooled to room temperature, diluted with EtOAc (20 mL), sequentially washed with H₂O (10 mL) and brine (10 mL), filtered through Na₂SO₄, and concentrated. The crude residue was purified by column chromatography (SiO₂, 0 to 10% MeOH gradient in dichloromethane) to afford the desired product.

Step d: The reaction was performed in a similar fashion example 41, step d, using 6-[1-[(2R)-2-methylmorpholin-4-yl]ethyl]-3,5-dihydropyrrolo[3,2-d]pyrimidin-4-one and [2-(2-chloro-6-cyclopropylpyridin-4-yl)-5-fluorophenyl]-(3-fluoroazetidin-1-yl)methanone (prepared according to example 21). The titled compound was obtained as a mixture of diastereomers in 1:1 ratio. ¹H NMR (400 MHz, CDCl₃) δ 9.42 (s, 1H), 8.41-8.34 (m, 1H), 7.62 (d, J=1.5 Hz, 1H), 7.47 (dd, J=8.4, 5.2 Hz, 1H), 7.30 (d, J=1.4 Hz, 1H), 7.26-7.20 (m, 2H), 6.37 (d, J=2.0 Hz, 1H), 5.27-4.99 (m, 1H), 4.48-4.32 (m, 1H), 4.25-4.10 (m, 1H), 3.96-3.71 (m, 3H), 3.71-3.57 (m, 2H), 2.58-2.47 (m, 2H), 2.34-2.08 (m, 2H), 2.03-1.93 (m, 1H), 1.41 (d, J=6.8 Hz, 3H), 1.15-1.10 (m, 4H), 1.10-1.01 (m, 4H). C₃₁H₃₂F₂N₆O₃, calcd 575.2, found 575.2.

### Example 48: 5-[6-cyclopropyl-4-[4-fluoro-2-(3-fluoroazetidine-1-carbonyl)phenyl]pyridin-2-yl]-2-[[(3S)-3-methylpiperidin-1-yl]methyl]-7-(trifluoromethyl)-3H-imidazo[4,5-c]pyridin-4-one

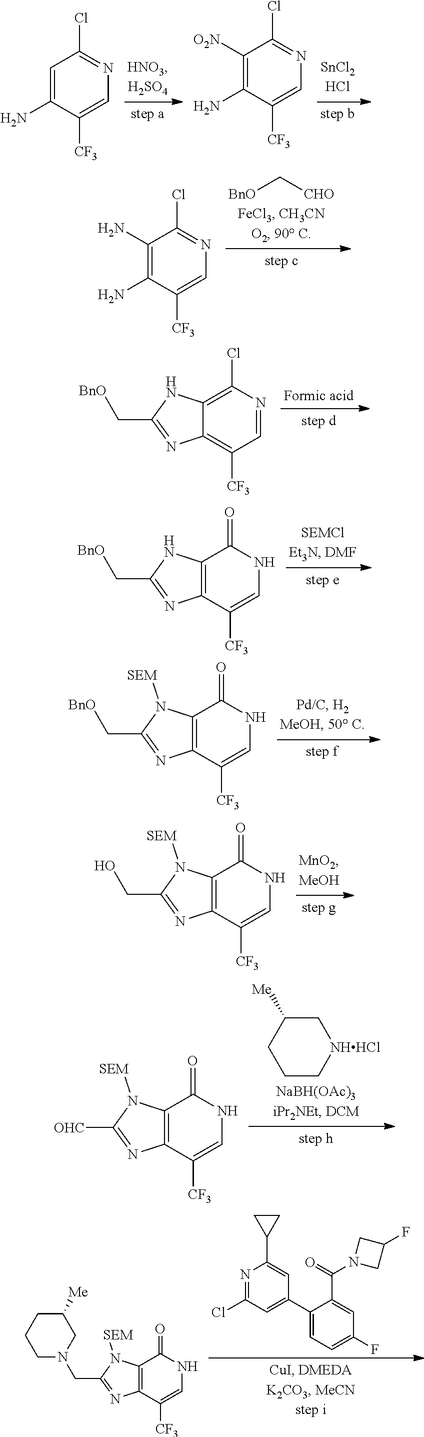

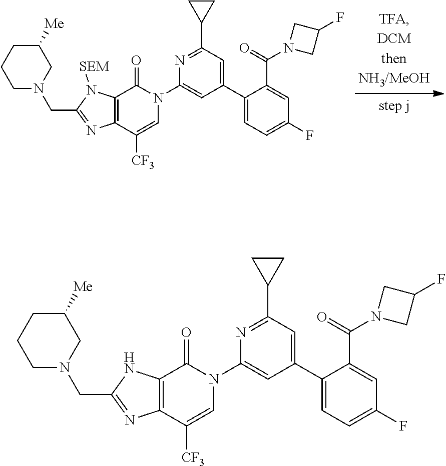 [SMILES: C[C@H]1CCCN(Cc2nc3c(C(F)(F)F)cn(-c4cc(-c5ccc(F)cc5C(=O)N5CC(F)C5)cc(C5CC5)n4)c(=O)c3n2COCC[Si](C)(C)C)C1 ; C[C@H]1CCCN(Cc2nc3c(C(F)(F)F)cn(-c4cc(-c5ccc(F)cc5C(=O)N5CC(F)C5)cc(C5CC5)n4)c(=O)c3[nH]2)C1]

Step a: 2-Chloro-5-(trifluoromethyl)-4-pyridinamine (5.0 g, 25.5 mmol, 1.0 equiv.) was dissolved in H₂SO₄ (30 mL) and fuming concentrated HNO₃ (10 mL) was added dropwise over 10 min period. The resulting mixture was stirred at 75° C. for 4 h, then the mixture was cooled to 23° C. The resulting solution was basified with aq. NaOH (2 M) to pH>7, and the product was extracted with CHCl₃/i-PrOH mixture (3×50 mL, 3:1 v/v). The combined organic extract was dried over Na₂SO₄ and concentrated to dryness under reduced pressure. The crude product was purified by column chromatography (SiO₂, 0-20% EtOAc gradient in hexanes) to afford 2-chloro-3-nitro-5-(trifluoromethyl)pyridin-4-amine.

Step b: The product from step a (6.2 g, 25.5 mmol, 1.0 equiv.) was dissolved in concentrated HCl (30 mL), and the obtained mixture was preheated to 90° C. Then SnCl₂ (19.4 g, 102 mmol, 4.0 equiv.) solution in 15 mL of aq. HCl (37 wt. %) was added dropwise over 10 min. period. Once the addition was complete the reaction mixture was heated in a sealed vial at 75° C. for 1 hours. The mixture was cooled to room temperature and slowly neutralized with aqueous. NaOH (2 M). The product of the reduction was extracted with CHCl₃/i-PrOH (3×50 mL, 3:1 v/v). The combined organic extract was dried over Na₂SO₄, concentrated and the crude residue was purified by column chromatography (SiO₂, 0-30% EtOAc gradient in hexanes) to afford 2-chloro-5-(trifluoromethyl)pyridine-3,4-diamine.

Step c: To the product from step b (2.96 g, 14.0 mmol) in CH₃CN (80 mL) was added 2-phenylmethoxyacetaldehyde (3.15 g, 21.0 mmol) and FeCl₃ (453 mg, 2.8 mmol). The resulting mixture was heated overnight at 90° C. under air atmosphere. The obtained mixture was cooled to room temperature and concentrated to dryness under reduced pressure. The crude residue was directly fractionated by column chromatography (SiO₂, 0-40% EtOAc gradient in hexanes) to afford 4-chloro-2-(phenylmethoxymethyl)-7-(trifluoromethyl)-3H-imidazo[4,5-c]pyridine.

Step d: The product of step c (163 mg, 0.48 mmol) was dissolved in formic acid (5 mL, 0.1 M), and the resulting solution was stirred at 90° C. overnight. The obtained mixture was cooled to room temperature and formic acid was evaporated under reduced pressure. Purification by column chromatography (SiO₂, 0-10% MeOH gradient in dichloromethane) furnished 2-(phenylmethoxymethyl)-7-(trifluoromethyl)-3,5-dihydroimidazo[4,5-c]pyridin-4-one.

Step e: The product from step d (78.6 mg, 0.24 mmol) was dissolved in DMF (1.1 mL) followed by the addition of Et₃N (0.1 mL mg, 0.72 mmol) and SEMCl (50 mg, 0.3 mmol). The reaction mixture was stirred at room temperature overnight followed by partitioning between EtOAc (15 mL) and water (15 mL). The organic phase was separated, and the aqueous layer was additionally extracted with EtOAc (2×10 mL). The combined organic phase was washed with water (2×20 mL), dried over Na₂SO₄ and concentrated to dryness under reduced pressure. The crude material was fractionated by column chromatography (SiO₂, 0-100% EtOAc gradient in hexanes) to yield 2-(phenylmethoxymethyl)-7-(trifluoromethyl)-3-(2-trimethylsilylethoxymethyl)-5H-imidazo[4,5-c]pyridin-4-one.

Step f: To the solution of the product from step e (246 mg, 0.54 mmol) in methanol (5.4 mL, 0.1 M) palladium on carbon (246 mg, 10 wt % Pd) was added. The mixture was placed in sealed vial, degassed and backfilled with hydrogen. The resulting mixture was vigorously stirred at 50° C. under hydrogen atmosphere overnight. The obtained suspension was filtered through a pad of Celite® that was additionally washed with methanol (5 mL). The filtrate was concentrated to dryness under vacuum to afford crude product that was used for the next step without purification.

Step g: The crude alcohol product from step f (approx. 0.54 mmol) was dissolved in MeOH (2.7 mL, 0.2 M) followed by the addition of MnO₂ (139 mg, 1.6 mmol). The resulting mixture was stirred at 50° C. overnight. The obtained solution was passed through a Celite® pad upon cooling to 23° C. The precipitate was additionally washed with MeOH (5 mL), and the combined filtrate was concentrated to dryness under vacuum. The crude product was purified by column chromatography (SiO₂, 0-10% MeOH gradient in dichloromethane) to afford 4-oxo-7-(trifluoromethyl)-3-(2-trimethylsilylethoxymethyl)-5H-imidazo[4,5-c]pyridine-2-carbaldehyde.

Step h: The product from step g (48 mg, 0.13 mmol) was dissolved in dichloromethane (1 mL), and (3S)-3-methyl-piperidine hydrochloride (26.3 mg, 0.2 mmol) and i-Pr₂NEt (34 mg, 0.26 mmol) were added. The reaction was stirred at room temperature for 30 mins before NaBH(OAc)₃ (41 mg, 0.2 mmol) was added. The resulting reaction was stirred for another hour, then diluted with water (5 mL) and dichloromethane (5 mL). The organic phase was separated, and the aqueous phase was additionally extracted with dichloromethane (5 mL). The combined organic phase was dried over Na₂SO₄, and the solvent was removed in vacuum. The crude product was purified by column chromatography (SiO₂, 0-10% MeOH gradient in dichloromethane) to afford 2-[[(3S)-3-methylpiperidin-1-yl]methyl]-7-(trifluoromethyl)-3-(2-trimethylsilylethoxymethyl)-5H-imidazo[4,5-c]pyridin-4-one.

Step i: To a solution of the product from step h (67 mg, 0.15 mmol, 1.0 equiv.), [2-(2-chloro-6-cyclopropylpyridin-4-yl)-5-fluorophenyl]-(3-fluoroazetidin-1-yl)methanone (52.2 mg, 0.15 mmol, 1.0 equiv., prepared according to example 21) and DMEDA (32 μL, 0.3 mmol, 2.0 equiv.) in CH₃CN (2 mL), K₂CO₃ (62.1 mg, 0.45 mmol, 3.0 equiv.) was added. The reaction mixture was degassed by purging nitrogen for 10 min followed by the addition of CuI (28.5 mg, 0.15 mmol, 1.0 equiv.). The reaction was stirred in sealed vial at 100° C. overnight. After cooling to room temperature sat. aq. NH₄Cl (5 mL) was added, and the product was extracted with EtOAc (3×10 mL). The combined organic phase was washed with water (2×20 mL), dried over Na₂SO₄ and concentrated under reduced pressure. The crude product was purified by column chromatography (SiO₂, 0-5% MeOH gradient in dichloromethane) to afford the desired coupling product.

Step j: To a solution of the product from step i (91 mg, 0.12 mmol, 1 equiv.) in dichloromethane (1 mL) trifluoroacetic acid was added. The mixture was stirred at 23° C. for 1 h, concentrated to dryness and redissolved in 7 M NH₃ in MeOH (1 mL). After 30 min the solvent was evaporated under vacuum, and the crude product was purified by preparative HPLC (SiO₂ C18, 10 to 90% CH3CN in water with 0.1% formic acid). ¹H NMR (400 MHz, CDCl₃) δ 8.19 (d, J=1.5 Hz, 1H), 7.70 (d, J=1.3 Hz, 1H), 7.45 (td, J=10.1, 9.2, 6.2 Hz, 1H), 7.28 (d, J=1.4 Hz, 1H), 7.26-7.18 (m, 2H), 5.26 (tt, J=6.1, 3.2 Hz, 1H), 5.12 (dq, J=6.1, 3.1 Hz, 1H), 4.40 (ddd, J=20.3, 12.0, 6.1 Hz, 1H), 4.25-4.08 (m, 1H), 4.03-3.87 (m, 3H), 3.81 (dd, J=23.3, 10.9 Hz, 1H), 2.88 (dd, J=24.4, 11.1 Hz, 2H), 2.31-2.18 (m, 1H), 2.13 (tt, J=7.5, 5.4 Hz, 1H), 1.93 (t, J=10.8 Hz, 1H), 1.73 (dd, J=36.5, 15.3 Hz, 3H), 1.14-1.04 (m, 4H), 0.95 (dd, J=12.3, 5.0 Hz, 1H), 0.89 (d, J=6.3 Hz, 3H). ESI MS [M+H]⁺ for C₃₂H₃₁F₅N₆O₂, calcd 627.2, found 627.2.

### Example 49: 5-[6-cyclopropyl-4-[4-fluoro-2-(3-fluoroazetidine-1-carbonyl)phenyl]pyridin-2-yl]-7-methyl-2-[(2R)-pyrrolidin-2-yl]-3H-imidazo[4,5-c]pyridin-4-one

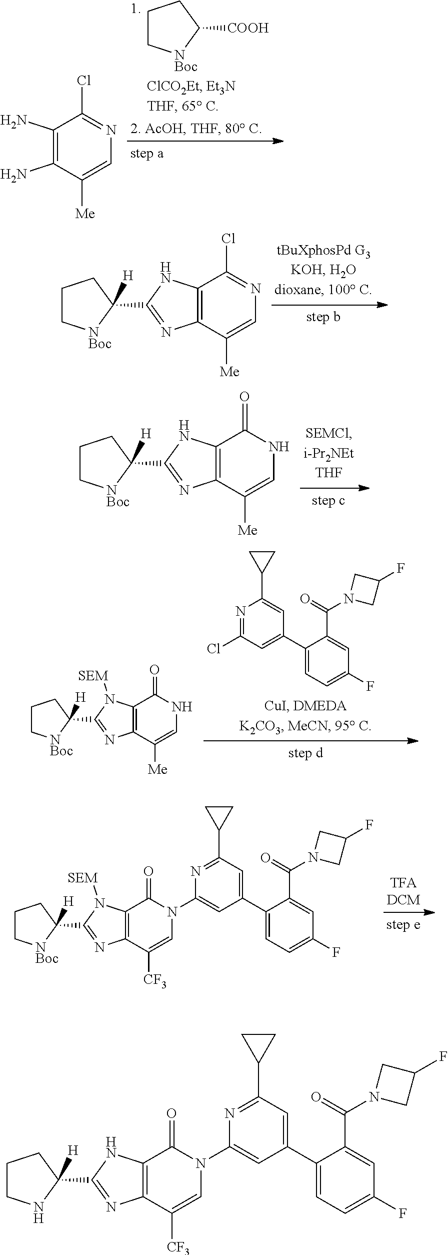

Step a: To a solution of (2R)-1-[(tert-butoxy)carbonyl]pyrrolidine-2-carboxylic acid (516 mg, 2.4 mmol, 1.2 equiv.) in 5 mL THE was sequentially added Et₃N (0.67 mL, 4.8 mmol, 2.4 equiv.) and ClCO₂Et (0.23 mL, 2.4 mmol, 1.2 equiv.) at 0° C. After 30 min of stirring 2-chloro-5-methyl-3,4-pyridinediamine (314 mg, 2.0 mmol, 1.0 equiv.) was added, and the mixture was stirred at 65° C. overnight. The resulting mixture was concentrated to dryness, and the residue was directly fractionated by column chromatography (SiO₂, 0-100% EtOAc gradient in hexanes) to afford corresponding acylation intermediate. It was dissolved in THF (5 mL) followed by the addition of AcOH (5 mL). The resulting mixture was stirred at 80° C. overnight. The excess of THF and AcOH was removed by evaporation under vacuum. The residue was partitioned between EtOAc (20 mL) and water (20 mL). The organic phase was separated, washed with aq. sat. NaHCO₃ (10 mL), dried over Na₂SO₄ and concentrated to dryness under reduced pressure. The crude product was purified by column chromatography (SiO₂, 0-100% EtOAc in hexanes) to yield tert-butyl (2R)-2-(4-chloro-7-methyl-3H-imidazo[4,5-c]pyridin-2-yl)pyrrolidine-1-carboxylate.

Step b: The product from step a (480 mg, 1.42 mmol, 1.0 equiv.) was dissolved in dioxane (14 mL). Aqueous KOH solution (4.7 mL, 14.2 mmol, 10 equiv., 3 M) and t-BuXPhosPd G3 (222 mg, 0.28 mmol, 0.2 equiv.) were added, and the reaction mixture was refluxed for 1 h before it was cooled to room temperature, diluted with water, and extracted with CHCl₃/i-PrOH mixture (3×15 mL, 4:1 v/v). The combined organic extract was dried over Na₂SO₄, concentrated under vacuum, and the crude residue was purified by column chromatography (SiO₂, 0-100% 0-10% MeOH gradient in dichloromethane) to furnish tert-butyl (2R)-2-(7-methyl-4-oxo-3,5-dihydroimidazo[4,5-c]pyridin-2-yl)pyrrolidine-1-carboxylate.

Step c: To a solution of the product from step b (550 mg, 1.7 mmol, 1.0 equiv.) in THF (5 mL), i-Pr₂NEt (0.6 mL, 3.5 mmol, 2.0 equiv.) and SEMCl (0.45 mL, 2.6 mmol, 1.5 equiv.) were sequentially added. The resulting solution was stirred at room temperature overnight followed by the dilution with water (10 mL) and EtOAc (20 mL). The organic phase was separated, dried over Na₂SO₄ and concentrated to dryness under reduced pressure. The crude residue was fractionated by column chromatography (SiO₂, 0-50% MeOH gradient in dichloromethane) to give tert-butyl (2R)-2-[7-methyl-4-oxo-3-(2-trimethylsilylethoxymethyl)-5H-imidazo[4,5-c]pyridin-2-yl]pyrrolidine-1-carboxylate.

Step d: A solution of the product from step c (110 mg, 0.25 mmol, 1.0 equiv.), [2-(2-chloro-6-cyclopropylpyridin-4-yl)-5-fluorophenyl]-(3-fluoroazetidin-1-yl)methanone (85.0 mg, 0.25 mmol, 1.0 equiv., prepared according to example 21), DMEDA (54 μL, 0.5 mmol, 2.0 equiv.) and K₂CO₃ (103 mg, 0.75 mmol, 3.0 equiv.) in CH₃CN (2 mL) was degassed by three cycles of vacuum/backfilling with nitrogen followed by the addition of CuI (47.5 mg, 0.25 mmol, 1.0 equiv.). The resulting mixture was heated in a sealed vial at 95° C. overnight. Once cooled to 23° C. the mixture was partitioned between aq. sat. NH₄Cl (5 mL) and EtOAc (15 mL). The organic phase was separated, and the aqueous phase was additionally extracted with EtOAc (2×5 mL). The combined organic phase was dried over Na₂SO₄ and concentrated to dryness under reduced pressure. The crude product was directly used for the next step without purification.

Step e: To a solution of the product from step d (assume 0.25 mmol) in dichloromethane (2 mL) was added trifluoroacetic acid (2 mL). The reaction mixture was stirred at 23° C. for 1 h, solvent was removed under reduced pressure, and the crude product was directly purified by preparative HPLC (SiO₂ C18, 10-90% CH₃CN in water with 0.1% formic acid) to yield the title compound. ¹H NMR (400 MHz, CDCl₃) δ 8.47 (s, 1H), 8.03 (d, J=7.5 Hz, 1H), 7.72 (d, J=5.0 Hz, 1H), 7.51-7.44 (m, 1H), 7.41 (d, J=10.0 Hz, 1H), 7.25-7.16 (m, 3H), 5.23-5.01 (m, 1H), 4.83 (s, 1H), 4.41-4.25 (m, 1H), 4.12 (dd, J=24.3, 12.0 Hz, 1H), 3.92 (s, 1H), 3.76 (dd, J=23.6, 11.1 Hz, 1H), 3.31 (s, 1H), 2.40 (s, 2H), 2.29 (s, 3H), 2.12 (td, J=8.1, 4.0 Hz, 1H), 2.02 (s, 3H), 1.16-0.99 (m, 4H). ESI MS [M+H]⁺ for C₂₉H₂₉F₂N₆O₂, calcd 531.2, found 531.1.

### Example 50: 5-[6-cyclopropyl-4-[4-fluoro-2-(3-fluoroazetidine-1-carbonyl)phenyl]pyridin-2-yl]-7-methyl-2-[(2S)-pyrrolidin-2-yl]-3H-imidazo[4,5-c]pyridin-4-one

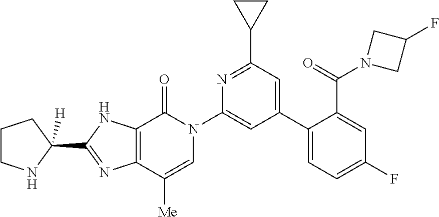 [SMILES: Cc1cn(-c2cc(-c3ccc(F)cc3C(=O)N3CC(F)C3)cc(C3CC3)n2)c(=O)c2[nH]c([C@@H]3CCCN3)nc12]

The title compound was prepared in a similar fashion to example 49 starting from 2S)-1-benzylpyrrolidine-2-carboxylic acid on step a. ¹H NMR (400 MHz, CDCl₃) δ 8.47 (s, 1H), 8.03 (d, J=7.5 Hz, 1H), 7.72 (d, J=5.0 Hz, 1H), 7.51-7.44 (m, 1H), 7.41 (d, J=10.0 Hz, 1H), 7.25-7.16 (m, 3H), 5.23-5.01 (m, 1H), 4.83 (s, 1H), 4.41-4.25 (m, 1H), 4.12 (dd, J=24.3, 12.0 Hz, 1H), 3.92 (s, 1H), 3.76 (dd, J=23.6, 11.1 Hz, 1H), 3.31 (s, 1H), 2.40 (s, 2H), 2.29 (s, 3H), 2.12 (td, J=8.1, 4.0 Hz, 1H), 2.02 (s, 3H), 1.16-0.99 (m, 4H). ESI MS [M+H]⁺ for C₂₉H₂₉F₂N₆O₂, calcd 531.2, found 531.2.

### Example 51: 6-[6-cyclopropyl-4-[4-fluoro-2-(3-fluorocyclobutanecarbonyl)phenyl]pyridin-2-yl]-7-oxo-4-(trifluoromethyl)-1H-pyrrolo[2,3-c]pyridine-3-carbonitrile

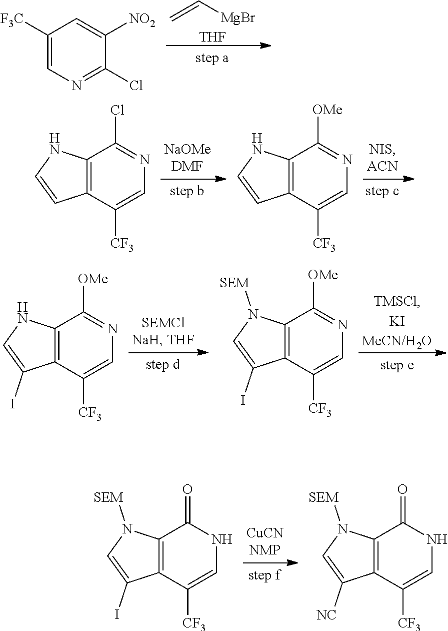

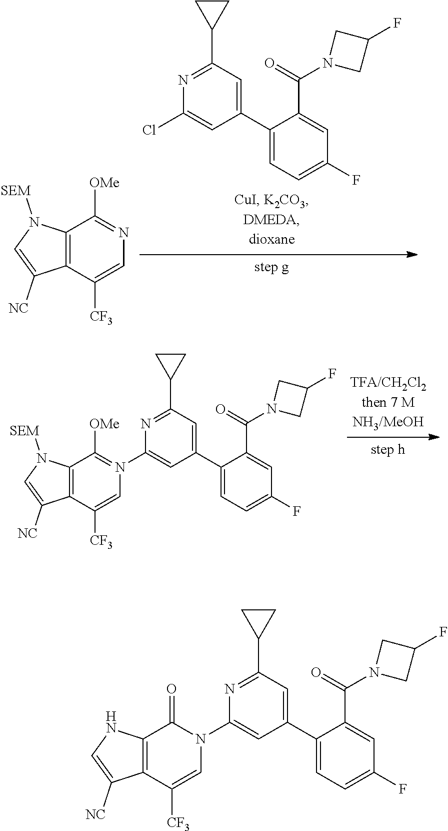

Step a: To a solution of 2-chloro-3-nitro-5-(trifluoromethyl)pyridine (5.3 g, 23 mmol, 1.0 equiv.) in THF (50 mL) was added vinylmagnesium bromide (1M in THF, 76 mL, 76 mmol, 3.3 equiv.) at −78° C. over 30 min. The resulting mixture was stirred at this temperature for another 30 min before being quenched with saturated aqueous NH₄Cl solution (20 mL). The product was extracted with EtOAc (2×80 mL). The combined organic phase was washed with brine, dried over Na₂SO₄ and concentrated. The crude residue was then purified by column chromatography (SiO₂, EtOAc in hexanes, 0 to 25%) to give the bicyclic product.

Step b: To a solution of the product from step a (1.02 g, 4.46 mmol, 1.0 equiv.) in DMF (22 mL) was added NaOMe (2.70 g, 44.6 mmol, 10 equiv.). The resulting mixture was heated at 130° C. for 1 h. After cooling back to room temperature, the mixture was diluted with EtOAc (70 mL) and washed sequentially with water (2×60 mL) and brine (60 mL). The combined organic extract was dried over Na₂SO₄ and concentrated. The crude product was purified by column chromatography (SiO₂, EtOAc in hexanes, 0 to 20%) to afford the product of nucleophilic substitution.

Step c: To a solution of the product from step b (1.50 g, 6.94 mmol, 1.0 equiv.) in MeCN (28 mL) was added N-iodosuccinimide (2.0 g, 9.0 mmol, 1.30 equiv.). The resulting mixture was stirred for 1 h when LCMS showed the completion of the iodination. The reaction was diluted with EtOAc (20 mL) and sequentially washed with aqueous saturated Na₂S₂O₃ (20 mL), water (20 mL) and brine (10 mL). The organic extract was concentrated under reduced pressure, and the crude product was purified by column chromatography (SiO₂, EtOAc in hexanes, 0 to 60%) to yield iodination product.

Step d: To a solution of the product from step c (500 mg, 1.46 mmol, 1.0 equiv.) in THF (7.30 mL, 0.2 M) was added NaH (120 mg, 60% in mineral oil, 2.92 mmol, 2.0 equiv.) at 0° C. SEMCl (370 mg, 2.19 mmol, 1.5 equiv.) was added after 15 min, and the reaction was stirred for 12 h at room temperature. The resulting solution was quenched with water (10 mL), and the aqueous layer was extracted with EtOAc (2×10 mL). The combined organic phase was dried over Na₂SO₄, concentrated and the crude residue was purified by column chromatography (SiO₂, EtOAc in hexanes, 10 to 50%) to afford SEM-protected bicyclic product.

Step e: The product from step d (670 mg, 1.42 mmol, 1.0 equiv.) was dissolved in a mixture of MeCN (7.10 mL, 0.2 M) and H₂O (77 μL, 4.26 mmol, 3.0 equiv). KI (380 mg, 2.27 mmol, 1.6 equiv.) and TMSCl (0.29 mL, 2.27 mmol, 1.6 equiv) were added sequentially, and the reaction mixture was stirred for 1 h at 45° C. Upon cooling to ambient temperature the mixture was diluted with EtOAc (20 mL) and quenched with water (5 mL). The organic phase was separated, and the aqueous phase was extracted with EtOAc (2×10 mL), the combined organic phase was then washed with brine (10 mL), dried over Na₂SO₄ and concentrated. The crude residue was purified by normal phase column chromatography (SiO₂, EtOAc in Hexanes, 0 to 60%), followed by additional purification by reversed-phase column chromatography (C18, 10-100% CH₃CN in water with 0.1% formic acid) to afford the corresponding product.

Step f: The product from step e (120 mg, 0.26 mmol, 1.0 equiv.) was dissolved in NMP (0.64 mL, 0.4 M) followed by addition of CuCN (48 mg, 0.52 mmol, 2.0 equiv). The reaction mixture was degassed by applying vacuum followed by backfilling with N₂. The reaction mixture was stirred for 3 h at 120° C. After cooling to room temperature the resulting mixture was diluted with EtOAc (10 mL) and washed with sat. ammonium chloride (4 mL). The organic phase was separated, washed with water (2×5 mL) dried over Na₂SO₄ and concentrated. The crude residue was purified by column chromatography (SiO₂, EtOAc in Hexanes, 0 to 30%), to the cyanation product.

Step g: A solution of the product from step f (35 mg, 0.1 mmol, 1.0 equiv.), [2-(2-chloro-6-cyclopropylpyridin-4-yl)-5-fluorophenyl]-(3-fluoroazetidin-1-yl)methanone (44 mg, 0.13 mmol, 1.3 equiv. prepared according to Example 21) and K₂CO₃ (41 mg, 0.29 mmol, 3.0 equiv.) in dioxane (2.0 ml) was degassed with a stream of bubbling nitrogen for 10 min. CuI (19 mg, 0.098 mmol, 1.0 equiv.) and DMEDA (19 μL, 0.20 mmol, 2.0 equiv.) were added, and the reaction was heated for 16 h at 100° C. Then the mixture was cooled to room temperature, diluted with aq. NH₄Cl (5 mL) and extracted with EtOAc (2×5 mL). The combined organic phase was washed with water (2×5 mL) and brine (5 mL), dried over Na₂SO₄, and concentrated to dryness. The crude residue was purified by reversed-phase column chromatography (SiO₂ C18, 10-100% CH₃CN in water with 0.1% formic acid) to afford desired coupling product.

Step h: The product from the step g (60 mg, 0.072 mmol, 1.0 equiv.) was treated with trifluoroacetic acid/dichlorormethane (v/v 1:1, 1 mL) at room temperature for 3 h. The mixture was concentrated to dryness under vacuum. The dry residue was treated with 7M NH₃ in methanol (1 mL) for 30 min followed by solvent evaporation. The crude residue was purified by reversed-phase column chromatography (C18 SiO₂, 10-100% CH₃CN in water with 0.1% formic acid) to afford the title compound. ¹H NMR (400 MHz, CDCl₃) δ 11.19 (s, 1H), 8.17 (d, J=1.5 Hz, 1H), 7.69 (d, J=19.7 Hz, 2H), 7.46 (dd, J=8.5, 5.1 Hz, 1H), 7.29 (d, J=1.1 Hz, 1H), 7.23 (s, 1H), 5.21 (d, J=59.9 Hz, 1H), 4.54-4.39 (m, 1H), 4.24-4.10 (m, 1H), 4.04-3.90 (m, 1H), 3.82 (dd, J=23.1, 10.8 Hz, 1H), 2.21-2.07 (m, 1H), 1.14-1.05 (m, 4H). ESI MS [M+H]⁺ for C₂₈H₁₉F₅N₄O₂ calcd 539.1, found 539.1.

### Example 52: 6-[6-cyclopropyl-4-[4-fluoro-2-(3-fluorocyclobutanecarbonyl)phenyl]pyridin-2-yl]-2-(hydroxymethyl)-4-(trifluoromethyl)-1H-pyrrolo[2,3-c]pyridin-7-one

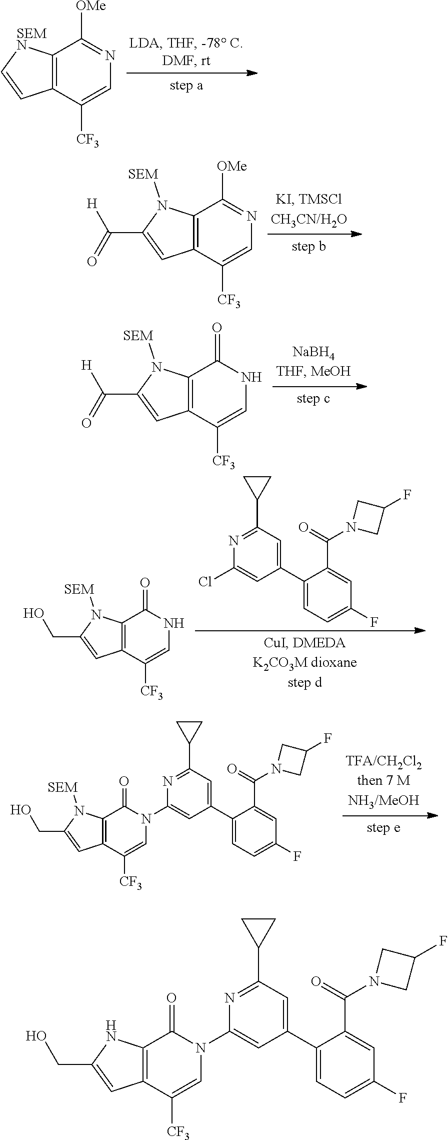

Step a: To a solution of 2-[[7-methoxy-4-(trifluoromethyl)pyrrolo[2,3-c]pyridin-1-yl]methoxy]ethyl-trimethylsilane (1.1 g, 3.2 mmol, 1.0 equiv., prepared according to example 51) in THF (11 mL, 0.3 M) at −78° C. was added LDA (2.0 M, 1.90 mL, 3.84 mmol, 1.2 equiv.). The mixture was stirred for 30 min at −78° C., then DMF (0.35 g, 4.8 mmol, 1.5 equiv.) was added in one portion. The reaction was stirred at −78° C. for 15 min and warmed up to room temperature and stirred for 2 h. The resulting mixture was quenched with saturated NH₄Cl aqueous solution (7 mL), and the product was extracted with EtOAc (2×10 mL). The combined organic phase was washed with brine, dried over Na₂SO₄ and concentrated. The residue was then purified by column chromatography (SiO₂, EtOAc in hexanes, 0 to 60%) to yield corresponding aldehyde product.

Step b: The product from step a (0.96 g, 2.56 mmol, 1.0 equiv.) was dissolved in MeCN (22 mL, 0.2 M) and H₂O (0.14 mL, 7.68 mmol, 3.0 equiv) followed by the addition of KI (672 mg, 4.04 mmol, 1.6 equiv.) and TMSCl (0.52 mL, 4.04 mmol, 1.6 equiv). The reaction mixture was stirred for 6 h at 45° C. The reaction mixture was allowed to cool to room temperature and was quenched with water (10 mL). The organic phase was separated, and the aqueous phase was extracted with EtOAc (2×20 mL). The combined organic phase was washed with brine (10 mL), dried over Na₂SO₄ and concentrated. The crude residue was purified by column chromatography (SiO₂, MeOH in dichloromethane, 0 to 20%) to afford the product.

Step c: To a solution of the product from step b (3.9 g, 8.28 mmol, 1.0 equiv.) in THF (40 mL, 0.2 M) and MeOH (40 mL, 0.2 M) was added NaBH₄ (630 mg, 16.6 mmol, 2.0 equiv.) at 0° C. The resulting mixture was stirred at 0° C. for 1 h. Once TLC analysis indicated complete transformation the reaction mixture was diluted with EtOAc (30 mL), carefully quenched with 1M HCl (10 mL) and warmed up to room temperature. The organic layer was separated, washed with brine (10 mL), dried over Na₂SO₄ and concentrated. The crude material was used directly for the next step.

Step d: A solution of the product from step c (300 mg, 0.83 mmol, 1.0 equiv.), [2-(2-chloro-6-cyclopropylpyridin-4-yl)-5-fluorophenyl]-(3-fluoroazetidin-1-yl)methanone (380 mg, 1.08 mmol, 1.3 equiv. prepared according to Example 21) and K₂CO₃ (340 mg, 2.50 mmol, 3.0 equiv.) in dioxane (17 ml, 0.05 M) was degassed with a stream of bubbling nitrogen for 10 min. CuI (160 mg, 0.83 mmol, 1.0 equiv.) and DMEDA (0.16 mL, 1.66 mmol, 2.0 equiv.) were added, and the reaction was heated for 16 h at 100° C. Then the mixture was cooled to room temperature, diluted with aq. NH₄Cl (10 mL) and extracted with EtOAc (2×10 mL). The combined organic phase was washed with water (2×10 mL) and brine (10 mL), dried over Na₂SO₄, and concentrated to dryness. The crude residue was purified by reversed-phase column chromatography (C18, 10-100% CH₃CN gradient in water with 0.1% formic acid) to afford desired coupling product.

Step e: The product from the step d (200 mg, 0.30 mmol, 1.0 equiv.) was treated with trifluoroacetic acid/dichloromethane (v/v 1:1, 3 mL) at 0° C. for 30 min. The mixture was concentrated to dryness under vacuum. The dry residue was treated with 7M NH₃ in methanol (3 mL) for 30 min followed by the solvent evaporation. The crude residue was purified by reversed-phase column chromatography (C18 SiO₂, 10-100% CH₃CN in water with 0.1% formic acid) to afford the title compound. ¹H NMR (400 MHz, CDCl₃) δ 11.57 (s, 1H), 7.90 (q, J=1.4 Hz, 1H), 7.64 (d, J=1.4 Hz, 1H), 7.52 (dd, J=8.5, 5.2 Hz, 1H), 7.26-7.16 (m, 4H), 6.37 (s, 1H), 5.17 (dtt, J=56.7, 6.3, 3.3 Hz, 1H), 4.66 (d, J=5.5 Hz, 2H), 4.46-4.27 (m, 1H), 4.22-4.07 (m, 1H), 4.02 (m, 2H), 3.81 (dd, J=23.2, 10.8 Hz, 1H), 2.15 (tt, J=7.6, 5.4 Hz, 1H), 1.10 (ddt, J=7.5, 4.3, 1.8 Hz, 4H). ESI MS [M+H]⁺ for C₂₈H₂₂F₅N₃O₃ calcd 544.2, found 544.2.

### Example 53: 6-[6-cyclopropyl-4-[4-fluoro-2-(3-fluorocyclobutanecarbonyl)phenyl]pyridin-2-yl]-2-(hydroxymethyl)-7-oxo-4-(trifluoromethyl)-1H-pyrrolo[2,3-c]pyridine-3-carbonitrile

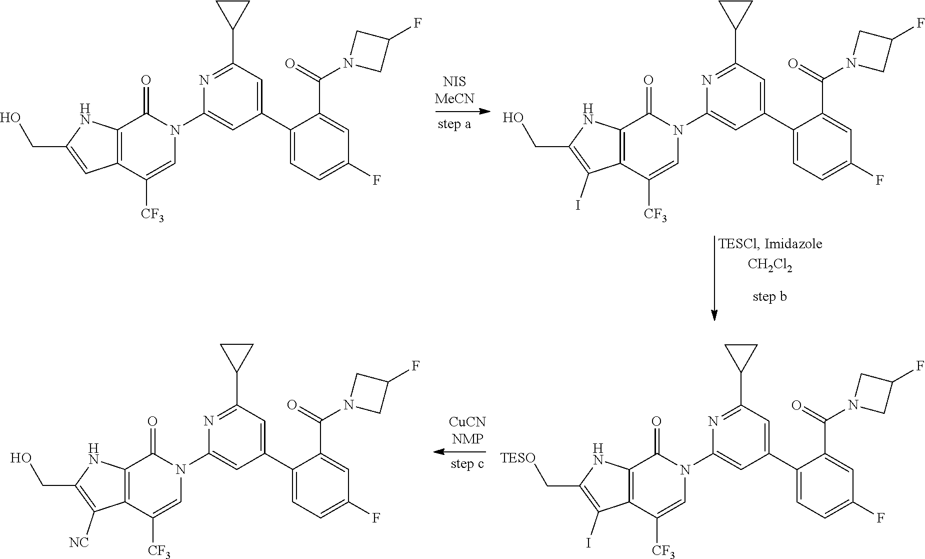

Step a: To a solution of 6-[6-cyclopropyl-4-[4-fluoro-2-(3-fluorocyclobutanecarbonyl)phenyl]pyridin-2-yl]-2-(hydroxymethyl)-4-(trifluoromethyl)-1H-pyrrolo[2,3-c]pyridin-7-one (160 mg, 0.29 mmol, 1.0 equiv., prepared according to example 52) in MeCN (1.20 mL, 0.25 M) was added N-iodosuccinimide (92 mg, 0.32 mmol, 1.40 equiv.). The resulting mixture was stirred for 1 h before LCMS analysis showed complete iodination. The mixture was diluted with EtOAc (20 mL), sequentially washed with sat. aq. Na₂S₂O₃ (20 mL), water (20 mL) and brine (10 mL). The organic extract was dried over Na₂SO₄ and concentrated to dryness under vacuum. The crude product was directly used for the next step.

Step b: To a solution of the product from step a (80 mg, 0.11 mmol, 1.0 equiv.) in dichloromethane (0.65 mL, 0.2 M) was added imidazole (27 mg, 0.40 mmol, 3.0 equiv.) at 0° C. The reaction was stirred for 10 min before TES-Cl (33 μL, 0.19 mmol, 1.5 equiv.) was added at 0° C. The resulting mixture was stirred for 1 h at 23° C. before being quenched with aq. hydrochloric acid solution (0.3 mL, 1 M). The organic layer was separated, and the aqueous layer was extracted with dichloromethane (2×3 mL). The combined organic phase was dried over Na₂SO₄ and concentrated under vacuum. The crude residue was purified by column chromatography (SiO₂, 10 to 80% EtOAc gradient in hexanes) to afford the desired product.

Step c: The product from step b (25 mg, 0.032 mmol, 1.0 equiv.) was dissolved in NMP (0.16 mL, 0.2 M), and CuCN (6 mg, 0.064 mmol, 2.0 equiv) was added. The mixture was degassed by three vacuum/backfilling with nitrogen cycles and stirred for 3 h at 120° C. The resulting mixture was cooled to room temperature, diluted with EtOAc (2 mL) and washed with aq. sat. ammonium chloride (2 mL). The organic phase was separated, dried over Na₂SO₄ and concentrated to dryness under reduced pressure. The crude residue was purified by reversed-phase column chromatography (SiO₂ C18, 10-100% CH₃CN in water with 0.1% formic acid) to afford the title compound. ¹H NMR (400 MHz, CDCl₃) δ 11.57 (s, 1H), 7.90 (q, J=1.4 Hz, 1H), 7.64 (d, J=1.4 Hz, 1H), 7.52 (dd, J=8.5, 5.2 Hz, 1H), 7.26-7.16 (m, 4H), 5.17 (dtt, J=56.7, 6.3, 3.3 Hz, 1H), 4.66 (d, J=5.5 Hz, 2H), 4.46-4.27 (m, 1H), 4.22-4.07 (m, 1H), 4.02 (m, 2H), 3.81 (dd, J=23.2, 10.8 Hz, 1H), 2.15 (tt, J=7.6, 5.4 Hz, 1H), 1.10 (ddt, J=7.5, 4.3, 1.8 Hz, 4H). ESI MS [M+H]⁺ for C₂₉H₂₁F₅N₄O₃ calcd 569.2, found 569.2.

### Example 54: 6-(6-cyclopropyl-4-{4-fluoro-2-[(3-fluoro-1-azetidinyl)carbonyl]phenyl}-2-pyridyl)-2-(hydroxymethyl)-7-oxo-1,6-dihydro-1,6-diaza-4-indenecarbonitrile

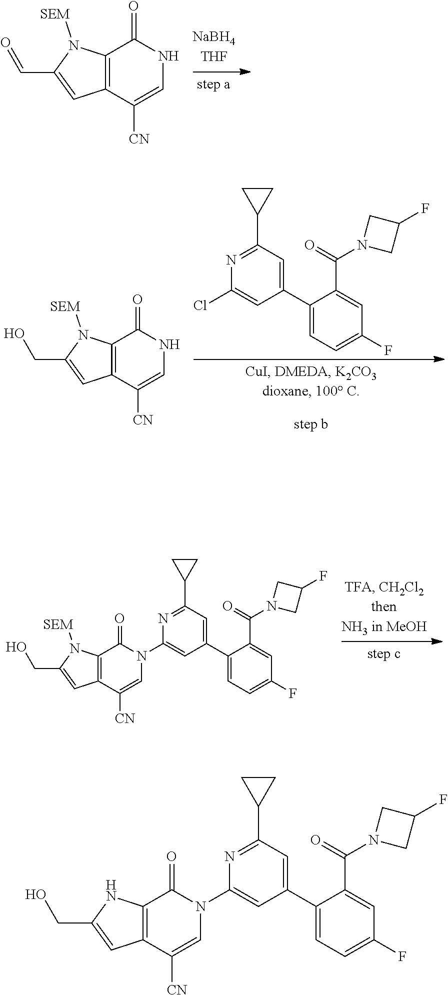 [SMILES: C[Si](C)(C)CCOCn1c(C=O)cc2c(C#N)c[nH]c(=O)c21 ; C[Si](C)(C)CCOCn1c(CO)cc2c(C#N)c[nH]c(=O)c21 ; O=C(c1cc(F)ccc1-c1cc(Cl)nc(C2CC2)c1)N1CC(F)C1 ; C[Si](C)(C)CCOCn1c(CO)cc2c(C#N)cn(-c3cc(-c4ccc(F)cc4C(=O)N4CC(F)C4)cc(C4CC4)n3)c(=O)c21 ; N#Cc1cn(-c2cc(-c3ccc(F)cc3C(=O)N3CC(F)C3)cc(C3CC3)n2)c(=O)c2[nH]c(CO)cc12]

Step a: To a solution of 2-formyl-7-oxo-1-(2-trimethylsilylethoxymethyl)-6H-pyrrolo[2,3-c]pyridine-4-carbonitrile (100 mg, 0.32 mmol, 1.0 equiv., obtained according to example 19, step f) in THF (2.1 mL, 0.15M) was added NaBH₄ (35.8 mg, 0.94 mmol, 3.0 equiv.). The resulting mixture was stirred at room temperature for 16 h. After complete consumption of the starting material was observed by TLC analysis the reaction was quenched with aq. sat. NH₄Cl (1 mL), diluted with water (5 mL), and the product was extracted with EtOAc (3×7 mL). The combined organic extract was dried over MgSO₄ and concentrated to dryness under vacuum to afford crude product that was used for the next step without purification.

Step b: To a solution of the product from step a (74 mg, 0.23 mmol, 1.0 equiv.) and [2-(2-chloro-6-cyclopropylpyridin-4-yl)-5-fluorophenyl]-(3-fluoroazetidin-1-yl)methanone (79 mg, 0.23 mmol, 1.0 equiv., obtained according to example 21, step b) in dioxane (4.7 mL, 0.05M) was added CuI (44 mg, 0.23 mmol, 1.0 equiv.), N,N′-dimethylethylenediamine (41 mg, 0.46 mmol, 2.0 equiv.), and K₂CO₃ (96 mg, 0.70 mmol, 3.0 equiv.). The resulting mixture was sparged with nitrogen for 10 min and heated at 100° C. for 6 h in a sealed vial. After cooling to room temperature, the reaction mixture was diluted with EtOAc (20 mL) and sequentially washed with aq. NH₄Cl (10 mL), water (10 mL) and brine (10 mL). The organic phase was separated, dried over MgSO₄ and concentrated to dryness under reduced pressure. The crude residue was fractionated by reversed phase prep-HPLC (SiO₂ C18 column, 10 to 100% CH₃CN gradient in water with 0.1% formic acid) to afford the desired compound.

Step c: The product from step b was dissolved in trifluoroacetic acid/dichloromethane mixture (3 mL, 1:1 v/v) and stirred at room temperature for 4 h. The resulting solution was concentrated to dryness, the residual trifluoroacetic acid was removed by co-evaporation with additional dichlorormethane (3 mL). Then the crude material was dissolved in 7M NH₃ in methanol (4 mL). The obtained solution was stirred for 1 h at 23° C. before solvent evaporation. The crude product was purified by reversed phase column chromatography (SiO₂ C18, 0 to 100% CH₃CN gradient in water) to furnish the title compound. ¹H NMR (400 MHz, CDCl₃) δ 10.45 (s, 1H), 8.15 (s, 1H), 7.68 (s, 1H), 7.45 (d, J=7.7 Hz, 1H), 7.22-7.10 (m, 2H), 6.40 (s, 1H), 5.11 (dtt, J=56.6, 6.3, 3.2 Hz, 1H), 4.79 (s, 2H), 4.33 (s, 2H), 4.39-4.29 (m, 1H), 4.20-4.06 (m, 2H), 3.96-3.80 (m, 2H), 2.11 (s, 1H), 0.86 (t, J=7.9 Hz, 4H). ESI MS [M+H]⁺ for C₂₇H₂₃F₂N₅O₃ calcd 502.2 found 502.1.

### Example 55: 6-[6-cyclopropyl-4-[4-fluoro-2-(3-fluoroazetidine-1-carbonyl)phenyl]pyridin-2-yl]-2-morpholin-3-yl-4-(trifluoromethyl)-1H-pyrrolo[2,3-c]pyridin-7-one

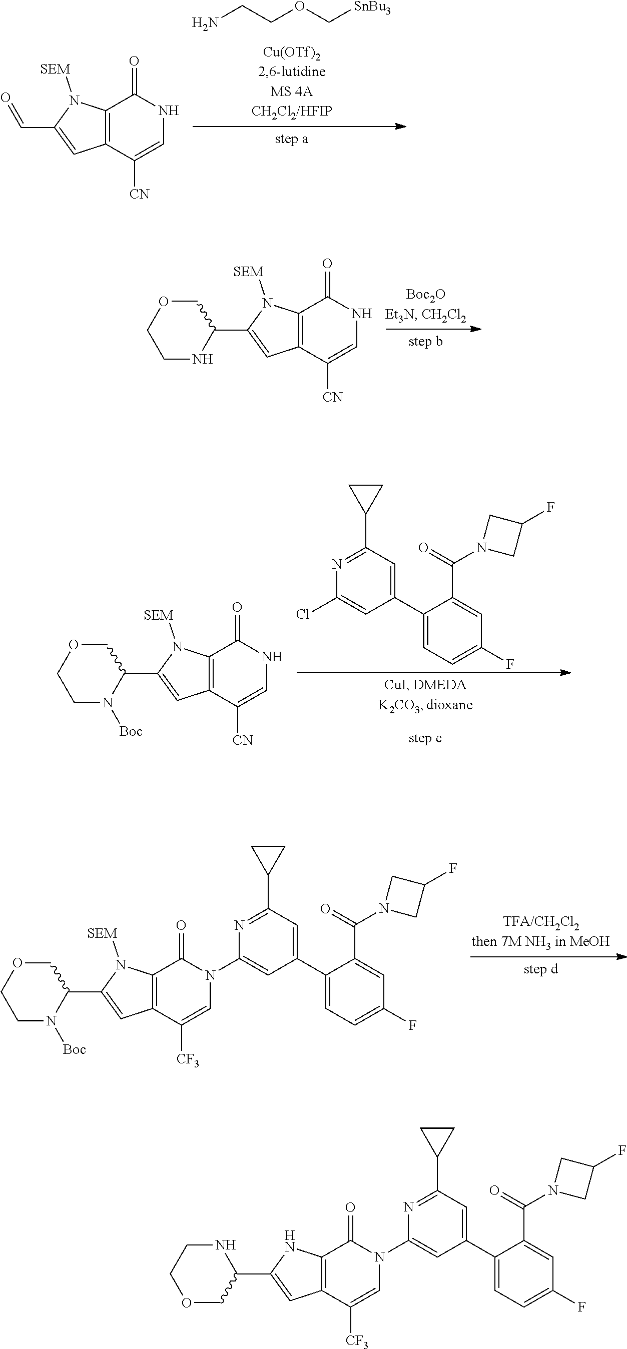

Step a: A mixture of 2-formyl-7-oxo-1-(2-trimethylsilylethoxymethyl)-6H-pyrrolo[2,3-c]pyridine-4-carbonitrile (0.3 g, 0.95 mmol, 1 equiv., obtained according to example 19, step f), 2-(tributylstannylmethoxy)ethanamine (0.35 g, 0.95 mmol, 1 equiv.) and activated MS 4A (100 mg, freshly prepared powder from granular material) in dichloromethane (4.8 mL, 0.2 M) was stirred overnight at room temperature. The mixture was filtered through a Celite® plug and concentrated to dryness under vacuum to afford corresponding crude imine product. In a separate flask hexafluoroisopropanol (4 mL) was added to a suspension of anhydrous Cu(OTf)₂ (0.34 g, 0.95 mmol, 1 equiv.) in dichloromethane (16 mL) followed by the addition of 2,6-lutidine (110 μL, 0.95 mmol, 1 equiv.). The resulting dark blue suspension was stirred for 1 h. The imine product described above was dissolved in dichloromethane (1 mL), and the resulting solution was combined with the solution of Cu(OTf)₂ in dichloromethane/hexafluorisopropanol mixture. The reaction was stirred for 4 h followed by partitioning between aq. sat. NH₄Cl (10 mL) and dichloromethane (15 mL). The organic phase was separated, and the aqueous phase was additionally extracted with dichloromethane (2×15 mL). The combined organic extract was dried over Na₂SO₄ and concentrated to dryness under reduced pressure. The crude product was purified by reversed phase column chromatography (SiO₂ C18, 0-100% CH₃CN in water with 0.1% formic acid) to afford 2-morpholin-3-yl-7-oxo-1-(2-trimethylsilylethoxymethyl)-6H-pyrrolo[2,3-c]pyridine-4-carbonitrile as a mixture of enantiomers.

Step b: The product from step a (0.21 g, 0.56 mmol, 1 equiv.) was dissolved in a mixture of triethylamine (0.16 mL, 1.12 mmol, 2 equiv.) and dichloromethane (3 mL, 0.2 M). Boc₂O (0.19 g, 0.84 mmol, 1.5 equiv.) was added, and the mixture was stirred at 23° C. for 48 h. Once complete consumption of the starting material was observed by TLC analysis the solvent was removed under reduced pressure. The residue was dissolved in MeOH (4 mL), aq. conc. NH₄OH (1 mL) was added, and the mixture was stirred at 23° C. for 16 h. The solution was diluted with EtOAc (15 mL) and washed with brine (10 mL). The organic layer was separated, dried over Na₂SO₄ and concentrated to dryness under reduced pressure. The crude residue was purified by column chromatography (SiO₂, 0-100% EtOAc gradient in hexanes) to afford tert-butyl 3-[4-cyano-7-oxo-1-(2-trimethylsilylethoxymethyl)-6H-pyrrolo[2,3-c]pyridin-2-yl]morpholine-4-carboxylate.

Step c: A mixture of the product from step b (0.18 g, 0.38 mmol, 1 equiv.), 2-(2-chloro-6-cyclopropylpyridin-4-yl)-5-fluorophenyl]-(3-fluoroazetidin-1-yl)methanone (0.17 g, 0.49 mmol, 1.3 equiv., obtained according to example 21, step b), DMEDA (0.16 mL, 0.76 mmol, 2 equiv.) and K₂CO₃ (0.16 g, 1.14 mmol, 3 equiv.) was degassed by three vacuuming/backfilling with nitrogen. CuI (72 mg, 0.38 mmol, 1 equiv.) was added, and the mixture was heated at 100° C. for 48 h. The resulting mixture was allowed to cool to room temperature and partitioned between aq. sat. NH₄Cl (5 mL) and EtOAc (10 mL). The organic layer was separated, dried over Na₂SO₄ and concentrated to dryness under reduced pressure. The crude product was purified by column chromatography (SiO₂, 0-100% EtOAc gradient in hexanes) to afford tert-butyl 3-[6-[6-cyclopropyl-4-[4-fluoro-2-(3-fluoroazetidine-1-carbonyl)phenyl]pyridin-2-yl]-7-oxo-4-(trifluoromethyl)-1-(2-trimethylsilylethoxymethyl)pyrrolo[2,3-c]pyridin-2-yl]morpholine-4-carboxylate.

Step d: The product from step c (0.3 g, 0.38 mmol) was dissolved in dichloromethane (2 mL), trifluoroacetic acid was added, and the resulting mixture was stirred at 23° C. for 1 h. The solvent was removed under reduced pressure, and the residue was dissolved in 7M NH₃ in MeOH (2 mL). After 1 h the solution was concentrated and directly purified by reversed phase column chromatography (SiO₂ C18, 0 to 100% CH₃CN in water with 0.1% formic acid) to afford the title compound as a mixture of enantiomers. ¹H NMR (400 MHz, CDCl₃) δ 11.68 (br. s, 1H), 8.07 (s, 1H), 7.62 (s, 1H), 7.54-7.38 (m, 1H), 7.38-7.12 (m, 3H), 6.42 (s, 1H), 5.13 (d, J=56.5 Hz, OH), 4.53-4.26 (m, 1H), 4.24-4.18 (m, 1H), 4.16-4.05 (m, 1H), 4.00 (dd, J=11.2, 3.2 Hz, 1H), 3.96-3.83 (m, 2H), 3.83-3.68 (m, 1H), 3.67-3.58 (m, 2H), 3.23-2.94 (m, 2H), 2.23-1.99 (m, 1H), 1.07 (s, 4H). ESI MS [M+H]⁺ for C₃₀H₂₆F₅N₅O₃ calcd 600.2 found 600.2.

### Example 56: 6-[6-cyclopropyl-4-[4-fluoro-2-(3-fluoroazetidine-1-carbonyl)phenyl]pyridin-2-yl]-2-(4-methylmorpholin-3-yl)-4-(trifluoromethyl)-1H-pyrrolo[2,3-c]pyridin-7-one

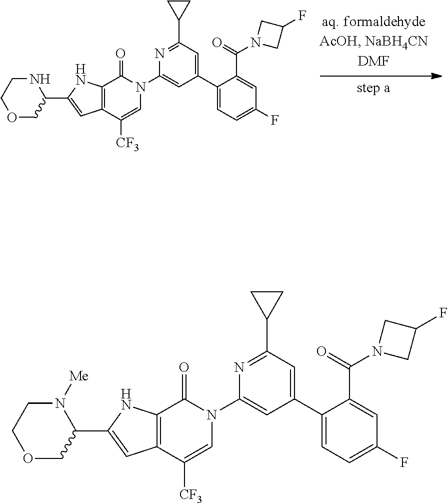 [SMILES: O=C(c1cc(F)ccc1-c1cc(C2CC2)nc(-n2cc(C(F)(F)F)c3cc(C4COCCN4)[nH]c3c2=O)c1)N1CC(F)C1 ; CN1CCOCC1c1cc2c(C(F)(F)F)cn(-c3cc(-c4ccc(F)cc4C(=O)N4CC(F)C4)cc(C4CC4)n3)c(=O)c2[nH]1]

Step a: A solution of 6-[6-cyclopropyl-4-[4-fluoro-2-(3-fluoroazetidine-1-carbonyl)phenyl]pyridin-2-yl]-2-morpholin-3-yl-4-(trifluoromethyl)-1H-pyrrolo[2,3-c]pyridin-7-one (50 mg, 0.09 mmol, 1 equiv., prepared according to example 55) in DMF (1.5 mL) was charged with aqueous formaldehyde (100 mg, 1.2 mmol, 40 equiv., 37 wt % solution), AcOH (31 μL, 0.54 mmol, 6 equiv.) and NaBH₃CN (23 mg, 0.36 mmol, 4 equiv.). The reaction mixture was stirred at 23° C. overnight before partitioning between EtOAc (10 mL) and aqueous 1M NaOH (10 mL). The organic phase was separated, sequentially washed with water (5 mL) and brine (5 mL), dried over Na₂SO₄, and concentrated to dryness under reduced pressure. The crude product was directly purified by reversed phase column chromatography (SiO₂ C18, 0-100% CH₃CN in water with 0.1% formic acid) to afford the title compound as a mixture of enantiomers. ¹H NMR (400 MHz, CDCl₃) δ 9.90 (d, J=17.6 Hz, 1H), 8.17 (d, J=0.9 Hz, 1H), 7.71 (d, J=1.4 Hz, 1H), 7.47 (dd, J=8.3, 5.1 Hz, 1H), 7.34-7.09 (m, 3H), 6.47 (s, 1H), 5.18 (d, J=56.5 Hz, 1H), 4.51-4.30 (m, 1H), 4.28-4.07 (m, 1H), 4.02-3.58 (m, 5H), 3.57-3.38 (m, 2H), 2.87-2.67 (m, 1H), 2.46-2.33 (m, J=14.2, 12.2, 2.9 Hz, 1H), 2.18-2.00 (m, 4H), 1.15-0.99 (m, 4H). ESI MS [M+H]⁺ for C₃₁H₂₈F₅N₅O₃ calcd 614.2 found 614.2.

### Biological Assay Examples

The affinity with which compounds of the present disclosure bind to Cbl-b was assessed using probe displacement homologous time resolved fluorescence (HTRF) assays. The assays used a BODIPY™ conjugated probe (Example 54 from WO 2020264398) and biotinylated Cbl-b. The assays were performed in assay buffer consisting of 20 mM Hepes, 150 mM NaCl, 0.01% Triton X-100, 0.5 mM TCEP, 0.01% BSA. On the day of the assay, a 20 point, 1:2 master serial dilution of each compound was prepared in DMSO to span a final concentration range of 10 μM to 0 nM. Two hundred nanoliters of diluted compound was added to each well of a 384-well plate. Fifteen microliters of biotinylated-Cbl-b resuspended in assay buffer were added to each well and the plate incubated for 60 minutes at room temperature prior to addition of 5 μL of BODIPY™ probe in assay buffer. Final assay conditions included 0.4 nM of Cbl-b and 150 nM of BODIPY™ probe. After a further 15 minutes of incubation at room temperature, 5 μl of Streptavidin-Terbium cryptate reagent at 5-fold final concentration was added to each well of the 384-well plate. Binding of a probe to Cbl-b results in an increase of HTRF signal. HTRF signal was measured by Envision plate reader, while competition of a compound of the present disclosure with probe results in a decrease of signal. Percentage maximum activity in each test well was calculated based on DMSO (maximum activity, 0% displacement) and no protein control wells (baseline activity, 100% displacement). Binding affinity was determined from a dose response curve fitted using a standard four parameter fit equation. See Table 3 for data for compounds (Cbl-b Binding (IC₅₀)).

Certain compounds were also evaluated in an IL-2 secretion assay. On the day of the assay, a 16 point, 1:2 master serial dilution of each compound was prepared in Opti-MEM to span a final concentration range of 10 μM to 300 μM. Assays were set up in CORNING® tissue culture-treated 384-well microplates containing 60 nL of each dilution. Jurkat cells grown in RPMI-1640 supplemented with 10% FBS, 1% Glutamax, and 1% Pen/Strep were collected, resuspended in Opti-MEM. 50,000 cells/well and added to the compound plates. After a short spin (1200 rpm for 1 min), the plates were incubated at 37° C. for 1 hour. The cells were activated by adding 15 μL of IMMUNOCULT™ Human CD3/CD28 T Cell Activator (STEMCELL Technologies) diluted in Opti-MEM. After 24 h of incubation at 37° C., aliquots of culture supernatants were transferred to OptiPlate-384 (PerkinElmer) microplates. The level of JIL-2 secretion in the supernatants was then determined using the IL-2 (human) AiphaLISA Detection Kit (PerkinElmer) according to the manufacturer's recommendations. The AiphaLISA signal was measured using an EnVision plate reader (PerkinElmer). EC₅₀ values were determined by fitting the data to a standard 4-parameter logistic equation. See Table 3 for data for select compounds (IL-2 Secretion (EC₅₀)).

TABLE 3
Potency of select compounds.

| Example # | Cbl-b Binding (IC₅₀) | IL-2 Secretion (EC₅₀) |
|---|---|---|
| 1 | +++ | +++ |
| 2 | ++ | + |
| 3 | +++ | ++ |
| 4 | +++ | ++ |
| 5 | +++ | ++ |
| 6 | +++ | ++ |
| 7 | +++ | +++ |
| 8 | ++ | − |
| 9 | +++ | − |
| 10 | +++ | +++ |
| 11 | +++ | ++ |
| 12 | ++ | ++ |
| 13 | +++ | ++ |
| 14 | +++ | +++ |
| 15 | +++ | ++ |
| 16 | +++ | +++ |
| 17 | +++ | +++ |
| 18 | ++ | ++ |
| 19 | +++ | ++ |
| 20 | ++ | + |
| 21 | +++ | +++ |
| 22 | +++ | ++ |
| 23 | ++ | + |
| 24 | ++ | ++ |
| 25 | +++ | +++ |
| 26 | +++ | +++ |
| 27 | ++ | + |
| 28 | +++ | ++ |
| 29 | +++ | +++ |
| 30 | +++ | +++ |
| 31 | + | − |
| 32 | +++ | + |
| 33 | ++ | ++ |
| 34 | +++ | +++ |
| 35 | +++ | +++ |
| 36 | +++ | +++ |
| 37 | +++ | ++ |
| 38 | +++ | ++ |
| 39 | +++ | +++ |
| 40 | +++ | n.d. |
| 41 | +++ | ++ |
| 42 | +++ | +++ |
| 43 | +++ | ++ |
| 44 | +++ | +++ |
| 45 | +++ | ++ |
| 46 | +++ | +++ |
| 47 | +++ | ++ |
| 48 | +++ | +++ |
| 49 | +++ | − |
| 50 | ++ | − |
| 51 | +++ | ++ |
| 52 | +++ | ++ |
| 53 | +++ | ++ |
| 54 | +++ | ++ |
| 55 | +++ | ++ |
| 56 | +++ | ++ |

Less than 100 nM (+++),
100 nM to 1 μM (++),
greater than 1 μM to 5 μM (+),
>5 μM (−),
n.d. = not determined

Particular embodiments of this disclosure are described herein, including the best mode known to the inventors for carrying out the disclosure. Upon reading the foregoing, description, variations of the disclosed embodiments may become apparent to individuals working in the art, and it is expected that those skilled artisans may employ such variations as appropriate. Accordingly, it is intended that the disclosure be practiced otherwise than as specifically described herein, and that the disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All publications, patent applications, accession numbers, and other references cited in this specification are herein incorporated by reference for the purpose described herein.

1. A compound having a structure according to Formula I: 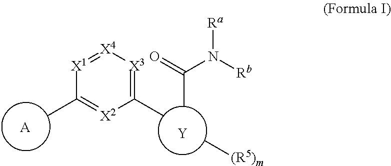
or a pharmaceutically acceptable salt thereof, wherein:
A has a formula selected from the group consisting of: 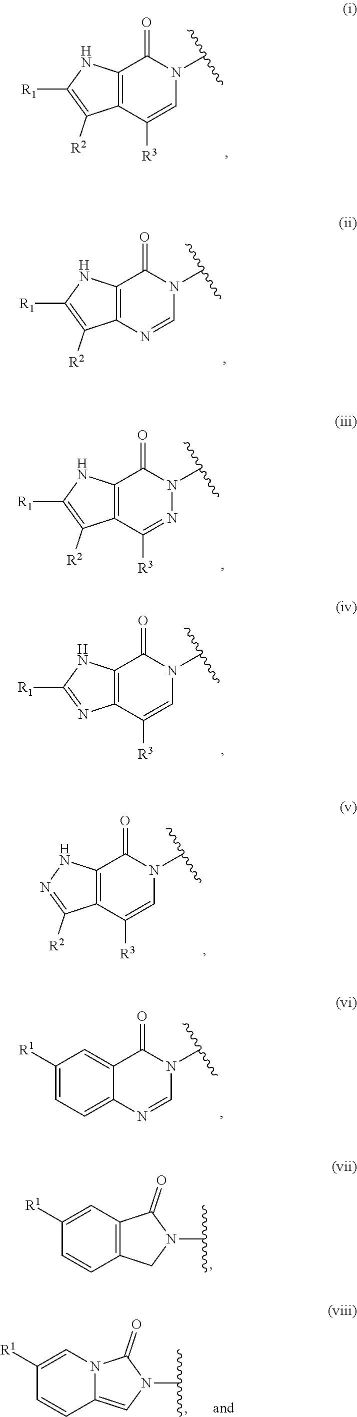 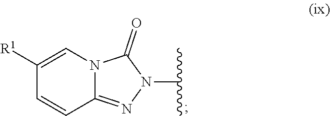
wherein:
R¹ is selected from the group consisting of —H, —C₁-C₆ haloalkyl, —C₁-C₆ hydroxyalkyl, —C(O)NH₂, —C(O)—(C₁-C₆-alkyl), -(Q¹)-NR^{1a}R^{1b}, -(Q¹)-(C₃-C₇ cycloalkyl), -(Q¹)-(5- to 6-membered heteroaryl), and -(Q¹)-(4- to 8-membered heterocycloalkyl); wherein said 5- to 6-membered heteroaryl has 1-3 ring heteroatoms independently selected from N, O, and S;
said 4 to 8-membered heterocycloalkyl has 1-3 ring heteroatom or heteroatom groups independently selected from N, O, S, and S(O)₂; and said C₃-C₇ cycloalkyl, 4- to 8-membered heterocycloalkyl, and 5- to 6-membered heteroaryl are unsubstituted or substituted with 1-2 substituents independently selected from halo, —OH, —C₁-C₃ alkyl, and —C₁-C₃ alkoxy;
Q¹ is absent, unsubstituted —(C₁-C₃ alkylene)-, or —(C₁-C₃ alkylene)- substituted with 1-3 R^{q};
each R^{q} is independently halo, —OH, or —NH₂;
R^{1a} and R^{1b} are independently selected from the group consisting of H, —C₁-C₆ alkyl, —C₁-C₆ haloalkyl, phenyl, —(C₁-C₃ alkylene)-O—(C₁-C₃ alkyl), —C₃-C₆ cycloalkyl, —(C₁-C₃ alkylene)-(C₃-C₆ cycloalkyl), —S(O)₂(C₁-C₆ alkyl), 5- to 6-membered heteroaryl having 1-3 ring heteroatoms independently selected from N, O, and S, and 4- to 8-membered heterocycloalkyl having 1-3 ring heteroatom or heteroatom groups independently selected from N, O, S, and S(O)₂; wherein said phenyl, —(C₁-C₃ alkylene)-O—(C₁-C₃ alkyl), —C₃-C₆ cycloalkyl, —(C₁-C₃ alkylene)-(C₃-C₆ cycloalkyl), 5- to 6-membered heteroaryl, and 4- to 8-membered heterocycloalkyl are unsubstituted or substituted with 1-3 R^{1c};
each R^{1c}, when present, is independently halo, —OH, —C₁-C₃ alkyl, —C₁-C₃ hydroxyalkyl, or —C₁-C₃ haloalkyl;
R², when present, is —H, halo, —CN, —C₁-C₃ alkyl, —C₁-C₃ haloalkyl, —C₃-C₄ cycloalkyl, —S(O)₂(C₁-C₃ alkyl), —C(O)—NR^{2a}R^{2b}, or 5- to 6-membered heteroaryl having 1-3 ring heteroatoms independently selected from N, O, and S;
R^{2a} and R^{2b} are independently —H, or —C₁-C₃ alkyl;
R³, when present, is —H, —CN, halo, —C₁-C₆ alkyl, —C₁-C₆ haloalkyl, —C₁-C₆ hydroxyalkyl, —C₂-C₃ alkenyl, —C₃-C₄ cycloalkyl, —S(O)₂(C₁-C₆ alkyl), —C(O)OH, or 5- to 6-membered heteroaryl having 1 to 4 ring heteroatoms independently selected from N, O, and S; and said 5- to 6-membered heteroaryl is unsubstituted or substituted with 1-3 substituents independently selected from —C₁-C₃ alkyl;
X¹, X² and X³ are each independently N or CH;
X⁴ is N or CR⁴;
R⁴, when present, is —H, halo, —CN, —OH, —C₁-C₆ alkyl, —C₁-C₆ haloalkyl, —C₁-C₆ alkoxy, —NR^{4a}R^{4b}, or —C₃-C₈ cycloalkyl;
R^{4a} and R^{4b} are independently —H, —C₁-C₃ alkyl, or —(C₁-C₃ alkylene)-NR^{4e}R^{4d};
R^{4c} and R^{4d} are independently —H, or —C₁-C₃ alkyl;
Y is phenyl, or 5- to 6-membered heteroaryl having 1-3 ring heteroatoms independently selected from N, O, and S;
m is 0, 1, 2, or 3;
each R⁵ when present, is independently halo, —CN, —C₁-C₆ alkyl, —C₁-C₆ haloalkyl, or —C₁-C₆ alkoxy;
R^{a} and R^{b} are each independently H, —C₁-C₆ alkyl, —C₁-C₆ haloalkyl, phenyl, or —(C₁-C₃ alkylene)-O—(C₁-C₃ alkyl); or
R^{a} and R^{b} taken together with the N atom to which they are attached form a 4- to 8-membered heterocycloalkyl optionally having one additional ring heteroatom selected from N, O, and S; wherein said 4- to 8-membered heterocycloalkyl is unsubstituted or substituted with 1-3 substituents independently selected from the group consisting of halo, —CN, —C₁-C₆ alkyl, and —C₁-C₆ alkoxy.

2. (canceled)

3. The compound of claim 1, or a pharmaceutically acceptable salt thereof, wherein:
R¹ is —H, —C₁-C₆ hydroxyalkyl, -(Q¹)-NR^{1a}R^{1b}, -(Q¹)-(C₃-C₇ cycloalkyl), or -(Q¹)-(4- to 8-membered heterocycloalkyl) having 1-3 ring heteroatoms independently selected from N, O, and S; and said 4- to 8-membered heterocycloalkyl is unsubstituted or substituted with 1-2 substituents independently selected from —C₁-C₃ alkyl, and —C₁-C₃ alkoxy;
Q¹ is absent or unsubstituted —(C₁-C₃ alkylene)-;
R^{1a} and R^{1b} are independently —H, —C₁-C₆ alkyl, —C₁-C₆ haloalkyl, —(C₁-C₃ alkylene)-O—(C₁-C₃ alkyl), unsubstituted —C₃-C₆ cycloalkyl, or —C₃-C₆ cycloalkyl substituted with 1 R^{1c}; and
R^{1c}, when present, is —OH or —C₁-C₃ alkyl.

4. The compound of claim 3, or a pharmaceutically acceptable salt thereof, wherein:
R¹ is —H, —C₁-C₆ hydroxyalkyl, -(Q¹)-NR^{1a}R^{1b} or -(Q¹)-(4- to 8-membered heterocycloalkyl) having 1-3 ring heteroatoms independently selected from N, O, and S; and said 4- to 8-membered heterocycloalkyl is unsubstituted or substituted with 1-2 substituents independently selected from —C₁-C₃ alkyl, and —C₁-C₃ alkoxy.

5.-6. (canceled)

7. The compound of claim 1, or a pharmaceutically acceptable salt thereof, wherein R², when present, is —H, halo, or —CN.

8. (canceled)

9. The compound of claim 1, or a pharmaceutically acceptable salt thereof, wherein R³ is —CN, —C₁-C₆ alkyl, —C₁-C₆ haloalkyl, or —C₃-C₄ cycloalkyl.

10. (canceled)

11. The compound of claim 1, or a pharmaceutically acceptable salt thereof, wherein the ring formed by X¹, X², X³, and X⁴ is selected from the group consisting of:

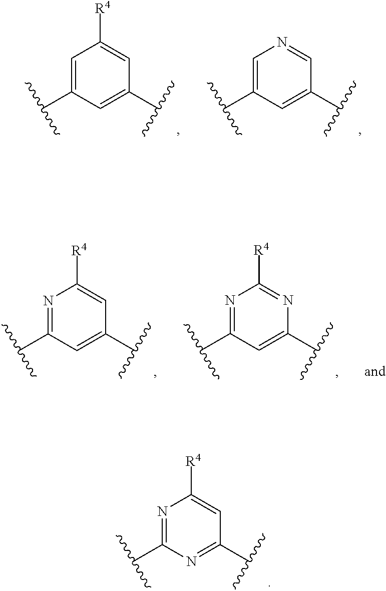

12. The compound of claim 1, or a pharmaceutically acceptable salt thereof, wherein the compound has a structure according to Formula Ia or Formula Ib:

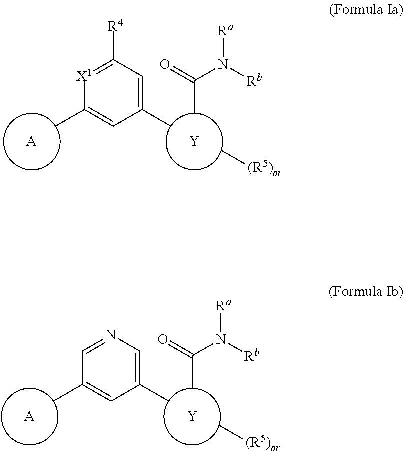

13. The compound of claim 1, or a pharmaceutically acceptable salt thereof, wherein R⁴, when present, is —CN, —C₁-C₆ haloalkyl, or —C₃-C₄ cycloalkyl.

14.-15. (canceled)

16. The compound of claim 1, or a pharmaceutically acceptable salt thereof, wherein Y is phenyl, pyrazolyl, or pyridyl.

17.-19. (canceled)

20. The compound of claim 1, or a pharmaceutically acceptable salt thereof, wherein the compound has a structure according to Formula Id-1: 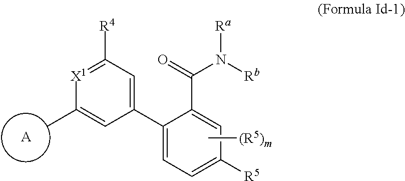
wherein m is 0 or 1.

21. (canceled)

22. The compound of claim 1, or a pharmaceutically acceptable salt thereof, wherein each R⁵ is independently halo or —C₁-C₆ alkyl.

23. (canceled)

24. The compound of claim 1, or a pharmaceutically acceptable salt thereof, wherein R^{a} and R^{b} are each independently —H, —C₁-C₆ alkyl, —C₁-C₆ haloalkyl, phenyl, or —(C₁-C₃ alkylene)-O—(C₁-C₃ alkyl).

25.-26. (canceled)

27. The compound of claim 1, or a pharmaceutically acceptable salt thereof, wherein R^{a} and R^{b} taken together with the N atom to which they are attached form a 4- to 8-membered heterocycloalkyl optionally having one additional ring heteroatom selected from N, O, and S; and wherein said 4- to 8-membered heterocycloalkyl is unsubstituted or substituted with 1-3 substituents independently selected from the group consisting of halo, —CN, —C₁-C₆ alkyl, and —C₁-C₆ alkoxy.

28. The compound of claim 27, or a pharmaceutically acceptable salt thereof, wherein R^{a} and R^{b} taken together with the N atom to which they are attached form 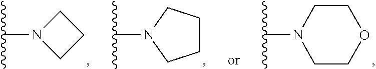
each of which is unsubstituted or substituted with 1-2 substituents independently selected from halo, —CN, —C₁-C₆ alkyl, and —C₁-C₆ alkoxy.

29. (canceled)

30. The compound of claim 1, or a pharmaceutically acceptable salt thereof, wherein A is:

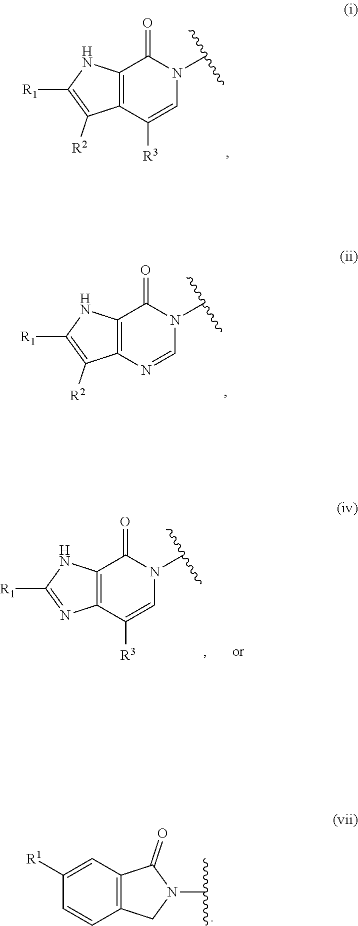

31. The compound of claim 1, or a pharmaceutically acceptable salt thereof, wherein A is:

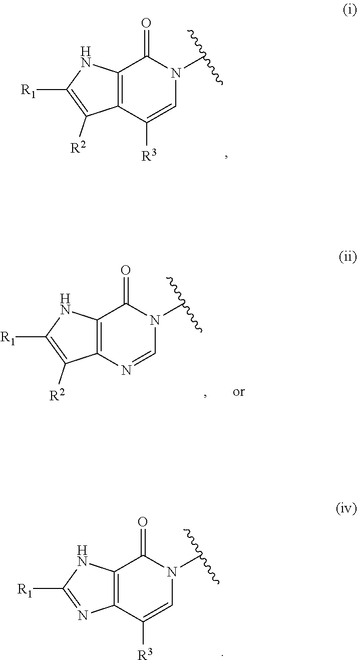

32. The compound of claim 1, or a pharmaceutically acceptable salt thereof, wherein A is

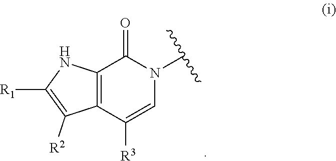

33. A compound selected from the group consisting of 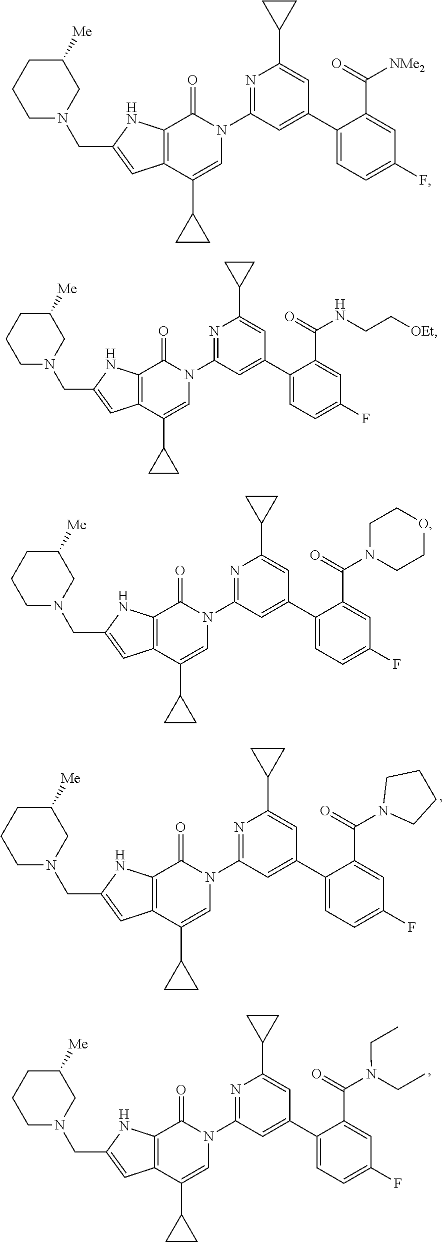 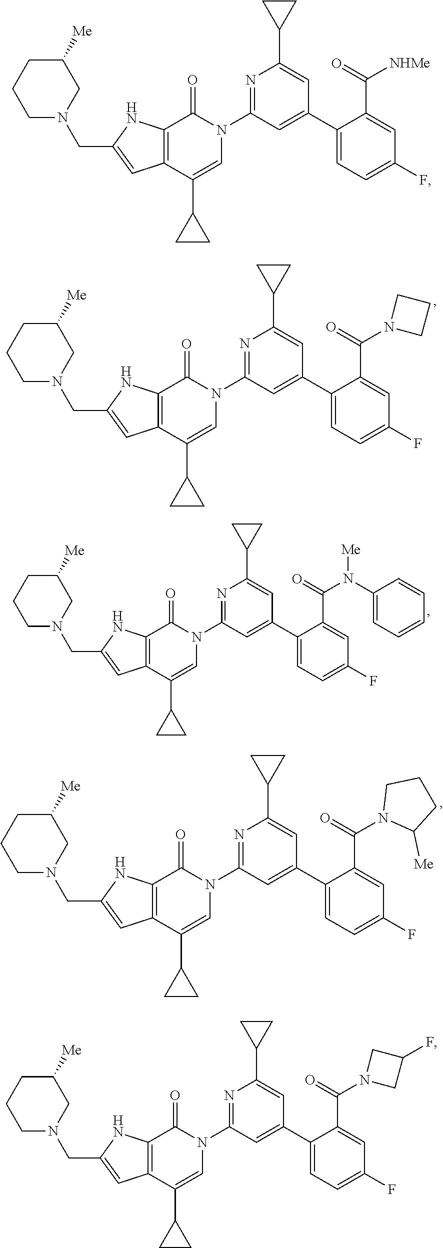 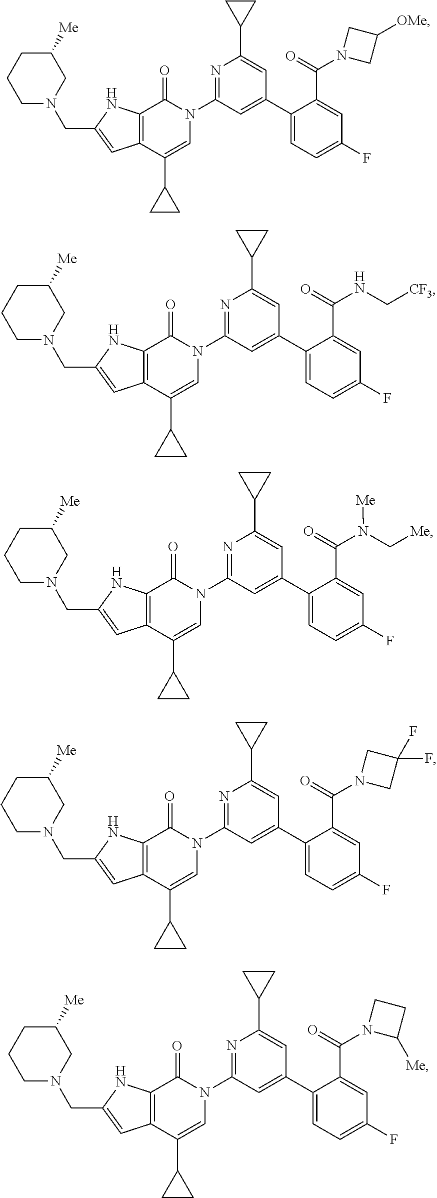 [SMILES: COC1CN(C(=O)c2cc(F)ccc2-c2cc(C3CC3)nc(-n3cc(C4CC4)c4cc(CN5CCC[C@H](C)C5)[nH]c4c3=O)c2)C1 ; C[C@H]1CCCN(Cc2cc3c(C4CC4)cn(-c4cc(-c5ccc(F)cc5C(=O)NCC(F)(F)F)cc(C5CC5)n4)c(=O)c3[nH]2)C1 ; CCN(C)C(=O)c1cc(F)ccc1-c1cc(C2CC2)nc(-n2cc(C3CC3)c3cc(CN4CCC[C@H](C)C4)[nH]c3c2=O)c1 ; C[C@H]1CCCN(Cc2cc3c(C4CC4)cn(-c4cc(-c5ccc(F)cc5C(=O)N5CC(F)(F)C5)cc(C5CC5)n4)c(=O)c3[nH]2)C1 ; CC1CCN1C(=O)c1cc(F)ccc1-c1cc(C2CC2)nc(-n2cc(C3CC3)c3cc(CN4CCC[C@H](C)C4)[nH]c3c2=O)c1] 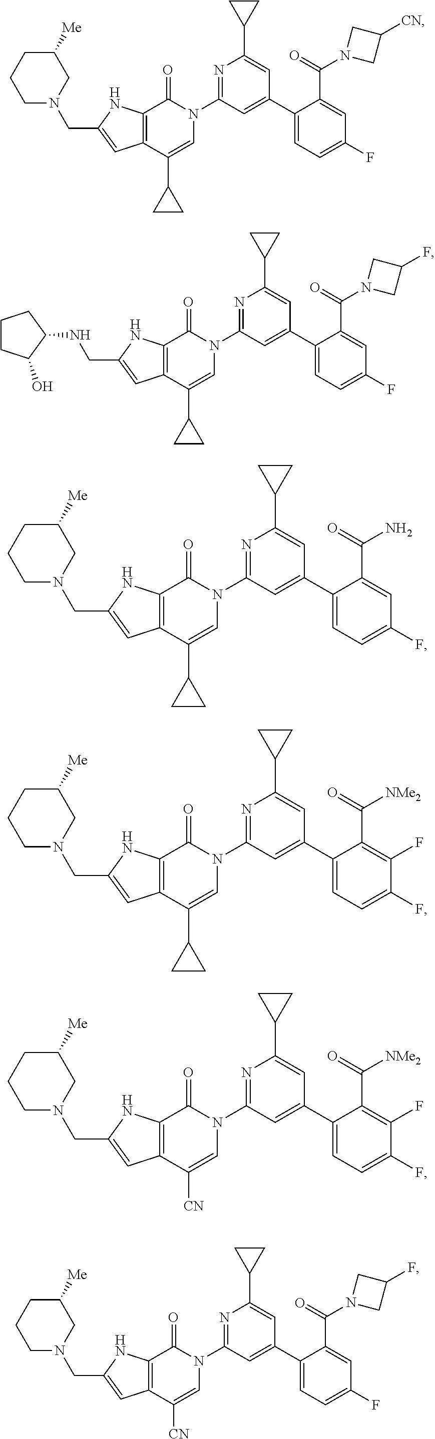 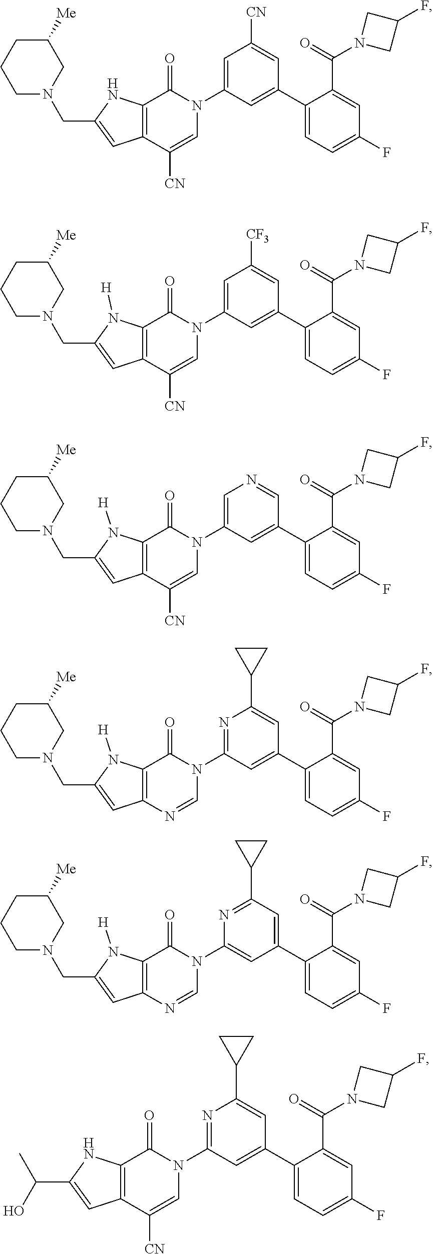 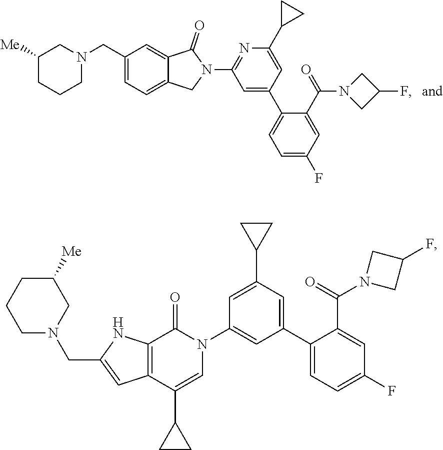 [SMILES: C[C@H]1CCCN(Cc2ccc3c(c2)C(=O)N(c2cc(-c4ccc(F)cc4C(=O)N4CC(F)C4)cc(C4CC4)n2)C3)C1 ; C[C@H]1CCCN(Cc2cc3c(C4CC4)cn(-c4cc(-c5ccc(F)cc5C(=O)N5CC(F)C5)cc(C5CC5)c4)c(=O)c3[nH]2)C1]
or a pharmaceutically acceptable salt thereof.

34. A compound selected from the group consisting of 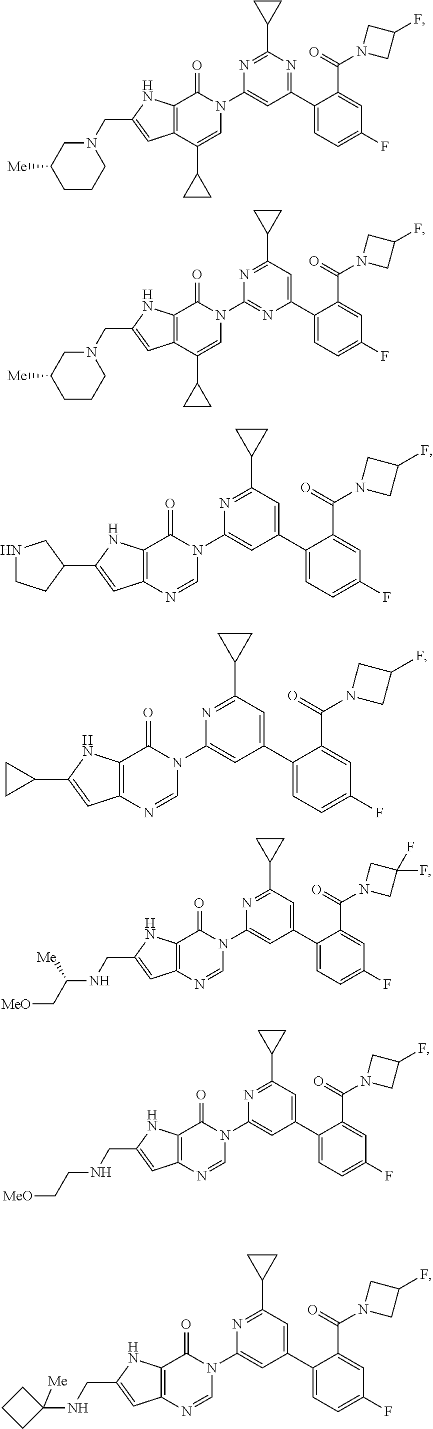 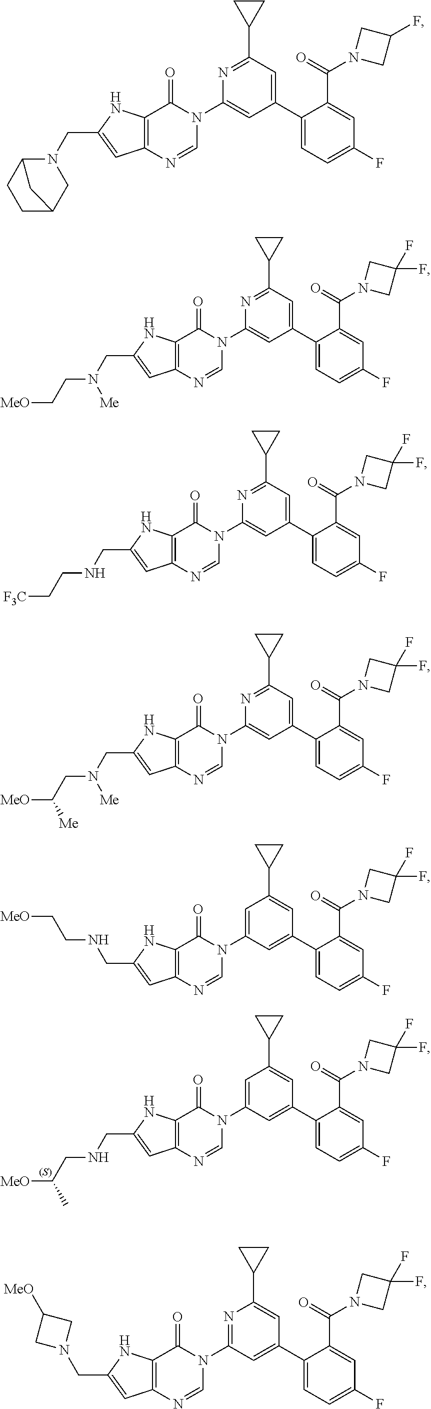 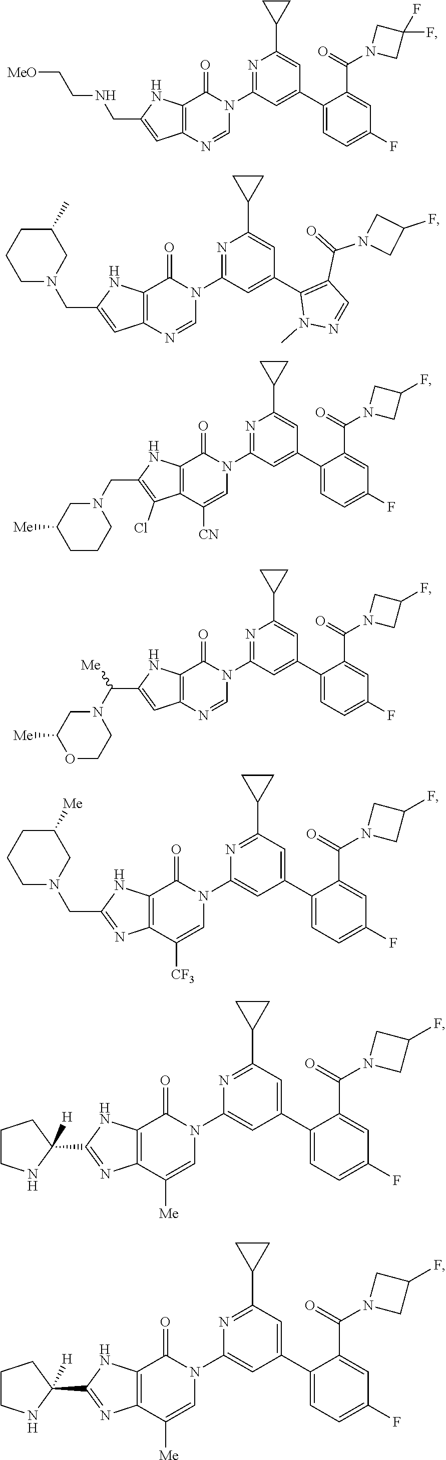 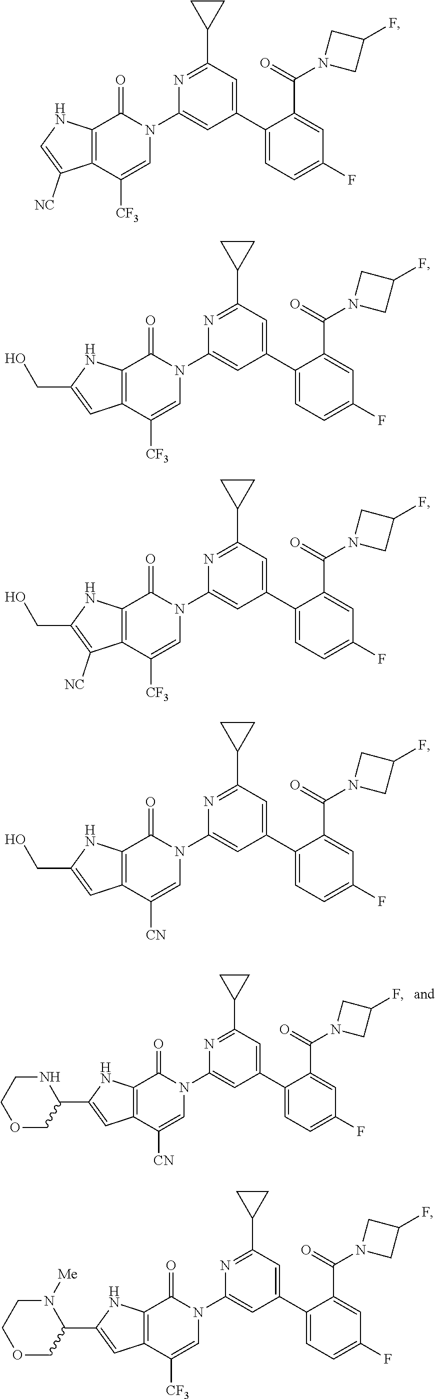 [SMILES: N#Cc1c[nH]c2c(=O)n(-c3cc(-c4ccc(F)cc4C(=O)N4CC(F)C4)cc(C4CC4)n3)cc(C(F)(F)F)c12 ; O=C(c1cc(F)ccc1-c1cc(C2CC2)nc(-n2cc(C(F)(F)F)c3cc(CO)[nH]c3c2=O)c1)N1CC(F)C1 ; N#Cc1c(CO)[nH]c2c(=O)n(-c3cc(-c4ccc(F)cc4C(=O)N4CC(F)C4)cc(C4CC4)n3)cc(C(F)(F)F)c12 ; N#Cc1cn(-c2cc(-c3ccc(F)cc3C(=O)N3CC(F)C3)cc(C3CC3)n2)c(=O)c2[nH]c(CO)cc12 ; N#Cc1cn(-c2cc(-c3ccc(F)cc3C(=O)N3CC(F)C3)cc(C3CC3)n2)c(=O)c2[nH]c(C3COCCN3)cc12 ; CN1CCOCC1c1cc2c(C(F)(F)F)cn(-c3cc(-c4ccc(F)cc4C(=O)N4CC(F)C4)cc(C4CC4)n3)c(=O)c2[nH]1]
or a pharmaceutically acceptable salt thereof.

35. A pharmaceutical composition comprising a compound of claim 1, or a pharmaceutically acceptable salt thereof, and one or more pharmaceutically acceptable excipients.

36.-37. (canceled)

38. A method of treating a disease, disorder, or condition mediated at least in part by Cbl-b in a subject in need thereof, the method comprising administering to the subject a compound of claim 1, or a pharmaceutically acceptable salt thereof.

39. (canceled)

40. The method of claim 38, wherein the disease, disorder, or condition is cancer.

41. The method of claim 40, wherein the cancer is:
a) a cancer of the genitourinary tract (e.g., gynecologic, bladder, kidney, renal cell, penile, prostate, or testicular), breast, gastrointestinal tract (e.g., esophagus, oropharynx, stomach, small or large intestines, colon, or rectum), bone, bone marrow, skin (e.g., melanoma), head and neck, liver, gall bladder, bile ducts, heart, lung, pancreas, salivary gland, adrenal gland, thyroid, brain (e.g., gliomas), ganglia, central nervous system (CNS), peripheral nervous system (PNS), the hematopoietic system (i.e., hematological malignancies), or the immune system (e.g., spleen or thymus), or any combination thereof;
b) breast cancer, genitourinary cancer, gastrointestinal cancer, lung cancer, skin cancer, or a combination thereof; or
c) a hematological malignancy, optionally selected from leukemias, lymphomas and myelomas.

42.-43. (canceled)

44. The method of claim 40, further comprising administering at least one additional therapeutic agent to the subject.

45. The method of claim 44, wherein said at least one additional therapeutic agent comprises one or more agents independently selected from the groups consisting of immune checkpoint inhibitors, agents that target the extracellular production of adenosine, inhibitors of HIF (e.g., a HIF-2α inhibitor), kinase inhibitors, radiation therapy, and chemotherapeutic agents.

46.-79. (canceled)
